(19) United States
(12) Patent Application Publication
(10) Pub. No.: US 2012/0004118 A1
(43) Pub. Date: Jan. 5, 2012

(54) METHODS FOR THE SUBCLASSIFICATION OF BREAST TUMOURS

(75) Inventors: Sitharthan Kamalakaran, Pelham, NY (US); Angel Janevski, New York, NY (US); James Bruce Hicks, Cold Spring Harbor, NY (US)

(73) Assignees: COLD SPRING HARBOR LABORATORIES, COLD SPRING HARBOR, NY (US); KONINKLIJKE PHILIPS ELECTRONICS N.V., EINDHOVEN (NL)

(21) Appl. No.: 13/147,105

(22) PCT Filed: Jan. 25, 2010
(86) PCT No.: PCT/IB10/50316

§ 371 Date: Sep. 20, 2011

Related U.S. Application Data
(60) Provisional application No. 61/148,413, filed on Jan. 30, 2009.

(51) Int. Cl. C40B 30/00; C12Q 1/68; B01J 19/00; C40B 60/12; C12M 1/34; G01N 33/50; H01J 49/26

(52) U.S. Cl. 506/7; 436/94; 435/6.12; 250/282; 506/39; 435/287.2; 422/68.1

(57) ABSTRACT

Provided is a method for the analysis of breast cancer disorders, comprising determining the genomic methylation status of one or more CpG dinucleotides. Furthermore, a computer program product stored on a computer-readable medium comprising software code adapted to perform the steps of the method when executed on a data-processing apparatus is provided. A device comprising means for supporting a clinician is also provided.

### FIELD OF THE INVENTION

This invention pertains in general to the field of biology and bioinformatics. More particularly the invention relates to the field of categorization of cancer tumours and even more particularly to identifying methylated sites, which may aid in categorization of cancer tumours.

### BACKGROUND OF THE INVENTION

Worldwide, breast cancer is the fifth most common cause of cancer death, after lung cancer, stomach cancer, liver cancer, and colon cancer. Among women, breast cancer is the most common cancer and the most common cause of cancer death.

Breast cancer is diagnosed by the pathological examination of surgically removed breast tissue. Following diagnosis, it is important to analyze the tumour type in order to aid clinicians when choosing the right therapy. Within the art, such analysis is performed according to two categories.

The first category involves the use of immuno-histopathological variables, such as tumour size, ER/PR status, lymph node negativity, etc. to define a clinical prognostic index such as the Nottingham Prognostic Index (NPI). The problem with such an index is that it has been shown to be very conservative, thus typically causing patients to receive aggressive therapy even when they are a low risk of disease recurrence.

The second category involves the measurement of the expression levels of a large number of genes, typically around 500, and calculating probability of a subtype based on the relative expression levels of the genes. This method is very costly in terms of tissue handling requirements. It is also hard to perform in a clinical setting, due to the demand of laboratory equipment.

DNA methylation, a type of chemical modification of DNA that can be inherited and subsequently removed without changing the original DNA sequence, is the most well studied epigenetic mechanism of gene regulation. There are areas in DNA where a cytosine nucleotide occurs next to a guanine nucleotide in the linear sequence of bases called CpG islands.

CpG islands are generally heavily methylated in normal cells. However, during tumorigenesis, hypomethylation occurs at these islands, which may result in the expression of certain repeats. These hypomethylation events also correlate to the severity of some cancers. Under certain circumstances, which may occur in pathologies such as cancer, imprinting, development, tissue specificity, or X chromosome inactivation, gene associated islands may be heavily methylated. Specifically, in cancer, methylation of islands proximal to tumour suppressors is a frequent event, often occurring when the second allele is lost by deletion (Loss of Heterozygosity, LOH). Some tumour suppressors commonly seen with methylated islands are p16, Rassf1a, and BRCA1.

There are reported epigenetic markers for colorectal and prostate cancer. For example, Epigenomics AG (Berlin, Germany) has the Septin 9 as a marker for colorectal cancer screening in blood plasma. A method for using methylation sites to predict differential therapy responses in cancer and recommending an appropriate therapy has been disclosed in US20050021240A1. However, the results predicted by this method are limited, since they cannot be directly applied in clinical practice. Therefore, it would advantageous to have a method for the analysis of breast cancer disorders, which is time efficient, reliable and cost-effective.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a method for the analysis of breast cancer disorders according to the appended patent claims.

According to an aspect a method for analysis of breast cancer disorders is disclosed. The method comprises determining the genomic methylation status of one or more CpG dinucleotides in a sequence selected from the group of sequences consisting of SEQ ID NO. 1 to SEQ ID NO. 600. The method provides for improved abilities to characterize cancer tumours using methylation patterns.

The regions of interest of the sequences SEQ ID NO. 1 to 600 are designated in table 1 (as “start” and “end” on respective “chromosome”).

This aspect presents improvements over the state of the art in that it enables a highly specific classification of breast cell proliferative disorders.

In an aspect a computer program product is disclosed. The computer program product is stored on a computer-readable medium comprising software code adapted to perform the steps of the method according to an aspect when executed on a data-processing apparatus.

In an aspect a device is disclosed. The device comprises means adapted to carry out methods according to som embodiments. An advantage with this is to support a clinician.

Herein, the sequences claimed also encompass the sequences, which are reverse complement to the sequences designated.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a method according to some embodiments;

FIG. 2 is a schematic illustration of a dataset 20 of five measurements 1 to 5;

FIG. 3 is a schematic illustration of a first subset 30 of five measurements 1 to 5;

FIG. 4 is a schematic illustration of a second subset 40 of five measurements 1 to 5; and

FIG. 5 is an illustration of clusters 51, 52, 53, where FIG. 5A is a first cluster 51, FIG. 5B is a second cluster 52 and FIG. 5C is a third cluster 53.

FIG. 6 is a schematic illustration of a computer program product according to an embodiment.

FIG. 7 is a schematic illustration of a device according to an embodiment.

### DETAILED DESCRIPTION OF EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

An idea according to some embodiments is a method using a small selection of DNA sequences to analyze breast cancer disorders. The analysis is done by determining genomic methylation status of one or more CpG dinucleotides, in either sequence disclosed herein, or its reverse complement.

It was surprisingly found that some DNA sequences, SEQ ID NO: 1 to SEQ ID NO: 600 act as epigenetic markers that may be used to analyze breast cancer by subtyping tumours. In prior art, it is possible to subtype breast cancer based on gene expression. Five different subtypes have been reported; luminal A, luminal B, basal, ERBB2 overexpressing, and normal-like. The inventors have identified the same subtypes using DNA methylation.

The DNA SEQ ID NO: 1 to SEQ ID NO: 600 were identified by analysing 150 000 individual genomic loci for methylation, across a set of 83 breast tumours. The availability of clinical information regarding tumour specimens allowed for an investigation of DNA methylation in the context of breast cancer subtypes, histology and tumour aggressiveness. The five major breast cancer molecular subtypes (luminal A and B, basal, ERBB2 overexpressing, and normal-like) were identified. First, an investigation was performed regarding however unsupervised clustering of the tumour set using methylation recapitulates the major Luminal and basal classes that were identified by expression analysis or not. A filtering criterion was used to identify the features to be used in clustering. This criterion was the top 500 loci that varied most across the 83 tumour samples. Then, the top 100 loci that distinguished tumours from normal tissues from were added. These 600 features, displayed in table 1, were used to cluster the 83 tumours for which the expression subtype data was available. Hierarchical clustering with Pearson correlation and complete linkage of the samples based on these six hundred loci gave a dendrogram that is surprisingly similar to the one produced by expression analysis.

TABLE 1
600 features for categorization of cancer

| SEQ | | | | |
|---|---|---|---|---|
| ID NO: | Frag ID | Chromosome | Start | End |
| 1 | MspFrag4633 | 1 | 32374307 | 32374791 |
| 2 | MspFrag757 | 1 | 1702806 | 1703222 |
| 3 | MspFrag1173 | 1 | 2518915 | 2519285 |
| 4 | MspFrag1211 | 1 | 2622522 | 2623091 |
| 5 | MspFrag1212 | 1 | 2629273 | 2629613 |
| 6 | MspFrag1241 | 1 | 2871558 | 2871896 |
| 7 | MspFrag1242 | 1 | 2873712 | 2874055 |
| 8 | MspFrag1249 | 1 | 2944491 | 2945100 |
| 9 | MspFrag1311 | 1 | 3036436 | 3036818 |
| 10 | MspFrag1321 | 1 | 3103884 | 3104234 |
| 11 | MspFrag1324 | 1 | 3113132 | 3113448 |
| 12 | MspFrag1326 | 1 | 3118212 | 3118636 |
| 13 | MspFrag1339 | 1 | 3163795 | 3164122 |
| 14 | MspFrag1340 | 1 | 3165605 | 3166112 |
| 15 | MspFrag1359 | 1 | 3218362 | 3218653 |
| 16 | MspFrag1377 | 1 | 3296147 | 3296524 |
| 17 | MspFrag1391 | 1 | 3338689 | 3339191 |
| 18 | MspFrag1534 | 1 | 3642624 | 3643184 |
| 19 | MspFrag1601 | 1 | 4360224 | 4360668 |
| 20 | MspFrag1649 | 1 | 5478055 | 5478432 |
| 21 | MspFrag1650 | 1 | 5490384 | 5490940 |
| 22 | MspFrag1775 | 1 | 6285179 | 6285570 |
| 23 | MspFrag1823 | 1 | 6445812 | 6446063 |
| 24 | MspFrag1961 | 1 | 6949999 | 6950306 |
| 25 | MspFrag2123 | 1 | 9031495 | 9031958 |
| 26 | MspFrag2643 | 1 | 14669841 | 14670071 |
| 27 | MspFrag2886 | 1 | 16695727 | 16696176 |
| 28 | MspFrag3066 | 1 | 18043936 | 18044316 |
| 29 | MspFrag3084 | 1 | 18205071 | 18205589 |
| 30 | MspFrag3535 | 1 | 22625307 | 22625790 |
| 31 | MspFrag4109 | 1 | 27008738 | 27009387 |
| 32 | MspFrag4389 | 1 | 29281582 | 29281828 |
| 33 | MspFrag4819 | 1 | 33768108 | 33768404 |
| 34 | MspFrag4820 | 1 | 33769727 | 33770434 |
| 35 | MspFrag4823 | 1 | 33955400 | 33955873 |
| 36 | MspFrag5071 | 1 | 36908888 | 36909106 |
| 37 | MspFrag5104 | 1 | 37589882 | 37590168 |
| 38 | MspFrag5190 | 1 | 37995046 | 37995631 |
| 39 | MspFrag5455 | 1 | 40267780 | 40268103 |
| 40 | MspFrag5525 | 1 | 40916307 | 40917083 |
| 41 | MspFrag5644 | 1 | 41941498 | 41941965 |
| 42 | MspFrag5980 | 1 | 44977457 | 44977763 |
| 43 | MspFrag6197 | 1 | 47408542 | 47408713 |
| 44 | MspFrag6914 | 1 | 62496120 | 62496646 |
| 45 | MspFrag7116 | 1 | 65646887 | 65647674 |
| 46 | MspFrag7153 | 1 | 67312523 | 67312727 |
| 47 | MspFrag7228 | 1 | 71223914 | 71224499 |
| 48 | MspFrag7359 | 1 | 79184005 | 79184422 |
| 49 | MspFrag8101 | 1 | 101535648 | 101535994 |
| 50 | MspFrag8168 | 1 | 108527701 | 108527992 |
| 51 | MspFrag8169 | 1 | 108675712 | 108676003 |
| 52 | MspFrag8273 | 1 | 109749595 | 109750084 |
| 53 | MspFrag8710 | 1 | 115926101 | 115926763 |
| 54 | MspFrag8778 | 1 | 116868496 | 116868706 |
| 55 | MspFrag8956 | 1 | 120551325 | 120551421 |
| 56 | MspFrag9029 | 1 | 142697968 | 142698037 |
| 57 | MspFrag9245 | 1 | 145643787 | 145644444 |
| 58 | MspFrag9273 | 1 | 146010092 | 146010549 |
| 59 | MspFrag9278 | 1 | 146064945 | 146066503 |
| 60 | MspFrag9601 | 1 | 148893238 | 148893494 |
| 61 | MspFrag9703 | 1 | 150968906 | 150969531 |
| 62 | MspFrag9928 | 1 | 152077757 | 152078037 |
| 63 | MspFrag9937 | 1 | 152103832 | 152104033 |
| 64 | MspFrag10189 | 1 | 153690285 | 153690897 |
| 65 | MspFrag10393 | 1 | 158225523 | 158225819 |
| 66 | MspFrag10421 | 1 | 158232050 | 158232295 |
| 67 | MspFrag10427 | 1 | 158232923 | 158233174 |
| 68 | MspFrag10490 | 1 | 158246841 | 158247086 |
| 69 | MspFrag10496 | 1 | 158247714 | 158247965 |
| 70 | MspFrag10537 | 1 | 158307786 | 158308067 |
| 71 | MspFrag10623 | 1 | 162330700 | 162331269 |
| 72 | MspFrag10916 | 1 | 172907883 | 172908042 |
| 73 | MspFrag11354 | 1 | 194611559 | 194611928 |
| 74 | MspFrag11474 | 1 | 197984459 | 197984775 |
| 75 | MspFrag11782 | 1 | 202229373 | 202229833 |
| 76 | MspFrag12301 | 1 | 217252591 | 217253153 |
| 77 | MspFrag13394 | 1 | 227605182 | 227605359 |
| 78 | MspFrag13583 | 1 | 232131677 | 232132379 |
| 79 | MspFrag14197 | 2 | 1248326 | 1248943 |
| 80 | MspFrag14202 | 2 | 1293040 | 1293404 |
| 81 | MspFrag14203 | 2 | 1296483 | 1297255 |
| 82 | MspFrag14231 | 2 | 1703105 | 1703374 |
| 83 | MspFrag14254 | 2 | 1833149 | 1833914 |
| 84 | MspFrag14278 | 2 | 2676636 | 2677246 |
| 85 | MspFrag14289 | 2 | 2812784 | 2813304 |
| 86 | MspFrag14290 | 2 | 2825618 | 2826147 |
| 87 | MspFrag14334 | 2 | 3326870 | 3327299 |
| 88 | MspFrag14451 | 2 | 5957756 | 5957971 |
| 89 | MspFrag14457 | 2 | 6749495 | 6749988 |
| 90 | MspFrag14487 | 2 | 7440522 | 7441007 |
| 91 | MspFrag14609 | 2 | 9553132 | 9553410 |
| 92 | MspFrag14656 | 2 | 10133476 | 10133666 |
| 93 | MspFrag14921 | 2 | 15857512 | 15857896 |
| 94 | MspFrag15066 | 2 | 20312835 | 20313215 |
| 95 | MspFrag15478 | 2 | 26785546 | 26785870 |
| 96 | MspFrag15644 | 2 | 27515565 | 27515896 |
| 97 | MspFrag15771 | 2 | 29699956 | 29700602 |
| 98 | MspFrag17091 | 2 | 65021553 | 65022078 |
| 99 | MspFrag17159 | 2 | 66264144 | 66264933 |
| 100 | MspFrag17697 | 2 | 73589558 | 73590193 |
| 101 | MspFrag17841 | 2 | 74642481 | 74642761 |
| 102 | MspFrag18355 | 2 | 91199543 | 91199793 |
| 103 | MspFrag18856 | 2 | 100492801 | 100493089 |
| 104 | MspFrag19245 | 2 | 108982952 | 108983175 |
| 105 | MspFrag19926 | 2 | 121038231 | 121038980 |
| 106 | MspFrag19965 | 2 | 121259357 | 121259763 |
| 107 | MspFrag20024 | 2 | 122816085 | 122816353 |
| 108 | MspFrag20134 | 2 | 128138182 | 128138536 |
| 109 | MspFrag20225 | 2 | 128792924 | 128793466 |
| 110 | MspFrag20706 | 2 | 139372061 | 139372477 |
| 111 | MspFrag20895 | 2 | 155380949 | 155381434 |
| 112 | MspFrag21537 | 2 | 175420626 | 175420995 |
| 113 | MspFrag21600 | 2 | 176773874 | 176774399 |
| 114 | MspFrag22036 | 2 | 191710645 | 191710851 |
| 115 | MspFrag22213 | 2 | 200159441 | 200159639 |
| 116 | MspFrag22546 | 2 | 209899069 | 209899548 |
| 117 | MspFrag22928 | 2 | 220021958 | 220022344 |
| 118 | MspFrag23536 | 2 | 233077827 | 233078119 |
| 119 | MspFrag23738 | 2 | 236183911 | 236184343 |
| 120 | MspFrag24273 | 2 | 241696154 | 241696568 |
| 121 | MspFrag25023 | 3 | 13136633 | 13137251 |
| 122 | MspFrag25164 | 3 | 14826516 | 14826916 |
| 123 | MspFrag25187 | 3 | 15081919 | 15082508 |
| 124 | MspFrag25517 | 3 | 28529966 | 28530450 |
| 125 | MspFrag25715 | 3 | 35760405 | 35760961 |
| 126 | MspFrag26073 | 3 | 42996257 | 42996879 |
| 127 | MspFrag26133 | 3 | 44016018 | 44016419 |
| 128 | MspFrag26295 | 3 | 46828327 | 46828820 |
| 129 | MspFrag26333 | 3 | 46909242 | 46909602 |
| 130 | MspFrag26774 | 3 | 50133302 | 50133713 |
| 131 | MspFrag27115 | 3 | 52543768 | 52544136 |
| 132 | MspFrag27268 | 3 | 55492383 | 55492977 |
| 133 | MspFrag27379 | 3 | 58042487 | 58042945 |
| 134 | MspFrag27495 | 3 | 62333914 | 62333971 |
| 135 | MspFrag27677 | 3 | 69184229 | 69184352 |
| 136 | MspFrag27685 | 3 | 69517625 | 69517852 |
| 137 | MspFrag28326 | 3 | 114643147 | 114643394 |
| 138 | MspFrag28887 | 3 | 128424361 | 128424622 |
| 139 | MspFrag29324 | 3 | 135097550 | 135098100 |
| 140 | MspFrag30803 | 3 | 185784594 | 185784860 |
| 141 | MspFrag31913 | 4 | 1192879 | 1193371 |
| 142 | MspFrag32174 | 4 | 1719620 | 1719949 |
| 143 | MspFrag32611 | 4 | 3571688 | 3573129 |
| 144 | MspFrag32624 | 4 | 3776452 | 3776818 |
| 145 | MspFrag32667 | 4 | 3914642 | 3915363 |
| 146 | MspFrag32966 | 4 | 7107197 | 7107478 |
| 147 | MspFrag33006 | 4 | 7629573 | 7630026 |
| 148 | MspFrag33110 | 4 | 9006410 | 9006713 |
| 149 | MspFrag33134 | 4 | 9459349 | 9459626 |
| 150 | MspFrag33136 | 4 | 9459777 | 9459956 |
| 151 | MspFrag33338 | 4 | 15333834 | 15334201 |
| 152 | MspFrag33381 | 4 | 16273567 | 16273855 |
| 153 | MspFrag35700 | 4 | 111901776 | 111901955 |
| 154 | MspFrag36595 | 4 | 152604344 | 152604681 |
| 155 | MspFrag36661 | 4 | 154574444 | 154574685 |
| 156 | MspFrag36683 | 4 | 154962375 | 154962925 |
| 157 | MspFrag37395 | 4 | 187400622 | 187401021 |
| 158 | MspFrag38281 | 5 | 1011369 | 1011836 |
| 159 | MspFrag38417 | 5 | 1302864 | 1303240 |
| 160 | MspFrag38457 | 5 | 1348431 | 1348617 |
| 161 | MspFrag38485 | 5 | 1440104 | 1440605 |
| 162 | MspFrag38491 | 5 | 1496943 | 1497332 |
| 163 | MspFrag38714 | 5 | 2166920 | 2167677 |
| 164 | MspFrag38815 | 5 | 2919629 | 2920003 |
| 165 | MspFrag38821 | 5 | 3156410 | 3156769 |
| 166 | MspFrag38910 | 5 | 3907742 | 3907967 |
| 167 | MspFrag39470 | 5 | 31716178 | 31716614 |
| 168 | MspFrag39539 | 5 | 33927617 | 33927999 |
| 169 | MspFrag39543 | 5 | 33972064 | 33972687 |
| 170 | MspFrag39760 | 5 | 40871578 | 40871991 |
| 171 | MspFrag40505 | 5 | 71888649 | 71889360 |
| 172 | MspFrag40858 | 5 | 77304521 | 77304932 |
| 173 | MspFrag42441 | 5 | 134394818 | 134395156 |
| 174 | MspFrag42953 | 5 | 140187999 | 140188260 |
| 175 | MspFrag42983 | 5 | 140216007 | 140216482 |
| 176 | MspFrag44192 | 5 | 174111126 | 174111339 |
| 177 | MspFrag44328 | 5 | 175956200 | 175956454 |
| 178 | MspFrag44767 | 5 | 178348383 | 178348602 |
| 179 | MspFrag45007 | 5 | 179673647 | 179673858 |
| 180 | MspFrag45338 | 6 | 1311232 | 1311666 |
| 181 | MspFrag45409 | 6 | 1530339 | 1531041 |
| 182 | MspFrag45501 | 6 | 1625429 | 1625752 |
| 183 | MspFrag45650 | 6 | 3401937 | 3401968 |
| 184 | MspFrag46110 | 6 | 11152853 | 11153148 |
| 185 | MspFrag46277 | 6 | 16237147 | 16237395 |
| 186 | MspFrag46721 | 6 | 27449907 | 27450504 |
| 187 | MspFrag47196 | 6 | 31804402 | 31804867 |
| 188 | MspFrag47435 | 6 | 33353475 | 33353858 |
| 189 | MspFrag47510 | 6 | 33708897 | 33709149 |
| 190 | MspFrag48491 | 6 | 44373563 | 44374341 |
| 191 | MspFrag49687 | 6 | 101001787 | 101002201 |
| 192 | MspFrag50444 | 6 | 123359218 | 123359439 |
| 193 | MspFrag50717 | 6 | 134539380 | 134539767 |
| 194 | MspFrag50853 | 6 | 137860054 | 137860272 |
| 195 | MspFrag52027 | 6 | 168452341 | 168452651 |
| 196 | MspFrag52146 | 6 | 169670215 | 169670603 |
| 197 | MspFrag52434 | 7 | 580841 | 581190 |
| 198 | MspFrag52666 | 7 | 989299 | 989808 |
| 199 | MspFrag52792 | 7 | 1206082 | 1206625 |
| 200 | MspFrag52897 | 7 | 1460124 | 1460484 |
| 201 | MspFrag53338 | 7 | 4884663 | 4885032 |
| 202 | MspFrag54143 | 7 | 21829594 | 21830366 |
| 203 | MspFrag54400 | 7 | 26916475 | 26916913 |
| 204 | MspFrag54424 | 7 | 26935561 | 26936019 |
| 205 | MspFrag54796 | 7 | 30494831 | 30495180 |
| 206 | MspFrag54824 | 7 | 31149657 | 31149980 |
| 207 | MspFrag54975 | 7 | 35070796 | 35071213 |
| 208 | MspFrag55218 | 7 | 43062129 | 43062415 |
| 209 | MspFrag55275 | 7 | 43877824 | 43878339 |
| 210 | MspFrag55475 | 7 | 47902671 | 47903123 |
| 211 | MspFrag55611 | 7 | 54506521 | 54507157 |
| 212 | MspFrag55649 | 7 | 54862496 | 54862960 |
| 213 | MspFrag55941 | 7 | 63786704 | 63787372 |
| 214 | MspFrag56289 | 7 | 72093180 | 72093418 |
| 215 | MspFrag56402 | 7 | 72563341 | 72563657 |
| 216 | MspFrag56504 | 7 | 73646860 | 73647098 |
| 217 | MspFrag56540 | 7 | 74018306 | 74018544 |
| 218 | MspFrag56922 | 7 | 87208109 | 87208310 |
| 219 | MspFrag57002 | 7 | 90540824 | 90541294 |
| 220 | MspFrag57206 | 7 | 97246402 | 97246843 |
| 221 | MspFrag57442 | 7 | 99419846 | 99420214 |
| 222 | MspFrag57677 | 7 | 100240230 | 100240525 |
| 223 | MspFrag58680 | 7 | 128125215 | 128125598 |
| 224 | MspFrag59067 | 7 | 136989204 | 136989443 |
| 225 | MspFrag60291 | 7 | 155610859 | 155611142 |
| 226 | MspFrag60445 | 7 | 156703792 | 156704149 |
| 227 | MspFrag60779 | 7 | 158289060 | 158289297 |
| 228 | MspFrag60966 | 8 | 1008907 | 1009401 |
| 229 | MspFrag61003 | 8 | 1239397 | 1239831 |
| 230 | MspFrag61051 | 8 | 1470634 | 1471413 |
| 231 | MspFrag61099 | 8 | 1759273 | 1759325 |
| 232 | MspFrag61152 | 8 | 1982797 | 1983256 |
| 233 | MspFrag61161 | 8 | 2062616 | 2063197 |
| 234 | MspFrag61169 | 8 | 2197099 | 2197693 |
| 235 | MspFrag61173 | 8 | 2324899 | 2325526 |
| 236 | MspFrag61350 | 8 | 7917174 | 7917432 |
| 237 | MspFrag62044 | 8 | 22045386 | 22045723 |
| 238 | MspFrag62294 | 8 | 24826373 | 24826927 |
| 239 | MspFrag62605 | 8 | 29266511 | 29267015 |
| 240 | MspFrag63030 | 8 | 41702523 | 41702937 |
| 241 | MspFrag63043 | 8 | 41774590 | 41774866 |
| 242 | MspFrag63267 | 8 | 49697557 | 49697886 |
| 243 | MspFrag63271 | 8 | 49810071 | 49810539 |
| 244 | MspFrag63597 | 8 | 59220858 | 59221324 |
| 245 | MspFrag64684 | 8 | 97242768 | 97243023 |
| 246 | MspFrag64725 | 8 | 98359395 | 98359772 |
| 247 | MspFrag65670 | 8 | 135559922 | 135560190 |
| 248 | MspFrag65671 | 8 | 135560191 | 135560433 |
| 249 | MspFrag66071 | 8 | 144225273 | 144225476 |
| 250 | MspFrag66146 | 8 | 144444026 | 144444368 |
| 251 | MspFrag67369 | 9 | 988973 | 989201 |
| 252 | MspFrag67459 | 9 | 2613599 | 2614303 |
| 253 | MspFrag68271 | 9 | 34362590 | 34362891 |
| 254 | MspFrag68663 | 9 | 37743792 | 37744031 |
| 255 | MspFrag68970 | 9 | 64167952 | 64168281 |
| 256 | MspFrag69380 | 9 | 76862972 | 76863247 |
| 257 | MspFrag69976 | 9 | 93159730 | 93160221 |
| 258 | MspFrag70538 | 9 | 98551494 | 98551667 |
| 259 | MspFrag71074 | 9 | 112913792 | 112914149 |
| 260 | MspFrag71089 | 9 | 112919236 | 112919593 |
| 261 | MspFrag71090 | 9 | 112920067 | 112920611 |
| 262 | MspFrag71104 | 9 | 112924678 | 112925035 |
| 263 | MspFrag71105 | 9 | 112925509 | 112926053 |
| 264 | MspFrag71120 | 9 | 112930124 | 112930481 |
| 265 | MspFrag71121 | 9 | 112930955 | 112931497 |
| 266 | MspFrag71216 | 9 | 114346043 | 114346380 |
| 267 | MspFrag71581 | 9 | 124112526 | 124112954 |
| 268 | MspFrag71700 | 9 | 125589095 | 125589132 |
| 269 | MspFrag72003 | 9 | 127768596 | 127769001 |
| 270 | MspFrag72461 | 9 | 130337856 | 130338298 |
| 271 | MspFrag72674 | 9 | 131728566 | 131728859 |
| 272 | MspFrag72675 | 9 | 131728907 | 131729282 |
| 273 | MspFrag72740 | 9 | 132391939 | 132392575 |
| 274 | MspFrag72750 | 9 | 132485893 | 132486113 |
| 275 | MspFrag73062 | 9 | 134431953 | 134432427 |
| 276 | MspFrag73586 | 9 | 136866193 | 136866519 |
| 277 | MspFrag73907 | 9 | 137307963 | 137309295 |
| 278 | MspFrag74424 | 10 | 521032 | 521557 |
| 279 | MspFrag74598 | 10 | 1740057 | 1740811 |
| 280 | MspFrag75026 | 10 | 11420347 | 11420872 |
| 281 | MspFrag76120 | 10 | 35968545 | 35968856 |
| 282 | MspFrag76422 | 10 | 43464543 | 43465148 |
| 283 | MspFrag76467 | 10 | 44201213 | 44201571 |
| 284 | MspFrag76619 | 10 | 47227978 | 47228669 |
| 285 | MspFrag76797 | 10 | 50489052 | 50489405 |
| 286 | MspFrag76801 | 10 | 50489790 | 50491027 |
| 287 | MspFrag77115 | 10 | 64248087 | 64248491 |
| 288 | MspFrag77199 | 10 | 69760469 | 69761198 |
| 289 | MspFrag77777 | 10 | 76836478 | 76837103 |
| 290 | MspFrag78440 | 10 | 94811337 | 94811966 |
| 291 | MspFrag79123 | 10 | 102798099 | 102798651 |
| 292 | MspFrag79169 | 10 | 102883661 | 102883938 |
| 293 | MspFrag79207 | 10 | 102972749 | 102973047 |
| 294 | MspFrag79636 | 10 | 107141635 | 107141970 |
| 295 | MspFrag80112 | 10 | 119291788 | 119292000 |
| 296 | MspFrag80168 | 10 | 120344860 | 120345112 |
| 297 | MspFrag80169 | 10 | 120345113 | 120345331 |
| 298 | MspFrag80343 | 10 | 123771228 | 123771724 |
| 299 | MspFrag80645 | 10 | 126830955 | 126831650 |
| 300 | MspFrag80726 | 10 | 128183447 | 128184143 |
| 301 | MspFrag80728 | 10 | 128234723 | 128235166 |
| 302 | MspFrag80854 | 10 | 131646461 | 131646892 |
| 303 | MspFrag80954 | 10 | 131878295 | 131878616 |
| 304 | MspFrag80975 | 10 | 132947917 | 132948395 |
| 305 | MspFrag80989 | 10 | 133000558 | 133000818 |
| 306 | MspFrag82654 | 11 | 2002464 | 2002798 |
| 307 | MspFrag82859 | 11 | 2864180 | 2864505 |
| 308 | MspFrag82920 | 11 | 3199023 | 3199589 |
| 309 | MspFrag83839 | 11 | 19323892 | 19324489 |
| 310 | MspFrag84490 | 11 | 43921200 | 43921449 |
| 311 | MspFrag84518 | 11 | 44286856 | 44287176 |
| 312 | MspFrag85089 | 11 | 58487399 | 58488005 |
| 313 | MspFrag85656 | 11 | 63640294 | 63640522 |
| 314 | MspFrag85976 | 11 | 64496008 | 64496486 |
| 315 | MspFrag86495 | 11 | 65945827 | 65946236 |
| 316 | MspFrag86866 | 11 | 67527006 | 67527364 |
| 317 | MspFrag86939 | 11 | 67937373 | 67937857 |
| 318 | MspFrag87160 | 11 | 69602771 | 69603307 |
| 319 | MspFrag87185 | 11 | 69863028 | 69863693 |
| 320 | MspFrag87210 | 11 | 70329201 | 70329876 |
| 321 | MspFrag87698 | 11 | 76059797 | 76059981 |
| 322 | MspFrag88140 | 11 | 93774380 | 93774585 |
| 323 | MspFrag88235 | 11 | 95551592 | 95552011 |
| 324 | MspFrag88395 | 11 | 106833824 | 106834052 |
| 325 | MspFrag88411 | 11 | 107304811 | 107304985 |
| 326 | MspFrag88517 | 11 | 110916170 | 110916785 |
| 327 | MspFrag88655 | 11 | 113989177 | 113989682 |
| 328 | MspFrag88982 | 11 | 118710713 | 118711261 |
| 329 | MspFrag89183 | 11 | 122571813 | 122572088 |
| 330 | MspFrag89408 | 11 | 126267744 | 126268359 |
| 331 | MspFrag89444 | 11 | 128007477 | 128008054 |
| 332 | MspFrag89848 | 12 | 432342 | 432620 |
| 333 | MspFrag89865 | 12 | 440326 | 440703 |
| 334 | MspFrag90004 | 12 | 1887654 | 1887972 |
| 335 | MspFrag90137 | 12 | 3472552 | 3472916 |
| 336 | MspFrag90140 | 12 | 3473198 | 3473610 |
| 337 | MspFrag90376 | 12 | 6626277 | 6626591 |
| 338 | MspFrag91076 | 12 | 28018747 | 28019241 |
| 339 | MspFrag92237 | 12 | 50913530 | 50913916 |
| 340 | MspFrag92520 | 12 | 52761839 | 52762613 |
| 341 | MspFrag92533 | 12 | 52831831 | 52832592 |
| 342 | MspFrag92849 | 12 | 56290306 | 56290717 |
| 343 | MspFrag93471 | 12 | 76221553 | 76221851 |
| 344 | MspFrag93929 | 12 | 100105780 | 100106149 |
| 345 | MspFrag94051 | 12 | 103034912 | 103035336 |
| 346 | MspFrag94345 | 12 | 108603802 | 108604232 |
| 347 | MspFrag94367 | 12 | 108636999 | 108637342 |
| 348 | MspFrag95107 | 12 | 119463497 | 119464156 |
| 349 | MspFrag95724 | 12 | 126397709 | 126398319 |
| 350 | MspFrag95754 | 12 | 127714235 | 127714816 |
| 351 | MspFrag95908 | 12 | 130037881 | 130038220 |
| 352 | MspFrag96210 | 12 | 131593486 | 131593921 |
| 353 | MspFrag96227 | 12 | 131632939 | 131633353 |
| 354 | MspFrag96587 | 13 | 19666287 | 19666805 |
| 355 | MspFrag97775 | 13 | 43876711 | 43877202 |
| 356 | MspFrag98223 | 13 | 52674273 | 52674824 |
| 357 | MspFrag98264 | 13 | 57102098 | 57102284 |
| 358 | MspFrag98985 | 13 | 99421760 | 99422234 |
| 359 | MspFrag99113 | 13 | 102224202 | 102224673 |
| 360 | MspFrag99150 | 13 | 104803836 | 104804393 |
| 361 | MspFrag99310 | 13 | 109676095 | 109676754 |
| 362 | MspFrag99457 | 13 | 111003520 | 111003741 |
| 363 | MspFrag99472 | 13 | 111623681 | 111623969 |
| 364 | MspFrag99554 | 13 | 111836670 | 111837162 |
| 365 | MspFrag99668 | 13 | 112696646 | 112696951 |
| 366 | MspFrag100018 | 13 | 113964379 | 113964675 |
| 367 | MspFrag100061 | 14 | 18719759 | 18720152 |
| 368 | MspFrag101138 | 14 | 44792484 | 44793174 |
| 369 | MspFrag102005 | 14 | 64078276 | 64078714 |
| 370 | MspFrag102061 | 14 | 64638719 | 64638995 |
| 371 | MspFrag103295 | 14 | 92767021 | 92767589 |
| 372 | MspFrag103518 | 14 | 97286503 | 97287063 |
| 373 | MspFrag103793 | 14 | 100262666 | 100262888 |
| 374 | MspFrag104383 | 14 | 103840309 | 103840685 |
| 375 | MspFrag104955 | 15 | 19487742 | 19488254 |
| 376 | MspFrag105085 | 15 | 22223532 | 22223950 |
| 377 | MspFrag105101 | 15 | 22751446 | 22752129 |
| 378 | MspFrag105266 | 15 | 26323073 | 26323406 |
| 379 | MspFrag105873 | 15 | 38437638 | 38437690 |
| 380 | MspFrag105880 | 15 | 38446968 | 38447392 |
| 381 | MspFrag107570 | 15 | 66794080 | 66794622 |
| 382 | MspFrag108016 | 15 | 72805958 | 72806255 |
| 383 | MspFrag108348 | 15 | 76073603 | 76074094 |
| 384 | MspFrag110494 | 16 | 807095 | 807318 |
| 385 | MspFrag110545 | 16 | 954593 | 954879 |
| 386 | MspFrag110579 | 16 | 972953 | 973346 |
| 387 | MspFrag110668 | 16 | 1094736 | 1095111 |
| 388 | MspFrag110793 | 16 | 1333585 | 1333929 |
| 389 | MspFrag110848 | 16 | 1408921 | 1409435 |
| 390 | MspFrag111358 | 16 | 2226616 | 2226830 |
| 391 | MspFrag111585 | 16 | 2756264 | 2756492 |
| 392 | MspFrag111802 | 16 | 3149326 | 3150003 |
| 393 | MspFrag112325 | 16 | 10387218 | 10387406 |
| 394 | MspFrag113247 | 16 | 27656752 | 27657519 |
| 395 | MspFrag113614 | 16 | 30112985 | 30113118 |
| 396 | MspFrag113989 | 16 | 31133694 | 31134196 |
| 397 | MspFrag114087 | 16 | 32003855 | 32004417 |
| 398 | MspFrag114107 | 16 | 32172277 | 32172824 |
| 399 | MspFrag114108 | 16 | 32172825 | 32173259 |
| 400 | MspFrag114138 | 16 | 32593842 | 32594268 |
| 401 | MspFrag114139 | 16 | 32594269 | 32594593 |
| 402 | MspFrag114140 | 16 | 32594594 | 32594816 |
| 403 | MspFrag114205 | 16 | 33113217 | 33113439 |
| 404 | MspFrag114206 | 16 | 33113440 | 33113764 |
| 405 | MspFrag114207 | 16 | 33113765 | 33114191 |
| 406 | MspFrag114218 | 16 | 33169752 | 33169974 |
| 407 | MspFrag114219 | 16 | 33169975 | 33170299 |
| 408 | MspFrag114220 | 16 | 33170300 | 33170726 |
| 409 | MspFrag114804 | 16 | 52881971 | 52882449 |
| 410 | MspFrag115251 | 16 | 65017842 | 65018293 |
| 411 | MspFrag115442 | 16 | 65776185 | 65776573 |
| 412 | MspFrag115870 | 16 | 67977524 | 67977617 |
| 413 | MspFrag116223 | 16 | 74023655 | 74024439 |
| 414 | MspFrag116804 | 16 | 85098845 | 85099404 |
| 415 | MspFrag117255 | 16 | 87152490 | 87152873 |
| 416 | MspFrag118129 | 17 | 1424860 | 1425069 |
| 417 | MspFrag118132 | 17 | 1425742 | 1425962 |
| 418 | MspFrag118488 | 17 | 3262975 | 3263712 |
| 419 | MspFrag118491 | 17 | 3380201 | 3380549 |
| 420 | MspFrag118551 | 17 | 3742185 | 3742440 |
| 421 | MspFrag118936 | 17 | 6557888 | 6557950 |
| 422 | MspFrag118976 | 17 | 6866584 | 6867057 |
| 423 | MspFrag118998 | 17 | 6888109 | 6888394 |
| 424 | MspFrag119665 | 17 | 11841560 | 11842309 |
| 425 | MspFrag120286 | 17 | 19588958 | 19589326 |
| 426 | MspFrag120416 | 17 | 21214632 | 21214932 |
| 427 | MspFrag120581 | 17 | 23756303 | 23756683 |
| 428 | MspFrag120745 | 17 | 24917063 | 24917287 |
| 429 | MspFrag121117 | 17 | 29507543 | 29508230 |
| 430 | MspFrag121187 | 17 | 30501738 | 30502428 |
| 431 | MspFrag121238 | 17 | 31115713 | 31116237 |
| 432 | MspFrag121549 | 17 | 33919151 | 33919636 |
| 433 | MspFrag121727 | 17 | 34635687 | 34635916 |
| 434 | MspFrag122371 | 17 | 39446974 | 39447439 |
| 435 | MspFrag122729 | 17 | 41181205 | 41181664 |
| 436 | MspFrag122955 | 17 | 43222694 | 43222900 |
| 437 | MspFrag123151 | 17 | 44073827 | 44074263 |
| 438 | MspFrag123180 | 17 | 44159203 | 44159574 |
| 439 | MspFrag123393 | 17 | 45425386 | 45425933 |
| 440 | MspFrag123622 | 17 | 46894551 | 46894949 |
| 441 | MspFrag123625 | 17 | 47100530 | 47100939 |
| 442 | MspFrag123786 | 17 | 53294503 | 53294919 |
| 443 | MspFrag123890 | 17 | 54187494 | 54188029 |
| 444 | MspFrag123955 | 17 | 55397186 | 55397616 |
| 445 | MspFrag124390 | 17 | 60203136 | 60203426 |
| 446 | MspFrag124400 | 17 | 60205707 | 60206091 |
| 447 | MspFrag124610 | 17 | 63706209 | 63706660 |
| 448 | MspFrag124812 | 17 | 69147185 | 69147915 |
| 449 | MspFrag124831 | 17 | 69408959 | 69409615 |
| 450 | MspFrag124844 | 17 | 69615375 | 69616058 |
| 451 | MspFrag124893 | 17 | 69990739 | 69991183 |
| 452 | MspFrag125612 | 17 | 73648109 | 73648558 |
| 453 | MspFrag126928 | 17 | 77787428 | 77787810 |
| 454 | MspFrag126936 | 17 | 77793664 | 77794026 |
| 455 | MspFrag127220 | 17 | 78629464 | 78629723 |
| 456 | MspFrag127254 | 17 | 78640698 | 78640912 |
| 457 | MspFrag127669 | 18 | 7278710 | 7279418 |
| 458 | MspFrag127886 | 18 | 11365685 | 11366062 |
| 459 | MspFrag128414 | 18 | 19973409 | 19973979 |
| 460 | MspFrag128737 | 18 | 31331934 | 31332447 |
| 461 | MspFrag128850 | 18 | 33320380 | 33321106 |
| 462 | MspFrag128857 | 18 | 33399522 | 33399998 |
| 463 | MspFrag129193 | 18 | 44375040 | 44375381 |
| 464 | MspFrag129644 | 18 | 55091846 | 55092225 |
| 465 | MspFrag130161 | 18 | 72334956 | 72335293 |
| 466 | MspFrag130261 | 18 | 73091680 | 73092166 |
| 467 | MspFrag130315 | 18 | 74367316 | 74367647 |
| 468 | MspFrag130916 | 19 | 356947 | 357309 |
| 469 | MspFrag131108 | 19 | 562513 | 563000 |
| 470 | MspFrag131234 | 19 | 626106 | 626794 |
| 471 | MspFrag131881 | 19 | 1225717 | 1226067 |
| 472 | MspFrag132131 | 19 | 1454713 | 1455193 |
| 473 | MspFrag132416 | 19 | 1856758 | 1857148 |
| 474 | MspFrag132985 | 19 | 2839734 | 2840151 |
| 475 | MspFrag133397 | 19 | 3884765 | 3885169 |
| 476 | MspFrag133709 | 19 | 4736010 | 4736531 |
| 477 | MspFrag133765 | 19 | 4987710 | 4988218 |
| 478 | MspFrag133773 | 19 | 4999483 | 4999813 |
| 479 | MspFrag134007 | 19 | 5865969 | 5866340 |
| 480 | MspFrag134481 | 19 | 8278100 | 8278802 |
| 481 | MspFrag134495 | 19 | 8304633 | 8304844 |
| 482 | MspFrag134595 | 19 | 8566758 | 8567128 |
| 483 | MspFrag134630 | 19 | 9334315 | 9334667 |
| 484 | MspFrag134826 | 19 | 10264682 | 10265092 |
| 485 | MspFrag135107 | 19 | 11354200 | 11354601 |
| 486 | MspFrag135257 | 19 | 12746871 | 12747166 |
| 487 | MspFrag135413 | 19 | 12996583 | 12996817 |
| 488 | MspFrag136002 | 19 | 16298270 | 16298496 |
| 489 | MspFrag136153 | 19 | 17263933 | 17264231 |
| 490 | MspFrag136763 | 19 | 18868351 | 18868732 |
| 491 | MspFrag137207 | 19 | 35627974 | 35628220 |
| 492 | MspFrag138344 | 19 | 43973696 | 43974028 |
| 493 | MspFrag138522 | 19 | 44618313 | 44618420 |
| 494 | MspFrag138648 | 19 | 45421947 | 45422225 |
| 495 | MspFrag138677 | 19 | 45593831 | 45594133 |
| 496 | MspFrag138910 | 19 | 46878438 | 46879162 |
| 497 | MspFrag139579 | 19 | 50974863 | 50975544 |
| 498 | MspFrag140214 | 19 | 53833482 | 53834000 |
| 499 | MspFrag141334 | 19 | 60185911 | 60186130 |
| 500 | MspFrag141818 | 19 | 61770691 | 61770887 |
| 501 | MspFrag142017 | 19 | 63157706 | 63158406 |
| 502 | MspFrag142439 | 20 | 648609 | 649321 |
| 503 | MspFrag142458 | 20 | 773559 | 773845 |
| 504 | MspFrag142557 | 20 | 1875786 | 1876205 |
| 505 | MspFrag142940 | 20 | 4150615 | 4151066 |
| 506 | MspFrag143616 | 20 | 21441106 | 21441427 |
| 507 | MspFrag143733 | 20 | 22976137 | 22976617 |
| 508 | MspFrag143736 | 20 | 22976785 | 22977176 |
| 509 | MspFrag143825 | 20 | 24569612 | 24570322 |
| 510 | MspFrag143827 | 20 | 24742336 | 24742752 |
| 511 | MspFrag143864 | 20 | 25012556 | 25012953 |
| 512 | MspFrag144226 | 20 | 31770902 | 31771540 |
| 513 | MspFrag144360 | 20 | 33144476 | 33145268 |
| 514 | MspFrag144651 | 20 | 36509200 | 36509785 |
| 515 | MspFrag144826 | 20 | 39792506 | 39792745 |
| 516 | MspFrag144856 | 20 | 41569277 | 41569661 |
| 517 | MspFrag145015 | 20 | 43424513 | 43425108 |
| 518 | MspFrag145066 | 20 | 43896344 | 43897081 |
| 519 | MspFrag145069 | 20 | 43952201 | 43952384 |
| 520 | MspFrag145238 | 20 | 44977062 | 44977342 |
| 521 | MspFrag145431 | 20 | 48273066 | 48273379 |
| 522 | MspFrag145469 | 20 | 49009098 | 49009532 |
| 523 | MspFrag145587 | 20 | 52525004 | 52525348 |
| 524 | MspFrag145647 | 20 | 54635914 | 54636293 |
| 525 | MspFrag145717 | 20 | 55399273 | 55399609 |
| 526 | MspFrag145731 | 20 | 55533586 | 55533993 |
| 527 | MspFrag145848 | 20 | 56850090 | 56850439 |
| 528 | MspFrag145928 | 20 | 57131598 | 57132025 |
| 529 | MspFrag146021 | 20 | 59404205 | 59404898 |
| 530 | MspFrag146035 | 20 | 59903253 | 59903692 |
| 531 | MspFrag146294 | 20 | 60809849 | 60810182 |
| 532 | MspFrag146425 | 20 | 61188038 | 61188341 |
| 533 | MspFrag146427 | 20 | 61189329 | 61189632 |
| 534 | MspFrag146564 | 20 | 61463569 | 61463852 |
| 535 | MspFrag146589 | 20 | 61523181 | 61523518 |
| 536 | MspFrag147018 | 20 | 62158835 | 62159160 |
| 537 | MspFrag147620 | 21 | 33327565 | 33327930 |
| 538 | MspFrag147887 | 21 | 36990800 | 36991207 |
| 539 | MspFrag147896 | 21 | 36992311 | 36992534 |
| 540 | MspFrag148458 | 21 | 43964947 | 43965429 |
| 541 | MspFrag148624 | 21 | 44930972 | 44931714 |
| 542 | MspFrag148771 | 21 | 45568987 | 45569301 |
| 543 | MspFrag148921 | 21 | 46119009 | 46119510 |
| 544 | MspFrag149461 | 22 | 17536199 | 17536687 |
| 545 | MspFrag149605 | 22 | 18168920 | 18169266 |
| 546 | MspFrag149782 | 22 | 19034057 | 19034356 |
| 547 | MspFrag149784 | 22 | 19035655 | 19035873 |
| 548 | MspFrag149785 | 22 | 19035874 | 19036170 |
| 549 | MspFrag149787 | 22 | 19036333 | 19036659 |
| 550 | MspFrag149788 | 22 | 19036660 | 19037337 |
| 551 | MspFrag149790 | 22 | 19038177 | 19038476 |
| 552 | MspFrag149791 | 22 | 19038477 | 19039097 |
| 553 | MspFrag149792 | 22 | 19039098 | 19039826 |
| 554 | MspFrag149794 | 22 | 19039962 | 19040676 |
| 555 | MspFrag149824 | 22 | 19109258 | 19109530 |
| 556 | MspFrag150393 | 22 | 24071950 | 24072354 |
| 557 | MspFrag150632 | 22 | 28031149 | 28031471 |
| 558 | MspFrag151442 | 22 | 37421867 | 37422481 |
| 559 | MspFrag151528 | 22 | 37962171 | 37962758 |
| 560 | MspFrag151564 | 22 | 38109182 | 38109628 |
| 561 | MspFrag152094 | 22 | 41917375 | 41918092 |
| 562 | MspFrag152213 | 22 | 43445922 | 43446102 |
| 563 | MspFrag152321 | 22 | 44582503 | 44582872 |
| 564 | MspFrag152480 | 22 | 45091310 | 45091573 |
| 565 | MspFrag152489 | 22 | 45194587 | 45195050 |
| 566 | MspFrag152494 | 22 | 45250387 | 45250713 |
| 567 | MspFrag152496 | 22 | 45250831 | 45251397 |
| 568 | MspFrag152632 | 22 | 47145509 | 47145882 |
| 569 | MspFrag152655 | 22 | 47247350 | 47247678 |
| 570 | MspFrag152681 | 22 | 47331247 | 47331652 |
| 571 | MspFrag152714 | 22 | 47818757 | 47819111 |
| 572 | MspFrag152716 | 22 | 47821576 | 47822084 |
| 573 | MspFrag152736 | 22 | 48119202 | 48119610 |
| 574 | MspFrag152748 | 22 | 48288961 | 48289335 |
| 575 | MspFrag153027 | 22 | 48991342 | 48991874 |
| 576 | MspFrag153087 | 22 | 49023037 | 49023473 |
| 577 | MspFrag153362 | 23 | 106714 | 106947 |
| 578 | MspFrag153363 | 23 | 106948 | 107207 |
| 579 | MspFrag153364 | 23 | 107208 | 107441 |
| 580 | MspFrag153365 | 23 | 107442 | 107957 |
| 581 | MspFrag153563 | 23 | 407042 | 407560 |
| 582 | MspFrag154875 | 23 | 39303900 | 39304278 |
| 583 | MspFrag155418 | 23 | 47418801 | 47419138 |
| 584 | MspFrag155823 | 23 | 52912797 | 52913213 |
| 585 | MspFrag156275 | 23 | 71242026 | 71242406 |
| 586 | MspFrag156306 | 23 | 72006660 | 72007155 |
| 587 | MspFrag156308 | 23 | 72081592 | 72082087 |
| 588 | MspFrag156440 | 23 | 82569986 | 82570585 |
| 589 | MspFrag156491 | 23 | 90495771 | 90495990 |
| 590 | MspFrag156922 | 23 | 114782761 | 114783003 |
| 591 | MspFrag157076 | 23 | 117741123 | 117741602 |
| 592 | MspFrag157770 | 23 | 135838695 | 135839395 |
| 593 | MspFrag158624 | 23 | 154810057 | 154810810 |
| 594 | MspFrag158646 | 24 | 106714 | 106947 |
| 595 | MspFrag158647 | 24 | 106948 | 107207 |
| 596 | MspFrag158648 | 24 | 107208 | 107441 |
| 597 | MspFrag158649 | 24 | 107442 | 107957 |
| 598 | MspFrag158845 | 24 | 407042 | 407560 |
| 599 | MspFrag158867 | 24 | 554703 | 554798 |
| 600 | MspFrag158958 | 24 | 1628781 | 1629129 |

In an embodiment a method 10 is provided, according to FIG. 1. Said method 10 comprises selecting 100 a feature subset comprising at least one post from the methylation classification list according to SEQ ID NO. 1 to SEQ ID NO. 600.

Selecting 100 a feature subset may be performed based on hierarchical clustering with Pearson correlation and complete linkage to characterize the fitness of each feature subset, given a dataset with methylation characterization for of each sample (sᵢ, i=1 . . . M) in a form of a vector mᵢ of N values, where m_{i,j} provides the methylation status for the i-th sample and the j-th probe. Typically, some statistical analysis of the measured signal will produce a set of probes (features) to be input to the hierarchical clustering method above.

The feature subset selection 100 uses a Genetic Algorithm (GA), which repetitively evaluate feature subsets based on a fitness function that in some way characterizes some property of the feature subset. In an embodiment, hierarchical clustering with Pearson correlation and complete linkage is used as the fitness function to assess how good a feature subset is.

The following example is used to illustrate the principle.

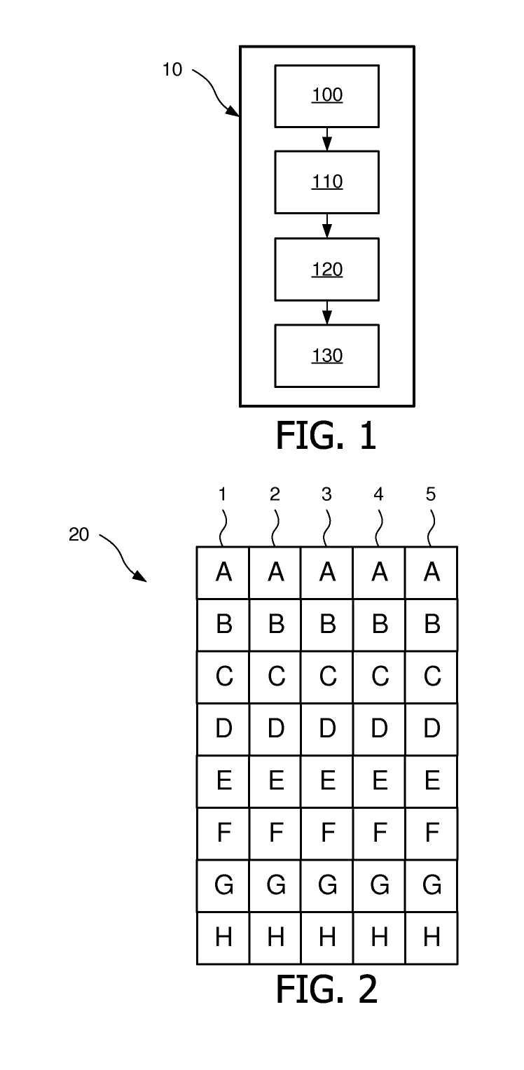

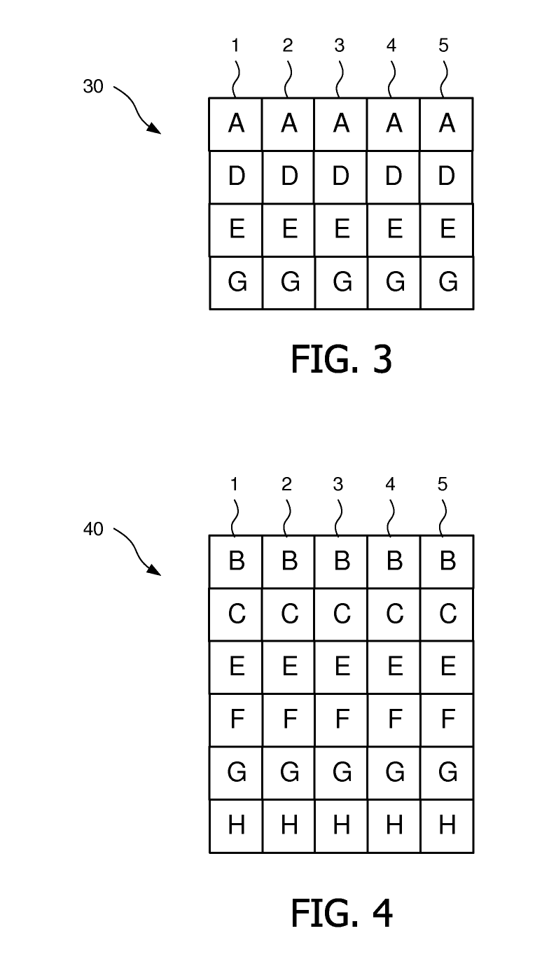

FIG. 2 show a dataset 20 of measurements, in this case 5 samples, which are displayed as 1 to 5 are characterized with 8 features, which are displayed as letters A to H. FIGS. 3 and 4 show two feature subsets, generated from the measurements dataset by selecting rows (features) from the dataset. FIG. 3 shows a first feature subset 30 with the 5 samples, which are displayed as 1 to 5, but only four of the features. FIG. 4 shows a second subset 40 with the 5 samples, which are displayed as 1 to 5, but only six of the features.

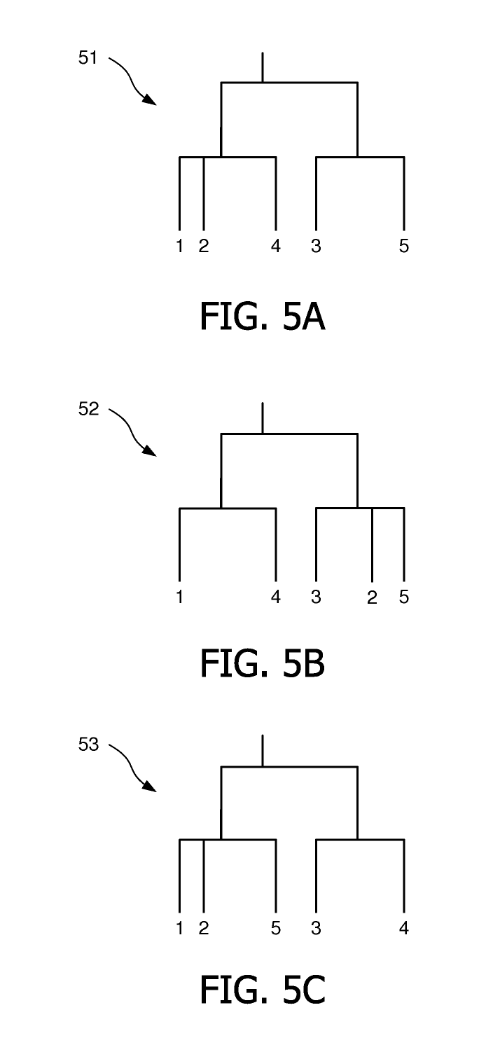

Next, clustering may be performed. FIG. 5 show clusters, or dendrograms, based on the datasets from FIGS. 2 to 4, when subjected to hierarchical clustering with Pearson correlation and complete linkage. FIG. 5A shows a first cluster 51 based on the total dataset 20. FIG. 5B shows a second cluster 52 based on the first feature subset 30 and FIG. 5C shows a third cluster 53 based on the second feature subset 40.

After having clustered the datasets, a ranking of all clustering results is performed. In one embodiment, a cluster analysis method is used for the ranking. For example, it is possible to characterize and rank individual clusters based on their validity, for example in terms of cluster cohesion or separation. This may be done in one of multiple ways well known to a person skilled in the art. Thus, it is possible to rank two or more feature subsets based on the quality of the clusters they generate when used to cluster the samples.

In another embodiment, some property of the samples (e.g. cancer subtype based on pathology) is used for ranking. From this property, the same or related subtypes are grouped together. For example, if the five samples from FIGS. 2 to 4 have the following subtype labels associated with them {1=X, 2=X, 3=Y, 4=Y, 5=X} respectively, this would then produce the following label groupings for the three clusters shown in FIG. 5: A: {XXY, YX}; B: {XY, YXX}; C: {XXX, YY}. In this case, the second subset 40, represented by FIG. 5C, is clearly better compared to the first feature subset 30 or the clustering based on the entire dataset 20, since it correctly cluster the subtypes together.

In an embodiment, two clustering outputs D₁ and D₂, are compared based on the clusters. First, N (C₁, C₂, . . . C_{N}) clusters are obtained based on the dendrogram, produced by the clustering. Then, a property is computed based on the clusters, such as the popular method of silhouette width—SIL(Cᵢ). Now a single-number characterization of a clustering is obtained by the formula:

AVGSIL(D)=(SUM[i=1 . . . N]SIL(Cᵢ))/N

By comparing AVGSIL(D₁) and AVGSIL(D₂), it may be determined which clustering is preferable. In another embodiment, build a data structure G is built in form of a matrix with dimensions N×L, where L is the number of distinct labels available for the samples. With labels {X. Y}, L=2, or for labels {normal, aggressive cancer, non-aggressive cancer} L=3. Then for each cluster i (i=1 . . . N) L values are obtained in the following manner for each element gᵢⱼ from G:

gᵢⱼ=count(sample in cluster i and has label j)

Now, it is possible to compute uniformity of each cluster Cᵢ:

UNIFORMITY(Cᵢ)=max(counts in row i in G)/sum(counts in row i in G)

Finally, the clustering is characterized with:

AVGUNIFORMITY(D)=SUM[i=1 . . . N](UNIFORMITY(Cᵢ))/N

as a single-number characterization of a clustering. By comparing AVGUNIFORMITY (D₁) and AVGUNIFORMITY (D₂) it may be determined which clustering is preferable.

Iterative repetition of this selection process gradually refines the quality of the clustering of the feature subsets discovered by the GA. After a number of repetitions, all evaluated features subsets can be further filtered based on their performance during the GA execution. In one embodiment, feature subsets are sorted by the average clustering performance in stratification of the clinical samples. In another embodiment, feature subsets, in addition to the average performance, are filtered based on their persistent re-evaluation. In other words, feature subsets that are repeatedly selected for further evaluation are preferred to feature subsets that are dropped from consideration only after a few iterations. The final output of a GA feature subset selection is to run multiple instances with different initial conditions, and merge the filtered feature subsets from each of these instances. Feature subsets from one such evaluation are listed in Table 3A. Furthermore, a cumulative characterization of a collection of GA runs can be obtained and used to generate feature subsets that aggregate the feature subsets in single set of subsets. In one embodiment, the appearance of each feature in feature subsets is counted and a total histogram is obtained giving the degree of utilization of each of the 600 features. Based on this information and for example in one embodiment the frequencies of the pairwise occurrences of the 600 features are used to build feature subsets that summarize the GA run in a single set of subsets, a so called trend pattern. Table 3B provides such feature subset of lengths 45 and 60.

Examples of feature subsets are provided in Tables 2, 3A and 3B. Thus, in an embodiment, the feature subset comprises the CpG dinucleotides according to one of the selections listed in Table 2.

TABLE 2
Feature subsets. Each subset comprise a selection of sequences
indicated by numbers corresponding to the FragID:s in table 1.

| Selection | |
|---|---|
| number: | FragID:s |
| 1 | 152494, 110545, 1212, 55649, 102005, 129193, 86866, 89848, 1601, 153363, 158647, 1311, |
| | 128850, 19926, 123622, 149824, 72674, 150393, 10496, 17697, 95107, 85656, 65670, |
| | 55275, 149782, 124610, 124844, 49687, 14334, 757, 157076, 79207, 11782, 120745, |
| | 127220, 114108, 22036, 11474, 52434, 136153, 110848, 90376, 145015, 80728, 99113, |
| | 158958, 110494, 47510, 26073, 71105, 20024, 10537, 145717, 146294, 1534, 50717, 24273, |
| | 143733, 71090, 92849, 111358, 57442, 80168, 61099, 80989, 22213, 141818, 71700 |
| 2 | 152494, 1650, 102005, 14197, 21537, 110668, 158646, 13583, 73586, 38815, 19926, |
| | 114107, 103295, 80645, 149824, 127886, 115442, 151564, 113247, 38281, 126936, 121549, |
| | 74598, 65670, 55275, 80954, 1241, 118491, 142017, 1377, 105085, 120745, 3535, 36661, |
| | 87210, 110848, 138677, 145015, 143616, 8778, 26073, 25164, 9703, 145717, 72461, 1339, |
| | 122371, 133709, 27379, 56289, 17091, 153087, 5525, 146564, 57442, 80112, 28326, |
| | 113989, 157770, 147896, 98985, 121727, 73907, 9029 |
| 3 | 152494, 110545, 55649, 133765, 114140, 129193, 5071, 86866, 99554, 72675, 45501, |
| | 52027, 1173, 19926, 153364, 103295, 123622, 149824, 5104, 151564, 118551, 98223, |
| | 14203, 147018, 65670, 4389, 105101, 147620, 149788, 55218, 118491, 118129, 152681, |
| | 64725, 39543, 87210, 38910, 80728, 153563, 71121, 71105, 152094, 50717, 87160, 71090, |
| | 33136, 76797, 78440, 26333, 145587, 63043, 50444, 5980, 9937, 7359, 158867, 141818 |
| 4 | 110545, 86939, 55649, 102005, 152632, 129193, 86866, 103518, 153363, 158647, 145928, |
| | 7228, 67459, 19926, 10427, 4823, 149824, 14609, 149605, 47435, 92237, 152489, 85089, |
| | 98223, 108348, 65670, 105101, 118491, 149792, 757, 10623, 118129, 27685, 99472, 36661, |
| | 87210, 90376, 138677, 152716, 158624, 149787, 148624, 60779, 71105, 152094, 123955, |
| | 50717, 73062, 42953, 80169, 42441, 78440, 119665, 113989, 10916, 118998, 145587, |
| | 102061, 151528 |
| 5 | 152494, 110545, 55649, 102005, 25023, 158649, 130916, 114218, 74424, 80975, 73586, |
| | 1173, 114107, 32667, 103295, 126928, 115442, 127254, 134481, 147018, 121549, 110579, |
| | 65670, 14202, 147620, 96587, 149788, 14254, 757, 121238, 1377, 120745, 120286, 87210, |
| | 38910, 25187, 90376, 149787, 55475, 99113, 8778, 99150, 71121, 92533, 71105, 9703, |
| | 82920, 149785, 14451, 122371, 1534, 29324, 10916, 145587, 63043, 87698, 27677, 156491, |
| | 20225 |
| 6 | 152494, 110545, 80343, 55649, 1650, 114140, 102005, 129193, 144651, 99554, 158647, |
| | 149824, 115442, 71104, 52792, 113247, 126936, 52897, 85656, 65670, 68271, 55275, |
| | 147620, 96587, 38714, 130315, 757, 121238, 5190, 116223, 148458, 87210, 110848, 90376, |
| | 145015, 8778, 31913, 26073, 99150, 149790, 122729, 92520, 71105, 2123, 15066, 152094, |
| | 72461, 130161, 73062, 94051, 5525, 4820, 1391, 108016, 157770, 46277, 134630, 7153, |
| | 158867, 9029 |
| 7 | 110545, 114140, 102005, 25023, 130916, 129193, 99554, 65671, 153363, 158646, 128850, |
| | 13583, 7228, 19926, 158648, 45007, 149824, 47435, 92237, 152496, 138648, 116804, |
| | 65670, 4389, 147620, 140214, 14231, 99472, 148458, 1249, 87210, 26133, 152716, 93471, |
| | 115251, 71121, 25164, 71216, 133709, 123786, 25517, 94051, 36595, 5525, 80169, 108016, |
| | 103793, 146564, 54796, 156440, 35700, 2643, 143864, 115870, 11354, 71700 |
| 8 | 110545, 86939, 55649, 1650, 129193, 99554, 62044, 152321, 72675, 120416, 128414, |
| | 60291, 152655, 80645, 149824, 72674, 127886, 56402, 132985, 95107, 152496, 117255, |
| | 138648, 134481, 147018, 121549, 65670, 55275, 4389, 124610, 20895, 66071, 136002, |
| | 1377, 118129, 127220, 36661, 11474, 145015, 39760, 48491, 99113, 94345, 125612, 47510, |
| | 31913, 122729, 71105, 27268, 82920, 149785, 154875, 1534, 123955, 133709, 50717, |
| | 142439, 71090, 80989, 72750, 46277, 14656, 121727, 113614, 27495, 88140 |
| 9 | 152494, 110545, 1211, 55649, 152714, 129193, 114087, 152321, 153363, 80854, 128414, |
| | 13583, 45501, 63267, 60291, 80645, 9601, 4823, 14921, 115442, 151564, 132985, 47435, |
| | 92237, 95107, 152496, 114207, 65670, 55275, 4389, 66146, 38491, 149788, 114206, |
| | 118132, 757, 71581, 99668, 136002, 76422, 123180, 148458, 87210, 136153, 110848, |
| | 137207, 45409, 7116, 60779, 1324, 131108, 138910, 15478, 138344, 149785, 60445, 68970, |
| | 42953, 71090, 80169, 59067, 80112, 131234, 10916, 118998, 63043, 87698, 156491, 113614 |
| 10 | 152494, 55649, 158649, 33381, 129193, 38485, 86866, 1601, 153363, 158646, 72675, |
| | 128850, 13583, 4109, 38815, 63267, 19926, 103295, 79123, 4823, 80726, 115442, 25715, |
| | 71104, 92237, 152496, 134481, 1359, 65670, 55275, 77777, 114219, 118132, 149792, 757, |
| | 27685, 71089, 120745, 3535, 36661, 52666, 148458, 56504, 87210, 110848, 39760, 152716, |
| | 94345, 47510, 87185, 156306, 71105, 89865, 54424, 95724, 153087, 42953, 71090, 57442, |
| | 76797, 70538, 156440, 113989, 13394, 46277, 14656, 20225, 9029, 89183 |
| 11 | 152494, 110545, 12301, 14289, 61152, 1650, 129193, 99554, 153362, 72675, 120416, |
| | 149794, 13583, 19926, 32667, 103295, 150393, 92237, 45338, 95107, 96587, 149788, |
| | 66071, 14254, 757, 37395, 99668, 14231, 118129, 152681, 155418, 36661, 146589, 148458, |
| | 1249, 55611, 110848, 71074, 88982, 32624, 47510, 31913, 26073, 71121, 71105, 145717, |
| | 72461, 15478, 118488, 153027, 154875, 133709, 144856, 60445, 73062, 5525, 152213, |
| | 92849, 80168, 63043, 90137, 56922 |
| 12 | 152494, 110545, 114218, 129193, 86495, 86866, 99554, 45501, 38815, 19926, 158648, |
| | 103295, 60291, 10427, 149824, 115442, 151564, 152496, 98223, 147018, 65670, 77777, |
| | 55218, 118491, 118132, 33338, 142017, 54824, 55941, 36661, 145238, 87210, 138677, |
| | 39760, 45409, 123890, 99150, 71121, 25164, 1324, 71105, 82920, 1534, 123955, 133709, |
| | 24273, 60445, 94051, 71090, 80169, 108016, 70538, 78440, 39539, 131234, 134630, 50444, |
| | 87698, 143864, 90137, 64684, 45650 |
| 13 | 152494, 110545, 55649, 1650, 102005, 158649, 129193, 86495, 86866, 128414, 128850, |
| | 146035, 1173, 19926, 153364, 4823, 149824, 14609, 72674, 56402, 118551, 45338, 65670, |
| | 114220, 61161, 118491, 130315, 18856, 118129, 148458, 87210, 110848, 134826, 145015, |
| | 93471, 48491, 80728, 125612, 46110, 110793, 99150, 71121, 96210, 10393, 2123, 15066, |
| | 152094, 27268, 28887, 1339, 133709, 111802, 76797, 42441, 145731, 26333, 147896, |
| | 63043, 87698, 11354, 73907, 27495 |
| 14 | 114205, 129193, 86866, 99554, 152321, 52027, 80645, 72674, 76619, 151564, 71104, |
| | 113247, 47435, 95107, 126936, 136763, 147018, 84490, 65670, 55275, 105101, 20895, 757, |
| | 99668, 50853, 27685, 148458, 56504, 110848, 145015, 144226, 89408, 99113, 158958, |
| | 125612, 144360, 7116, 26073, 99150, 96210, 71105, 124831, 152094, 71216, 1339, 14451, |
| | 88395, 142439, 71090, 92849, 103793, 57442, 119665, 88411, 46277, 10916, 134630, |
| | 11354, 90137, 27495 |
| 15 | 110545, 102005, 129193, 158646, 153362, 73586, 27115, 114138, 127886, 56402, 5104, |
| | 115442, 150632, 151564, 71104, 152496, 53338, 114207, 134481, 116804, 65670, 55275, |
| | 118132, 130315, 96227, 71581, 118129, 79207, 155418, 123180, 114108, 52666, 1249, |
| | 84518, 64725, 87210, 136153, 135257, 145015, 156308, 48491, 152480, 45409, 88982, |
| | 26073, 71121, 152094, 40505, 149461, 54424, 28887, 14451, 123955, 56289, 83839, 1391, |
| | 108016, 39539, 119665, 88411, 9278, 102061, 27677, 115870, 14656, 56922 |
| 16 | 152494, 110545, 86939, 55649, 102005, 25023, 128737, 129193, 14197, 99554, 152321, |
| | 153362, 72675, 13583, 39470, 61003, 103295, 79123, 80726, 118551, 114139, 147620, |
| | 96587, 55218, 38714, 8273, 757, 54400, 1823, 15771, 46721, 157076, 71120, 3535, 52666, |
| | 11474, 148458, 87210, 57206, 152480, 55475, 89408, 99113, 148624, 7116, 8778, 110793, |
| | 47510, 26073, 76120, 25164, 71105, 124831, 127669, 9928, 27268, 154875, 144856, 60445, |
| | 88395, 94051, 36595, 71090, 111358, 76797, 50444, 27677, 23738, 76467, 71700 |
| 17 | 110545, 114140, 102005, 129193, 99554, 152321, 128850, 5455, 124390, 149824, 80726, |
| | 126928, 56402, 151564, 17697, 47435, 152496, 38417, 147018, 116804, 84490, 65670, |
| | 4389, 118491, 757, 99668, 15771, 46721, 118129, 79207, 105085, 127220, 36661, 22036, |
| | 148458, 64725, 52146, 87210, 136153, 145015, 31913, 26073, 71105, 15066, 145717, |
| | 20134, 130161, 14451, 50717, 17091, 60445, 87160, 33136, 54796, 57442, 76797, 59067, |
| | 61099, 20706, 28326, 72750, 76801, 82859, 105873, 27677, 113614, 9029 |
| 18 | 152494, 110545, 55649, 153365, 129193, 21537, 86866, 99554, 72675, 120581, 52027, |
| | 19926, 103295, 114138, 1340, 151564, 128857, 132985, 118551, 95107, 152748, 98223, |
| | 14203, 65670, 149788, 55218, 118491, 118132, 142017, 118129, 11782, 27685, 99472, |
| | 36661, 87210, 38910, 55611, 135107, 135257, 149787, 48491, 80728, 7116, 110793, 99150, |
| | 71105, 9928, 40858, 58680, 1534, 133709, 60445, 94051, 5525, 71090, 70538, 80112, 2643, |
| | 9937, 98985, 64684 |

In an embodiment, the feature subset comprises the CpG dinucleotides according to one of the selections listed in Table 3A.

TABLE 3A
Feature subsets. Each subset comprise a selection of sequences
indicated by numbers corresponding to the FragID:s in table 1.

| Selection | |
|---|---|
| number: | FragID:s |
| 1 | 145469, 158845, 1211, 110545, 133397, 99554, 114107, 151442, 99150, 6914, 14609, |
| | 74424, 130315, 152714, 115251, 25023, 96210, 117255, 147887, 124390, 135107, 152716, |
| | 14457, 149605, 134595, 158958, 86939, 158624, 20895, 56289, 150632, 54400, 114205, |
| | 99310, 120416, 123890, 115870 |
| 2 | 145469, 158845, 1211, 110545, 133397, 99554, 114107, 151442, 99150, 6914, 14609, |
| | 74424, 130315, 152714, 115251, 25023, 96210, 117255, 147887, 124390, 135107, 152716, |
| | 14457, 149605, 134595, 158958, 86939, 158624, 20895, 56289, 150632, 54400, 114205, |
| | 99310, 120416, 123890, 115870 |
| 3 | 145469, 158845, 1211, 110545, 133397, 99554, 114107, 151442, 99150, 6914, 14609, |
| | 74424, 130315, 152714, 115251, 25023, 96210, 117255, 147887, 118998, 135107, 152748, |
| | 14457, 133709, 149605, 1321, 110848, 134595, 158958, 86939, 158624, 20895, 56289, |
| | 150632, 54400, 47196, 114205, 99310, 123890, 115870 |
| 4 | 145469, 158845, 1211, 110545, 133397, 99554, 114107, 151442, 99150, 6914, 14609, |
| | 74424, 130315, 152714, 115251, 25023, 96210, 117255, 147887, 110848, 135107, 152748, |
| | 14457, 149605, 134595, 158958, 86939, 158624, 20895, 56289, 150632, 54400, 47196, |
| | 114205, 99310, 120416, 123890, 115870 |
| 5 | 145469, 158845, 1211, 110545, 133397, 99554, 114107, 151442, 99150, 6914, 14609, |
| | 74424, 130315, 152714, 115251, 25023, 96210, 117255, 147887, 123955, 135107, 47196, |
| | 14457, 149605, 134595, 158958, 86939, 158624, 20895, 56289, 150632, 54400, 114205, |
| | 99310, 120416, 123890, 115870 |
| 6 | 145469, 158845, 1211, 110545, 133397, 99554, 114107, 151442, 99150, 6914, 14609, |
| | 74424, 130315, 152714, 115251, 25023, 96210, 117255, 147887, 118998, 135107, 47196, |
| | 14457, 133709, 149605, 1321, 110848, 134595, 158958, 86939, 158624, 20895, 56289, |
| | 150632, 54400, 114205, 99310, 123890, 115870 |
| 7 | 145469, 158845, 1211, 110545, 133397, 99554, 114107, 151442, 99150, 6914, 14609, |
| | 74424, 130315, 152714, 115251, 25023, 96210, 117255, 147887, 124390, 135107, 47196, |
| | 14457, 149605, 134595, 158958, 86939, 158624, 20895, 56289, 150632, 54400, 114205, |
| | 99310, 120416, 123890, 115870 |
| 8 | 145469, 158845, 1211, 110545, 133397, 99554, 114107, 151442, 99150, 6914, 14609, |
| | 74424, 130315, 152714, 115251, 25023, 96210, 117255, 147887, 124390, 135107, 47196, |
| | 14457, 149605, 134595, 158958, 86939, 158624, 20895, 56289, 150632, 54400, 114205, |
| | 99310, 120416, 123890, 115870 |

In an embodiment, the feature subset comprises the CpG dinucleotides according to one of the selections listed in Table 3B.

TABLE 3B
Feature subsets. Each subset comprise a selection of sequences
indicated by numbers corresponding to the FragID:s in table 1.

| Selection | |
|---|---|
| number: | FragID:s |
| 1 | 145469, 158845, 1211, 110545, 133397, 99554, 114107, 151442, 99150, 6914, 14609, |
| | 74424, 130315, 152714, 117255, 96210, 149605, 146589, 77777, 115251, 25023, 120416, |
| | 124390, 147887, 123955, 79123, 152716, 134495, 118998, 133709, 91076, 14457, 110848, |
| | 54400, 158624, 134595, 1321, 80728, 146294, 136763, 158958, 115870, 20895, 56289, |
| | 59067, 104383, 114205, 130161, 152748, 123890, 142557, 86866, 26333, 80726 |
| 2 | 145469, 158845, 1211, 38910, 133397, 99554, 114107, 151442, 99150, 6914, 14609, 74424, |
| | 130315, 152714, 117255, 96210, 149605, 146589, 77777, 115251, 114220, 120416, 124390, |
| | 147887, 123955, 79123, 47196, 134495, 118998, 133709, 91076, 14457, 110848, 54400, |
| | 158624, 134595, 1321, 80728, 146294, 136763, 158958, 115870, 20895, 56289, 59067, |
| | 104383, 114205, 130161, 152748, 123890, 142557, 86866, 135107, 26333, 80726 |
| 3 | 145469, 158845, 1211, 110545, 133397, 99554, 114107, 151442, 99150, 6914, 14609, |
| | 74424, 130315, 152714, 117255, 114220, 149605, 146589, 77777, 115251, 25023, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 115870, 20895, 56289, |
| | 59067, 104383, 114205, 130161, 152748, 123890, 142557, 86866, 26333, 80726 |
| 4 | 145469, 158845, 1211, 110545, 133397, 99554, 114107, 151442, 99150, 6914, 14609, |
| | 74424, 130315, 152714, 117255, 96210, 149605, 146589, 77777, 115251, 114220, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 115870, 20895, 56289, |
| | 59067, 104383, 114205, 130161, 152748, 123890, 142557, 86866, 135107, 26333, 80726 |
| 5 | 145469, 158845, 1211, 38910, 133397, 99554, 114107, 151442, 99150, 6914, 14609, 74424, |
| | 130315, 152714, 117255, 96210, 149605, 146589, 77777, 115251, 114220, 120416, 124390, |
| | 147887, 123955, 79123, 47196, 134495, 118998, 133709, 91076, 14457, 110848, 54400, |
| | 158624, 134595, 1321, 80728, 146294, 136763, 158958, 115870, 20895, 56289, 5190, |
| | 104383, 114205, 130161, 152748, 123890, 142557, 86866, 135107, 26333, 80726 |
| 6 | 145469, 158845, 1211, 110545, 133397, 99554, 114107, 151442, 99150, 6914, 14609, |
| | 74424, 130315, 152714, 117255, 96210, 149605, 146589, 77777, 115251, 114220, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 115870, 20895, 56289, |
| | 5190, 104383, 114205, 130161, 152748, 123890, 142557, 86866, 135107, 26333, 80726 |
| 7 | 145469, 158845, 1211, 38910, 133397, 99554, 114107, 151442, 99150, 6914, 14609, 74424, |
| | 130315, 152714, 117255, 114220, 149605, 146589, 77777, 115251, 135107, 120416, |
| | 124390, 147887, 123955, 79123, 152716, 134495, 118998, 133709, 91076, 14457, 110848, |
| | 54400, 158624, 134595, 1321, 80728, 146294, 136763, 158958, 115870, 20895, 56289, |
| | 59067, 104383, 114205, 130161, 152748, 123890, 142557, 86866, 26333, 80726 |
| 8 | 145469, 158845, 1211, 110545, 133397, 99554, 114107, 151442, 99150, 6914, 14609, |
| | 74424, 130315, 152714, 117255, 114220, 149605, 146589, 77777, 115251, 135107, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 115870, 20895, 56289, |
| | 5190, 104383, 114205, 130161, 152748, 123890, 142557, 86866, 26333, 80726 |
| 9 | 145469, 158845, 1211, 110545, 133397, 99554, 114107, 151442, 99150, 6914, 14609, |
| | 74424, 130315, 152714, 117255, 114220, 149605, 146589, 77777, 115251, 25023, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 25517, 20895, 56289, |
| | 59067, 104383, 114205, 130161, 152748, 123890, 142557, 86866, 26333, 80726 |
| 10 | 145469, 158845, 1211, 110545, 133397, 99554, 114107, 151442, 99150, 6914, 14609, |
| | 74424, 130315, 152714, 117255, 96210, 149605, 146589, 77777, 115251, 135107, 120416, |
| | 124390, 147887, 123955, 79123, 47196, 134495, 118998, 133709, 91076, 14457, 110848, |
| | 54400, 158624, 134595, 1321, 80728, 146294, 136763, 158958, 25517, 20895, 56289, 5190, |
| | 104383, 114205, 130161, 152748, 123890, 142557, 86866, 26333, 80726 |

In an embodiment the method 10 comprises determining 120 the methylation status of one or more CpG dinucleotides in a sequence selected from the group of sequences corresponding to the marker panel, resulting in a methylation classification list. There are numerous methods for determining 120 the methylation status of a DNA molecule of a subject, corresponding to the feature subset. The DNA may be obtained by any method for purifying DNA known to a person skilled in the art. In an embodiment the methylation status is determined 110 by means of one or more of the methods selected form the group of, bisulfite sequencing, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), microarray-based methods, msp I cleavage.

In an embodiment, the method 10 also comprises statistically analyzing 120 the methylation classification list, thus obtaining a category of the breast cancer of the subject. This may be done by jointly clustering the subject methylation data and the samples from the clinical study. The resulting clustering is then split in N groups (e.g. by cutting the clustering dendrogram into N sub-trees). The sub-tree containing the subject is evaluated for the categories of breast cancer present in the study samples and the subject sample is assigned the category of the majority samples in the sub-tree.

In an embodiment, the method 10 further comprises classifying (130) the subject as belonging to one of the five major subtypes of breast cancers.

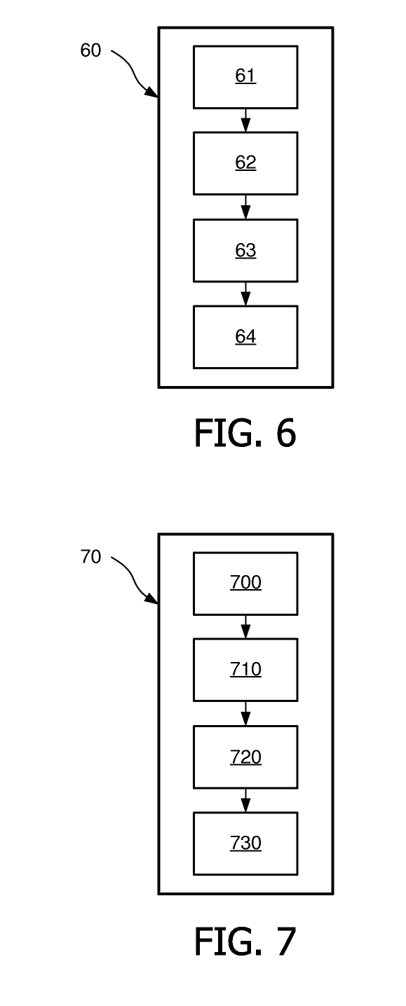

In an embodiment according to FIG. 6, a computer program product 60 is provided. The computer program product 60 is stored on a computer-readable medium, which comprises a first 61, second 62, third 63 and forth 64 code segments arranged, when run by an apparatus having computer-processing properties, for performing all of the method steps defined in some embodiments.

In an embodiment according to FIG. 7, a device 70 for supporting a clinician is provided. Said device comprising means for selecting 700 a feature subset comprising at least one post from the methylation classification list according to SEQ ID NO. 1 to SEQ ID NO. 600. Furthermore, the device 70 comprises means for determining 710 the methylation status of one or more CpG dinucleotides in DNA of a subject, corresponding to the feature subset. Furthermore, the device 70 comprises means for statistically analyzing 720 the methylation classification list, thus obtaining a category of the breast cancer of the subject. Furthermore, the device 70 comprises means for classifying 730 the subject as belonging to one of the five major subtypes of breast cancers. Said means 700, 710, 720, 730 may be operatively connected to each other.

The invention may be implemented in any suitable form including hardware, software, firmware or any combination of these. However, preferably, the invention is implemented as computer software running on one or more data processors and/or digital signal processors. The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

### LIST OF REFERENCE SIGNS

10 A method
100 A selecting step
110 A determining step
120 An analyzing step
130 A classifying step
20 A dataset
30 A first feature subset
40 A second feature subset
51 A first cluster
53 A second cluster
60 A third cluster
60 A computer program product
61 A first code segment
62 A second code segment
63 A third code segment
64 A fourth code segment
70 A device
700 Selecing means
710 Determining means
720 Analyzing means
730 Classifying means
1 to 5 Sample numbers

1. Method (10) for the analysis of breast cancer disorders, comprising determining the genomic methylation status of one or more CpG dinucleotides in a sequence selected from the group of sequences consisting of SEQ ID NO. 1 to SEQ ID NO. 600.

2. Method according to claim 1, wherein the analysis is categorization of breast cancer in a subject and wherein the following steps are performed,
a. selecting (100) a feature subset comprising at least one post from the methylation classification list according to SEQ ID NO. 1 to SEQ ID NO. 600;
b. determining (110) the methylation status of one or more CpG dinucleotides in DNA of a subject, corresponding to the feature subset; and
c. statistically analyzing (120) the methylation classification list, thus obtaining a category of the breast cancer of the subject.

3. Method according to claim 1, wherein additionally following steps are performed,
d. classifying (130) the subject as belonging to one of the five major subtypes of breast cancers.

4. Method according to claim 1, wherein the methylation status is determined (110) for a subgroup of sequences where in the specific subgroup is selected from Table 2, 3A or 3B.

5. Method according to claim 1 wherein the methylation status is determined (110) for a subgroup of sequences determined by selecting (100) a feature subset.

6. Method according to claim 5, wherein the feature subset selection (100) is a genetic algorithm with hierarchical clustering.

7. Method according to claim 1, wherein the methylation status is determined (110) for a subgroup of sequences determined by a summarization of output of feature subset selection (100).

8. Method according to claim 7, wherein the summarization of output of feature subset selection (100) is the count of appearance of each feature in feature subsets and pairwise occurrences of sequences selected from the group of sequences consisting of SEQ ID NO. 1 to SEQ ID NO. 600.

9. Method according to claim 8, wherein the count of appearance of each feature in feature subsets and pairwise occurrences of sequences are of size 45.

10. Method according to claim 8, wherein the count of appearance of each feature in feature subsets and pairwise occurrences of sequences are of size 60.

11. Method according to claim 1, wherein the methylation status is determined (110) by means of one or more of the methods selected form the group of,
a. bisulfite sequencing
b. pyrosequencing
c. methylation-sensitive single-strand conformation analysis(MS-SSCA)
d. high resolution melting analysis (HRM)
e. methylation-sensitive single nucleotide primer extension (MS-SnuPE)
f. base-specific cleavage/MALDI-TOF
g. methylation-specific PCR (MSP)
h. microarray-based methods and
i. msp I cleavage.

12. A computer program product (60) stored on a computer-readable medium comprising software code adapted to perform the steps of the method according to claim 2 when executed on a data-processing apparatus.

13. A device (70) for supporting a clinician, said device comprising means for
a. selecting (700) a feature subset comprising at least one post from the methylation classification list according to SEQ ID NO. 1 to SEQ ID NO. 600;
b. determining (710) the methylation status of one or more CpG dinucleotides in DNA of a subject, corresponding to the feature subset;
c. statistically analyzing (720) the methylation classification list, thus obtaining a category of the breast cancer of the subject; and
d. classifying (730) the subject as belonging to one of the five major subtypes of breast cancers.
said means being operatively connected to each other.


SEQUENCE LISTING (600 sequences)

SEQ ID NO: 1 - DNA, 584 bp, Homo sapiens
gcaggtgcagccctcagggagggggtgcgtgtgtgcagggaggttgttccgggaagacaaatttcagtttaatgtaaagaggtgctttttcttcccaggcaaagttggggtgcctaaagagtaaggagctctctcagtggtggtgtgcaagtggggttgctcatgtggagggatgacaccaggaaaggcttgagtcttcaaggttcctggaggaccccccgcctcccgccaacacatactcacacacatgtacattcacagagccctgcaggagagggcctgggagagggtgggaggccccttacctctccagggaccagtgcaaccagccaggttaagcggccttggtctctgtgcctccgccagtcacgcgggcttgtttcgcggcctgggccgagagcgtgtccatgatgaagccccgcgcccaaggacgagcccccgccagcgcagttcgcagcagctcctggtccctgtagcagcgccgcttctggggaccagttaggggctggctggagcgtagcagaacctgcggaccggactcagttctggaagtgacccctcagccttaggggtcctctaagcca

SEQ ID NO: 2 - DNA, 516 bp, Homo sapiens
cggccctcccgggtggttgctggctgctccctgtggggtggcaggtggccggcttccaccctgcccgagccgccgcctacctagcatgctgggctcgcctcccagcagggacaggatgtacttgttgaggaagagcgtgcagaagctgaagaagaaccacagcgtgaggtagagcagcgcccgcgagctccacacacccaagtctgactcgatgaccgtggtctccgtgatggtgacggtcagtacgttctcatctgtgccgccgtcgctcttggcaaaaacaatcttctcacttcggtgaccaaacagagagccccagctgagaggcgacctgcctttcggcttctcttcggagccaggaaccagctcttccagtgctggggttttcaccgaggacgacatgctgaagccacagccacgaacgattttacctccaggctgggcagcatgggtcaccgtgaccgcccgggggtggggccgcagcagggactccgggcgccaggaacgaggccacca

SEQ ID NO: 3 - DNA, 470 bp, Homo sapiens
cccacactgaatggattgaatggatgaaggagtgagtgctgtacttagccgggggcgctaggcgctgcgtcgggagcctgggacatttcctcctgctgcctgggaggtaacaccctggactgctggagtctgcattccaggagagcgggcagaccacagaaaagggagaaaaggggtggagggtcaaggcgggcggacacagggaacagggagcggggacagggcttactcagcaatcagagaagacctcacggaggaggtgaggtgtttaggtggttggtgaaggcggaagccccaggaatgggttagtgcccttaaaaaagagacctagaaggctccctcaccccgaccactgtgtcaagacacaatgaggagccatctaggaaccaggaagagaccctcaccagaccccgaatctaccggcaccttgatcttggacctccagcctccagaaatgtgagaaataaatt

SEQ ID NO: 4 - DNA, 669 bp, Homo sapiens
gtgagcatttgacagcctggaacagcactcacacccccaggagagcatccggcagcctggagcggaacccacggccacaggcgagcatctgagagcctgggtcggcacccacacccccaggtgagcatctgatggtttgcggcaacaccgacacccacaggtgagcatctgacagcctggaacagaacccacacgcccaggtgagcatctgacagcctggaacaacagcctgcaccaccaggtgcgcatgtgacagcctggaacagcaccaacacccccaggcgagcatctgacggcctggaacagcacccacacccccaggtgagcatcagacagcctggaacaacacccatacccacaggtgagcatctgacatcgtggagcagcaccccacacccacaggtgagcatctgacagcctggagcagcacccacacccccaggtgagcatctgacagcctggaacagcacccacacccccaggtgagaatctgattgtctggagcatcacacacaaccacaggtgagcatcggagagtctggagcagcaccaacatcccaaggtgagcatctgacaacctggagcagcacccacacctcgaggtgagcatctgtcctcccggagcaggacccatacctccaggcgagcatctgaacccatggagcagca

SEQ ID NO: 5 - DNA, 440 bp, Homo sapiens
ctgacaacctggagcagcacccacaccccgaggtgagcatctgacctcccggagcaggacccatacctccaggcgagcatctgaacccatggagcagcacccacgcccccaggcgagcatctgaccgaacagagcagcacccacaaccccatgcgagcatctgacagcctggaacagcacccacaaccccaggtgagcatctgacagcccgcagcagcaaccacacgcacaggtgagaatctgacagcccgtagcagcacccacacccccaggggagcatctgacatcctggagcagcaccgacaaccccaggtgagcatatgagagcctggaacagcacccacaaccccaggtgagaatctgacagcctggaagagcaccccacatcaccgggtgagcatctgacagcctggaacagcaaccataccctcaggtaagca

SEQ ID NO: 6 - DNA, 438 bp, Homo sapiens
tatagggcagggggaagcaagcaaggggcagaccaagacctccccccgccggtcttggtggaaaggcctggacttcgttctgtccacccctgccaggtggggtgcagtgtcactgcagctcccgccccgtccctgagtcctcctcaccccatcccccaggcaagaagcgttgttccttcttggtgacctgggcacaatgtacagcctttggcacacaggcatctggctttgtttttgtgccctcctctccctcaaggctgagtgtggatttccatcatctctggacacccctcagcccaaagacgctctgtgccatggagaagcccccaggataaagctcgggactgcgcagatccccgctaagagtagatgagaggcccagtgtggccggacgtcccctcagacacagtgaagtgagcacctccatgcaagcaccgg

SEQ ID NO: 7 - DNA, 443 bp, Homo sapiens
cccagccgcgcctgcacacgctcattacacatggcctgcaccggccagccgggctctaacatgtcctcaactttcaggaaaccagagcctccgagtaagtatcagatgagttgctaatgttggaaaaaaagcgagaaacatgcttgggggctctgttttacggctgcacttgaagtcggaatctgcgatcgtaacccaatgctggcctcgccaacgtgttttcggtattgtcgggataattgaaagtccagacaaatttggctatcagggctccaggcagcggaatcccatggtggcccctggtgtgatgagctcgccgaggccgtctcaggtcctgcgcccaccacggtcttcgggagcagctgtcggctttgcaaaagccccaggactttccgggtggacatggggggcttggctttgtcaccgggacctcttgaaaggca

SEQ ID NO: 8 - DNA, 709 bp, Homo sapiens
aggagtccacagaagcgatttcatgttgggacgtgccctgtgcccgctccggctcagcctggctctcaggctgtggggagaggtccccaaaggctcacaagccctgctcctgcccaccagactgagcccaagccagggctcagggacctcttgacagagctgattggcaggcacagacagatcccctgggactgaggcttttctctggagcctcccacattctgggggcgaggaccccagctgtggctgcatccacaggcaccaggggaagggactggcaccaaggcacctgtgcagaaagtgtggggccccccaccgagcagggctgcctccagctggggttgcgtggcacttccccagcaggaacctctctcatgtaccaggtcaaggctggttgagaaacatagagccagggcttgggatgagcctagaaccaggaagggctgacagcgctcctccacaaggctttgcctgggtgttccctccctgtgcaggaatgcgctgcttcctggtctcccatggcatcctccacctgcccaccccgtttaactcgccctcagctcagtctgtctcccgatcttttcattttcttcatggggcatatcccacttcagcactgtcttgttttgaatttgctgacacctgcctcaccccacccccggcccctgctctcgactgcaagccaatgatgtgaagggtgtcctgacct

SEQ ID NO: 9 - DNA, 482 bp, Homo sapiens
agcgaggaagacggccccgtcccagggctgctgagccctccaaggcttccggagcaggggtggatggtgagtgggccctctccctacgagagccgagcagggtgggtgtgctgtggccctcatcttcctcagttttcccagtgcctgtctttccttgtgccaaggacgggagtctgggcggcttctgcccaccaccaggtgagacagctccgtggtttctaggaagaagccacccgtggaaccatgtaggtgatttcgtgcccatttaaaactacgccgaggctgctctctgtgccccttctctcccaggctttccagtagagtctcggctccctctgggagccacctgctgcagccatgggtgcatcgggtgcgtgtgctcctgggggacccttttgctggggctggggtcagacagcgcttcacccaggccgggattctagggagagcggtgccgagaagttgccctcgtagccaaggtc

SEQ ID NO: 10 - DNA, 450 bp, Homo sapiens
tcaacttccagaggccgccgcttcatgaattacacaccatgcaaacgtccggccagcggcttaagtataaagcgtgagaaacacaggagatcattccgtgttcgtggttttgtaaggtttgaaaagcgatttaaaataattttaggttgtcctggggttggcgaacagtgtaataggaaatttgagaaggcaaagtttttaaactgaaaggttttgcctccttgtttctaatttttagaaacatgatggatgtcagccgtgacgttgggccgacagcttctgggaacgatttcccagctcgctcggacccaggcgggggaccctgccaggagggcaccgagggccatggccacgtacagaaagaggacggcttccagtcttcaaattggaaacctgcacccggcctcccttggctaacctcaatcccataagcaatccgaaggcattgct

SEQ ID NO: 11 - DNA, 416 bp, Homo sapiens
ggcgaggaaatacaggatgcgggctctgaaatgggaacgctgctgtccccggagcattgcacggataaataatcgtcttagaataatgcatcggctttaggtagggccaggtggcctgagctgcccgacagaggcctccgtggccctgcgtgagggccagattcagctcggctggtcaggggagattccaggggctgggaggggcgttcgtgggcagtggcttcgtggaccccagtgggactttgtggtggtctgtgcagcgcagccaggcccacccgcctgccccagaggcgtgggctgcgcagaggctggggctgtttgcccttccccttggcagcggatgtgcttctctccgagcttcagggccggcccctggtggggtgaatggcagtcacacatcctccaggctgtcatgg

SEQ ID NO: 12 - DNA, 524 bp, Homo sapiens
tttggatgggaaaagtgctttgcagcccagctgctgttctcccggaggccggggctcctggaggctgccgcttgcttttctccgcctgggtggcaaacgtgtgtacttaacttgaaaaacctcagggtcaataagggtttcagtccacattttcagagcgatcttctcgacattttagaaaagcctagttccctctgttaaaccctgtttcccccaggatgcacccttgtcactcagcacttccccatctgtccccaccacccacttcccaaactcgagggccctgctccttgagggcacccctcctggaggctttgctgaagcaccgattgcctgcacacaccagccccgccccaggtcagtgggcctgggtggacctgtgaattacaggtctagcaagtcctcaggggatgcaggggctgctggcggggactccactcagactcctgagccacaggggctgaaacccacagccgggtccacccaacctctaaaaccttccggtcccacctcgctccaaacac

SEQ ID NO: 13 - DNA, 427 bp, Homo sapiens
tcttccagggaagctattgtgtcaggtaaagaggcggccatacatcagccggctctgcccctcaaatagataatcaatttccctgaaagcatcaataaccagagtggacatttattgccccgtggatggtttatctttggggctggggacagcagggccgtgatgaataccaaatctgccagggaacacaattaaatgctgcgaagataaaggattttatatcgtgcacaaaaaagcaatcaaattcataattgaactccattgcatgaaaccagccagagataaatatcacacccaaaagtgtataacctgggtgccgtgttatgggtcaggcgacagtgttgccaatacccagtagtgtctccctctggaggccccggtgcgcccctctcgctggggtaccagcttccaattgccactctctctc

SEQ ID NO: 14 - DNA, 607 bp, Homo sapiens
gacggagccgccggggcagccagatgtgtgggcaccgcacaggcaaagccggtggcatgctccaatgcaaggccatcttctttacctctgcaggaacaaatcggatcaggtgaggctgattctcctgcatccgtgcctcagcccttagtggtcccagatcattgtggtggccacttttaggacagggccagagccttggcctcgacctcgcccagcaggcaggcagccctcccaggacccttgggtgaaactccagcttcctcctccaggtagctcaggtgccagcaggtaaggctgggagcagctatccctgaagccaacccacttccttcctgttcagagctaagagctgccaagaagcacggggtggctgctcagattcatgaggtctgctctgcacttttacaacctaataaaatttgggggcatctgagaggaacaccgaggggcacacggggacgttgacatgttgtggtggggtcattcagacaggacctgagtgccgctcttcatttggtcctgaattcgggtgtgcggtgaggctgcgcctggcaccccggatccctccacggcttcccccttatctagagcatctggccccatcttg

SEQ ID NO: 15 - DNA, 391 bp, Homo sapiens
gcacccggcaccctgctgcccggcggggcctgcagtcagccaggacacccggcagccacaaggccgccctctctggagcctgtggtctggttcggctagggtattgtgatctgaggacccttagaaaactggccacccgagaacttttccagtgtgactgctggcctctctggcagacgtgtccaaactcagaaaacctctgctgctctgaccacagggaacgtcagaggggacggggcagcggaacaaagcacaaagcgtccctgcgtcctctcaacgtggcccagctcctgcagacactcagcgagccgtggcctctcccatcctcccacgcttccctccggccggccctggagatccgtggagccacacggtgccgagcgcggcgctg

SEQ ID NO: 16 - DNA, 477 bp, Homo sapiens
catcaggcggagggaggtccgacgccgtgtccacacccaatctgccctccggggagacgggagtgggggaggcaagcatttgaaatcctccctttccaacagagaaacactgttggggagacggtcagaggaggctgcccccagctctgcttttccttgcctgggtgcagagtatttaaaaataaaagccgcagctgccaagacagcccatggagcgtttgctgtgtctggacgctgttctaaggagcgcttcacatagatgatcttagcctgccccagagcggccccaggaggaggcactgtctctaaacccactttacagaccagcaagctgacagcaggagatgtgaagtcactgggcagatcgagcagctcgggaaggtgggcacgggacagatccaggctcagaggtgagagtcctggagaccgggccagtgtggcggttccttatctccagctctgggatcgataaactgt

SEQ ID NO: 17 - DNA, 602 bp, Homo sapiens
cttgggggccagcccgcttcgggagaaccgagcctctgccctgccgtcccggggctggacacattccctctgcagagctgcagctctgtcaagaatgcgaggcccattttcacacaagtgattcagaccacggggaagccactttcagatgcattcgactttgtgttttgcttttacacttacaatgaagaggaaagaattctctttcgtgagaactttttgctcttgggagaaaataaagaaagtcacttccattcccatatgcttttcttttgagaaagcctcacgataatttgcttttcctctctccaaaagaggtcaagaggaggcagtgcgtggaggacagagcgcatgagccagccagatcgtgagatcagagataataagatgggaaacattatcagaataggacatgttctttcagcatttctttaaagcagaagtttcttgtaccgagaaagtgaaaatgccataaactatgcccccgtttctggcccctccatgagccccaggggtgtattcagggccagccttacctgcctggaagacagccgggagacctgtccataccactcagtaagggcaggccaggctcaggtgtg

SEQ ID NO: 18 - DNA, 660 bp, Homo sapiens
ccgggttcctccccgtggcttccgggggccctctggttgtgggagaccccgggctcctgtgcaggcctgtgttaggttcagggttccctgtttcagcagcttgaccccgagcaggagccaggccccagggaaagctccttgccgccaggcaggaagcgaaagggaagaccagagtcccccgagccaggtggaggggcttgtggaggatgcctggggaggcctaagggggaggtcagagacccagaaagccccctacacttcccagatgctgcgttcccagctgcattcataactgcagctgcaaagcctctgccacccactgggggtgtgacaggggcggtgaccacaggcagggccagcgtgcagggagagaggtcccctggaagcaggaggagaggacagcgtcccctgggcagaggacaggcccagtaaccctggggaggagggggcttcagaggcccccaccccgtgtcactgagcctgaggatgagaggggaccagagggagaggccgacatcggggccctgcctgaggaaggccctgctaagggagcctgcctggggccctgggccacagaggacggcaccgagagggcttcaggcctgctgtccggacacttgtccactaattaggattcccgcgtggggccgcctgccgcaa

SEQ ID NO: 19 - DNA, 544 bp, Homo sapiens
ctgcctaaaacagtgtccttggtctataaaatgggctagctgtgatgaccgggcagggccgtggagaagatggaggttcactgcacacagtggcacctgcttagggatgctatatttttatatcgtcttggctggggatttcctccttgattttctagtgagttctcttcaaagtggcccttgtcacagaactctaggctatggattggcatcgtcattctcccagttggcactgcctgctgtccagcccctgcaggccacatggaagccgcctttctctccacaccagccagtcctttccagcacagctccgaactcagtgcgattggcaaggcgagctggctgactgacttttgggatagggccacaccaagcctacgcccagcaatgcctacacaggtctggcaggctggctctgtggctccaggcatgtgggcaggatggagcaggcgggacaggggttgtcttttcttaatcacctcccctggtgcagccggccatgctggactgtggtgaccccagagcctggctccctggttggtct

SEQ ID NO: 20 - DNA, 477 bp, Homo sapiens
gaagcaaggagctcgggccttggggagttccctcatgtcattctcatgccggataaggtgctcctgtgtccaccctcctaccttggtccttacttacatgagcccaactgggaatgtgctccgtgattcaggtccacttaatttctagaatgctcaggtagaaactaatcattaaaaacatgtaaattaatttaccccaggagcttaatagattagccatcgacttttttcctccttttggattctaacctttcacggaaattaatcacaaagggctcaaaatagccaccgaggattaacatgctctgttcagtcttggtgagtgggaactcaggcccagcccctcagagaggaggaaggatccccgcatcttcacattcccttttcccctcccagcagtcctctgttggattcgatacagccaccccgggagtggctggaagcccctggccacctgactcttccttcacagcagtt

SEQ ID NO: 21 - DNA, 656 bp, Homo sapiens
aatgaatcatggccccatctgtagaagcttaaggaaacgctcagggagccggctgttctgggccaacagtgaaggacaggatacatcttccaggctgctatatccactctacaaggagcaggtcagagactcaggagacagcccagggctcggacagggagcctggcttgatttaggtcagtcttaaaacatcactggaattgaatgacaacgtccagctgcagcctctgttgctggttagaaggttattcacccagggcggatgggcagcgccactcggggtgaggcactggggcacccgtgctcctctgtgtccccccagctggaggttagcaatgacctggagatcagctaatgcatcacagtgatgctgtttgtggggctctctgtgccagaccaagtgtgttttgtaagtatctgctcatcctgtgcttgtcactgactcctaacccagcaaaagttgggggaagagcaaacatttcttgaagcaccccctgagctctggcagagcagatgggagagggatctaccttcagcccaggtctttgtttttcatgcactgtgtttatccacaggcacctgaagctggagtccctctccttcacccgggccctaccccagctagatgaagggcttctcactccactcccgggcac

SEQ ID NO: 22 - DNA, 491 bp, Homo sapiens
tagagactctcatttggctctagtgaggctgctctctgaagtcagggtccggagctagccccttgccagctgtatgactgctcttctcaaaagtggggggcccctgggggactgctggggtcactctccctgcattccttagagtggccccttctccaggctgacgtcaatttcactgctggctctcacaagggcaggtcccacatccacagagacagaggcagcttggggctggtgaatcccagaggggattttatcaagttcatgattttataaaagtggaagtgagctagtatctacatgagttgagctttaaggagacaaagcatttctagtgggttcccaaatgatacatctactcacaacttcagagtggggccttattggaataaaggtcttagcaggtataattaagcaaaaggtcttgagatgagatcaccccggattctccaggtgtgtcctaaatcctatgacaagtgtccttataagag

SEQ ID NO: 23 - DNA, 351 bp, Homo sapiens
cctgaaacctgcctgcaggacctgggatctggagagctgcccgctggcccggaggatgggcacccatccaatcttggcttaggaaaggggctgcagaggggcgggtgaggggtggcggggatgcagccccaccctggccagtgcctcatctcctgcctccgcataggcaccaagtctttcaacatgatgtccccgacgggcgacaactcggagctactggctgagattaaggcaggcaagagcctgaagccgacgccccagagcaaggggctgaccacagtgttctcaggcatcgggcagccggccttccaggtaggcgggcccagcaggagcctgcgacccggcttccct

SEQ ID NO: 24 - DNA, 407 bp, Homo sapiens
cagatcaggtttctcctgaaggccctacctggagtgatgttgctgtgcccggtccctagctccgtgcctggtaagatgttcacggggcttagtgaacacgtgtctagctgagccccactgagctctgatgggggcattttgttgcttgtggagcaagaagccatttggatggggcagaggtcagctgccaggactgtgtggcctgacctccatcctgcaccatgcccagcagagtcaaggctgtcagagggattttctcccgcgggtcttcctgtgcaaatcaataacccttcaggcagcagcagggagggaagcatgcactgaggaggcaagggtcagggctgttcctgcagcttccggcaagtcctttgcagggtttttcagtatcttccgctcgagcaggtgct

SEQ ID NO: 25 - DNA, 563 bp, Homo sapiens
aggatctggtaagaggatgaatttggctctaaacaaatgcccatggccccggaagacctgtcggggccaccaagttagtttctctggcaaaatttgattccttccatctcaccactatagtaactgttgttgttatgttaccaggagccacacaaaggttgacccatcaaggtggagccacgttaccaggagccacacgaaggctgaaccagcaaagtggaggaccagctgtttaattgactcgggtctggtgacaggccaacatggagagagacagcccagcttcaagaacagcaaggcagccctttgcacagttcccactggggtggggaggctggagatgaggactgcattccacatcagagttgttttatttctggatattcacagacagctagaagtcagaaaaataagccaaagtgggaagcccctggcagaccagctccactggcttcctctccagagtcactgttccgaagtgacaggtggaagctctttcagttcctccttttccgggtacacttcagacaccttattcatcttggtgaaaatctggttgatct

SEQ ID NO: 26 - DNA, 330 bp, Homo sapiens
cttccttcttatctctcaacctcatctctgtccccgccaatattcagcccggctaagaagcttaggagctggctaaggctgctcctccacctggatgcacacacagggctagggctgtggagaggcgggagcaccagcggcccacgccgaggaagataggcgctccctcctggggtgggcagcgcaggtctggcctgcgggtggggggtggagagtgagaggcacgcggggcaccagctcagctcccctctggggatttcgtactcgcagtttaaggaaccggtggagcacgaactccactcctgagctggcgcaagcgtctcccttcca

SEQ ID NO: 27 - DNA, 549 bp, Homo sapiens
ggtctccttggccagcagcagggcctgcccctcggcttccagctgctcccggcgggcctcaagctgggccagctgccattgcacctcaaacaggctgccctccagggcttccttctccaagctgcagcacatacagtgcgtgaggccctgggactcagcctccctggccccaggaaactgtggatctcagggagtggccaggcatgggccaagtgccaatctacctgcctggccaggcccacactccctggggaagcctgagcccacagctaggacgagttggcaagccagactccttagtaactgcccctgggggcccgtgcctgggtgaggctgttcatggcgtggccccaggaggaccgcccagggcgggttgggcagagcccaaggccttactgcaggtgtgtggcctcctctgacacggtcctgccttcacgctccgcaggccgccagctgcacagccaggccagcgtgctccttggctagcgcctccttctcccgggccacttgccccagcgtctccacctgtcactgggcctcccaccgggc

SEQ ID NO: 28 - DNA, 480 bp, Homo sapiens
catgacaaatacatagaaactaaagctctattattttgctgtaatgggccgggggaatagctctgaaataatattcttctgtctgcaataaaggcttttctctgaagctgtcttaatgctgtgaacaaagcctggagatctctcaagggctccttctcttgaattactgcccctctcaaggccttctgccatgataagttcaccaaccacctggcgggtggatagggaccgagacggtgaagcagcggggttgagggagcataagctgccacgcacaggctggtaggggtggagaggatgggtgagtgtgagaaggcacacccagaaccaacagtgtctgggtggcagttgtgccatgctcagtccccacactctggagatgcaccttatctgtcaacatctacacttaggcagcagccctcaggaacaccggaggtcaattcagcttgagagctggactcctcttaagtgaccatgtgg

SEQ ID NO: 29 - DNA, 618 bp, Homo sapiens
ccccagccccaaccttcctgaggcccccagagcatgtgagccagggctccggttccatctcgcctctgctgagcagcggagcctttctggtctaacccccatgtcacggcttccctgattccaatttggcggaagacagtgggtttgcagagtggctgggtctgcactcggcttcactacacagccgctgtgtggccctggacaagtcgcttcacctctctgggcgcccccgcttctccatctgacaaatgcggctgtaatttacccttcatggcattgtccagtgggatcatctgagcaaatgtgctttgtaaatggacaggtgctccatgctgccgctttcaggtaatctacagcctcctccacctgactgcactgccagcccccttgcaccaccatctccttcagatcttcccctaccaacagtctgccccatggcacggagctcagaatacaaagtaaccactttcctcccgtgacccctgcctcggtttataatgacacgtcctcaggccaaatccttcactattggacaactgccgatccccgttatggggctggaagcctccggtggggggctttgttggcttggtcactgcaggcacgttgtaggggctc

SEQ ID NO: 30 - DNA, 583 bp, Homo sapiens
tcagccagggagggcgggacatgctagactgagaagctgcctttcttcccggaaaaaatgaccttgggccaggactggcaatagatcggctgggatccagacagcctgggtccccagcggccctgggtgctgcagtcagggcggaaatgggctctcaccccctcttgcactcgcatccgagggagtaaccctgcatttgggacaaatccaaattaaactagactcccgagaactgctcctaggtgatccctgaaccacatgggtctccagtgtccaagtgaccctctatctccctcccaacaaacctgccccagggagttctctggccaaggggacatacgcgggctggagggttcacatcttagttcggttgcttcctggctgggtgaccttggacaggttgctcccctgctctgagactctgggcagcctaatgatgtgatgccccagcagccttggtgagaggtgaggggaggaggggaattgcaaagcacccagctcatgttgggcaccgcatagatgacagccccttccggctgctgctttccacctgccctgctggggatcagcacacagactcccc

SEQ ID NO: 31 - DNA, 749 bp, Homo sapiens
atcaccggctccctaggggcgggaaccctctttgaaaggggagcaaggccgggtgcggtggctcacacctgtactcccaacactttgggagtttgaggcgggcggatcacgaggtcaggagatcgagaccatcgtgactaacatggtgaaaccccgcctctactgaaaaaaaaaggggggagcaaaggaagctcctttggttgggctgagcctggaaccccagggagaagacccattaggaaagacttccagaaaagacaggtagcccttgttggggtcactatcaggataaaagaccagagttattttgtattcacagatttgttacactaattgatcagaacccagggtcccactgccagtggtggccagtgttgcctcttagagattaggctggagccacagaacctggccaaggtggtccaagcctgcagccgctcccctgttggtgggttcttcaagaacaactcgggtgcttaggcttctcagagctgaaggagaggcctgctgtcatgagagcggtgcaggaggaggtatttgggaaagcctggagatccccaacctacttcttcccccattggtccttgactgcaccctggccaggggcgatgggggaagggaactcttggttctggaataatatcttaaccacctcctctatgccaggcccctgagcactttacacccattaatctgttccggttctcccaaccactctgcaaagtaagggttattattcccatttccac

SEQ ID NO: 32 - DNA, 346 bp, Homo sapiens
gtgaggccgcgccccgccgcgttcagcgaagggaagcaggcaccccgcccggctctccgctgcctccacactcggggaccctgggcgctcacgtgttcccctctgggcctcaggattcaaggatgcgccctcttgcaaacagtccgtggcctgcgacagaaagaaaacccctgggagcggtcggggtggggacccacaaaacgctgtggctaaaccgcatggcgccaagcccgatcggagcgtggcgtgggaatcgagtcaaatttaccctgctaggtgcggctccaacaaggtcccgggtactcctctaacgcccagtgagggcagcactgcctttgtccctgcc

SEQ ID NO: 33 - DNA, 396 bp, Homo sapiens
ccggacgtgatgcccccggcccacaaccatccccacactggccgagccccggatacctgtatgggagcccgagagggagctgaggagccgcgagtgctggctgtggccgtcgtgaacctccaccacgtcgttgagggccgtgtgaaagaaggcgaactggccaaacaccactgtggaggagacacagtgtggggactgggcaggcacgctgggggcaggagaggggatctaggggtctaggggcccaagccaggaggagggcagccctggggaagttgttggttcctgggctgtggcttgctgcactggttagtgcagtggcacagtctggcagtggccagacacccggcacagggaggcggggagctgggaaggccgggctggccttcacaaggg

SEQ ID NO: 34 - DNA, 807 bp, Homo sapiens
ccccttggttctggcggggctcacttgtgcccttctgccctcttgtgtccggaatgtattctgcagcccgtttgagaaacgtcagcactcggccgtccttttggggagactgttctgcgattcccacaatggggctgtatataattgggaggcgtctgaggaattgctcattttggaatgagggtgaggagtcagaaaacatatgaacaacaacaaccaaaacccataccatagtccttgggcacagtaacaaaatacaagcagatctgtccactggtgtagttctgggggtagttgggggacaagaccactccgtccgaacccacatactgtcccccacagggggctgaaagagaaaccagatagagagtcaggtgaccttgtgggcctcttaccagtgaccatcctctgttctgccccagcctgagcttctggccccagtagggctgccctgaggtgggcaggccctagcttggagaaggcaagtcctgcggaccctgccctgcccaagaacgtgcacccctggcctggcatgtagcctgaaacttgccctgaagctaccgagggccctcttgggcaggtgtcagttcttgccctctacctattaaccctggcattggtccctgaagtcacagccttagggagcacattaggaacctcttctgcttacacgtggagcctagaaatgatctgctgccctggctgacatctaacctggacagtgagtgtcagtgtgaatagggagctgaagcttccccggactaaccccatggggagtggccagcccatgtgacccagccaccttct

SEQ ID NO: 35 - DNA, 573 bp, Homo sapiens
aggcagagacagaatgaggccctgctttgttggggctgtgtggggtggccggagtctactggcctggggtttcttggatgtgcaagataggaggctgcttttgacaaattcctttgtgagcaaagtcatcacgagtgtgtgggctcagagacaggccctccatgccaaccaccacctgcttccctcgtcccacaccagcagccagaagtggaccatccacagcccctggtgacctccccccaccacgcctatgcttcagatctggggtggagactctagatgaaatgagccccccacctcagccccagatggagaaatgactcaaagaaagttaaataaaaggtgggtgcatgatcagaaacggtaaagaatgtaagagaaagctgcagtcaaagttcaaagctgggacttcagctcaaacacagacatttcttctgagaaacctcttcccatcagatacttctcgagccagagtaagcagtcaccagagcatcccctacccagtgccacccatcaagggcaccgggcagagatggcacctgtgatggttctggtaggtggtgcaatgctctg

SEQ ID NO: 36 - DNA, 318 bp, Homo sapiens
gagtctttattgtttgtggggcaggaggagtgatcatttaccaccgctccggttcgcagggagaagcatttccactgaaacatcctccagacctagctcttcactcgctcacccccacctccccttgggaagaggaggaactcctctgattagtgggtttgggtgaaggcactgaacatgcttagctcctgccctgcctctctctgggttctgcctgcctgcagaaaattccctgcatcttttttccactagcggagcgggctctgcccgggcgaggcagatctgcagttccagggaggaggttctgtgttctcctgt

SEQ ID NO: 37 - DNA, 386 bp, Homo sapiens
tcaaagcagccagaccacctggtgggaggcggcacacactctgccagtccggacacgggttagggagtggcatcacattccgccgagtcattgcctgcgaagactaacgtcacctctgatgtcagggctccaaggggcttcgttccagctccaggctccaggaaagggcgtcgcagggcccagaggttacacaacttcccccaagatcgctgtggaaatgactggcagagcagggattcaaacccaggtccctgtgggtgcctctgcccagccgcctgcagggtctgcaggtggccatgtcccacgtgtcacctttacagacccacggctgagagccggcccagccttggcctggcttccttctttctcatctctaaaaatgcctt

SEQ ID NO: 38 - DNA, 685 bp, Homo sapiens
ggacgcggctacggcgggactcatgcccgggccagttagcgccacctaccggccactcccttcctttcaccgttcacattgtcctgctggcccgcgtcttaaacccatttcacagtgcccagaagtagtgggcattcgtacccaggaagcgattctgccttcgttatgcccctgtcctttccaaatgcctggaggatctagctgttagatcctcccagctgccactgcctgttttaaccataagctgcttctctcgaatattcagagtgggacaactccaaagaccagaccaggggtcaaggggttcaggtcagtgccataaccatttcttgctggaagtatgtgaatagtgactggtacccaccagactgccaggaatatgtctctttggccccaaggcttgagccacaaaatccagctgcagcgaactgccatctcttaagacataaaaacatacacaaccgccactgttaaaaatacatttatcattaaaatatattacacatggagacaggatgcatcatatacagtttggaagacttgctggcccagaaaatcccacttgtttcaccgaacactcattttttcagtgattttacattttatttttagagacggggtctccctctctcacccgggctggcgtacagtgatgtggtcataggtcactgcagcctcaaactcc

SEQ ID NO: 39 - DNA, 423 bp, Homo sapiens
tgatgcccctcgcgcacccctgatgccggtcgcccacccctgaagattccgggtgggcgagggaatcgtcagagggatcacgatctttcagctactctactcctatgatcgactgcatgtaacagagattctaacgttcaacgacaagaggattctcaacgtccactccaggctcacagatgccaaactggcattaactggcgagccgtgctgagcggcggcagcctctgcatcggagattcctggtggtgcgctgccccaggggagcgaggatagctttgcagttctccagcagttcgccgaggagcagctgagcgccgaccatgtcttcatttgcttccacaagaaccacgaggacagagccgccttgctccggaccttcagcttttggggctttgagattgtgagaccccttgtccccaa

SEQ ID NO: 40 - DNA, 876 bp, Homo sapiens
cgtcctcccagtcatgtcctgggaatccggatggcgcaggaaggccacccggtgaccctaagagtggccacctgtcctctctgaactggactttctcttctggcccttccactcttccatgtgtacacatatgccaagtaaatttgctgtggcctcctggaaccatctcccccatccctctcaccccttaggaagagttctgtgtccctccattacccatacgcccgcaggagaaacaggcttctatgccctatgaaagtccttcctgagatctaacttccatccctctagctgcaaccacagtctctttcctctttctgctgcctttgagcaggtaatctggagctggagttgggaatgatgagacactccctgcagtgagggctcctcacaaggtggggaagggacctttgtagtcccccagctgggaagagagccatgggatccagaggtgatgctgagcttgaagattagatttcccagaacagacccagtttcagatattctgggtgttaagggcaacctgaattcaaattccatttctgctacagaacaagagcctcaaaaagaccccctagaaacacccccagggcgcttgcaactgctttcctgtgcacctttcatgtcctagaccttagtcccatttcccaggatctatgattgagaagaaccagccaggcagttccacagagggaggacagtgctgcatattttgggggtgccatcttcagctcatagagagaagatgggttaactcagcccaggcaccacatccctagtggggccttggggtccagaaactgtccccagcttccagtagaggatccgggcataacctgccctggggcacccggctctggccctgggcacacctgg

SEQ ID NO: 41 - DNA, 567 bp, Homo sapiens
cccctctgtggcactccagtgggcaaggaggccttggaaggcgcagggccggggccagcgggaccactcaggctcacctgggactccagagggaaggggcatctcaggacgactccacagggtagggggctcctagctcaacatcttccctgctttacccaagagaggtcaagacgacaggactgagagtatgatgtgctcgccaccccttgccctcaccccacgccccacccacacagcccctacaccttcaggaggatgggatgagtcatcattaggtcactgggtcatcgagtaagcagtgggggccacagggtgcagtttcctcccttcctccccgcccagcccacctacggctctcctcctactctctgcccccgctactttcctgtgctctgatgtgggagggatatcgctcattgacaggtttcaaaactaaaagtagagcgcatttagtcttctcctgtggctgagctcagctcccagctcccagaactttgccatgctgctgcagggccggcatgccccctcctcaccctcacggggagggcccccatggtgttagcc

SEQ ID NO: 42 - DNA, 406 bp, Homo sapiens
ggtgaacccacagacattcattctcactggccagacgatgaggagacaccggcacagacacaccgacacacacgcacaacttgcttgcggtgctggctctcacttgccttcagacatctaatgacactcggcacttcaagacatcacaaaccttgcagaggcccgagtgacagacctggctccgctctcttccttcttccagctccccctctactcacctggggtgctgcggttcgagctgggggtgtgtaactcgggggcagctctctgaggggcggcgatcgagtcatgctggcggggatggggggcgcgggcgcccccaacccgcgttatctgggcgctcccataggctagcccggtctcccggtaccgctgggccccgcgtagggattcagtggggccgcca

SEQ ID NO: 43 - DNA, 271 bp, Homo sapiens
ttctttcaacccgagctagggccccactaacccggtccctcccagagcccggattcggcctcggtctcggatgcgacgggtatcagacccaagcggcaacgtcgctcacccgaagagggggcgggaaaatcttcccaggaggtgatcccgaatgtccccacccgaggctcgaggcctggtgaatgtgcccattgtcctttcggggcctctcccttggagcccggcggccgcacccacgaaactgaccagaaatggatgaagccggagtgca

SEQ ID NO: 44 - DNA, 626 bp, Homo sapiens
tctggccagtctgcggggtcaaagtacccagtcacagatttgaccttaccggagaggcagaagggaaaggagcccacaggaggcatgcgggctgggcaaagcgccctcggcggcgtctagagcactcggagctgccccattgcctccgcccaaggtcgcggcccaaagggagccctggaaagttcagccaggggccgaggagtgccagggttggcactacggggagacttttgcgacaggagagagactaagagctcgattccaggtagcagtgggagcccgcggcaaagaacgcccagtcccgcaggctgcatcccagaccgcagcgcctagctcggaaaagctgggcgggggctgcaccacctgagtcgcaaggacagcacggcctccccctcctcaccagtgttctccacccccagaattggggcgcggagagccaatctgggctggaaagggagcggcgcccacctttgggcagcccgagcagccccgctccttttctgtcgcagaccccaggctgggaggcgggggcgccaggtcggcccaagctgccgcagccactgctttaaagggagccggggacgcggcgctggggactgggcgaggcgggtggcagaggcaagagg

SEQ ID NO: 45 - DNA, 887 bp, Homo sapiens
cataccaccaggttgcaattatttctgtcaacctgagctatgctgagtccggtgggctaatacagatgaggcagggctgtgtctgtgtggtgtagagcatggagtcagtctgtgtcctgagctgtgatgagatcattctgtgagagtaaataactgagcttgtacttgaaagttgaggacaatgtggagaacaaagagctaagagcacaagcaaaggtccagaactagaacatcacagggtgtgttttggagactggatgatttgagtaaagaatcatgaagagcagaagtagttatcaggctagaaagttaggctggagccagataaggatgggcctgagtatttggaatatatactgttggcaacagatgactaaatactcgggttctcttgaaggagtacagggcagaaatcaggagatagaatgagaaatttgacatttacaccagaactttccagccacaaagagcaaggtagagtcaattaatgcactgagtctagttagaatgattgggttaaggtgagagtggccattcctaacacttagcaaaacagtgtgtatgcatatgcctgtgtgcagttatttgattatacatacactttccagctgggctaggctggcctatctgggcccagtcaactgctgtggacactgagctttaacaccaataagtgcgtgccgtattcatcctcattctcatttctttctcagtgcctcttgaccctccaaggattattgcactgatccagaactgctaaaatggagaaagccaggcctgacctgtgcgcacagcacacagcattgatatttgatgtgagattaggtcagagtcagccgggaagccttctaaagcgtggcatacagccaacaaaatgaaaaggcttt

SEQ ID NO: 46 - DNA, 304 bp, Homo sapiens
aaccacactctcccggtccaggccccgctggagaacgtcccaccgactccggggacagaaaggccgtttatgtaaaacgacgtttttcctatttcgcctcccaccctcatgcaaattttgattttcaactcttccaacccttccgcaccctgaaacaaacctcatctcctttcctccctcttctggctccgcgggttactcgccccgcccccacccttcagtcccttagcaacgcgtcctcggtccgtagaaaccggttgccgagctctgcctacctctgaaaggcctagagagcagagttctc

SEQ ID NO: 47 - DNA, 685 bp, Homo sapiens
tgtagtcccagctactcggagaggctgaggcaggagaatggcgtgaacccgggaggcggagcttgcagtgagccgaggctgcgccactgtactccagcctgggtgacagagcgagactccgtctcaaaaaaaaaaaaaaaaaaaaaccccacaatatcaggctacagttatcatagcaattctgcttttaatgcactcaggacaaacttctagctctgcatatatgtcatttcaatgacacatcctgtctcctacccgactagaatgtgagcagccctgtgatctctatgtgctcataaacagtgcctaatgcgaatcctggctcttggaaggcgctcagctagtaagtgaggagctcagctctgaagctgtaaccaagaccgcgcgggcaggaggaaaggacacttcagcgctctgcggttgcaaacacacccctgcgggtgctgccacttagcaaagtcttaggttcccgcttactcgcacacgcatcctgacttcccccaaccctggaactacctaccaggagcggagaccagcagaccgacagcacgcacatgatccccataagctgaatggccgtctcggtcgtgatgcggccccactgggcactggactgagatgccgtggccttggcccggcagcgggacaccagggccttaatggtggccaggttgcaggaaaaggt

SEQ ID NO: 48 - DNA, 517 bp, Homo sapiens
tttactatctttctgcacagtatgttcaaaatgaagctcaaatctggcccggcaatccccttaaaatgccaggaaacctctcatgggtgttaatggtctggatttcccaaatttcttcggggcagaacagaagaaaggtgaaaggggatgctggacacttcctcaagaggaataaaaaggtaacattccctggaccttggaatattcaactttagagttgaaaggtcaaggagaaaaacatcctgagaagtactaaatcaattctcctcggagattttgccaaatacacttgccatgtgagctccacctcttaaaaaatccagtcccctacaagggattggtccctccgacgacctcggcgaccaggggcgctgagcactcacctaggagcgggaggcgtttcattggcggtggccgcagtggtggcggtggcggagagcgcggcggagtgagtgcggctgtggacccgggaccgggcgccgctgggcgggcgcggcagggtcccggatccgtcctc

SEQ ID NO: 49 - DNA, 446 bp, Homo sapiens
gctaattttttttgtagttttactagaggcagggttccttcatgttggccgggctggtttagaactcctgaccttaagggatcctcccgcctcggcctcgcaaagagctaggattacaggtatgagccaccacgggaagagcattctttagacaaccaggaaaacttgaatttagtgtttatccttaactgagctttgtggatcatgactaacaatcgccaatagcttgggaagtttaagaaggactttttgcgtttaggaagtagagagggcagagataaacaaaggaaccagaaacggagcattgggttccaggaggcacaagaggcagcagaagtgactgaacattgatatcttgagctcaagagctctaaataagcccaggcagttcaaagccgggctctaactgtggctgaaattgctgacctgcgtatagtttataatct

SEQ ID NO: 50 - DNA, 391 bp, Homo sapiens
tccgaactgcggcggctccgctagacagttgcagaagctggcggacggccgggaccccctgccttgcgcagccaggcgtcgaggaggcggcggcgagtgctgcggtgctggctgggtggccgagtggtccccgcagcctctggacccagccatgctgctctggatgcagggcttcgtgctggaggcggtggcctgccaggataacgatgactacttacgctacgggatcctcttcgaagacctggattgcaatggggacggcgtggtggacatcattgagctccaggaggggctgagaaactggagctccgcgtttgaccccaactccgaggaggtgagaccggaaggtggcacaggcagcctggggagacgttgggcagagggagaagca

SEQ ID NO: 51 - DNA, 391 bp, Homo sapiens
ttgcttctccctctgcccaacgtctccccaggctgcctgtgccaccttccggtctcacctcctcggagttggggtcaaacgcggagctccagtttctcagcccctcctggagctcaatgatgtccaccacgccgtccccattgcaatccaggtcttcgaagaggatcccgtagcgtaagtagtcatcgttatcctggcaggccaccgcctccagcacgaagccctgcatccagagcagcatggctgggtccagaggctgcggggaccactcggccacccagccagcaccgcagcactcgccgccgcctcctcgacgcctggctgcgcaaggcagggggtcccggccgtccgccagcttctgcaactgtctagcggagccgccgcagttcgg

SEQ ID NO: 52 - DNA, 589 bp, Homo sapiens
aaggggcagaggactgggctgccagctgagaggtgctgaacggggaacccgggccaaactgttgtatgtgtgtggataaggaggccctcttggttgctcaccctggggctccttggctgctccactttgtccttagttcctgtgcccgattaggattggggatggtacttttctcctgtggctagattctttgggttccacctagctacaggggcaggaacaatggtaccaagccctcagcctgttctctttgattcttaggtcttttctcctggcaccctgtattcatggccttggcggtgagtttaggcttctatctgatttgtttgggtattcctcacctcattctcccagggaagcacccagctttcagaaagctcagcctttctaaggctaagacaggtggaggaaacatctagggctcatctttccctgcagcctatgggattgggaaacctggagggctgggccataggcagttgtcagatggttgaaaagggcgggagggcatggaagataatttctttctccattggggccggaaaggagaaggggggagaggtgttatccggtcatctcccgacttgtc

SEQ ID NO: 53 - DNA, 762 bp, Homo sapiens
actctgctccagcccaggtttcggggagccgggatccacttgggcaggccgggagcctcagactccagacttttcatggtgcgctccttcctgcttactcaggaggaaggcgaggcagtccagcatcctgggtggagtggaggtgtttcgagtccatatctaaatctttttcttagagcaccctaagcaggctgctgtctttgatccccatgcctctgctgtttatcttggtgtgattcatcactgtaatttaagacgtggagagagcaaagttcccatcccaggcaggggaggcgcagtgcaggttggactgtgtaaggaaatggcagcatggcgaggtttgtgccgcggcctacaagcagggctgcatgctccccaggcagactgtggcagagccaagcccctcacttgtagggaagcgtgtctcctaggtgccccagtaggggaggtctgccagcatcgctgtgctgtggggaaggcagccagagggcttcatccataactggctcagctcctcaggaggagaccagcagtgtgtctctgcactcagaactgccactgggtcgtggtgttaagcccaggaggggtgcatatgttgacaaccttgtattgcttagttgctgagaccagaagatccaggtaattgcatgagctattagctacttctggttctcacaaactcctcccagtgttgatagagaatggtgtcctccgggcatgctctgggtatagttttatttgtattattagggactcatggag

SEQ ID NO: 54 - DNA, 310 bp, Homo sapiens
ccacggctccctcggaacgctttcgggtcatagcataccacgacccatccggatcctggatccactcggcggcctcgcagtagaattcgccctggtcagaaggctgcaggtggaagatggtgaggcggaaggtggtcctccccagcttgtccagccgcacctcccccaggctctgcctctgggcatattcgctgctggagtgaagcatgaaatctcggctcagggagatgacctccacgggcttctcgccaactttctgccggagccaggccacagacaggtggctgtgctgaatggtctctgaggccac

SEQ ID NO: 55 - DNA, 196 bp, Homo sapiens
gcagagcggaggaaggtccgggagaaaaggggcgggacggaggagaatccgggatcgcctggcagaaaaagagaagggagtttctgaatcctgggaagaggaggcgtgggtagggatgcttagcccgagatccgacagcagggaaccggagcgctccgggggaggggcttaatgctggggaagggatgtcttaaaa

SEQ ID NO: 56 - DNA, 169 bp, Homo sapiens
aagccagcgagccgctgcagccctgatcgagttaaggcgcggcggcccccggggccgctggagaaggatgcggacggggccagtgactcgtagtagatccctccgcgcggagctcgggccggcgcttcttcctgcgggaaacccctgggtgcccaaggcggcggggccg

SEQ ID NO: 57 - DNA, 757 bp, Homo sapiens
gtctgatggacctgagatgagaaaccagaactcctgcccctagttcccccggctttcctcaggctggggagtgagtgcggtgagatagaaagggcaagccctcctgctgtttctctctggacaggggcgggatgctgggtgaagggtgaaaggaagaggctggagaagggagaaaagctccagctcacactaagtctgaattttttttaaatgcggactccgtggcccctcccttacccgccccaatcctctctgaagtcctggttgtgaggggccaagtcccaaagtctgctgctccgcctctctgtgtgcagagccatggggccttcacaggctgcagtgggtcccgagcccccagggctgtgcctgctggtcctgaccaagatcgcggctgccgaggtcagtccagcgccaagggcacagggccagggcaggcggggcagggctacccgaagcgcatagaggctgctggtgtcaacgtgacgtcttctggggcgcctggcatccctaggagtggaagccgctgatgaagtcaaagctgcctcctccttcaggaagactttgctcccatagctggcgaacaggaagcggagcagcgccaggaggatctgcgggcgctgctgagggcttctttgcagggacagtgcagcaggcagccagggacaagactgcacggcagcgccccatggccaggggaagctcagaaccggagtcgcccgctgcccggcgattctccatccctggatcggtacagggg

SEQ ID NO: 58 - DNA, 557 bp, Homo sapiens
ggggctggcccttaacatggatcagtcataaattagtggcttggtactccgggaagataaaatcttccccatttgtctagtgattgacaatgcgtgaatgctttaaaagttgcaatcagcgtccctggatgggctcgaaccaccaacctttcggttaacagccgaacgcgctaacccattgcgccacagagacaagcactaccagttctaataggcaatataggaagggcacactcactaaacttcctccgtcccttctattctcagcgcccgcctggcaggacgactgagcaaggctttggaaaaccagagagattagagcggtgagttgcgctggtcacattggatacctgcgcgttaggagattctggagccagacggagagccgaatggccttcgcccgccctgcccctctcttgcttcagaagcccccagaaactccgcggtcggcgacccagcccgagccgcctgaggtcccaaggggaagctgaacgcctggtgggctcccgggatggttcttcccgttctttgtgccgccttcacccagtgaaggagcc

SEQ ID NO: 59 - DNA, 1658 bp, Homo sapiens
ggccgaggtgggtggatcactcacttgaagtcaggagttcgagaccagccgggccaacatggtgaaagcccgtctctactaaaaatacaaaaattagctgggtgtggtggcgacgcttgtagtcccagctactcaggaggctgaggcaggagaatcgcttgaacctgagaggcagaggtggcagtgagccgagatcgcgccactgccctccagcctgggcgacaaagcgaaattctgtctctcaaaaaaaatgcataaataaataaaagaggggtggggaagcaaaacgacgggcagtaggtgtggggcgcattgggattctatagtggttagtaccctgccttgtgcctgcagcaacctctgttctaatctgaatcctggtacagtcagactctatcttggacccactggggcgaacccacgagtcttttggtttgcttttaattcctgcaccagctgcggcctttatctgcagccagaaagcagggtttaccgctggccccacagcgccatacggtctggggaaaagaaggaaacccaatagtacacaaacaaaggcccaaagagaaaccttccaagtgctctatgcctcacggtttagcagaaaatatcaagcaactctcaacctagctggtctgtagcttccacaaatgaaatactgtattcattgcagcctttctggttgagatatttcaaatatttggtggagcttttaatgagacggagagacactctcgagtgtggaagaaaaacatgagggggtgtgaggataaggcgactttaggacagaaaaaacaaagagacaaggaagccacgtaaacgttttcgggtaggcgtgaggcgatgtcagttttgaaccccgtttatgttaggtagagagcgcagccctcttctagcacaaacaccgtttcccacattgaagaaatcacagagatcagcaactctagagtgcgatgaagaagcttcactctgggagaacccccttcgtgaccacggtctctttcctgccaggtaagtgggaatgagcgcatgccctgcagggacagcacagcgtcctcgccctggtcggacgctcagggtcaccaccctacccactgcccccttcgccattcttccaaaccactctctgccaaagattccaccgacagtcaccccacacgacaacccaggccgcctttcagcagtggctcccgccccgcaaccacgcgccctctcacccccgcggttctgcccgccgcctctgtccagtctgtgcacttcacctccctggctcccgctctcccctgagcttacagtggacgcggggttcttccaaacccctcttgggaatactgaatggaaaagggggagcgtgcgcaagtgcttggtagagtgtagacattgtgggatttgactgtggtaccatcgctttgacgtcctagtgctgatttttacacctgcattctgcttagggcactggcaacagttttccgtttgtgcctactccacctgctgtctttgttgggtcagcgaacatcgcctccctctaccgctcaatcagcaaaagggaccgcccttgaggacctcacccgccgctcactcccctcccaacttcgcgggcatcgcctccggtcgcctcttccgaaggcctaacgagcatgttagctgcgaacggaggt

SEQ ID NO: 60 - DNA, 356 bp, Homo sapiens
acgacgactcttctcctgcgtggcaaactgctcctccgcccggtactgccggtctcgctcctgccgcagcctctgctcttgttcctcaagttggagctgctcttcttcccagcgatactttccgtcacgctgttgggggcgcagctgctgttcttccctctcctggcgtagctgttcctcctcgcggaattttctgtcagactcttggctgcgcagctgctgttcctccctctcctggcggagctgttcctcctcgcggaattttctgtcgcgctcctggcggcgcagctgctgttcttccctttccggacggagctgctcttcctctaggatttttctgtagcgttcttggcggc

SEQ ID NO: 61 - DNA, 725 bp, Homo sapiens
gagccgcggcagggagtggatcctcccagtcgccctggagtacggctcccggccttaccttgggccagtaaactgggacggggttgggacgagggagtgttaccatggtaactgggcggcgcttcgtgccctagcctctcccgccaggctggctgcccacaatggttggcccctttgcaggtagtcggctgaccttacactcccgcaattgacccctcactcccgcaatgccgatacccgtcacccctcactgtatttgctccacaatataaacatcctacaagtctctgcttccactcaccccacgacaaactccaaaaccaagttataagaaaagtctaaaagactttctgggcctcaggttcaaaagagccccactcagtctcctttcaaagtcggctcaggtaggtaggttctctctcccgtctcctggaagtcaatgaatggaactgtcggagtggaagggatgggcaagatggcacaaattctcattctatttggcatggtatgaacttaagtgttctatttagaacagacttttctccttttttttttttttttttttttttttaagagacaagatctcgctctgttgctggagtgcggcagtggcgagatcacaactcactacagcttcgaactcctgggctcaagcgagcctcctacctcagcctccggtgtagctgggactacaggtgcacgccaccccgtcaggctccctgtta

SEQ ID NO: 62 - DNA, 380 bp, Homo sapiens
tcaggaaccatgtcctcctcatctctccgcccttgttctgggcttcgcccggaagaggcacagatggacgtccgtggaagaaacgacccacccatagtccagcccaaacccagggtgagcgcagagtctacacgctggggactcctgggacgggctggcaaaaacgcgtggccaggggaaggtgtgatcggtctgagggctgatgggggagccaaggagaagggaatgtgcccagcgcacggatgtggtcagggcgggaggcgcgtccgcaccgattttctggaccactagggagatgagcccgtcgtcaatgtagatgcggccccccaccggcacgacccggacaatattggggtagtccacccacacggtgttcgcgt

SEQ ID NO: 63 - DNA, 301 bp, Homo sapiens
ctgtgccccgccgggcggggaccgtgggagccgcggacaagcccaaggccggagcggttccaggaggaccctggtgaggagggctcggcccatgggtgtagaccgatggacctgggcacgaggggcggggcaggacaagggagcaggagaaatgaatagagaaagggtgcggtggtggctgaacgatggagacgaatgcggagagaatggacataccgctgttccgaggggcgagggcgcaggctggagcccggccgaggcagccagcagatgcggagttgacaagctgacggctgctcca

SEQ ID NO: 64 - DNA, 712 bp, Homo sapiens
cccgagaggcagacatgccacccgtggctctttgctcacccccacctgccggtgccagggtgtccctgccatttccagtcctggaaacctttgctttctgactcgacagtccccagcaggaaagagcaagtgtgaatcactaagtcctgaggtgggggttgtgggggcagtccttggaggcaagagaggaagcagttattgcttgtttgcacattgactccacccactctgtcctgcagctgtgacgtgtcctgttcccagggcacttctggcttcttctgccccagcacccattcttgccaaaatggaggtgtcttccaaaccccacagggctcctgcagctgcccccctggctggatggtatggagggtggggcctgtgggcatggggtgtgggtctggggagaattctgtgggtggtgctaagcagggctccaaggtgagtgaggggggtgagctctgtggttatgggggtgtcaaggggtcctgtggccttggggaggaggctgggtgcctggggccctgttgacctcttatccccacagggcaccatctgctccctgccctgcccagagggctttcacggacccaactgctcccaggaatgtcgctgccacaacggcggcctctgtgaccgattcactgggcagtgccgctgcgctccgggttacactggggatcggtgagtggcgtggggcgggcgggagacggga

SEQ ID NO: 65 - DNA, 396 bp, Homo sapiens
gcgctccagcctccctaccagcccagggggccggaccccaagtgcgagccggtggcgtgggtcagagcgcaggagcgaggcgcccacggacctggtctgcgtttctgagccgcacgccacggctgcgagacccgttccccatcgccgcccccgctcgctgacacacccatcccgcctctcacctgctggtgacacaagtgagaaggctggccccacggtggtgaaaaaaaaaaacaccacacgaaagaaagaaagaaagaaagaaagaaagaaagaaagaaagaaagaaagaaagaaagacagaaagaaacaacaaaaacaaaacacaaaaactctgggtctgtgccggggatccgcgctcagcaaggcccgccacagcaaatctgcccacacggg

SEQ ID NO: 66 - DNA, 345 bp, Homo sapiens
ccaaaacgccgccgcttctcaggcacgtccgttcttcctgcccacccgccggctgtcgcagaaacagcccaggaccatgcgccagcgcccgcgaccctctaccaattgcccttcggacagacgccctccccaccacctcacacgccctcttccctggccccacacacagcgagcgaccgcgaccaccttccacgctcttccctgcctatctcctccgcccgccttctcctcactcgcccaaacagacacagcccagattcttcccctattcctccttttccctccttcctcccaccggcctgcgcccaccgcccaccgccttgaatcgccgctgcgctgcccaga

SEQ ID NO: 67 - DNA, 351 bp, Homo sapiens
gcgctccagcctccctaccagcccagggggccggaccccaagtgcgagccggtggcgtgggtcagagcgcaggagcgaggcgcccacggacctggtctgcgtttctgagccgcacgccacggctgcgagacccgttccccatcgccgcccccgctcgctgacacacccatcccgcctctcacctgctggtgacacaagtgagaaggctggccccacggtggtgaaaaaaaaaacacctcacgaaagaaagaaagacagaaagaaacaacaaaaacaaaacacaaaaactctgggtctgtgccggggatccgcgctcagcaaggcccgccacagcaaatctgcccacacggg

SEQ ID NO: 68 - DNA, 345 bp, Homo sapiens; identical to SEQ ID NO: 66

SEQ ID NO: 69 - DNA, 351 bp, Homo sapiens; identical to SEQ ID NO: 67

SEQ ID NO: 70 - DNA, 381 bp, Homo sapiens
caagttcgcggacaccacggtgcagtcggacatgaagcactggcccttccgggtggtgagcgagggcggcaagcccaaggtgcgcgtatgctaccgcggggaggacaagacgttctaccccgaggagatctcgtccatggtgctgagcaagatgaaggagacggccgaggcgtacctgggccagcccgtgaagcacgcagtgatcaccgtgcccgcctatttcaatgactcgcagcgccaggccaccaaggacgcgggggccatcgcggggctcaacgtgttgcggatcatcaatgagcccacggcagctgccatcgcctatgggctggaccggcggggcgcgggagagcgcaacgtgctcatttttgacctgggtggggg

SEQ ID NO: 71 - DNA, 669 bp, Homo sapiens
gagataactggcaaagaagaggccaaattttttttgagactgcagtctccggtgtaaaatgagggtgcaaggtgcatccccgctcctgatttttgaatcatcccttccaccaccttggaatgacttcttacgtagaacagccagcaagtggagaaacttgcctcaataatgcaaaactgtctccctaacttacttcctttcaatgttcaagcaaaacaaagccctctctacatccaacatgacagaaacccaacatgacagaaagtatgagaatggttggaaattgctgaaacagcaacagtaaagatttaatgagctctcaaagtacctctagatgtgcaaatatgacaacccaataaaaagaaacacacacataacctagagccttcaaaaatctagatttctgcctttccacaccttaacttggaatcattcaggtgggttcctaagcattctctcggaggtgaggaatcctccaaagaggagactctaggaggggcccaggtgtggcaatcttcaaaaggatgagggatgtccagaggagaggtggcgggagggtgcacttaaaacacgcgaaaattggaaacgtgccttctgcagtcagttccttcggattttccggcatttagaagccagttctactccttcagcccgaccgtatctgcaggt

SEQ ID NO: 72 - DNA, 259 bp, Homo sapiens
cgcctcccctcccctcagcctcagtgcatcctgaggacgcccgccgagccggaggtggggctgaggaacaataaagttgcgtttttttttaaggcagccacacaacagcgtcccacgggagggggcggaggaaactaaaaaagcggagtagaaggcactaccgctgtcgaggccgccgccgccaccgcggtccctgtagcagccaaccccggcgcgccgtggccggccgtgcgcgcgcgcgcgagcggcggaagaggcg

SEQ ID NO: 73 - DNA, 469 bp, Homo sapiens
aggaaagtcaggtgtaggggagattaaaaggtaaactggtgggactgaccgggctggtgggactagttagcgaagccaagtgtttgggtagaaacagagatcttgtaggaggtgctccgagctacatttcgggatttgggatgaaaagagaaagccagggggttcgccgctcctggtttgttccttaccgaccctaaactgtatggcggagcttgtgggtcaggcgtggaggcggcatccactcccacgtgccccacatgcagcagcaatcccacgcggttttcgcaagcggctgggaggcaagcgggcgtccccccacaccccccccccactgcgccctggacccacgccacgcaccgccaaggagtagctatggtggcacgtggaggaggggcgggtaggcagggcggcaccatcaccgggaccagcatcttggaacggtcaatctcactgctcatactctcctccc

SEQ ID NO: 74 - DNA, 416 bp, Homo sapiens
cccgcccgctgcaccgcacctcgcctcgcctctctgctctcctaggccccggccgcgcgccacccgcctcccgccaccatgaaccactcgccgctcaagaccgccttggcgtacgaatgcttccaggaccaggacaactccacgttggctttgccgtcggaccaaaagatgaaaacaggcacgtctggcaggcagcgcgtgcaggagcaggtgatgatgaccgtcaagcggcagaagtccaagtcttcccagtcgtccaccctgagccactccaatcgaggtaaaggctcggcccccgcgtggtccgtgcgcccctttccagagcaccctggcccagtggcggggaccaagagacgcacgcatccccggggatgaggggaggcgaggggctgccggccccaggcagggtctacgca

SEQ ID NO: 75 - DNA, 560 bp, Homo sapiens
atgatttggtgtaaaatgaaaaatcagcccaaattgcaagccaagccgccggtggttatgaatggctgctgtgactccgccccaaggttccccaggagaatggatttatgtacttctcttccagtggggcacacagtggagagagtttgagacaacactgctgagagaattgccacatgggcgctggctgcacagacccaaacctcacagagtgaaaggggactttcctcacagagtgaaagtcgcagctttcccgagcccagggaagccccccagagaggggctgtagtgggggctgggcagttctaggagcctccactgccctgggccacccctccgccgccagagccacgatccctgcagccgcctgtggcaggtcgtcattggtgccctggtggagcagccctaggaagggcggccgcccctgcctgatgccgaccctaccccttgctcctcgcaggggcgtgggctgcatccactacgagatggccacagggaggcccctcttcccgggctccacagtcaaggaggagctgcacctcatctttcgcctcctcggt

SEQ ID NO: 76 - DNA, 662 bp, Homo sapiens
tctccgggctgcacctgcgacccctccgcccccgctcccattccctcgccggagggaagccgcgagttgcgagtcgtgatagaaacttaaatcgggggttgcgctgggtggtccctgctgtgcctgctattgcgctcctggcttcttaagaacattctcaaggggctcctaccgcggtggtttttatgtaagatcaggaatcagtttcctgattacagatgggaatactgaagcgttagcgcggcaggtgacttggagaaggtggcccagttggaaagaaagaaattgcaacccagtgtatttgttttccagtaacatcccctccaggatatcccactagttgagaaatgtttgggttactctcccaaatattcccaaggttatttcccgagcctgggagcgcaacaaatacataaataggcgctcgttgaatttttctcgtctagccaactatcagtgtactctggggccttcaaaacattctgccgtttgtctcggggagatggggttaggaagaatgaatatgggagaaaatgacagtttccttcgcggaaatcaaagcgcagatgcagttcccaggtgtctctgcatggaaacagtcgaagctgctgccggaagctctgctggcctggacaaggccacccagaataaagtcacagaag

SEQ ID NO: 77 - DNA, 277 bp, Homo sapiens
catcacctaaagctcctcgcaggccaccgcgagggcagccgaccggctccggaatctggccgcaggttgaagccgctggtgcgggaccctcgggcaggaggtgaggaccccctcccttctctcgcccctcaatcatcttagggcggtggctacaggacagagagaggggcgtgcccctcggctgtgaagtgggcatgcccgtgtgatgcccccgcccgtcgtctcaccggggcgcaccgcgctggtcctcctccgccagtctcccgagctccggcca

SEQ ID NO: 78 - DNA, 802 bp, Homo sapiens
agccctccgcctcgaggccactggtgcttgggggtgggaaaagagtgaccgggggcaactgagcaaaacatagaacagaaactccctctcgaatagagacccacgcacggaaggatgatacactgtgattccccgacgtgggcctgcagcaagagacgggtgctaacagcgtccgagccaatccgtaagcatcagagggacaaagaaatcataaacaagaggcaggatgcaactgcttcaacgcagaaagaatgagtcatccaggaaggcgaggtggtggcgggttggctgtggttttgcagaggcgggcaggaggggcagggggccagggagagggcagggttgggagggtctcttttaagggaatcctatgcatgaaagtctgtgtaagaaagggtgtgcctcctacagaaggatatttgttttgtcctaaacttgccgtgtgttcttggaggcgcgggcctccctgctgtcatcacacctcggatcaccccaggcaatctcgcccctgtggcttcacctgggtccagccagacctctgctcagctctgcacgtacatgccccctcgatgtcacctcctgacttggcaagtcatacaccctctctctacttgcccagcttcattcacaaaaccaggtcacattacccctcgtccatccatgctcagtctggaaaccctgggatcccagacaccagcctctccctggccctctgtttccaatcagacggccaccccgactgacgctggggccggggcacccttgggcgttatccagtctccctgtcctccctctcctgccc

SEQ ID NO: 79 - DNA, 717 bp, Homo sapiens
gctcatcccgaatcccgctctattccacgtgctagaagcagtggtgttccgggagcctgtcgcgagtggccgttaagagttctagaacgtgttctccacggatcttcggggtagtcctgtttggataatatcaacactgagaggaagaattcctaaatgattttcctaaagccatgtcaggacaaaactgcaccagagttaaaggcgaagaaggcaagaagaccttattcaagtctattatgataggagagagagagagattcagagattcaactccactgaaacaaaggatgagagggtttctacaagttgggatgggaaggagattagggccatcattgtctgttaattggcctcctgcaaagaaacagagagcttcccccatcttcatgacaggaggtggttgcacagcttagagccaagcacccaccaaggctgggctccatccttccacagggacaggagatgacgccatcttgccagtgatcacattttgaagggacagctcccagctccttgagaaagacagtcctgggttgtacagccagcaagagacttctagaaagattcaggtatcaaatggcagagaaagaacatacaataatacattttctaaagcaaatgctctaaagatagggagttccaggtcccagaggtgggaagaagccgggtcaagtctcagtccagacgaggaaacccaaaggctcagtccagacg

SEQ ID NO: 80 - DNA, 464 bp, Homo sapiens
ggaaggagcctgtgagctcctcctttccaggagcgaaacgctggcccaccggccccattgcctgggccagcactcaccaggggcatttatctgtacctgtgttgctgtgggtaatgtactcacaggcccttcccagccagcgcctctcacccatgctcggggctcagcatagtgtccagaggtcacgtgtctctctgttgtgattctgctagagggtctcagcacaggaagatcaactgagtctgcaaaaatgaaaagaacatccatcaccacattcagccaagccctgagccccgtacctgcaggggctgattttcacctggagtggccatggggttccttgggtgggagtgatgtttgtgagtttccaggccattgctcaacatgaagacacatttcctaccttcatttccccggtgagtttcctttcttcctgggctgccctgtgcctttcctcctcaagg

SEQ ID NO: 81 - DNA, 872 bp, Homo sapiens
ccaccacaggcgatttggaagggacgattatgagaggagccaccacgtccggccacacagctctgagaagggcagaaagagagtcacgcctgcgcccatggagccccaacttgtacctggaccacgctgcagcctgtcccaacccagagaagctgacgagatccttgtcctccatcacccagacaagagctagatggtaaaattaggacataattttaagcatatatactggatatgtatgcctcatgaggaagggttattctcaaatctgcaaatattgagtacagttgcagtgcaatatcaatggaccataaagacaataatcatatacctaaatcacttcactgtgaggaccattttgaattctttaaacaataaacaggacttttacatagatatttttgcactttttgtatataatgttttaatagcagtttacaataccatgcaaaatattaatttaggttattcccaggaccctatcctaataggggtagaccactactgcgctctttcttcaaaatcaaatttgaaccaaagactcttactgctgctcacagagtcgtttgttgcttattttgtgagaaattaggcaaaacctcaatagccaagactgcctggcttctcctgatggatgagcacatacctatgatcacaggtccctcctacaaatgatggataataggtccttttaaaggcagatgagacttacctgaatctaaggggctgaagtcacagccttcacttaacagctttcagagaaggtgctttggcgctgcctaggcaaggtggaattggtgtggagtgtgggaagaggctctccggaggcaggtgcagtgggtggagcagggcaggagtggctcgggcagctc

SEQ ID NO: 82 - DNA, 369 bp, Homo sapiens
ggcagggcgctgggctggctgctctgtgagctcctcccagtgcggggcccggtgtgactatgggtctggtaagaacagctgactgtgaggcagagatcaaacaagacacagacaagccacacggtgcgaactgtcagaacctgggataaagacagagctggcaaataaacatacatgacaggcacaaacgctagatggtttggctctcagggcataccgaaaatcaaaccatttgcagaaaaaacaaattgtaagaatcaaaatgcagtcactcagcaaagaaacatgagcacttacacaggctgtatttccaccatcccggggtcagagacggtgccttcagcccctgaccctcccttcagccctccc

SEQ ID NO: 83 - DNA, 865 bp, Homo sapiens
cctgggggagggtgctcttgtcactaagcttcagccactctgggtctcccgggaggctttgtccctgagattcttagagggtctcccattcggcttggtttcctgaaagtcaggtgagggttttttacaaaatgatgtctaggatattccagtattaggaaaacctacacatttcaaagtgtgaagtagacttgcctatatttgtggcatttaggtggaatgcttgctttctgatatatgtaacctgtaaacaaaaaacacctttctttcttttggttcccagcacttttctttttaaattacactagaaaataacaaaaaatacatgggaaataattttaaaagtacccttatcataattttttaaaaatttctttttcatgacgcagatgagattttggaatgggctaggccccaggttgctcagaacgtgcaagggtcatcatgcaaatgaaacaccaaagttctggttgtagggccttgtggctgagagtgtgcaagaactgtgaagttatggctggcaaaatagcaactgggcgagtgaagaagcattattagttgtatgatgcccaataactgtagtaaccagaacacacctaagtttatctggatgacatgcagatggtatcacccgctgacagcaggaatattggccaggggatttacaccgtggcttgcggctccagtgcctatattagcactgggtcagcctctcacctgcaccacgtcccatccagccgccctgccccctggccctggtgccctgataccctggaggacgcacgcatgctgccccagcgttggggacagcgcgccggatgagaggggctctgggaaaggggacccgggccgcgctggtgacagg

SEQ ID NO: 84 - DNA, 710 bp, Homo sapiens
ggtgggcggtgggggaagacaggatgcaaatcctgcagcctgctgaatccggctgcactcagcctctgctgctcgctctgtgactgggggcagttttctgggcagtgggattagtgacggtgctcccaggcgtgcacgcaggtgcccatcccatctctccatgcagtcatctggctgcccctgggagtggcgtggcctgtcctcctccctctctgctctctgagccctgtcctgtgcgctagcagcagagtcaggggctctgggtgggcttccccgctgtgagcgccttgaaggcaggggctttgtcgtattcatccttcaccaaacctgcaggatgctaacagcacatctgtccttgcaatttggaaaacagaggcggcataccttcttcatttccttgccaatttctcatgattaactgggctgatatgagtattgtgtttctctatgaaggaagaggacccgtattatagtttggctatttcagagtacaccaggcacattcggcaaggcttctactgaaaggcctggtcagccactctgtaggagctggtgcctgctcatgcagccaagcttggttccctgcagcctcccaagcacagtctggccaggttggttatggctgttcctcaggctgctgtggaaggctcttgttggctccggccccaatccctctttcaagtcacagtggtcagggtcacaaaactggt

SEQ ID NO: 85 - DNA, 620 bp, Homo sapiens
gccggggtacagtgggaggggcactaggatgcactgctggataggaccccgggcaggtgatgggcagtcatcaagaaggacttctgggtacctggtttagacaatgggtgggcagtggagacttctcaagacaggggatgaagcagtcatcaggtttggggtaaaagtcaggacaaactggcttcacgttggttggttttgaagaacctgtgagtcttccaagtagatagagccaaacacaggcctggaactctgcagagaaggcaggagctagaaataggcgtggagcccttgtgagggtgcagcaccaccagggactgtgcgcaggtgcaaagaggccaagaatggaactccagggagctgagagagagagagagggaaagaaagaaagagagagagagagaagaggtcggggaagacagggccgaactggatgacagtgatcatggggaggccacaaggaccaactgccccacgaggcgtgtggacagtgatacctaatactacagaaaccgtcctaggatgcaacacccagtggtgtggggactgggtgacttttgcactcctttccggcctgggcactctttcatcctctgagtgcgaaaaacacaccttttcta

SEQ ID NO: 86 - DNA, 629 bp, Homo sapiens
gggtttcaccctcctctcctcttcctcccagctgtccctccctcccctccgggacactgcataaggaattgccccaagactcaggagtgtacacaggccctgggcaggttcacagagcagctctgcttccagctgcgggtagttccctcagcgccccccgccccacctgcttgaaccaggggtcagccatgatgggtcgagaatgttccccaaaaggatatgtcaaagccctacccccaagtacctgtgaacctgactttatttggaaatacgatctttgcaggtgtcatcaagttaggaggtcactagggtgggccctggtccaaaacgattgatgtcctcagaagaggagagagactgacactcgggaaggagagcactctgtaaggacgaaggcggagacgggactgacgccagaaggcagggacccagagaagggtgcccccgtcagtgagtgagcaggccctgcagatgccttgatttgaacttcttgtgtccaggactgggagggagcacactcctgctgttttaagccacgcagttaacagtgctttgttccacagggcaaggcagaggctccggagaacagagatgcaggagacagcaggctgaagcaagctcagagcacg

SEQ ID NO: 87 - DNA, 529 bp, Homo sapiens
ctggggacttgaatcagctctgcctggccttggctgcttcagtacctgccggatgtgtggcttcgggaacactccttaaaacctgattgcctcagtttcctcctgtattcaaagtttgtctgtccaattgtttataacaaaatcaacctccaggagagaatatgtgtaaagtctgcttagcgttactacttttattcttattgttagaaatagctgacttgcaaaaatattcctgaccatattatgggggaggggaacacgtgtgagctgcgtcttgcctatgcacagagcctctggtgtggatgtgtcccaccgagcactgagcctcccatgcagatgtgtcccgcttgtgcacagggcctctcgtgtgggtgtgtcccgcctgtgcacggaacctctgatgtggatgtttcccgcccccgccctccacgtgcagtgaacctctcgtgcagatgtgtcccgcctgtgcacggagcctccggtgtgcgtgtgtcccgcctgtgcacggagcctccggtgcgggtgtgtc

SEQ ID NO: 88 - DNA, 315 bp, Homo sapiens
ggacactcccaggggtgatcttttcattatatgtgatgggctgggctgccgggccatctttgtggtgagactctttgcccctgttctagacaacagtgtgggctgcctaggtgtccgtctctggactccagcagggtggggctgctgaccaggctccacaagtgctctgggcagcagctgcagactcgacacttctgccaaggcggcctgtgtgggcagaaccagggcatggaaatagctgaaatccaatgggcgtgtatgcttccggctgctgccctgctcatgctctcatctagcagccccatctgctgcgtg

SEQ ID NO: 89 - DNA, 593 bp, Homo sapiens
caatccagggtcattctaatgatgaatgagatgctgcctgcaggagctccgggcactgggaccagggcagaagaggtgtggacagaggtcaggtccctgccttgttgttctggccaacacaggctcatgtatggaaaacagggaaggtgaggagactgcagctggagcaagtctagtattcatgcatgcgaagtaaatgatgaagcactccacaaacattcattgaacccttgtattggcttatgtagctggttgactgaaaaatcagtcctccttttttggtaagaaactctctgatgacagcatgtaatcttcatcaggtcttcataattccagaaaatgtcttaaaaattgcttttatttgagctgatgggccttgtcctgggttggggcactaggaatcctccagcacacaggaaggctgcctgcacaggtctccttgtatctgcacagctggccacccaggagccgatgcaattccaggtgagtgaagaagcctgtggatataaaggatggagacagactccaatcctttccattttccggttggatgttattagtagcgtttgatagaagaggaggccctgacaaag

SEQ ID NO: 90 - DNA, 585 bp, Homo sapiens
atggcttagtggatgcggggtcctcggggttccaaaggggacaaatgcccgggtccctcttcttttcacctcatttctgactgagccacaatctgctacttcctttcttttccatcaattttccaggaaggtgaatgcatgcctgggtcaccctcatgtcctcacggagcacagctcttgtctctgtgctctggaatacctctgggggaagctcataggaggggcccacccagcagcacagcttgaggtatatcttctgatgcagaacttcatcctgagccatcgcgtgccctgattctcctcatctctctgggtatttcctggttaccctgcaaggttcaggtctccatcactcctctgcctcagtcaccaagtgctgattcccttggcctaagcccctgtcctgattttgtttggatctctgagggagatgagggacaagggcatttgttccttttgccccaagaaggggagaggccctgcctggaaacaccaacagaactgcagcacaaagagagacctgaagaccaccatccggtgtttcccagctcttaatcacagagcacatcagttatctacagacag

SEQ ID NO: 91 - DNA, 378 bp, Homo sapiens
acttttctgaaaaatgaagtttctatgcaaaaatggcacttttttaggccggttaggatgttgtgtgactacaaggtctttggtgatgtttagtgtctgagctctgccagcctgttttccatgtggcagaggagggatcacagagatgttgtcaggatgtcatctgctgttctgatgagggctctgcctagcggtgatgctgaaaaacaagaggtgctgtggtatgacagataatctccacatagctcgtgtgtgttttatacgggaaggaatacatgacctgtcataggttggtttgcctttatcttgattatttctcttaatgctccggtccaaaatgaaatgtacctttactctgatctgcagtaattgaattta

SEQ ID NO: 92 - DNA, 290 bp, Homo sapiens
ggtgccctggacagcgggctagatgtctcctgcaggctcgaggacttcccggagggggcggcgttttctcggggcctgggaagaatgggcgggtttcagcgggcgccttctgggtgactgcccgtttcggcttcccctgaccccgtaacagagcggaagaggcccagagacgccagaggaggggcgtcagcgggacactcaggccaataagctctgactcccgctgtctgcaaaaatgaccggatgcaggaatgtcacctagaaccggccagctttccgacctctccggt

SEQ ID NO: 93 - DNA, 484 bp, Homo sapiens
ctggatggaggtggagatgggcaaccttgggaagaggactgtgccaggccggggcaggacagtgggtcctgtcctgcaaggtcctggtgaattcttcctcaactgcagaggaggggaggcatttggggacctcgaggtctcttcctggaacctggcccctgagagaaggctgcccatctgggatttggggagtggaagtctcttctctcggtgggcatttctagttattctacctcatggtggtgcagagcaagccaggacggggcctcaccaggctggatcctgaacaaaccactgctgtatggtcgcaggctcacagcgccccgagtctcagtttccccaagtgtaaatgggagttgcccctgtcctgcccgttctctggcatgtggtgagagtgacagtgcatgtggagtggcccagtgtcctcatagggccgggttcccaccctggctccatcgcagggggagtgtgcaagtgattgcac

SEQ ID NO: 94 - DNA, 480 bp, Homo sapiens
ggtgaagacccctgagcactgtgggtgtgagcgctctctggggaggggccggccctggctggtcagccttgtgtggcactgtactggagaggcacatctgagggacagctaagctccccctggcgggcagggattgtgccccatttcatgcacaaagcatgcttaagttccccctgcgagaagccctgctgtgtgtctggattcttaggcctgaaaggccttacctttcacatcaagcgaccagtcttttctaatgagttcaagtggtagcacatctgatagggcaaaacagcctcatttacaaagagaaaaataaataaaaatgttatatagccttgagaataaggccagacagtcagcccaaggtccctggagcaacacatagagaaaaagagttggtaaaagtctttcttcttctgtggactctgtccgggcagccatgagctggaactcagcccagccctcgtgccacgggatatt

SEQ ID NO: 95 - DNA, 424 bp, Homo sapiens
tccaaaagctgtctgttgagggcagccaggctggagagggcagggtccccgggggaagggaagcctgtgggtagcctcagagccactcttccccttgaggcatctgctgattcatgagcagcatgggggccaagaggctaaagccaagcagaaagtgtggataagaagctgagaacttgagtaggagactttggcaatttcgaagggctgggagataaatttggagttcaaactacaagacaagcagacctgggggccaagatgtctggggaagagaagcacgtggaggttagcttctaaggcccctttctctggaaggcattcacggatccgtaagcagcacccagccaaggagtgaagcatggctaagagaccgggaagctgagcagagtttttggcagtctcgtggggcttggaagacaga

SEQ ID NO: 96 - DNA, 431 bp, Homo sapiens
atttctggctgcaggagggggcgaggggcagccgcgacgcccgccaggccggcctaccagcagctcgctctgggctctcctatacacgcgcaggacacacacgagcacacgcacacactcacatgggggctcgccacatgctacagatgggaccaagtcgggggccacggcagcagaaaaaaaaggacacgatgcgtacgattaggttttggcctttagtctgaaaaagtgttgattgaaagtgtacaacagagagcgggtgcaagcggccgagggccatggagccgccaataaaaaagaatgtccttaaataaagttcacagagtaaaaaccagaaccgccagtccttccctccaacacaacagagcacaggcacagaaccggtgatgagcccgaggagcagaggcggctgggaaggacagcagaggctc

SEQ ID NO: 97 - DNA, 746 bp, Homo sapiens
cttagtgtttagcaagcccaaaggaagctcacagttattgaggggaggccggtggaaaggaaaggggaacagaggagtgctagaaaccaggtatcaagcaggtggattatggaaaagcaatctcagatagccacagcagcagagaataacaacagcagagcctaaacaaacaagaagagcccttgggatccactttttcatgaggttcttcagtggagcactcctgaggtagtggccaagctctcctaatcaccagccaccacgttgccctgggcaaggcttgtcacaaagaagagcagggccacagacaaaggcagccagcctcacctcgcctcagcacactgcacagcagccatctaatagcaccctgcaaaactgacagattgttcctctcaacataaactctgcttcactcctaaaaggattttattacctctcgcataggagtttgatattggcgagattaaattaacacagttaaaaaagggaggaaagaaaaagaagagaaaagaaagagaaaccctatgggcaggtgaggggactgctattagcactcagagctttgtgggacagcacctgtcagctcttacttcttcgagtgactctcccagtgtgtcaagctgtggaaggtctccaggagatgctccgtatgagaggttaaggctggttgagtgacaaacttacagggacaaaagccgggacaggatgccatgtgaagtttggagccagggccacccacccagggt

SEQ ID NO: 98 - DNA, 625 bp, Homo sapiens
tgtgtccctttctccctccctccaagcctgggcagagcagtgggcaggccgggagacgtcttgaagatgtttgcttcccttcttcagctctccaggctgctgacatcctggctccctgaagtctaacaggggagaaaagagtggattgaaaataagaaaatgctcgcgtgaaggagtaaaaaggcagctggaagtacaacattcatttggagatggaataaataaatgagataaaatcctaaataaatatcttacacggttctaattgcattttaaaagctgccaccagagaacagataaaatgatgctgtatttcattagtggctcctgtaattctatggtattcgccagcaagccctgttaaagtactctgtaattaaagaccagttctaggtcctgtaatttagagcattatcaaaatacggcaagtgacaggaggtaaagaaatggaaacaaagcctgctttcatgaggtcctggtagaccacatagaatgtgaatttttacagctcagcctttgtattgccctcaccaccagggccagtccacatcaatgctttccatttgacaggtgaccggttatgtttttgttttgttttgtttttagatgaagtctcactctgtca

SEQ ID NO: 99 - DNA, 889 bp, Homo sapiens
tgtcatcaccaaacctatcaggtgagcacgaccaaatttcaagcatgtccgggactactaacaagattgccaggtggaattgagactgcttaaagatttaatcaattgtctgcagtgcagcttctatttccaaatcaaagctggaatttgtttgtgtggttcctccacagctgtctgcacctgttccttagggaaaggaagaatcagtttccatgatttctgtcttttcctccacctcctcagagttcttgaacttggccaggccatgcttccaaactacatttggcccaaatcttacagtatctagtctccctgcttttgctcttttctgaaatgataacccagattgagctgggtttggtttcattcccacatttattgattttctttccataaataaatagctatggggtctattatgtgtccaaagagtcaataaagcacatagggaagcatgtcaaaaatgacgaagaaagttgaatccagcaagaatctttccagtgccacccatggagcgaaatcatccacatttgccgttcaccttcactgcctcccaaataccaaacatcagtcgctggcatgtgattagtgagggagctctgagttgattaagtgcggtgacattttgaaaaaagcaggacattcctcattatccgcaaatccgtgggtatgagagagctctctcgttcacatattagcatttccacatccgtctcctcttctctgcaacctctagcaaggcagcctgccttattagtaacatgtttgggattaacagctttgctattgtgttagcatctctgtcattacccagggagggaaattggcaagtgctaagccggttactaacagacctaaacagtgcccaggatggaattcacacatggaa

SEQ ID NO: 100 - DNA, 735 bp, Homo sapiens
tctaactgaagaggctaagaacgtttcagcggttcctggaccaggtgaccggaagactgggataccaactttaccctctactttctactcacacacagagaagcctggtagtttctaccaacaggtcttgccacatagtcatctacctgaagaggctttggaagtttcagttgctcctggaccagttgaccagacgattggcacaccaactgtaacctccccttccagctcatttggagagaagcccattgttatctacaaacaggcctttccagagggtcatctacctgaagagtctctgaaagtttcagttgctcctggaccagttggccagacaactggcgcaccaactataacctctccttcctactcacaacatagagcaaagtctggcagtttctaccaactggcattgctaggtagtcaaatacctgaagaggctctcagagtttcttctgctcctggaccagctgaccagacaactggcataccaaccataacctctacttcctactcatttggagagaagccgattgttaactacaaacaggcctttccagatggtcatctacctgaagaggctctgaaagtttccattgtttctggacctactgaaaaaaagactgacataccagcaggacctttaggttccagtgcacttggagagaagcccattactttctaccggcaggctctgctagacagtcctctaaataaagaggttgtgaaagtttc

SEQ ID NO: 101 - DNA, 380 bp, Homo sapiens
gcgcagaaactcctggagaacagccccggccgccaggtactcgcccgcccggaccgcaaagcccacagagcttcaattcgcaggaaggggagtggggagccctgagctggggtgttcctggaagaggtcggggagcggggggagaggcattccaggctggaggcctggaaacgtttggaagctttgctaaccttggagaccatgtcaggaaagaagcgtacccaggccgtctgctgtgtgcccaccccgatccccatcagtgccccacgcactcgagctcttcttcggcgtctgcagcctcttcatccgcgcggagaggcggaggtctcccggaaacagtgtccgactgtggcaccccagagcgtccaatgcgagctcag

SEQ ID NO: 102 - DNA, 350 bp, Homo sapiens
ccacctccctcgccgtcggccccgaggtccccggagtccccgggccccccggaggggcggtgtgcgcgccagcgtcccacagaagctggccgagatgccgagcagccagtatgggctgatcgtgttcgtggcggggctgctgctgctgctggcctgggccgtgcacgccgcgggcgtgagcaagagcgacctgctgtgcttcctgacggcgctcatgctgctgcagatgctgtggatgctgtggtacgtgggccgcagctccgcgcaccgccgcctcttccgcctcaaggacacgcacgccggcgccggctggctgcaccggctggtgagtccaggcaccgggcaagcgg

SEQ ID NO: 103 - DNA, 388 bp, Homo sapiens
ctccctttctgccgctgcaaaagcgttctttctgaccgagacctgcggccggaacaagtcccgctgggcctccatccagaaaacaggttcgctttttgagaaagaaaaaaaatcccaaacttggaaaaaggcaaaaaaccaaaccaaaacaacaacaaagtcacacacacacacaaaaccaccttggaaaaatacaaaaaatagataagtaaatttcttaaatccccaacttggaaaaaggccacttgcttgcgttaagggtaacttctagcattttctaataagcctacttagtaaaagggggacgttctattctttaaagaacccccttggctgtccgggttggggatgtaactgttgtctaggtacagtggattaacgaaggaca

SEQ ID NO: 104 - DNA, 323 bp, Homo sapiens
cggggagccctgcttgtcaggggcgggctcctcatcactgtcgacgctccggctcagcagctgctcattctcggatgaggcatcgttctcgctgcaaaaacaagagcgatggtcattagcagtgcctgggggggctgcagccctgacaagttctcctgtggtgaacttgtccattgcccagctgtggacagtggggggcaatgacttgtttttcaggtgtttactgggcaccttgtgggcacgggaccaggggacatgagacgagacatacaccggtggcttcctgcttctttccaaacaaacaagtccgtggtgggtgacct

SEQ ID NO: 105 - DNA, 849 bp, Homo sapiens
agaaacataaaaccccggatcagccatttcttccccctctggacaagcccggacgacacggggccactgttctgtgactctgacctttgccgtacattaaacaagaggagggcgggtcacatggacacctcattaattcgccctccctgtggcaggaggatggagggacaacagagggcagaactctccacactgcagccttatcaaggataacgagtgaccgtgttgggggctcaggggaggggcgagtatccctaggggcggggcctcagtgaagggataaattagtgcatcctttgtttgcctgccaataggtgcttccaggggagccctgggggctagggaagtttgtttgcccaaagcgtttaaacaatccccctgcaatcctgtttgcactcttcatttgctcagcaaacctgcccctcggatatagctttggagtaaactcaggcccagctgctgctgagctcagggatgttgtttggccctcccaagctggggcaggggctccaaatgtacacaatgcccccccaactaccaccaccacgcaaataacaccccatctgacctcatcctgctgggcaagtgagagttaaacatcctagttcaagttcattctgtctcttatagctaagtacctgacttttcaaaggtcagagctgctgcagggctgtctctgctctcaggaggacacccttcctcccactccctctttgccaggctgcagtggaggggcctggtggccctggggtgttgggtcgggccatcttgcttgcaacacgccccgcagtcctggggccggcggggttggggcggggagtgggggggtcctgaggtctgtttactaca

SEQ ID NO: 106 - DNA, 506 bp, Homo sapiens
agttggagcaggagaccaaaccaggagatgccagggccacacaccagcccggttctccctggagcctcgagaagcagagctaccaccctggatctgcagtcagcactcccacccctgccctgcccctgccccaagcccagcaccacccatggagagggtgacctcaggccttggagattgctggctcgtggctgcaggtggcttggatccatgcacttggatcctggtatgctcccctcacatgtcaaaattcctgtctccatccaatcatggcataacactgtactccctgggggtccagcctgcctatctcacacccagagttgcactgccagtctgggctgggatagcgatgtggttaactgcaaccaactgctgtggttataaaccaggtaaggggcccagtgacagccccgctgcatcattggcaaaggactgcttgggggctgagctgcccgggggctggatgagatgacccctggaggcctttctgggtgccagactct

SEQ ID NO: 107 - DNA, 368 bp, Homo sapiens
agaggaatttcaaccctggtgagagaggaggcctccttggtggtttcaccggtcccccagtgggaaatgccacatgagcacttgcatctgtgactgctccctgttgcctattatttccctaaagttgccctcacacagctatttttagtacaaaggctttattaccaagccaaagatgttggctctgagtgatccaagaatggacaaccaagtgagtgggtagtcagggaggcctgttcaggagccaattaggcatcgtgagctccaccagcatgccacggaagcacttctgggataggacatgaagaagagctgtgccgggggatggtttggggtgagggatgccaagactttaggtctctctttta

SEQ ID NO: 108 - DNA, 454 bp, Homo sapiens
ccggtgctggcgccgccggtacagccccgacgcggccagcgcacccagccgggccgccatggcgcggggcagccgccttgaggtcgcgggcgcgggccgcctggtgcaggggctatgggaccacctcggaggggaggcgcggccgcctggggccagacaccaagacgggacgggcgtgtgggcgcctcccccgcgctggtcgcgagctcacgttacgcgctgggatctccaaagggcagcagagtcaactccaaatagaaaggatattgttcgcgccgcgggggcagctcctgaaagctgaggctggcgggggttgggtcccgctgggaggggactggtctgggaaggccccagacagcactgaggtgaggtccacagtggcggcaggacacccaggaggtctccggctgccacctgccaggaggaaagcctgtgcccgtggacacactgagga

SEQ ID NO: 109 - DNA, 642 bp, Homo sapiens
agccagcgactgggaagtgattccgccctcaggggagtgggggaccaaccggcagtgcggagccgaggcgtgggaggctctcctggcgggggccgcggtgagggggcgcggcggctggcgcttctggagcctgggtccgtaccccctcctccccaagtcccagccgcccacgtccccgcagcccgcgcgggaggaaaggccttgaccttgcacgctggggcgctgaagttagacgctagcctactgggctgcacttccagggaccagggcggcgggggccgcccaccaccaggaccgagagggaaagcgaggctttcctttcctgctcgaccccctcacccctgcggctggctttgggacagtgccccgcagccccgcattgaagagatgttgggctgtctgcagaggcctgcggagcggctttatggcttcctccttgtttgaaaagaatatcagacgaggtgcattttcccccatccatgtttattatactccaatgaaagagttataaaacattgagccaattcttaagaatcgtgagtgcccaccaaatattctgtctttcgtgggttggttggagagtccagcggcccgggcccaacctggctgcggagggctcgctccacgttggctcgtgtgtgg

SEQ ID NO: 110 - DNA, 516 bp, Homo sapiens
gagccccgagaggccgggtgcctcctcccacccccgcccagcagaaggccggtccgagaggctccactgtgccaagcgccctgccaactgactggctctcgctttggctggggagattggggatgaggggaagaaaaggttctccctgggtggctgcaagaaagagtgcgcccgcctggagagcgcccagagagtttaggaggggaaggcgattgcagagtcctggggcggggcgtggggagctgcgaacggaaaaggctggggcggggaggtgaggctggagcccaggacgccctgggagagtgccctactctgccgcagcgccaagctcgcagagccgcgggtaaggggtacaaagtgcgcctggtgcggctggaaggatttctggggttggggagtaatggggacctcgagggaccaggagcagaactgtgagaaacggagctaggtgatgtctggtttactccggtttggggatggtatagacagctaggtgtcctgaagacagacaaagtt

SEQ ID NO: 111 - DNA, 585 bp, Homo sapiens
gcagcttgtgcccaagaagaagcggcagcggttcgtggacaagaacggccggtgcaatgtacagcacggcaacctgggcagcgagacaagccgctacctctcggacctcttcaccacgctggtggacctcaagtggcgctggaacctcttcatcttcattctcacctacaccgtggcctggcttttcatggcgtccatgtggtgggtgatcgcctacactcggggcgacctgaacaaagcccacgtcggtaactacacgccttgcgtggccaatgtctataacttcccttctgccttcctcttcttcatcgagacggaggccaccatcggctatggctaccgatacatcacagacaagtgccccgagggcatcatcctcttcctcttccagtccatcctgggctccatcgtggacgccttcctcatcggctgcatgttcatcaagatgtcccagcccaagaagcgcgccgagaccctcatgttcagcgagcacgcggtgatctccatgagggacggaaaactcacgcttatgttccgggtgggcaacctgcgcaacagccacatggtctccgcgcagattcgctg

SEQ ID NO: 112 - DNA, 469 bp, Homo sapiens
tcccgcgctgggaaacccggaggggaggggctgtggagggcgggagccccgggcagtaggtctgtgcttcttccttggtcccattacacgggaagggcaaggtgacgccactcagccacgcaggcctcgcattggtgccttcccttccctagccaggacgcggctattaaacgctgcttttagccgattgtatttcctgtctttgaatccgttttctttctcctctctggttggttggttctagctctaagcccatcaaagactttaaggatcctacacagagtgctgcaggctccctttgtcagaattggggcccctaggagctgagtgcggcaggtttcgcctgttcctggagctcgtagtcagggtcgctagtgcactgccactctgaggactggacggagttccagacctgcctccggggacctcctgcgtgagggcggaggccctttcatgaccgtgagatgct

SEQ ID NO: 113 - DNA, 625 bp, Homo sapiens
ccagagcgctgtgcaggggggcagacggcaggcgcgggcttaggcgagccgggtgctgcgaattttccagggcctcgagcacagcattgccagccgagttggacaaattggagaatctcttgcccgccgtagggctgtgcagccctctctccatcagaatcatctcttttcttattctttccatcatctcagctttcttaaaaatgtcacagtggccctgctgtcccgtcctaatgataggctgcgcctagggctaattctcattcagccccagcgaacgcctctaaatattattatcgctttcggagggaggcggaaaaattgtgggatgccagagcgagggaatgactggtcaaaatgacccatacatccttccgatcccgagatgtcattcatcaaaaagtagcgcccgctcgtcattaaggtacgaatgacgctgttcgaataatcatttattgtaacaggtttataagcaaataaatacaccctcctgtcagtgggtaatgaaggcagcttcagagcagacaaatagatccaggtaggaggcgagaagagacaagtgaggaggaaggctccggtcctttgcccgctccgagccagttctgctcgcccgggggtttggcct

SEQ ID NO: 114 - DNA, 306 bp, Homo sapiens
gacgcgagtgctcggaagagccgccccacgtgctttcggggaaactgcccgggagcgagaaggactgggattgagaggaaaccgaaaagcgaaagttagagcctgcgggagcagtacgcaggcgcagcacgcaggcgcagcggggctttcccgcagcccgcttcctcccaacggtttctccctggcggcgggcgcactgctcgcacgctcgccctggggctgcagatcggcttgcgggacggggtcccagagaggccggtccacgcggcccgcacgcgcgagtgttagctctgcacacgacggaag

SEQ ID NO: 115 - DNA, 298 bp, Homo sapiens
ggggagagcccactacaggcaaggcaggaaggttaatcattttgttaaccggaaagggtgctgccccgcctggccgtcgtcaccgagcgtgtcagtcacccgctctcgatttcgccgcagaattacctgctcaaagagccaccccaagaagccctcagctccgaggtcttcccgcccgtcgtctgcgggaagtaaacggcgtcctccctccaaagaagtggatgccccttcgactctcctccgccccccggcggttcctaaatctaggaaagccagccccggcatcgctcactactcc

SEQ ID NO: 116 - DNA, 579 bp, Homo sapiens
taaaaacaatatttccttcatctccctgcctcgcactcaatcctccacccggttgtttataaacaatcccctcgactgtggatgccaaattgttcctttgggctgtgccagccccaggacctcactcacctaaaggataccgttgcagctccaccaaacgtctgtgaaacggggccttacgcctctctcctggcccgtgtgtgtgtgtgtgtgtgtgtgtgtgtgtgtgtgtgtgtgtgtgtgtgtgtgtgtggtcagggcgtgtgtattcgcctggcccgtgtgtgtgtgtggtcagggcgtgtgtgttcgtgtgcgcgccgcttcgctaacgcagagtcctcgccagacgctctgggaaacaggtgttgcagggggcgcaagatccagcgacacctacagactgggggtgacgcccccgacttggccgtgtggggaacagccgaagcagaagcagggaccctcttcctggagcccaagagaagcttccagactcaccaatttggcgggcacggggcaaacttgtagccggaagcgaggcgcagctggggcagcccgggccgggtcctgccgcactgg

SEQ ID NO: 117 - DNA, 486 bp, Homo sapiens
ccaggactggtgggaaggagtggtgacccgctcgggctcggcgtggacccggagcgcgcctggcgctgtccgtggtgctgagaggtggggaggccagcgtggcggcggcgctgggcggaatgtgggcgagacatctccgcaaggcttggcggtccttccccacggccctcatcctctcattgccaccaggcctcctcccacagctgttcctgaatccgctgcattggcctctaaaaccaaaaatcagggagagggggcgacagactaggagtttgcgctctcttccccatgcgctctgcgcaggagttctcgagtgtgcgcagtctggcacgtgagtcagcgcggggtctacgcggattggtgcgggggaaggggagaaggatggtcccattccccctccatcctgctttccctcagcggtccccttattggaaaccggatcacagcacgaggaagccctggctcgtctcctggcccagccttctc

SEQ ID NO: 118 - DNA, 392 bp, Homo sapiens
cgggaaggacagaggcaggctaatgagacgccgcagcccgacggggttccggggcaccgcgaggagagacacaggcctgggtgcagaggccccagccgcgagcctcattcactggggaaaccagggaccaggagggctcggcggggccaccacccccgcgtgcacagtggagtcttctcccctgtccccctccctgcacacacgtgcgggtccctgggttgggagggccctgatgggaagggggaggagccaggcacggggcctggcacgtagtgggccttcattgaaaggctgtccctcttcccttcgcctttctggtccaggacctgccccagccaaggccgggcagaatgggggtggggggtggagaagcggaggctggagtgaggagg

SEQ ID NO: 119 - DNA, 532 bp, Homo sapiens
agccttccaagcgctgctgcagtgcggtcccagctccgcggggggtccccggggatgacctaaatcggggattgcggtgggacagacggtccaggggctaaggaaccccgtggagcaggcagcggcgctcaggaggcagcagcttgagatccacggctcgatttttaaaaattgtaagggagaaggatgcctcgaaagcctctgtggtattttgtcgcatctcgcacccctgtctaaacttcagtccactcgtggagtgtggacccccacgccgcatgggctcaggaggcgcttcgcctttggaaagaaatcgcatgtattaagggaaagcacgtgtgctactttgagaaaggaggcagaaagttaccaaacgcgggaaggggaggctcgccaaccaagcgaggccgtgggacactggcgagtcgcagaggctcctggagccagccgcttggaaacaaacaacaacgcggcgggcgaactcccgggcagcctcccccgtgccggggcgcgggggccgccgcgagaacccggg

SEQ ID NO: 120 - DNA, 514 bp, Homo sapiens
tcaggggagggaagggagatgcggatgggagtggctctcctgccgagtccggaggcagcggctgaggctccagcccctccctatgtctgcagcgtccgtgtgcctggccctgcgcccctgcctcaacggcggcaagtgcatcgacgactgcgtcacgggcaacccctcctacacctgctcctgcctctcgggcttcacggggcggaggtgccacctgggtgagtgactggcccagggcgggaccacccgctggctgcgctgggctcaggaggagcactgtaggctccgccagtggccctgggcgcccagggtcccaggtcaggagtctctgtccccagggtgtgggtgacttgcttaggggaccactggggaccaaaggccatggccccctggagtgagcaccatgggcgggtgcagttgctgccctctctgagccaatctcgggcttgctcctcctccgtccccggcccaacccgagcccttagagaaaactcctcatccgggttcccgccgc

SEQ ID NO: 121 - DNA, 718 bp, Homo sapiens
gacacctggggagctgaatgcacatggcttgctgccagcggctccctcccggggaggaggctgcacagagccagaacgcagtctgcagcagcaatgaccttccgacccccagggacaggacaaagactctgtccctctcacatcctcaccctccttacttctctggctcctcagcaccactcagtaaggtggtgactgtcattgcccccagtttacaaatgaggaactgaggcttggtaggagggggcacactcggtccaggacttcaaagcctgagctcttcacctctgctctctcccagaagggagacacgggccaagtagggcaaggatggaggaaaggtcccattcctgctctgctgcccgcaagctgtgtgccctcagtcaggaaagtaacctctctgggctccgtgtactcctccctaaggcctgcacgtggtacacactcgattagtgttaattcttcctcttccccgaggaggagctgctcacagtctagagggaggatctaaatatttaattaaggaaacttacaatgcaatctgagatgggactgaatagagcaatgagcaaagtgatctggtgcaccaagggaggagggccgagcaacgacgggctggccaggtagagaggcaggacgccaggcctgcttggctcccacctgcacaccggtaccctcagaggcacagcctaggcatccttcaccatgacagagaggg

SEQ ID NO: 122 - DNA, 500 bp, Homo sapiens
caatgtcacctcctcagggaggccttccttgatcacccagtctctactccggtggttctcaaacctggagggtttgataaaacacggggtttctgattcagtgaggctggggagggtcccaagaatgtgcggcgccatctgagagccatcgctcagccctattgccctggtttccctccctcgctttgaaatggtgtctccagcgacttcctcccgtctacgctccatgagggcaagaatctggggctgtcttgttccgtgctcagcgcagaggctggcgcggcactgaacgaatacccagagttaaaaataaagggatagatggtccgctgggcgaaggagccaggaacaccccagaaacccaactcgctgagttctcgtccttcgaacccccagcgcctcccgcggtgcccatggccacggcccctttaaatgtgcgcagggacgccccggccctcgcctccctgaatctctgaccgagcaggggaaaaaaaaatcct

SEQ ID NO: 123 - DNA, 689 bp, Homo sapiens
ggagcactctgggacttgtaggcggccttcgacccggcgcggcgagaaccggtgtgtgcagcttcgctttaggacgccttccccttggaaggtttaggttgtcgcgtcctgtaaattagtcatttccccatccacccacgggaaaatatttttacataggctaaaagtcagacattgcaataagaaataatcaggaaagctcagtttggataccaagtcagctgataacgtcctattgaacgtattgtccatttttctccccaaacatctccagttttatcatgaagagttcaaactgcactttctgacgttgacttaaacttcttagcaaaagaaaaaaagatggtttgggatcaattccttcctaaattcagccacttaactcaggacctcaattttttcgtaaacaaactatgggtgagatgtatttctagggccaggcgcggtggctcccgcctgtaaacccagcctttggggaagccgaggcggaaggatcgcttgaggtcaggaattcgagaccagcctgcccaacatggcgaaaccccgtctctactgaaaatacaaaaattagcctggtgtggtggcagcacctgtggtcccagctactcagaagactgaggtgggaggatcgctcgaacccgggaagttgcagtgagccgagatcgcgccactgcactccagcctgggcg

SEQ ID NO: 124 - DNA, 584 bp, Homo sapiens
atctaattccccatgtcagactgcgtactaccttattgcttttgtgtcccggtgagtgaagtttttcctattttcttgttatggacagaaaaaatccttggagtccctaggctttatggaacattattagcacagctctctaaccagtgtcaccctcaaaacatgtcacttatctttgcctagacatgaaaagcccagctcaaactcctttggccccttatcctggtacaaatttcttttgggccctcactatgtcagtttccacctatatattaatagctttgcatttcatgtgcttctggcatttgatttttttttctttatctaaaggttatatatgtgtgtacaactttaacgtgtttttattactatttttatgtgtttgtgaagttatgtaagggagagtggataatatgggaggtaggcatccaaatcaaatcagtttagcactcctgaaagatgagctgatccatgcaattgccgtgactatgcagtggttgactagacgtaaagatgttgtttagggttatcagccggggtgtttcttctgaccaaattacttgaactgcatgggacccattatt

SEQ ID NO: 125 - DNA, 656 bp, Homo sapiens
tctggtcagtaccctacctcaaccacgcaacagtaccggcccatggccccggttcagtacaacgctcagaggagtcaacagatgccacaggcagcacagcaagcaggtacttggaatctgtttcccatttgcttctcaacccagttatttttgctggtgaatcttgtactctttggatgagtgtccaagcttggcaatttaaaccagcacattttctctacccagagaagataacaaagcatttgattgactctgtgtggaaaagtagtaggtacaagaaggtgaagcatatgtcagactagcatccctggcatgcatgttaaacagtctccataaattattttttgatccttcttctgttaaagcagaaagtcaaccatgtccgtacctttctagttcataccttcttttaattttttttttcttttcaatttgaagagagtgcttcctctgttcttaaggctagggaaccaaattaggttgtttcaatatcgtgctaaaagatactgcctttagaagaaggctattgacaatccagcgtgtctcggtggaactctgactccatggttcactttcatgatggccacatgcctcctgcccagagcccggcagccactgtgcagtgggaaggggggccgatacactgtacgagagtg

SEQ ID NO: 126 - DNA, 722 bp, Homo sapiens
caacgcgcagcggcccagggcgcgcgcagcctgcgcggcctcccgaggccggagttgttccccgaaagttgcatggggcggcggcggctttcatttctgcaccagagctgggggcgatcggctttcctgccgctcttagtggggcggaccgtaataagggggcttttattactaatctgcactccttgacctaaccgtaacccactcgcaactctgcaatcttcatgcaatccacaaggcagggagaggggagcgttccctggggaacccacggcagcctctgatgagatgcttttgaagtcattttaatactctccaaagttcatgtgaattttatactatcttctctaatttagctatgtattgatattaaaaataatgtcctaacgtttattaacatttaataactaaattaacattgagttaaattggctctagtaaggttccaatccgccttcctctacctccccgacaggaggactgttgggcagatgtggaagcactggtgggtctgggatgtcacgatgaaaccaggatcttgtggacgtggcccttccattgatcaacttcacagacactaacaggcagaaaagaaaaacccatcgagaatgtggcccaggcggcttagctaaaggacacatgcaaccccaggaaaggggcgtccctcaggcccggccgctggttagttgctctgcttctcccgcagcagaaaacccctgcaa

SEQ ID NO: 127 - DNA, 501 bp, Homo sapiens
cggaggtcgacacggaagtctggaccgccgaggcgtgtggctcgtctaccggcaggtgagcgtgcaccgcgggcgtgggatgctgtggggacagtgcgggggacgtgtggggtcttcggatgcgaacgcgagtccgtgcgatgcccaggtcaagtttgcagagagagtctgcggtgtctgtctccttggcctcctcttgatgctcctgcgggttgggggctcggaccaaagcttctatttgggggaagggcttcaggacattcttgccttgagatacttgacaaaggttgaaaagacacatcaggtgggagcttgtagctgctgctgatggggtgagacaggtgcagtcgagtgtgcacgcatatgtgtgtggctcctgggtaagggtcagctcgagatggggagaagcctctgcgacgggtcattctgtccccaagagccacttagattccggagagtgaaattgagccgctctgaggtggtcactagatgttatggcag

SEQ ID NO: 128 - DNA, 593 bp, Homo sapiens
tgttgctggtctgcaggctcacctgccagggccacttcctgtctggagccggcaatcctccagttatccttgaagtccgctggccacaggctggggcaggtcaatgaaaggaagggactaagagtaacgctgctgcagcaccttcaccagagccaaatgcgcctcggaaccctcaaagcaaggcctgcatcccctgaggctcttcggaaaatggctgtgtcctcccaggtctcagggagccgccccgcccctggacagatcgtgtcccctgagcccctcctcctctagggcaggtcccctccccctcaaaccaggccagttctctgcggctgggcatggcccagaagcgtcacctgacgtttaatgctactgtccctttggtccatgttgatgatagtgtgtgtgggattgccagagcctggtaagaatcagactcacctgagggaagtaacaccgaagcggggcgcactgcgttccccctggactggggggccgctggggatcctggagcccctacaggtggcggctcccatccgctcgctccggggtcctggccgcctggccctgaggggagcgcggaggggaagagcagt

SEQ ID NO: 129 - DNA, 460 bp, Homo sapiens
tcctctgggtcctcccgcccttcacagccatgctcggactgcagggccccggccccgccttggcgcgtctgaaggatgcagctctgccgcggagctgaggagacgtagccttctggggtagggatggagggggtcgtctcaggggacatacccctgcccaggggtctgaggaaacacgaccctgaattaagagggatggggctgagggcttcacagccccgccctggggaggtttcagagaccgccttaacctctgcgggtcacattcacgggaatggctgggctaggcacaccaggctgctcccaagtcagaggacagaggctgagacccctcagcagccacctgctctgggtgagcctccacccgcatgatatttcatttcctttcttataatgaggccagtgagtcccggtgggtggcagccagggcccagggtgtgcaggttggagggaagggaaa

SEQ ID NO: 130 - DNA, 511 bp, Homo sapiens
attaataggagccagccctctagcagcctcagattgacttcccagcacccggaagggcacctcaacctgtcccacactgctggtgcttagagttacctgtcaaagctgctcaaggctctcccatgtgtgctgtggaccacggcatctgttgtcaacaccagcgtggcctttcctatgaacatgaagctgagggttcctgtgaatgtgagggtctgagccagctcctcagagatggaggctccctcctctggcctcacctgttaccagcatacctagccaggtgactgcagccttcaccctgcatctaaattgatggccccagtccttgaggtttgacaggctgttatcttgggcccaggtatcctgcaagaccatatttgggtgatccttctggtttggaagcactaggatcccaagcatcctcctgactggcccatcagggtttactccaatcccaagcccggttaaatgtgctgtgtgctgcccatagggccaccattcctcttagtcc

SEQ ID NO: 131 - DNA, 468 bp, Homo sapiens
tcagatccgtattccgagcccgcacgcataccaggctgtgctcagggtccggaggaggccgacccagtagccgtgggccctgtcggaggggagccagctgggcgcgcgttgttttctgaggtggccgcgctgtctttcagggtgtgtgtacattcgaaactccactgaaccttgattgaagacctacagtgtggcagtccatggcctggggactggtaaggattgaggccgtaggatgggctcgagtgtggcggtgtccccagggccccagtgagcaagtgggtgtgctgggaacctggccacaagtgaacctcctccaggcgaggggatgccctgcagcatccagccgtcaaaacctcctgtagaaacgggagcgagcatcaagcctgtgtgtgtgcatgcttgggcggagtctacccggaggagaaagaggcttccgggctggccctccaaagccggttgtacagg

SEQ ID NO: 132 - DNA, 694 bp, Homo sapiens
gaaaggagagcctcacgagtcgcatctccacttaaccccgccgcttgcccggaaacaccttggccctcttccctcttcaatcccagtgcaaccgagaaaaggccccacaagtttgagacactcaaagaactcacagcgaactcacacatacatcggctctgtagggtaaaacatccccgcctgtttaccaagagatcagatccgaaaagacagtatgaaatgagacctctggggagactgtcaaccccaggggtaaaacaaaaattctgatcagaaactgagtttcccaaagaaggggctaaatgttttccaacactttcggggctcagggaagatgactctgtaaggacactgagaatcttcctcgcgtgccacggggaggaggactgggggcgtttgaggggctcagcgcaccagaggagtgaggtggaggagggcgttcccgcgtcctcctcttcaatccagagcagctcaacgacgtggctccctttctatgtatccctcaaagccttcgcgtcggattaaaggtgttcttgatccttctttaccaaccacggtgcgggccaggcgtgatgagggatgagggagaggaaacctcagtagcaaaattgttcagaggacgttcggagggcgcggggagcagccggatgcacacctaaagtctcgcaacacccaactcctcctccgcaggcag

SEQ ID NO: 133 - DNA, 558 bp, Homo sapiens
cactcagttatgacttacctgtcccagttccccaaagccaagctcaagccgggggctcctctcaaacccaaactcaacccgaagaaagccagggcctatggcagaggtgagtgctggtcctctggtgttgtattggagacatgtcctctggtgttggagatgatttcatggcttcaagagtgatgttcttagaatcaaaaatagataggtgtaatcctcaaagagaccccaagcctcctttgtaacacattttatgactgttttattctgccttgtttttctaaggctttaagaaatgtttctgcttagatggaaagggcaagtttgctgcttggtgattttagtgcagtagcccattgctcccatttttcagaagaggaatcgcggggtagggagtcgggggagtttggtcttgccccagatcaccacagtcagtgatgggggtgggccatctggctgctgattcatttctccttctgttacactaagcctgcctcagatttccagccggagtgggagctattgttaacccctggcagatacttccttgctaagaca

SEQ ID NO: 134 - DNA, 157 bp, Homo sapiens
cggtgagaaaagagtctcaaattaacccccctgagcccttccctggacccgggcctctgccacgcgtgctgggagctggacagtgaaggggcaaagttaaagaggaccggagagccacctcgcatttacctctttcccccaccaccaaggagatgcg

SEQ ID NO: 135 - DNA, 223 bp, Homo sapiens
cagagttcggtcgacagacggaagaacagaagaacgttgacgtcacatccgggagcgcagggttgccgttagttaccgaggaacgcggaagggtttctattgatgtcgatttcgtgcttacagcagttcttagcgaaggggaagctatggctaccgatagaggtcgatttccccggaaggaatattcaagttttcgtgtgtgtgctttccaggaaaatagttc

SEQ ID NO: 136 - DNA, 327 bp, Homo sapiens
gctggggggaataaaatcatacaggtggagcctgcttcccacgagtgcccggcatcctgctggggccctcgggtgccgcgcgctccagctcacctggtacacgtcctgcagcccgcaccacgtccgcagcacctggtggcaagcccgcagcctctccgtgtaccatctccgcagcgtggacacggtcaagttccatacgaagcggctgccgctgaacagcaggtcctccacgccacacatgaacaccgaagccatgcctcctccttcgctctgaacccgggcgtcccggctctcgtacgtgcagccccgaccccagcggcctgcccg

SEQ ID NO: 137 - DNA, 347 bp, Homo sapiens
ggcgggccgtggggcgcatgcgcggccggcgtccagctctccgggaacccggtacctatccgccctttggtcgggccttctccgcctcatgacactggttcaaagccaaacagaaaagcccgacgagtttattatcccctaaaggacgtcatgtagataattaaatgacatgaataccgtcgaagatacctgcctgatattccaaaatggcccaacggagccctgatcgcggcgttcctatgttgaggttttaacttcgattttaagaggggtcctgggagatagtaggcagcttgccggcaacatcaacaacaaagatacatcgtgggatttttgttatttttaaa

SEQ ID NO: 138 - DNA, 361 bp, Homo sapiens
agtctgtgtccctcaacacctcctgaggcagctgatcgcattagaggcccggatccttcccctcgatgtaagctgcccccatgtcacgtgacccactcaggctctgggtgaagggggtgttagtaatgagtgcctctgcgactgccatgagaacatgccaggtcagcttgccagagtaggagaaacatatgcacagagctgaggcaccccaggcctccaggggcagcccacagcactcgacctgccgatgtgagcgagcccaggacgggtgggctcagtctcctaagcctcgaagtgctgggcaacttctccgggtgattctaatcttgctactgctgaggcgcactagcccaaaatggcc

SEQ ID NO: 139 - DNA, 650 bp, Homo sapiens
ccgcacccgggcaggagagaacctcctcgccgtacccctgagagccccccgggggggaccgtgtctcgctgccccgctgcgtctgtcttccccactctacccctcttttgaatccagaaaatgacagaaattgtcaggggacatttgtttctggtcttttaaacctagatgccagtcccttcctccacctcgcgagacaccgatgcacagcgcagtcgtccgaccgacagggcggaaaggaacgaaggtgggggagagccagggtgtggcgaggcccgtcagggcggtcagacctagggaaacctggcgtacttggcgcagatggggctcgactcttgccccccgtggcttctagcccgcccttccctctcgaggcctagaccctttatttgggcggcctcttgtggagtctgtgggtgcccccgcagacctggccagcgagagaggagctgccaatatcaggcgcgcctacctcaaaccagctgctcatttggatcattttatgccaaatcctaacctgccccatgggagagaaggaacggtgacgctctgaatgccagtgccactatctccaggacagtgccctgcgtttcttgatcccggggtcgccaggctgacaatgccactccccaccctggcaattaggtgta

SEQ ID NO: 140 - DNA, 366 bp, Homo sapiens
ggtccacttcctactctcgaagctagggaatggggagggatccgtggcccgggagtcctgcgaagccttcctcgccatcctgtgccatcgccctgacctttatagcgggagtctgggtggagggcgcggagatgctggggtgggggctccactcgcataggtgtgacatcgccctctactgggtccgcagggagggcaagatcgtggtcgtttagcgccaaggcgggaggggccctggaaaccacacgatcaggggaactcttggactgtgcgccccaggtcgaggatctcgagggatgtcttgagcccagaggtccgggggaagtaaggtactctgtcggctgtgccttgtgcagccaccacgca

SEQ ID NO: 141 - DNA, 592 bp, Homo sapiens
ccgttcaccacggttggcggccctggtcactcccacggtctttaccagccgggtgccagtggctgggaccaggttctgaccactgtgcccactcactgtggaatcgcgtgagcaggccagaccctgcgtcgtaaggacagagcaggacacgtcctgtcatggggcacccgctgtctgcgactttgtttccgtttgtttccgcctcagcttgggggagacagttggcaggcaccagaggcgctcctgtgaggtcacctggccagtctcaggattagctgcaggctttggctgattttcttgggtcttgtacacagatgcttgtcagcaaataccagagacttatctcttcccttccaacatgcaaaccttccttcatgttcttgtcttaagcgttttggcacaacctccagcctaataccagcctctagggagataaacagtgctgccgatttcacaggccaggaaaccgaggctgagggccccccacccacctcctgctaccccgcacccctcctggggccttgctgggcggggtgactccccggaggcggcaccaaggggaggttcgtttcgaggctcagagcacttttct

SEQ ID NO: 142 - DNA, 429 bp, Homo sapiens
gggcttctgcccagggcgtgtggtgacccacccttgccccctgcatgcccggtggctgaggccacaccccacctagagcatcctgacagcaggagccccgcttggtgccaggcagtcctggctggggtggggcaggtgccatacagttggccctgggtagaccctcaccccgacccccctgtgcccacagtgaagcccagtgtctccatcggtcaaggtagaggtagaaccctgagattcgaaggcttctccacacacactcattcattcacatgtcaaatgtgtactgagcaccatctctgttccctgcactgttccaggctctgggcatggcaaggacaggacagtcacaacaggcctgtggtgagaatcttctggccggggcggggtggggagcagcagcgtgaaggaaccagtaaacacacaggt

SEQ ID NO: 143 - DNA, 1541 bp, Homo sapiens
gccaggttgtcccggggacgcagggccgcgcagacgtccccctacgtcccggggcccctgccccaacccgcgtcccaagcgcctgctctcgcgcctgctgcaaggcaggacttcgcgcgcttttccccaacccatgacactctaggaaattcacagagccaaagttagcatagaccgctcgttgggggcttggccagtgagttgggaagtgccctggaggtagcgcgggaaacaaggggaaggagggagacagacagcagctgggcccctttgggctcagagaagggacaaggggagctcatctggcaagaatgatctgaaaaggcctccaagggtgggcgggattttgcggggggagggaggaatttccaactggagggacaatgacaaagggggtgaatagagcctgggcatcttgagaggaattaatgttaattcatttcctttttttgcgtggggggatagggtctctgtcatcgaggctggagtgcagtggcatgatcatggctcactgcagcctcgacctcctgggctaaagcgatcctcctgcctcagcctcccaagtagctggaactataggtgcgtgtcaccacgcccagctaatttttgaaattttttggtagaaattaggtctccctatgttgtccaggctgttctccaactcctgggctcaagcaaccctcccgtcttggtcccccaaagtgctgggattacaggcgtgggccactgggcctaccctaatttacatttaaattgtatcttgtgttgttccagagaagatttcaggtccctggaatcaaggtgatctgcttctgtcctgtgctgcttttctagtattttcagagccatcgtttcatttctttttttccagctgttgagttgtttcaggcaagagggtaaattcagtctgttactgcatcttagctggaagcacaagttcccaatatgggatgtatgggggagaggaggtaaaggaatcccccaggagggtggtggagggagaatgacctcatgcgaagtgactccagagacagatcaaatgctgtcattgccatcattgcatgtttttaagcctcaggacagaatggccaggtgagcctggcctagattctcacctgtgcctgccttccccttgtgttccctcgttcctgctcttctctccatctcctggtgcctagaatcagaggtcacctgttttaccccacatacatgttttgccctgaccccactcctgagagctacaggtaggtcttggtgggcttagaaggggatagtccaaattggcctatcccccccaagcatgtccctgttggtcatctggaatgtggtcctaactgcaaatgttccagctctgtgaatcctgcccccagtgtctctagaaaggcccgttataaacccacaggaattggaagctggactgggagcagagtcttctgggatcctgcatctgacagtggtgtggggttctaagagcccttttcctatacagcccggtttcccagataactaagcaatattttattcgtggctgcatacacagg

SEQ ID NO: 144 - DNA, 466 bp, Homo sapiens
ccagcccctcctctcacacctgcctttggctgtccctgggctctgagtccggagcccaccctgcttcacccctgcggccactcgtggagacgccaggctgtgggtttctcttccgttcaccgagagtccactcccattcctttccatggtccagaaactggttggcgtctctcactgcttggcacctgtatccctcccaagctccttcctggcatgaaccagttctcacttgttcttctgtggggtctcaggaggggtgagatgggcacagagcctggtaattccactggggtttggataagccaggaagggatacgatgagacaggcattttaggggggcctttggaggccgctcagaggatgggctggataggggaggcgctcacagtaaggagggcaggtaggaggtcaccaccggccccagggcagtgaggatgaggctgactgcggcttgagagggctgtc

SEQ ID NO: 145 - DNA, 821 bp, Homo sapiens
cacagtgagtggaagcatgccggtgcctctgctgctggaacccggctgccggaggatggaaaggacagctctgccacagagacacaacagatcccactagatagggtgggagacaaagagactcaagccagctgcaaccctgaaccagcttctctgccctatgcagaccctggaaagtcctctgaattccagggcaataaaacaaaacaaacaaaaaactctttactcctaagtaagagctcatgtgaccccagagctctcagaacccttcagggtgaccacagcacatgaaacgcactgcagaactcttggaaccctgcggggtaactcttggaatcctgcggggtcaccacagcacgtgacgtgcactgcagggctctcagaaccctttgggatgaccacagcctgtgaagtgcattgcagagctctgagaacctttcagggtggccacagcacgtgacgggctctgcagagctctgggaaccctgcggggtgaccacagcacgtgacgggctctgcagagctctgggaaccctgcggggtgaccacagcacgtgacgggctctgcagagctctgggaaccctgcggggtgaccacagcacgtgacgggctctgcagagctctgggaaccctgcggggtgaccacagcacgtgacgggctctgcagagctctgggaaccctgcggggtgaccacagcatgtgaagcgcactgcagagctctccaaacccgtcagggtgacaaaagcacgtgaagtgcactgcagagctccggccagacaagggtgtgagagcaggcatcccagggctgcaccttagaac

SEQ ID NO: 146 - DNA, 381 bp, Homo sapiens
ggtgagtcggggtgcgggccggtgcctctccctgcgccccaggcctgcccggtcccgtgggcctgtcctgtcctgggcctgccagccctgcggccccgcgcaggtggagggtgcgagtgtgcatgtgtgcggttgtgcgggtgtgcgagcatcacgtgtatctgtcctcaacagccgaagcttgctgtgcatgctgctgatgcgctctcctccctggtactctttgtgtctgtgtttccacgtattttcttctggttcacgtttactaggacgacgagagacagcgtgagcgagcacgtcctttgagtcctccttaccctgttttcagagccgggccaggcccagcagcccccttagaggtcagaagggcctcgcctgcga

SEQ ID NO: 147 - DNA, 553 bp, Homo sapiens
atctgggctctggagagaaggctggggtggttcagcctccaccgcccaccggggctgtgttctggggcttggggacccactgaggataggctctggctcctgtctcaggaggggccctgacatagggggttgggggtggcccctttgccccagctgtccaaatcacaagctccacaggcagctgcttcaggggcacgaaattacatctctctcaaaatacccctttctttcttctaattgccatgagtagaataatcccactctgtcttctctctcacccttgacggtgaatttaatttaatagactgtaactgtgctgcctctgatctttaaatgccatgtttatttgcctcgttagaatggactcgatgattggagaaagtgtggcgatgtcttgtgaagtgggttttgacctgcgcgggtttcggcattgctgcctcggcagtcccagattgaaaccgatgtggatgcaaacgccttcccttcccgtcgggtggagcggccggcggggctgaggctccctccgctgagtgtccgggtgggtcctgaactg

SEQ ID NO: 148 - DNA, 403 bp, Homo sapiens
tgggaaaatccctagagccaggatcttcattcctgctaagccagacagccggaagacacacccaaattctgtccctcttacttcagggaacatgtccactttcggcagcattacaattttggcaccaaatgtgctaactgcaattccaccatacaatgcgtaactggaaatggaggcaacatctccgatcctgaacgatcgatgcgagaatccaggatatgcacggcttattttggccttttcccactgaaacaagggccagtattaaaaatggcacgctatcctctgtttcactccctgcttttaaacgtctccgatgtttctccctgagacagggcctcacttccgtcagccgggcttttctacggtataattttccttgtttgcttttgtccaaattaga

SEQ ID NO: 149 - DNA, 377 bp, Homo sapiens
ggatgcgccaagcacccttcggttttcccggggagaattttccccggcccggggactagggtctggcgctggggcgcccctcggacctgcgggatcgcccctacactctggcgcgctgagggcggtgagcgagggcgccaaggcacaggtggggcgggagtcgagcgcggaggctcggggggcgggacgcggggcctgggagcggccagggaccgcggcagcgcctcagtgccagcctggcgcccgcgactgcctgccccagcccctcagtggcggcttgctctcttctctcgctccgaaccagacacagccgctgccgctgccgtccggcgcgctacagactcccgagaacagccctggctgtcagcgagcaccag

SEQ ID NO: 150 - DNA, 279 bp, Homo sapiens
gccagaggcgcttcaggaggcaagagaagtccccgcgcgctccgcagcccggcgcagctcatggtgagcgccctctggggctcgagggtcccttggctgagggggcgcatcctcggggtgcccgatggggctgcctgggggtcgcagggctgaagttgggaccgcgcacagaccgcccctgcagtccagcccgaaatgctgccgccaggcagcaacggcaccgcgtacccggggcagttcgctctataccagcagctggcgcaggggaacgccgtgggg

SEQ ID NO: 151 - DNA, 467 bp, Homo sapiens
cacccagcggttggtgaaccagagctgcgggctctgagagcttcggtcccgggcggcagcccacagcgggcgcggggcctgggtgcgaggggcggggcagagaccgcgagagagagttctcgtgaaattcgggagactcaacgtgggggaaaaacagtatctggggatccaggtgcggaaatgcacctcaagtgcaggaacgcgggtcagggataggcccgacgggccttgggggtggggagaagggtgaataatggaaggttggtgcgggaaagaaccttggtacaaatcccgccattttaagcggaggttcggagagaaaacaaggtcactaacccaggaaaatgtctgcctctttcccataaatcgcctggccctggccacagacctcctggagactctgttctctgctcacgccggtcagtcttagattggttggctggagtactgctggacagatagagaaa

SEQ ID NO: 152 - DNA, 388 bp, Homo sapiens
taggaaaaccataattattattctttaatgctctcagagtaggaaaagccgggcatgtgacagagcctgtcctgtaccttggtaaacatgggcttcccgtgctgggtgaccatggtacactggtcgcagtccaccgtgatggatgagttgtggtatgactctttcgagtgtttgagaatgtaatacaggtcggtcacccctccttcaaacacagtgctaaagtaacgggggatgagggtcctgccgatagctgggagagaaacacagagaaacaaaccattaacaaaaatatcccaccagccccacacacccaacaccattgccaaacctcctaaaaccggatactgatcatctcaagaaaccataccaggaggcaaatttctcttta

SEQ ID NO: 153 - DNA, 279 bp, Homo sapiens
accctccgcaacgcgcacacgcacacccctcgggcggtcgaacaggagccgggccttgccgcagctcagctccaggcacccaggcgagcgacggaccagatctgcggctccgcgcttccctgttggcctaacatcttaaaaccagaggcgggcttcctggtgccgagacgtcactccgccgcggccctccccagccctctccgcctccgcctcctcccagacccttctccgggtgcgactgacgtggctccgcaccaatcaggacgccccgagccgcgg

SEQ ID NO: 154 - DNA, 437 bp, Homo sapiens
cagcggcagggcgcgggccgagccgattccccgcctccttgcggcgtcccggcggcctcttaatccctggcctttcccactcccactcccgctctcttcccccttcctctttgccttccccgccccgtagagctccaccgcggatcgggcgggttcggggctggcctcgcctcctcagggaggggttatcggcccctaaggccgccccacagccacacacgctggaaacaccagctcgacccaaacaaacgtgtgcaaaataaataaacgggataggaagaggggaaagaggaggtggcctggcctcttcctgccttttcacgatattccagtgtcaacaggaaacccatttgtttgacacttaaacaaaaagaggaggaacccgaccggggccgaactgggagagatgggacagctgggagctgctgaactttgga

SEQ ID NO: 155 - DNA, 341 bp, Homo sapiens
tgtccgtcaccatcaccaccaaggacaaagacggtgagctgtgcaaaaccggcaacgcctacctcaccgccgaactgagcacccccgacgggagcgtggcagacggggagatcctggacaacaagaacggcacctatgagtttttgtacactgtccagaaggaaggggactttaccctgtctctgagactctatgaccagcacatccgaggcagcccgtttaagctgaaagtgatccgatccgctgatgtgtctcccaccacagaaggcgtgaagaggcgcgttaagtccccggggagcggccacgtcaagcagaaagctgtgaaaagacccgcaagcatg

SEQ ID NO: 156 - DNA, 650 bp, Homo sapiens
tagagatggtgcaccagtagggactgtcactggattggggaaaaaaatccggggctgtccttatctgctcccttaggcaaaatcgttctgtgttagtctggagggtgaacctattttaatgcaggagggcatagacttttgggtggaagcggaccctggagatgtactggcaggtgaatagagtgtggaaaagtggggaaaggagaagcacgattcatgagcctgagttcgggcttccatggatggaaaagcatcaggagtggaatttaaagacggtgtgtttcaggtgatgtgaggtcctggatgcccgaagcttcaagaaaatactggttgggcacttagctttccctgtggttgccaatcccacgcaggccgcctctagcgtctcgattcgcttttctctgacctggaacctccgccaagcccccagctctcttcttcgacccagccttgcacggggcagctgtcggggcaggacccgcctctggcctgtcgcagcctagctcacggtcaagggcgggagcctgctgggaagcacggccgcgggggctcgctcggggccacctgcctggaactcagcgcgaggtccagagttccctccggctgcgcagcggcgcacgtgccccgtggaaggggcggttcccgcaccc

SEQ ID NO: 157 - DNA, 499 bp, Homo sapiens
aggtgtttaaacaggtgtgcgtaggtctttctacttcagctctcagagccggtcttgtttgcaggttagtgggtggcgcacctcgcggttgctcccaccccacccttagtaacagggtttgagtaggaccctggggtgggggagtggatgctgctgtttcaagctcgttcgcccaaatacatctttctacccaccttccctatcgtcagagaggtggcctgaccccagcacgtgcaagcttgtcaggctgggtaagttccagaaccttctggagccaggattctctttcccagccctgggccctgagctcggcggatgggatctgcgaggctcggggcgcaggggaaatgggggcgtgttcctttaaggcggaaccaggcggggagtgggacgtcccccgcgggcacagcccgagccgcacctggggcccaattagcctggagcgcggccgggtgtgttgaacgtagcaaccgcgggcggcgggcggcgggcggcgggc

SEQ ID NO: 158 - DNA, 567 bp, Homo sapiens
cggggctgagaactggcgcatggctgccacctgctgtggtggtgaccaccgggagtctccctcgcaggctgtctggctcaaaaggtgacaaagttccagccacggggcccctgctcagccctgaggacacccacactggcacaggggacactgcggcagtcagggaagggcacctccccacgctggcacagggtctcagcacaggggacactgcggcactcagggaagggcacctccccacgctggcacagggtctcagcacaggggacactgcggcactcagggaagggcacctccccacgctggcacagggtctcagcgagagactacggcatttggggaagggccattttgtcattgactggaactttctcaagaaagcattttccacttgcgtttccatggaggtcggctttgctggaaaccacaggaccctgccctccaccagacacacagtgtgcagaggcgtgtacccgaaaagcaaagtgcggcctcacacggttctttaatctggagccggactcctgggaaacaaaccaaacagacgcaggtacgtgctcagctctg

SEQ ID NO: 159 - DNA, 476 bp, Homo sapiens
gcggagtgcccaacaccaggctccacaaacctgggtgacctgcactcgccgggggccctggtcaaagctggatccagggtgaagaggctccatcccttgcccgtcagtgtgaggactccgccagcggcctcggccattggagctggatgggcacaggagccctgctgccccagcacacacatttcatcatgaccccaggtgaccagcacgcagggaggctggcagccccactgtctctgttcctttctccctctcggcagcaactccacctgagctgtcccacaggatccccaggggtgggctgagagtcactctgagaactgtccctgtattcatttccctgtactgctattccatttgctgctgtgacgaatcactgcagcttagtggctgaacacaaacgcatcatcttacagctctggaggccggaaagctgaaacaggtcccactggcctgaaatcgaggcgtgggcaggg

SEQ ID NO: 160 - DNA, 286 bp, Homo sapiens
cactgggagcccggcctggccccgacagcgcagctgctccgggcggacccgggggtctgggccgcgcttccccgcccgcgcgccgctcgcgctcccagggtgcagggacgccagcgagggccccagcggagagaggtcgaatcggcctaggctgtggggtaacccgagggaggggccatgatgtggaggccctgggaacaggtgcgtgcggcgaccctttggccgctggcctgatccggagacccagggctgcctccaggtccggacgcggggcgtcgggctccgg

SEQ ID NO: 161 - DNA, 601 bp, Homo sapiens
tggtccccagaaatcctgaatccaggaaacccctcagtccggggcagaccgggatggccgaccaccctgactaggggagggccgtcagaagtctttgaccctgatctaagttctctctgctttgctgagtctctttctgtctctctcctcctctccctgtctgaatgtctgcctgcctctctcccacacacatctgagacacaggtctcacagaaccgcccttgcccttctgtctgtctgtctgcctgcctctctctcacacacacacctgagacacaggtctcacagaaccgcccttggccctctctgccaggagggtggttctcggaggttcccatcctctgtggggatgctgacagctgtgggtcatggctggtgccgcggttctcactcttggctgcagagagcagctgggggctgcaggtcaggccacacctagctccccatggccctggttcacttaagtgacaggctggggtgaggggaggaggaggccagggtgtgaccgtcggttccatttctgctttcccccagctccccaagtcaggcaccggcacagtggcagttggtttgtgggacacccatctgtcagtagggcatg

SEQ ID NO: 162 - DNA, 489 bp, Homo sapiens
gcacctcagcctcctgagcagctgagactacaggtgtgcaccacagcaccggctaagttatttttgtgtgtgtttttttgttgttgttgttgtttttttgtagagacagggtctgactattgttgccaaggctggttttgaactcctggtctcaagcgatcctcctgcctcagcctcccaaagtgctgagatgacaggcatgagccaccaagcctgggcttgtattgtttatactcagtaataaagagcaaaatttatataagcaaaatcagccactgtaaagagagtttagacataaccatcacaactgcttcctcaaaaagtgtctgtacaagccacatccacctagggcaggtggcaccaccccgcgtgagagagctgggcggaggatggacagggcttcatcgcgggaactagctcctgggccagcagggtaacccgggaaatcctgtccaaacggctacataaacccccctgcaacgctgagca

SEQ ID NO: 163 - DNA, 857 bp, Homo sapiens
aagactgcctgtttctgcaccagtctgctctggtgttgacagagcttgccggctagcacagccccatgctgggtcacagcgagggttccagtgagtgaagaaggtgcctgggggataatcatgtccctggttctagacttactttatattcctgtctgtcctttcctgcaaaactgaagtctttggaatcagcccttgcccacacctctgggatcgtgagtgggctggggtgtgcgctgcagctgggagaatccacttagcatcctgtgggctgtgaatgggatgagctcagctctacaagggtggagactttacagctatgaaccaaggtcattttttttttaaggaaaattccatattagtgaattggctttttgctaaactccatacgtatagaaagatatcagtatctgttgcaaatggcagatacaattttatgcacctaataatttctatagatactgagatgaactatgcaaatctagatttaagtgtataattaaactgtggcacttggtttctcctcttgcagttataactctcaaaattgcaacataattgatcattgaagattttgccttttcaggataaaagccaaagtgattgctccaaatggacatccatgccagtcatctgtgtaaggatcgctgagtgtctcacaaaagcacgtgggtatgaaggtggggttgacattgatgattaaaccaagcagtacagaagcatcggtgctggtcacttatccccgtccttcccatcattcgagatccccttccagcgaatggtgagtgagaaggagtcaatactccccggacctggagtgtgagattgctggtgggtgggtcgagtgggaggtctca

SEQ ID NO: 164 - DNA, 474 bp, Homo sapiens
gcgggcgtcactccttacctctgagcttgcttccttcccacgtcagccccggcaggtgtagccgccacacccagcccctggctccagtgccaggcattggccagggtgggtcagggcacacggcgttcatccaaccgcagaagacgctccttggcaaccgcaccgtagggtaatgatgcaggaggaacgttgtccaaaacgtcagattttgaacaaaatgaccgtgaataatgtattcccaggcactgcttcctgggcagcactgccaaggcgttctgtggcggctgcatctgtcatcggggaacacaaaacacacagggaagtcacccccacgcacggcccacacgtgtttgcaaacagaagctgcgctgttttgaggttcaaacgcgagaggcggcctccgacgtgactcgcctgccagctccgggaggaaagggtgtgtggtattaaaaattcatagagaacactgccttt

SEQ ID NO: 165 - DNA, 459 bp, Homo sapiens
taacgagtggatcattgctgcagaggaacgggagaaccctccagtcccccggcggctgcatgccacggtcggctgtgcagtaaccaggcagagcacaatgacatcccagcctcgctccccagataaaaatttctctccctccacttttgcaaaggtaatgaggaagtaatgaggcttgacttgaattttttcagccctggctttccctccttccccagcaggtcccccaggatggcataagacgctgggtttctataaaagctgtgctgtggctgggtggaggagcggagtcgggctctgtggcctcagaggccgcttgcaagagttcagataaaccgaggcccctttcagcgcgtccgtctgctctgatcggctccactgaataagacgatttgaggatgtgcaggcccggccgcgcagctccctgtttattttaccacttgatggctagaaagccct

SEQ ID NO: 166 - DNA, 325 bp, Homo sapiens
aaaatcagatccggccaccgtggctaggatgctcgtggccgggatgagccggagctgcagtgaaaggaagcccgttgagtgtcttcagctgatgttatcggccgctggagaaatgagatattggcagggcctgctgagtccctcgcagctgagcaggttaataaggtggggaggggggctgcaggctaattggatttggcctggcttgatcaacagtgtcaggcctgctgccagggaagcatacacagtggctttctctttgtttaaatttaagccgggcgaggaaaagtcgcactgctcttgtgaagctgagggtgtgctcccg

SEQ ID NO: 167 - DNA, 536 bp, Homo sapiens
tctaagcttcccttcacagaaaggctcttgtcagatggaaggatggagccggtggggaaatggcaaacttttgcttggtagacattgcaaaagaaagacttccatccatctatctgatcagagaggaaattagaagggttctgggctcaaataaaatttccgagaccaactgctaatgagaggtgaggatggctggctgtgggattcagcctcctaagatttgaggaggaacttcgcacttgggaagccaatttaaaaaaaaaatactcctcatttatgccagcctcaggaagctaagtcttcctctgctgccttgaggggctggaatcgacctgcatgtggctcccgtttctgtggccagaccctggtggtgaacacatttcaggcagccagaaacaggcttgttgtggctctgcagccagacctgccagccttctcgtcaggacgtgcgagcttaattattccactgccctctggaaatgatgccggcttgtccttgaaatccctgaccgggcgccaggccctgctggatttgt

SEQ ID NO: 168 - DNA, 482 bp, Homo sapiens
aagaaataaagaagagtcactaaccttgcctcctgtccgggaagcgaaccgggcctggctgaggctgtctcccatagcaaagcgccccaaagttaaggagctgagccaccacggaaaggtttgcaagccagctcctctgggcacatggcatgattcagatgttgaggagaagaaaagtcaaaaaagttttagccctcagctccagaaataaagcgctcgcctgtggcccagcaggaagatgcaggggtgcatggtcaggcgcgagaaggcagcgactgcaaagctgcccgcgatctccctgtgcttttttaagcagaaaggagctagacacctttttcccctccctcctcctaaaaggggagtgattcagctgacagtgtccgctttcggcggggtgtggaatgggccagagcgggagggcgcagcccaccccggtccccacccctccgcctcccgcatgccccgcggcctcgcagcccgcc

SEQ ID NO: 169 - DNA, 723 bp, Homo sapiens
tggccgtgcaagtgccaccgtaactggtgagagccgctggcaacccacccggagttgacaaccgcggagagacgcagacacccactgacctccaggaagctgagcgtggtggatggaactctacgatctctttctctccaaggacggaaacctcatccaagcagtcccagaggaaacggataaaggtatttgaaagggagcgagcggccccaaatcgcacaattgagcggctgggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagctt

SEQ ID NO: 170 - DNA, 513 bp, Homo sapiens
tcccttttaggagaaagcaacttctccagcagcctcctcattccgatcccggaggcttttggccgacgctgactttccctgcgagaggtcgctaggagtacggaggcatggagctggagcttagaatataaagggaaaagcagacagtgattttgttgttgttgttactgtagtaaatcagaggcaagttatctgatcccatatttcattgtgccaaggaatcacacatggaacttgttcaatatagtttccaagggcctggcgccgtggcttacgcctgtaatcccagcactttgggaggctgaggcgggcggatctcttgagcccaggcgttggagaccagcctgggcaacatagggagacactgtcgctacaaaaaaaatacaaaaaattatccaggcgtggtggcgcgcgcctgccaacccagctactcagggggtggaggtggagggaggcttgaacccggaaggtggaggttgcagtgagccgagattgcgccattgcactccagct

SEQ ID NO: 171 - DNA, 811 bp, Homo sapiens
gcttcgggcaggggcgggcgcctcctcctggccgcccgccaagcatgcccggatccccttagccgcgtcccaacacatgggcttgcttaacactctctgagagaaggaggagccaccccgcagtgctgtctggtcgctttttcccatttttgctccaggatccagagatttgagcaggggtggggtgaggtggggtggcagggttcctccccttccttaggcccagccagagaggggccctgtctctctgctcctttcctcgccgtcgccctctctctgccaggctctcctgttcgtcctccaggggcccacgctagcccctcacctcccctgaagccttttctaaccgctgcggcctccgtggtcccccacgcctgtgttcctgtaggacgtttacagttcacacgatgtctcatctctcggtatatgtctcccaggtaaattgtgaagcataaagagccatatgctttttttgctttaagcaatttttttttctgtagtttctgttgtaagcaataatcctgcccagccactaattgtgtgacctcaggaaaatcctttctgagttttctcatatgcaaaatgaaatttgtaaggtctcttccagcttttaaattcagggactttcctgaactagagaactggaagttggatagagctattctagaacagtggtttggggtctaaatacattgtttctgggggccacgggtaggtggcatctgtgagctgccctcaggaagaggggccaacagtgaccccgggtttcttctctcctggacactcaccacctgcctcagttcttctaaaa

SEQ ID NO: 172 - DNA, 511 bp, Homo sapiens
acgtggcccggcgcggcgcggcccccggggaccgcgagcaacaaaagcccggcccgcagcccctgctcctcgccttccttttcaattctcccctgatctcaggtggaattaggaggattaaagcagcaagtaaatgaggcgagtgtgcaaaatgactcctttctgaaccaaacggcatgctccgtgctggggaaatgagatgcacatttaaatctcatccatccaccgcctgcggccaccgccatgtacctgcctacttagcccaagggttaattaattgaggcttcaatgcactattgcaagagcattattgcatttgatactctacatctgtgatttaaaactctttcagtcccttgtcagcaagggagggtttttaactaggaaattagcattcagataagggaacgctctatttgcttctgaaaggaaggaaatattaaggatgctggtgcacaaaccgggctgtcatatcctgtcccatcctggagggagcgcgagggcccggctc

SEQ ID NO: 173 - DNA, 438 bp, Homo sapiens
aagttctgcttgtgggtctaagctttggagggctgcagcagaggcggcccgggagccctctgcgcgggtgcccttaggcgcgcaccccttgctcacccgcacgcgcggctcggtgaggttggtccacacggcgatctcctccctcatgctcatgtcggggtagcggttcctctggaacgtggcctctagctcttgcaactgctggcttgtgaagtgcgtacgttgccgccgctgcttcttcttcttggctgggtcgtctgcgccgccgcagcccgtgcctcccgcaccactgtcctcgggccccttgggttccccgccgcgctccttctctgcagtaggcaggacggggagcgggtcacctgggcttacgccccgttgcaccccgtcccggctccaccgaaccgctcgccaccagcgctggcggtcttccctccctcc

SEQ ID NO: 174 - DNA, 361 bp, Homo sapiens
gctccactactccgtacccgaggaggccaaacacggcaccttcgtgggccggatcgcgcaggacctggggctggagctggcggagctggtgccgcgcctgttcaggatggcctccaaagaccgcgaggaccttctggaggtaaatctgcagaatggcattttgtttgtgaattctcggatcgaccgcgaggagctgtgcgggcggagcgcggagtgcagcatccacctggaggtgatcgtggacaggccgctgcaggttttccatgtggacgtggaggtgagggacattaacgacaacccgcccttgttcccggtagaggaacaaagagtgctgatttacgaatctaggctgccagattct

SEQ ID NO: 175 - DNA, 575 bp, Homo sapiens
cgcgcaggacctggggctggagctggcggagctggtgcagcgcctgttccgggtggcgtccaaaagacacggggaccttctggaggtaaatctgcagaatggcattttgtttgtgaattctcggattgaccgcgaggagctgtgcgggcggagcgtggagtgcagcatccacctggaggtgatcgtggacaggccgctgcaggttttccatgtggacgtggaagtgaaggacattaacgacaacccgcccaggttctccgtaacagaacaaaagctctcaatacctgaatccagactgcttgactctcgatttccactagaaggcgcatctgatgcggatgttggagagaacgcattgcttacttacaaactcagtccaaatgagtattttgttcttgatattataaacaaaaaagacaaagacaaattcccagtgcttgttctgcggaagctgctggatcgtgaagaaaatcctcagctaaagttgttgttgacagcaactgatggaggcaaacctgaatttaccggatctgtttctctgctgatcctggtgttagatgccaatgataacgccc

SEQ ID NO: 176 - DNA, 313 bp, Homo sapiens
ctgtaaagagtgtcagccggggagcggggccgccgcgcctgcccggccccggcgcacaaaagcttcatatattctgaatgaaggcgaatcgcagcctcggcctccttgaaaagagccgtgagggagacttaattctctccctttcagctgtgctcagactgtgaaatccacacaatcgcgcccttctctttaaagggagggagaagaggggccgcggccgcgcgccccgccatcagctgcgcgcacagagggagagttcactccggggacaagggcggcgcgaccggcccggggctcttgggcggccgcgttt

SEQ ID NO: 177 - DNA, 354 bp, Homo sapiens
gggagtagagctgagggctgtgaggggacagtggggcgtcagggctggccgggttcgtggcctcgtggccacgagggagcttggtagaaggggttcttctgtatttgtgatgggaggggctggaaagacggggacgcaacagccctagaggctgcacaacccctcggaggcctgtggcacgcagaggggaccccttctaggggcctgtgatcaagaagggagcctgagaaggtcggaaactcgggcgtacaaaatgggggcagatcactcggccgtgggtcccgcccgcgagcagcaccgcggccggcgcacactctgcagcagcgcgcggaacaggccgggcggacgcttggc

SEQ ID NO: 178 - DNA, 319 bp, Homo sapiens
gcagggaacactgaagcctggggagggagcagtgttaaaaattggggaccggaacttgtctcatgtctttggcactcagccccaggcacccccattagaccactcagcctcacccagcccttccaccctgaggttgtcccaggcctcacagcaaaatccagccccccagctgtccttcactgctgcaggggggcaggcacccactcaccattgaagcggacagctcgaatgtactgcagaagcatccgcccatcagtgggttccatcgccgggcacaggcctgtgtgcccagggcagagcgcctggtgcatgctgtgga

SEQ ID NO: 179 - DNA, 311 bp, Homo sapiens
gcgctccgccagcctcggccccaccccaaaccccttcctccttcaccgccggctcttacagggaaatcggcgcccgagacacagccagcgggtgttcagaaaggaaaacgctcgctcaacagccttttcggttgattcttacattcaaggtgtgttactatttgtggcaagcatcttttcatttaaatctcgtgacaaccccgtgaggaccatcgtccccattttcctcttgaggatgagggctcagagacaggaggaaaccggtgctctgatcaaagccatacagctggtgacaggtgggcttggctccc

SEQ ID NO: 180 - DNA, 534 bp, Homo sapiens
ctgaagaagtccaggatttttctctgctggttttagtcttcttaaataccgggagccttaaaataccttcactgggggcaacagagaaaaacagaaatagcagcattgtgtcggccacagtaggccatcccattcacacagcagcctgcaaacaatctctcgtgccacatttagggaaaaacacaattttaggaaatccaaacaacccgtaaacccactgacctacagataacctattcggcaacaatgaaacttggaaaaagtaaaagggaaaagaaactcgcaaaggaaaaagaaggctttcctatctcaggaactaagtcaaaagcccaggtatttcaaatccctgccttcatgggtctgctctggttctgaagtcctgttgggtaatttacctgtgctatgtaagtcactatggaagacagctcagcaccgtggctctgaagtgcagggaatgccacacacacagctaacgcagagcaaccggtctccaggacacagacgaacctcgagttaaatttcacctcaaaagca

SEQ ID NO: 181 - DNA, 802 bp, Homo sapiens
gcgtgtcccactggccctcccacatggctttgcttgtggagacattgcccggcttcccccaggggcccctggaactctgtctcggtaagctcatacaatcacctttccagataattcataaacaaatacggaggtgagaagatcaacagcacaagagtgttggctgtcattcaacagagagggaattagaattgctccactgggtcttgtccaaaaaaccaactctaagcacctctcctcccctctgagccaccccagtgcccgaaggccacatgtacggactccatttgttctagttcttttcataagagagaagaaccgagtgtttcacatgcctgtaacacgggcaggaaatgctaggatccatgtgaaacccaccaccctcccccagaagctcctggctttagcatggaatactctgcagaacgtagctgccgtgaagatgatggccctgaactgccacactccccagcgaggtgacaggctagggactgaggctcgggcaatcccgccaagtatccccccactctccccacattctgacaccctactgggggctttcatgcaggtgaaatctggttaaaagaaacagttgggaagagactaaaacactcaccagaaagcccctgcagccaggaggaaagtgtctgtagaaggaggtggtgcatttccttggccaccgtgggttccccgactagctcaaggagaaagggtctttcatggatgcagcagcaaaggtaactctgccgacccggcaggggaccatggctactgctcagcgcccacagggcagggcctctcg

SEQ ID NO: 182 - DNA, 423 bp, Homo sapiens
agaggaaattctactgcctaagaaaataggcatccagatattcaaagaccgggcctcagaggtgaacttcacttatatgaggcatttctctgtgctaccatgaattcttgatgtgcgcccttcatccacctgcttcctcgtggcaccttccaaaccatccagacatgactctaagtcatcaggttaatgaacaggacaggcagagagctgagtggcagtaaataaacagggcctgacccctatcagaatttaaatgttttgggatcctgaccccaactatcagcatttgccaacattgctagttctcctcttatacctctgccttccaaagtaccacaccaatgagggtgcaggccaggtgttagttctgatccggcaccgtgacctgacttctcacacggaaagcagcgtggaccttctggc

SEQ ID NO: 183 - DNA, 131 bp, Homo sapiens
gctgccgccgaggagggcccgcggctcgagagagagcgctcggcggctccgggtgcgtcaggccgcccccatgtcacccgccggaccccgcgccccgggcgctgcggccccgctcgggcgccgggcagagg

SEQ ID NO: 184 - DNA, 395 bp, Homo sapiens
cgcggcgtcccctgccggccgggcggcgatttgcaggtccagccggcgccggtttcgcgcggcggctcaacgtccacggagccccaggaatacccacccgctgcccagatcggcagccgctgctgcggggagaagcagtatcgtgcagggcgggcacgctggtcttgcttacagttgggcttcggtgggtttgaagcacacattagggggaaatggctctgttcctgcaggtttgcgcagtctgggtttcttaggtttagggggttgggtgggtttctctgggggtgcggtgggaagcggatcagttcggataacggccctgagcaagagtctctgtcccgctcccggcctgacgcggggcttccagcggacacgaccaacggtgagcgcaccct

SEQ ID NO: 185 - DNA, 348 bp, Homo sapiens
ctcggcgcccgcaagctgccacctcccggggcggccagggaaactcagccggctctgggaggcgggggccgcggtcgaggggcgccgctattggctgatcgggcgggggaggaggggcagggaggccgtgattggcggggacccgagctgaagggggcggggccgctatatcagcagagtccctgtcgcagcgcaggcagttgggctgctggagtgcggcgccaccgcggaggacaggggcagctggcgggcagcgggtgagggggtggcggggacgcgagtggcggccgcggggccccggacaagggtccgcagagctgcagccttcgagggccagccctctccgag

SEQ ID NO: 186 - DNA, 697 bp, Homo sapiens
agttttctggagtgaatgaaacagctctgcatcctgtggtagtagttaccggaatctaaacatgttctagaacagtatgccccaccatgccaattttatgtatgataattttaaaattaactaaaccaggctgtttccattgtactatgatagataaaaggctggagtggagaaaggggtacgtacatttgaagtgacccagagaggggttttggtcgtcttcatacagcccctactacacaagcgtgaaatttcgctttcttcccactttctcacacgaaaactcgtgtgtgtgtgtgtgtgtgtgtgtgtgtgtgtgtgtgttttcctcagccttcaggtgaggaaagggaaagctctcctttctcaaatcagttcccagaacacactcgctcttgagctggtctatttcttatctctcttcaggcaaagtcgtatcctccaccttacctgctccagccgctgtgacatcacgcaggttccgttccctgcactaccgctggttatgtttccttagtgaccctaggcctcttgcaggagagtccctggtcccagccagcgggagcggcttctccagtttgtggctctcggagagttcgcctcgtccctccctctcagataaccacctatttctgacctctggaggccgggctgagcccggaattttatgaccagcgcccgctggccgtcgtcaccc

SEQ ID NO: 187 - DNA, 565 bp, Homo sapiens
ccaaactctacttccctgtgccaagacctatgcctccccccagcctcaccggtgaagagaacgtcagtgccatccagcgtcgcgttctcgtctcctatttccacaattcggagccccaggtcttgcagggctttgcggactccatcgaccttaggataggagaagagggcacggagctgtgacacccccatcctcaattcttccccaaagccccgacatccagttccttctgcctttccccataccacacccgcgccacggcgctcacctctggcctacgagcggggctccagggccgcgtgattagggccgtgtccccttggatcacggccgtgtcgccaagcagcggtcccagcggcaatgactcctcaggtggcagttctagcagctgtagccccagtcgttgcctcagtttacctcccagcaccccgtgctccctttgagctttggccagatccagagcgggaaggccagcccccgcaccttcccccgacgccaggctctctgggactccccggatcagggcatgggagcagcggcccagcccctcccccggcgtccccat

SEQ ID NO: 188 - DNA, 483 bp, Homo sapiens
gaaccagagaaacgccattcgggcttcgtggggcgggctgcgcgaggcccgggggctcagggtacagacgctattcttgctgggagagccgaacgcacagcaccccgtgtggggttcccaggggagtgacctggcctcggagtcagcagcccagggggatatcttgcaggccgccttccaggactcctaccgcaacctcaccctaaagaccctcagcgggctgaactgggctgagaaacactgccccatggcccgatacgtcctcaagacggacgatgatgtgtatgtcaacgtccctgaactggtatcagagctggtcttgcgagggggccgttgggggcaatgggagagaagcacggaaccccagagagaggctgagcaggaaggaggccaggttttgcacagcgaggaagtgcctcttctgtacttgggccgggtgcactggcgcgtgaacccctctcggacaccggggggcaggcaccg

SEQ ID NO: 189 - DNA, 352 bp, Homo sapiens
tttgtgatcttggctggggtaccaccggcctctaggtgaggggcgatgccggtggtgggcgtagcggggcgtgggcagggaaccacacgggagcctcagcttggccttgagatggcaggagtgggatggggcgtcatcctagtagaggctggacagtgagagatcggtgggctggtaaagggtgggaacaggtcttttctggggcccgtgggtggagggaccttgtggggaggcgttggcgagaggggggtgaagcgaagcggccacttacccctgtagagcgcggctctggtttcagcgcccggtgcgcactcagcagggcacctgtggtccggctagtttgataaacaca

SEQ ID NO: 190 - DNA, 878 bp, Homo sapiens
ccgctgcgcgccaggcacgcggcttactcgcgcgcagggtggccaggcccggcctcgggacggttccgtggaagcggccgctttgcgggtagtgggactgtggccttgcgctatgccctgtgcttccgtgggacgtgtctaggaacgggtgggctttctgcgggaggcgggggtcgggcagggctgctggtctggagtgcttgtgctctcttggtagcttttgtgtggcaatggatagggagctggtccaggaaaagcgtctgtggaagaagaggagggtggacgcaccttctccaaggcacctgtagaagtcactggaactcccgtgaggcctcctgttgaccttaggccctgcgggagggagggcaccttccacctttcccccagctgaaggtctggtaccagacgcaggaaggttccaggccaacacagcgctccttcacacagtccatgccctgtgccaaactctcttccagccctggcgcgacaagatgtggttggggccccagatctcctggagggcgagagggacagaccagagggacggctagtaaaaaaaaagaaattggccttcaggaagggagaggcagccagggtggcggggctttcaaagggtagctttgacttcagctctgccgaggactactagttggttaccctggatgaacatctcgaagctctgttaactcatcaataaatcaggggtgccaaaactcatcttggcgctcgcggaggagcttgaacctgggcctgtggtgagcactgctaaacaggctactgccccagtggccaggatgctgctttgcaagcagtgaggggccaggcacccgggattgggtcccttcaagccctcagcagtgccttaccagcaaatcatc

SEQ ID NO: 191 - DNA, 514 bp, Homo sapiens
cactcgagggtcaaaatggcattccccgtggcccgagccttgtctaacccggccgtgccgccacccgcactctcgcagagagcagggtcgcctggggtgggtgaaggggtctcagtcttgcactcacgtgaggacatagttgacgctgacgatacagtgtggcctggaggagcgactgttgtgcacgatggtcgcgtagctctgcacccatacccagccgccgtgtttcgccaggaacctgtagtacttggtggtcacctgtcccttcaccagcactgacggagagacaggaggcagatggtggtgaaccagcctgcctgggactggccccaaatgcaatccctggtggtaagaattgagggctccctgtgggccctaataactgcaccaggcaggagagagagagagacgacaggcaaattcccaaaggaactgattgtcccgtggtattgcatcgagccctccggtcacccttctagggatgatacaggggtggccagatgtcttcaccatt

SEQ ID NO: 192 - DNA, 321 bp, Homo sapiens
cagcgccgcgcagcagccgccccgctgcgcccacctccccggctgctcccggagggctcacaaaggcggtggccgcccgagtggccttctccatccaggcgttcgcgtcctcctccccaccttctctcccgaaggcgaaaatggcagggccaggcgagaacctgggacagcggtggccctagccctgcgatcctcacccctcctgctaggagaggctgcgggctgcccgcggacgatgtggccgcggctgctcccgagcgcatcttcggcccgggtccccgccgccacccctcttctctgctccttccatcccgcccagag

SEQ ID NO: 193 - DNA, 487 bp, Homo sapiens
ggacgtgagccagtgctggccgggaagccgcgagcacggcgactgggcccggagcgggcagttacaagttaccactattcgcagggccaggtacctggcgctcgctgttgctgtctgcggtggctgccctgccatgacctcttgctaccaaaggctctgccattttaaagttgcctactggatctggcacagtcccgaaataccagctgtcaaaccaggaatagcatgtgaaacgccttttccttggtcttgggcaggggtttgttttgcaggcaaaaccacccccacttgccctaaagtgagctctagtctcaactccttggtttgctttgttgcaacagccccaaaccgagagaagggaaacacattctctctcctggagtctggctgggaaagtttgctcagtgcccacacttataaaaagcatttactggggccgggcgcagtggctcacgcctgtaatcccagcactttgggaggccaaggc

SEQ ID NO: 194 - DNA, 318 bp, Homo sapiens
gaaatgggagatctgcggggaccaggccgggactccctggctccgaggccgggacgtggcgcgccctccgctcctcggtcaccctctgctttttcgaaggtccgtaatgcgtgagtgtgggtggtccaaggttcagggcctccttccagtaagtacttttagaagctagttcccccacttcccatccgttgcggggcttgacaactagacgaggggagtgagtaaaggtgccacaactttgcttaccgtaaaatgggttaatactcgccggggcccaaaaggctgagtcgccccgcacgtcttgcttcgtggagtctc

SEQ ID NO: 195 - DNA, 410 bp, Homo sapiens
ggcagaaccagaaccggctccccggggctgcccctgcttcccctgacaccggctgcgtctgtcatgggctgcggcacggtcatcttcgttttgactttcaggtggcaaccgcacggtgccacaggctgtcgctgtcacccaagcacaagtgcagtgacaatcctcgctacgaatttcctcgcggctccatacctgaaataagtatcactgtattatcacagctctgtggaaatccccacctttcaaggctcctcttgaaacacaggttattgtaagcggagaaacaactgcttttcaagatgttaatatctgttgggataaaggtatttctcactgaggattcaccctcaccaagcttcccggcatgtaaaaaacatcactgtcactgcaggcagatgtaaggaataaga

SEQ ID NO: 196 - DNA, 488 bp, Homo sapiens
cctgtgaagaaacggctgcagtgagagcagccgaggcgggacgcagccccggacgtggggtcagagaggagccgtgcgtggggtcagagaggagccgtgcgtggggtcagagaggagccgtgcgtggggtcagagaggagccgtgcgtggggtcagagaggagctgagttcaggacgggcatggcagccgcaggaggagggcaaggcggagcccaggaggggcaaagtggacaaagccagggctggagaggggagacccaggatcagcaccacgttccacggggatggcacagaagcctgagaggccacggccaaatgtggaaattggctttgccaaatcttgcatttcagagagaaaaggcaatatggcattaatgcttgccttgtttaattttcgtaattttccctttcttcctgccaatagcttgctttgttatccggagggtagccacggttttagctcacagtgatgtcaacttgaagcttct

SEQ ID NO: 197 - DNA, 449 bp, Homo sapiens
tatccttgcagcgagcaggagctggcgctgggagggcagacacgtggtccgggcggaagtgtccgtgcagccagcgggagctcgcgctgggagcggagacaggtgatctgggcggaagtgtccatgctgcgagtgctcgcggctgggagcgcagacacgtggtccaggcggaagtgtccatgctgcagcaggagctcgcactgggaggggagacacgtgatcagggcggaagtgtccgtgctgcgaggaggagctagcgctgggaggggagacacgtggtctgggcggaagtgtccgtgctgcgagcaggagctctcgctgggagggcagacacgtgatcctggcggaagtgtccgtgctgcgagccgcagctcacactgggagggaagacacgtggtccgggcggaagtgtctgtgctgcgagtaggcgctccggctgggagggcaga

SEQ ID NO: 198 - DNA, 609 bp, Homo sapiens
taagcattaacggcagcgtagacccccgtccagccgtccctgaagtgtccggcatctccttcgagggaggctgtagggtcctctcgtgcaaggcccaggcgcatcgtgacagcttgcacgcgctgtggttcggggaggtcttcctgcaggcactgagtcacaggtcaaaggctggctcaggtcaggaagccaagcaaagcaccgagacgttgccttgcagtggcttcccgccgccctctggtatccattgttgacttctcttggttcgatctttaagtaatattcttgttggctgtctgtctgtcccctaggaataccacgttttatgaaccatcttcatagccgtctgcagctcccgcttccccgtcaccactcagcctgctgccctcgcaaggattccacccacctggcttctgatgagtcagcactaccacctgaccccaattattctggggtcccatgattttcattgctactagggaaacctttctagaaccacgtcttgtatcgtctgctctggcttacagaggacagacactcctgagccccttgacagcaccggtgcccccggccagcccattccttgttgataccaggttgcattcccct

SEQ ID NO: 199 - DNA, 643 bp, Homo sapiens
ggtggagactgggccctgctgtctgtccttgcagctgttttagggccgccggccagtcttcctcacctccagatttcagattttcccctctgcagccaaacattgctcagtaagtgaaaggagaactcagccctctcaacaggtcctgggtacagccagagggatacccgtgggcctggcagtgaagagagatgtgaccacagtcctctgagccagggaccagggccagggccaggcggagggaggaaatcattgccctctcgctgagtctgaagagaactgggggcctggattcagggtaaggggacacagcatggccacagcgggaggttggcagttcctcaacgtggtggcagtggaggggcttaggatcaggagcctgagggggcaccatggcggcgagcgagccagccctactgtgtactgggagcagatgccccaagaggagtcagttcttttcaacataactccctcctgtgcctgagcaccacagccaggcctgggttggggtcatacaggagggcgacagccatgcttggccaaagtgatccagtgggaacccagacagaggaagccagagacagtggaggaaccggtgaggggcccccaacatgggcaggaggcagagagtggcagccaggat

SEQ ID NO: 200 - DNA, 460 bp, Homo sapiens
accgccaacatctgcggggacaatgacagaggaatgcctaatgagggcccggcgtggccccgccacggagcgcaggctgggacagatggtctatcattacccatttttattaattactgctccccgtgctgcgattggctccaacggctcaggcacgttggacgcgctgtggccgcagctattctggaaggaagaaacggcccttcatttagcacagcgtgcctttcctcccagcttcctcagaggccacaggcatttttggctcagcgaggtccctggggaactaggctgtgcggagggaggagtgtgcagctgccacctgactgatgagaaggggcggaggggctggtgcggggagaggcagagagcccctgtgactccagcctggagcgaggccctgcggggagacccgggacctctgcagacctggcccagaccacatctgcatcccacaggcttg

SEQ ID NO: 201 - DNA, 469 bp, Homo sapiens
tattccccgtatgttcaagaagaatggagaatgaacgagagtgagggaccggaaacgactgtgttccatgctgagggtggcagaggctggcctggatgctggatgaggctcaactcgggggcctgggctgttgcactggggctggactggggatcacggaagagggccaggcccgcgtgggtctccagggctgtgctcaggggcgtggtcttctgcagtgggggcttggctggtgagtggagactctgcgtgactctgggggcgtggccgagaatgggtgtagctctgtactagggcgtggtcatgcgtaggggtttgggtgaggcgttaacagtgggcgggcgtggctcgggctcgagggcgtggctcaggctcgtggaggcgtccgagctccagggcagctctggggcagaactgtccggatctaacaatccctggggagccggagaccggtgactggtccgtgggc

SEQ ID NO: 202 - DNA, 872 bp, Homo sapiens
tcctgctgacagctgggttcgttctgcctcattgaacagggctttgtaccggcattagctctgtgaccacagctttttctgcaggactggggatcccagtgactgtgaaagaggaagacctaaaagactgtgtcacatctctcacctgggtgtcctggttcaacaagcactgggctgggattgtcaaggtaccagggcttttgccacataaggcactgtccactcccagcatgagaggtctatctggaaacatggctctgaatgtagagggggccacaatttaaaacaattaaagcatttgcaacatatcaatcctctgtactacttaagtgggtttttttgtttttgtttttgttttttggttttgggttcttttcgtaaggtagctgaaaacaactctgctctcccactttccttcaaaaaggtagagatttattggaaagatatttgagaagctctcagtatctacagaaaggatgaacgaataatcctggggtcagaacagggccttctcccaggacgtcacatgataaagcacgtagacttgctctgtggagccctgacgttgatgagatttggctccaacccctcttgcctgcaagagtcttagatgatgcccacattccaggctcagctgcagtgaggtgtccacctctccattggtcagcggtggcctgggacacagcacaggctgaccatggccctctcctgtgtgttagagcattccagaagaagagcactcactgcagctataaatcacctcgtcattgtcttctacaatgttcttctgctctgcactccactctcgttacaagctgtccccggaccccagggctgcccagttgccagttaagacctgaggtcaagaactg

SEQ ID NO: 203 - DNA, 538 bp, Homo sapiens
ccgggcccattgttagcaggctattccacggcagctttgcatctggctccggcgggaagcggaaaacgggaggcggctctcgaagcttcccgaccttcctgcgccatcaacttctcaggagtggctggagaaagatgcatgtgccaagcgagacaacaaccccaggtccatgtgtccaaatccccgtagccagggcggcggccagccaaagaaatgccgccccgagcaggcgcgtgcggctcctggcattctgggtttcatacccgtagggctcgggtgcggtgagtatttccgatttccaggaagtctgttggaagttaccagcagggaaagagaagatgcttgctcctctctttccctccctctctcttttttttctacccttcttcttgtccttgtcgctctggttgggggctggttggtttagatacagcgagtgctccctggcagcctgaactgcctcccacaccttcctgatccttgggcaccggggatgtttgcttccgggttctgttgtgggaaacagcactgcggggga

SEQ ID NO: 204 - DNA, 558 bp, Homo sapiens
ataaacagcagaaggtgaatggcggctgcgagggaagggagttggaggccgggagtgggaagcgaggctcatgcgggcgccaaggtggccctgggggtgacgcgaacatcagggagtcaccagggcactcgtccgtgtggcgcactcattgctcgaaatgttccagtctacaaatggtccgaagatggtgatttatttttgtaaaataaatacatactccatctccacacccatactttctcctttttctttcttttttttggcgggggagtggggaaggaaaggacatgaagcaaacagaggaagctgagaaggtggaagtggctttgctggtggtgtgggctgctttcagagagctctctcctcaggcaccacaacttggagaatgtgaatgcttgctttattttcctttcctaacaaaaggaagccttgtctgggagcattcaatcacggactctcctgaaacctcactaatatgtgttcatttctgtaggcgcgaaatcagacccgggttcattccactggccagaggcagtcttgggaggatttgtgttctgg

SEQ ID NO: 205 - DNA, 449 bp, Homo sapiens
tacttgacgccgttgaagtactcggggcacggcctctccacgagggctccggcagcgctgcggggccagcacgttccgatctggtccaaggtcgtgttgcagtaggagtagggacctggcggcgggagagagcgcagtagggctcagaggggcccgcagggacgcggggctctcggagcgcggggtcaggggcgcacccagcgcgcgagagaaggagcccgcgcagcctccgaccgctcgcctcccgcctaccctcggggtccaggggtggcccccagccgtccaagagcagctcttcagccagcgccaggctgcagttggcctccagcaggctgtggagcagtgccgcgtccatcgcgtcccgcagccgcgtgcggagagggagtgggagtgcgcgcccggcgtgactgcgagggagtggacgcgagagtgagcggccgagagggcgc

SEQ ID NO: 206 - DNA, 423 bp, Homo sapiens
gccagcaacctctccccgcgccgcccgccaggccccgtcgggcctgcgccggctctggcggtgcccacaaagtccccgaggggcagcgttggccaggcctacccaagacctgccccgtcccgccaccactgcacccactttctgctgccctcatcggtctgcgcagctgtccccgctcctccccctgaggcaaaactgcaccagaaggcatcttggcgaggtcattgccaccccgcccatgattgtagaagagactcaatcctgtttaagaggtttagctccccaagcgctattaaaaataagtttttggtggcctttgtaaggaaacttttaatcacacagaatttagcctctgatcggccagacatttaaccggcgcagagtaaaaccttccatttttaacatccactctggtgtttgttg

SEQ ID NO: 207 - DNA, 517 bp, Homo sapiens
aagtggtcccggagaagcgctccctcgcagccaagctgcaagaagtggccgggaacctacaggcctcgggccgacccaggaagcctccgcaccagaaagctcgaggagcccttacccaagtcttgctcgaagggcaaagcaaagagccagcacccctgagtgtcactgaagttcctggatggggtgtgagtgcgcgcgttccgtccgagacctcagtctcgcccagctatagagccgataaagggatgtcttgtgggcgtaaggcgcttcgcgcccatctccaaggccgatgtggtcaggaggtgaggggaaatgtccttctggcagaagcccgcggtgctgcgacgttgacccgcctggcctcaggctcagggcggcgggcagcccagggcacatgtagtttcagcagccgcgctacgtgggcgggggaccccaggccaccccacgtgtccgccctgggcctcctccgggtcccaaggcgcggcgcctccaggccttgtagcgtcttccccgggtc

SEQ ID NO: 208 - DNA, 386 bp, Homo sapiens
aataaaactgtcctggtgagtcgctcacagcctgtcccagtaactgttccggagcgtcagcctgacgcagcacccctgaaggaccacgccgtgcgggctgcgagcctcgcccttaaccatgcccagcaggaccagagctgccagcacaaatgaaggtcctggccattgaacaacttctgagcctgcactttaagaaggcgctcttgttttgaatacgtagtctgtcctgttctttcaaagtaagtgcttttatcaagggcctggactgtcttttaaacatatttttttctcacttgtacattaaagggaaatgtatttctgtctagaaaaggagaccgggagaggaatgagactgtgcggtattcaggcttggtttaaactagggc

SEQ ID NO: 209 - DNA, 615 bp, Homo sapiens
agggcccgggcaatggtgtccttgaggctctcgcaggtggggagttccccgggcttcaggcctgcgtaccgacgctcctgggggacacaggcacgctgctttccctcccaagccagtgggcccccacccgactccccactctgcagcttcccagaggccttcccagcaaaggcagcccaccttgctaatggagttgtagtaggggtgcttctcgtccatcgggggcggcgggatgtcgaaggagcgcctccagatcttcacctgctcctccccgtgcttggcggccgtttctgccttgttgaggcctgtgaggcccccgtaatgccgctcattgaggcgccaagtgcgcaccacaggcagccacatctggtccgtgccgtccaggatggcccagagggtgcggatggcccgcttcagcactgacgtgtagcagatgtcaaactccatcttggcatccttgatggccttggctccccgcttggcctcctcggtccccttttcactcagctctgcatcgaaccagccacagaaacggttctcctggttccatgtgctctcgccgtgccggaccatcacgaggcggtgagtggccatggtggcagcagggaccacaga

SEQ ID NO: 210 - DNA, 552 bp, Homo sapiens
agggtcaggtcccgcagggcgcaggcgggcgcttctcgcagcccaaacccgggcggggagtgcgggaacccctagaactatgctgggagccccagataagaggaaggagatggtgacttaagtgacgaagtgatgttttgtccccaccgccttttaaagggagaagggggtaaaatttcatttggaggatgattgttacgcgagctttcagagttcagatggcgtcaagtggccatcgagtcaacatttaacaaaatgttaccgcataaaatattgtcatcgagttatctggcagctccgcagagaaacttcacaggcatatgcgttatatgacataatttgcatggggaagggggtcccttcacgattcttttatatggcaacctgttcttggacctggcagggcccagagaagcgaggaactccgcagctcgtcgacacgtctcgtctcctgtcccaattcagggcttggtgaggtgactcgcggtcgcgggtgactcgccggcaggacactgcctggaacgcctggagcgcctcccactgcagacgtct

SEQ ID NO: 211 - DNA, 736 bp, Homo sapiens
ccgcgagaaagactgtgcctgacctgcacggaggatgacgctgaccggccggtgatgaggaagccaccaagggaagcaattagacacaggacgtgctggacctgcaggctgcacaggccaagggagggccccaggcgcagcgggcggatctgcgggtccgcgggctgcgtccctgccctgtggccgagactgtgacagtctccgcagttggcagccgagtgcactgacacttaggctgagtccgcaaacagaaaagcaccaaatctcagtggctccacgcaacaggtgtgtttctcactctcatatccaggttaggtcacgggacacgccaacaagtgatttagggaccaggctctttccctctctgtaccaccttaaaaatgcagcctccaagttcactgtctaaaggaaaaagtaatgatcaagggtgagagattttgccgcggcctgtgacacagctccacctaatgtaaaggtgttagaaagctcggttttcccgcgtggccaggaagaggagaaaaagaagtgtcgctggatcactaagacttggccagacccttctgtgcctcctaaacattctcccgtccatgcagagagcaccctcacgccttcccccagaggggcaaccacagtcccaccagtcactgcccaatgcaaagggcccccaggcaatctcactccatggccgggactcctcttgggctgccaccaatcacctagagggagagtggttgct

SEQ ID NO: 212 - DNA, 564 bp, Homo sapiens
tattaaacctgtcttacacacacacacacacacacacacacacacacaccggattgctgtccctggttcaagtgtgccaagtgtgcagacagaacatgagcgagtctggcttcgtgactaccgaccataaacccacttgacaggggaaacatgccttggaaggtttaattgcacaattccaaccttgagctgcgcgggttccaagagccaggcccgtacttgctgttgatgtcattggcttggggagttggggtttggtgcccagcgcggtcgttgggggaggggcaaggcatagaacagtggttcccagaccttgctgcacattggaattacctgggattaaaaaaaaaaaaatcaaaacaaaaaccagtgtctggctcccgcccccagacattctgatttaattggcatggggcaagacctggacttgggattttttttaatgctcttcatgtgatctgttgggcagccagatttggggatcactagacggaagaaggattgttaaagtctccggagatgttacttgccaatgctaagagctctttgaggacatctggaatt

SEQ ID NO: 213 - DNA, 768 bp, Homo sapiens
atcttccgctgacaccctcctctggggtcgcaggtatgaatccatcacccggagacccctcaacaactcaccagactgcattcccaaccccctgggacccgattcctgcacacagcctttttcgggaagggagtcccaagagcagctttcagcgcccacctcacggtctccaatcgcctcctcctccagggggagccgaccacggagacgccccaggaggccctgtggtcgagacttttatagtcccgcaatggtagtcgcaggcagcccttttcctgaccaggccgtctctgcctgcaccattgtccttgcttaggcgggctgacagccccgacagcctggcgcccgaggcagcctcacgaatgcgcatgtgccagtggcggggcacaaggcgccagcggcgagcactggcttgcttcacagtgaacgccttcgttgcctggcgactagtccctgcggcttagcggagagggggcgtcggcgttgggctctcgcctgtcttctctggggggatcctcgggatactcccacgatctaggacagggcaggggcgagccagcctgaggaaacgtcaagggaaggcccagggatacaccgcgaaatccctaagaatccaaaaggatccgcaggaggcgtcaggcctgcctagacggagtggggtgagtctccttaaacgttgcccccctgtgatttctgactacagcaggcctgtgttccccggggctgccctctcccgggaggggcttcccggcggagcagaaccgcgct

SEQ ID NO: 214 - DNA, 338 bp, Homo sapiens
ggtctcagttgcacagaccaggaaactgaggcccacagaggcaggtgtccggttgtttgcaacctctcagcctgtgctaaccccaattgttcagagagagccctgaaaccctctcctctgggcgcccccaggtgactgccccagcctcaagggctgcctctgttgcaggaaagacgacgtcacaggctacttcccgtccatgtacctgcaaaagtcagggcaagacgtgtcccaggcccaacgccagatcaagcggggggcgccgccccgcaggtaagcgggggtccccggggctgggcggggtcgagcggggcgcaccacgggttcgctctgtctag

SEQ ID NO: 215 - DNA, 416 bp, Homo sapiens
gactcaggtgcccgcccatcctagactccgtggccgcacctgtgtcttccggatcctcaggtcagcggaggagcggttcaggccttcctcttgctcgatggactgctcgatgcctgggggcacaggtggctgctaagtcataaccaggccccttcaaccccgtcccctacccgcacacccagcatctatccaccttcccacatcccctagggtatgtgtgtggcctgtggctggtgggatcagtgccaggcaccgatggaggaggggcctacagaagcccttccagctgtgacgctgtgtggtgtgtgcatgtgtgtaaatgcacacccactccaagctctgcgtgtgtgtgaggtctggtagctccggccaccctatgcctgggtaccactccttttctgctcagaaaggctgtg

SEQ ID NO: 216 - DNA, 338 bp, Homo sapiens; identical to SEQ ID NO: 214

SEQ ID NO: 217 - DNA, 338 bp, Homo sapiens
cctagacagagcgaacccgtggtgcgccccgctcgaccccgcccagccccggggacccccgcttacctgcggggcggcgccccccgcttgatctggcgttgggcctgggacacgtcttgccccgacttttgcaggtacatggacggaaagtagcctgtgacgtcgtctttcctgcaacagaggcagcccttgaggctggggcagtcacctgggggcgcccagaggagagggtttcagggctctctctgaacaattggggttagcacaggctgagaggttgcaaacaaccggacacctgcctctgtgggcctcagtttcctggtctgtgcaactgagac

SEQ ID NO: 218 - DNA, 301 bp, Homo sapiens
gctgactctcctgcgcgcgcccttgcctgcgcgcggggggcgagtgcaccgggcgctcgctccctgccgagctcttgtcgcccgcccgagcctttgccaggggatgaggtgggaggcgctagaaaccacttagctacagccaaccgcccaatgcagcactcgctcgctccccccgccagcggaagcgtccgcgaagcacaatgcagcactgagccgcggtggaggttgcagcgccacggccgccgcagcaccggccggggctgggtggaggtggccgcggggaccccgggggcgcggagcg

SEQ ID NO: 219 - DNA, 570 bp, Homo sapiens
ggagactacagtctgagacccggggctcagcccatgcccaggcctcggccggggcgcagcgatcccccaaagccagcgccgtggagttcgtgccaatcctgacatctcgaggtttcctcactagacaactctctttcgcaggctcctttgaacaactcagctcctgcaaaagcttccgtccctgaggcaaaaggacacgagggcccgactgccagagggaggatggacagacctcttgccctcacactctggtaccaggactgttcgcttttatgattgtaaatagcctgtgtaagatttttgtaagtatatttgtatttaaatgacgaccgatcacgcgtttttctttttcaaaagtttttaattatttagggcggtttaaccatttgaggcttttccttcttgcccttttcggagtattgcaaaggagctaaaactggtgtgcaaccgcacagcgctcctggtcgtcctcgcgcgcctctccctaccacgggtgctcgggacggctgggcgccagctccggggcgagttcagcactgcggggtgcgactagggctgcgctgccagggt

SEQ ID NO: 220 - DNA, 541 bp, Homo sapiens
tcgcagcgtcctcttctggcaccccagggtggccttgttgtctgtttcccggccgagtttgtgttttgctggctcgcggagttttctcctggctacgggcgagggttgcgcgtttcaggaaaaactgagtggggcactgtttgtttgtttgtttgttttttgataggatctcagtttgtcatccagtctggagtgtagcggcgggatctcagctcactgcagcctcaacctctgggactcaagccatccccctgcctcagcctcccaagtagctaggattacagggcgcgccatcaaggccagttttattttttgtagaggtggggtctgtgttgcccaggctgctctcgaactcctggcttcacatggtctcccatttcgacctcccaaagtgctccgattataggcgtgagacaccaccgcccaacctgttgttgttttggtggtattgtttttaatttaatttagagacaggatctcgctttgtcacccgggttggagtgcagtggtgccatcatagctcactgcagcctcaactccc

SEQ ID NO: 221 - DNA, 468 bp, Homo sapiens
aggaggttggtgaaataggggcgtggtctccctcgggggcgtggcgatccggctcaaaaggcggggcctgggtggaacctagcctatggcaattgagaagccagattaaaggggttactagcagtctggtccaaaacccgcccaatggagtaggagattgagggtagcgggggtgggacctgggtgggggcggggctaaagcgggccgcccgcgtgcccaagcccacgtgagagggcaggacgcctgagagcttgaggccacacgaggctgtggagcggcgtgactcaaacgtggcgcgcatcagctcgcacacttccaaacctcgcgatagctactggccctgggcgagccgttgggattgcgcttgcgcacagcgtgtcttgtacgttcccgcgcttttctggaatctttcgcccccggaagggcagcggcgggcgcctgtgtggaaggtggggtggccagagaca

SEQ ID NO: 222 - DNA, 395 bp, Homo sapiens
cacccgtccacagcagcactggatcgccacacacaacactgtcccctgccggctctacaacccgtcagggagaatctaccaccttccagagctggccaaactcgaaggacactacccctgcacctcctactaccacatcagcctttgttgagctatctacaacctcccacggcagcccgagctcaactccaacaacccacttttctgccagctccacaacattgggccgtagtgaggaatcgacaacagtccacagcagcccagttgcaactgcaacaacaccctcgcctgcccgctccacaacctcaggcctcgttgaagaatctacgacctaccacagcagcccgggctcaactcaaacaatgcacttccctgaaagcaacacaacttcaggc

SEQ ID NO: 223 - DNA, 483 bp, Homo sapiens
gcagccgaggggtgcggcccctacctgggcactgcgggcagcactctcccggcagcaggacaggctctggacagggcagcggcacgcagcggcgggccaggcactgcacgttgccgctctacggaccgacagacgcaccacgggtcagtgggttagttgggggaggccgaggagacccctcccacccagtgccaccaggtcccacccctcccacctgggcgggcctctcctggcctctgcaagtcccgcctccactcccctgggcgcctcccacccaggtgcagactcaccagacagcgacacagacggcaggggtcagaggggtgggggaagtccgctccgctggggtactctttcccgccaaaggcacagcctgggggagggaggggctatagcctgggagagggcgactgccccagggcgtgctgccttccgggggccatgccatcccatcccagaaggccgccctgacccgcccacctc

SEQ ID NO: 224 - DNA, 339 bp, Homo sapiens
gagtggccgcgccctgggtcctgcaccgtgaggctggggggaccaggaccggcgggcgcccaccttggtcctgagcccagctgccgacgctgggccagcaccctctccaccgccgccctccagtgagcagagctgggctcaggcagcagcccctgggcgcgggaaggtagcgggagctgcatttaggggaggggggaggtagcaggagctccgccagtgggaaccttggagtgaattcgtcggcgggtgccaagtcgaagcgccgacctcctgtctctggactggggcccggacccctgcgtccccaaactgcgtccccgccttgctcgtcgcccggtg

SEQ ID NO: 225 - DNA, 383 bp, Homo sapiens
gcacacggacctggagatgaagccccttcctgacttgcgcacacggacccggagatgaggccccttcctgcctagtgcacacgatcacggggatctataggcacacgatcacggggagagaagcagaaccactcggagagcacattgcaggggttcaacctcacgcagtgatgggagcgggctggacactgtgtgtgaagactttctttctgtgtctggtgctgggctctgaaatccatggagcacacagccaggaaggggaaaatgaaaatgaagtgaggaaaacaggcacaaccatgacacggaggctgagctggacccgccagggcagcccggaactgcatcctctttctcccctggcaaggcccatgcttccttaccgg

SEQ ID NO: 226 - DNA, 457 bp, Homo sapiens
gcagggttgtagctgtagtgtccctgaaccactggggttaggaggccaccggaagcctggcctgctaggggccttgtgatttcgcaggccccgagaacgtggtgtgcaggtgcgggccgcctgggggagccatgtttaaagtgcggtgaggctcctggaacggcacccaccgcaggcccgaggccacacctgtatgtcagaactgggcgggtcttctcctcccaggccttcagcgcctgtggctatggctgtgggcgggactgatacgggcaggcaggcgctgttctccccacgtattctcggcttccccgccatacaccgtttctgagtggggagaactggttgactgctgaatcccaacacaaatccttagagcaaagaggccagtgatggcttttgtttctgcccggatcctctttgtggatcttccgttttgcggcttttcctctcatttgtt

SEQ ID NO: 227 - DNA, 337 bp, Homo sapiens
aggacccatcctcggccgcttcccgtccccctcacccgccaggacctgccggcccagcctcttcacaccccgcggcctctcaccgcgtggctgagcctgggccgccgttcggaccgcgcggcagccttcgattctgtgactctcctcgcctcgaaccccccacggttctcagcataaagcccagactccgtggctggtggaagcagcagctccctggatcggtcctgggcatatttacagcttcgctttttaccctgggatccactgttcgactcgcaacacctttccggagcccggcgcttcactcgcagcctcgcagcctcgcaaacgcccgtcc

SEQ ID NO: 228 - DNA, 594 bp, Homo sapiens
gcagctgtcctcagctaccagtcaggagaacacttgctcctgggcgtcccggtgccagacaggatccagatttcccttcttatgatgtgcctttcacaaaacagcgctagcctgattctttccattttctattttctgtgtctaattttttccatatccattttccacctggttcatttagtgataccgatttttaagccttatttttttaaggatttgtgaatattgtacaacttgttaggataacagtggaatactggctccaaggatgtgtgcaattttgatttattttcctaatatgtgtgtgtatagataaatgtcagtgaagtgaatatgtgtgtgtgtcaataaaagaaatatacttaatcacccagttatttagtcaaacatttaaaaagtgacatgtccttctgacctggccctgcaagctgcaggaggctccctggcctgtgagtttagggaactcgtgtcagtctggttaagtgggtggtttgggtgccgtgctggctgtacgtggtttctcgtcagtgtgctggacattggccggccagcgctgcctgggccttgaaggcagaaatggctgcagacaccact

SEQ ID NO: 229 - DNA, 534 bp, Homo sapiens
tccggtgctcgtccagccctgcgctgcgctgaggagaaggcgggactcccgggaggcaccctgcaggtctggatggcctgcctctggccagagcctgggaggtggagggagatgggattttgaggccattcacctccctcgtaaatggagccgctgatgcactctcacagaagggaagaggtaatttttccatttgtgaggagagtgcagaccctggaaggctgcttagccactcagccttttccagacagctcatttctcagactgcaaaggtcagcagcagaattgggggagaggggttttactgcattcttcagggcagaggccattttaatattcatgaaattgcaagtgaagccactggaatgttccatgggagcaggccagcgtgtccttgaaaggaactgagagctgacagcttttgtgtccacgctggactcttcctgtaggagccctgagcatgggctatggagacacaggacaccggccaccgtggccccacacccaggttggcaggtgcagctggccctgatg

SEQ ID NO: 230 - DNA, 879 bp, Homo sapiens
ccaagcgttcatttcattaagctctttctggttgcaaagggcaggcacccggagccaccttctgtagggcaaggtcctgtggagccacgggaggaatcgagggaggcaggccctgtctcagcaagtgctggccgctccccgacagatgcccagaagtccccccaggggtagtggccgcctctgtgaagacactcagagggctgggctgtagccctggtcatcacagcctcagccctggacagctgaattgagcctggcagctgaattgttctccatgttgctgtgagcccactgacaaactcctgccctcagtgtctggatttcacacgccctgagaaagaggatgtgatttatcctctcaatcacccacaaacatgaaatgcgcttgcctggcctcaccccacgatgggtcaggaacgatggccaagagggcaggattttacggggctgagtgtgtcgacacacatgttgttcatgctgtcagatgggcatgcctagcacagacgtgttcaagcggtcagatgggcatgcctggcacacacgttgttgtgaactcttgcattgttggtaagatgtgtgatggatttggttttaccaaaaatggcaaatgtggagttatttttaaggcatacttcgtgtgctatttcttgatgtggttttatcacacagtgttgttttcgtccaccttggtgcaacggccctggtgttgcaagaggaggtgagctctgagcagaattctcacatatctccacttaggacacattgttgagcatcgatgtgtgccagagacttgcagggcattgaaggcagacagtgggctgccctgtgccggctctcaggtcccaagcctggatgactagggcatggacggtgctgtgg

SEQ ID NO: 231 - DNA, 152 bp, Homo sapiens
cccctcccgaagtgagcgcggcccggggtccgcggggcaggagatgatccgggggcgggagcctctgccgcgcgccccctcccacgcgtgcgcagcctgggccgggcgttgggcagcgggagggggcgcgggcggccacgtggcctcgaagg

SEQ ID NO: 232 - DNA, 559 bp, Homo sapiens
agcacgtgctgagtcagtggggatttgcttgcaaagacgtcatcacacccggccaggaggtggtggcccaggctgggtgagggacactggggaagttctaacataggaagtcccccaaggagtgaagagattgcggaggggacacacattcagggtgggtgaggctggacgtgcagtcctgctggttttctgggttgggcatgtgattgataaaagtggagttgagggcacattggcactgggagtcaaatgcgcagaagacttgagcttgctactctcccgagccaggagctctaaggtgttttcattgttgaaatccaagggttctgatttaggcttggctggcacgttgcaagtgaccccatacttgtggaatcaactgaggctgatggattctgcaaagcctctgacacctcctgattttggtggtcctgagcatgaagtgagcagggaatacatgtggggataaacgtggcaaagtgccctgttacccccatccaaagacttcccggggagacttccacagtaaggtaccattggagtgaggattgcaggaagt

SEQ ID NO: 233 - DNA, 681 bp, Homo sapiens
gtcagggatctctgccttacagattttcaataaccctcctgtctgcagccggcccagatgcagggaccctccaagtcctgagctcactcggttccctgcgcactttttggctttcttagccccgcacagttccctagacctcatgcccctccctccacctccagaagctctggggatgggctcaaattctgtcatttcctcccctgttgtccctcaggctcctgggtggtttcctcctttgcagtggactagcggggtttagggaggtgtggaagtccaggcaggcagtacctcggcgtgacgcggtgacgcagccgcaggcaggcagtacctcggcgtgacgcggtgacgcagccgcaggcaggcagtacctcggcgtgacgcggtgacgcagccgcaggcaggcagtacctcggcgtgacgcggtgacgcagccgcaggcaggcagtacctcggcgtgacgcggtgacgcagccgcaggcaggcagtacctcggcgtgacgcggtgacgcagccgcaggcaggcagtacctcggcgtgacgcggtgacgcagccgcaggcagtacctcggcgtgacgcggtgacgcagccgcaggcaggcagtacctcggcgtgacgccatgacgcagccggagagagcttcacgcacaccatctcttcgttcttcctgaactctgtca

SEQ ID NO: 234 - DNA, 694 bp, Homo sapiens
aggactcctcgaaacgcccagagaccctatagaccctgcagcctctgcccggcatatccattggattcaattaggcacaacccaactcggctccagtcagcaccatttcttgggatgtgcagcatttctgtgcctcttgtgaagacaaagataaattagacacagacggcattcgagacacttgccatgttgaagaggagaggagcacagaaacagctagcaatggtgccagacagaaagttgtgtgcctgacagggatgagaaactggggggatttgaagaggagacacgattaattctgaccagagtaacagagaaggcttcatggatgacacagaacttcagagggaggattaaagcacaagtaaagcaggattttttattttagcaaaagaaacagatgtgaattggaggcagtggctgagttgttcccatcgatgtctggaagtccgagaagctccactggggctgctagctgacggctgacccacctgttatcaggggcatttggacggcgcatgtgccacaccagggacgtagcgtgtggcacctgccccctctgtgttttcctctcacatgggccaggggctctgtcccattaaataaccagaagattaaatgcagactttacaaagttcctcatccggatcactctatgcccacatcctagaaacatctatggaacttaattttc

SEQ ID NO: 235 - DNA, 727 bp, Homo sapiens
acaactttcttaagacttactgattgtgtgggttttttcctgagccaaccggcttacagcatatgtataaaagcaatgatagaagtataatttggtgtagtaattttaccctaattcacactccccttccatgtgtcctgccgatgattttggtaaccttctgctttcacaaatatgaagtcaacaacattttctttcatttttattgtctgacaaaagggaaagaaacattcattaagaacaagtcacctgttttcactgaggagttatagacaataacagaatgaagccaccgcacctttcccacacgctgctaaaggctgaccattccaaatactgtcaatgcgatgctggcatcctgtgagatgagcgggaaaaataccttggaagcactggttacccacaacactttgaaatacttaattgggtttaaatttcatcagagaaaaggttctttcatgaaaactcaattaaataactacataaagggcgtggggggaagtgttcattactaattacgcagaaagcaaaaaatacgtatattaaatattacatgtctcccctcctcaaacacccaagtacttcaaaataaatggacgacgaatatgtgggtttcattcctaaggaacgtcgggctactcagcaaagcaaaataccctcttcgctcttcacctccccggacggcgttgcgtgtgttgaaagtcacaggcgtaattcaagcaggagg

SEQ ID NO: 236 - DNA, 358 bp, Homo sapiens
ttccttctctcccttatccttgttcaagggccccgggttggcttcaccccggggcttccatggtttcaggttttccttcccttcctttttccccaaggtcgctggaaccagggctgccttccagcacttcatggggcacctggtacttctggccgtgtggccaaaggccccgcagtttttgcacttgagctgtgggtggaaaggaagtgatgtcagtgagtgagctgaagccacaggcagcgatcccacgtcaacattgggacggattgtgaattcagagctgaataaggattccaaagaggggacaccggcatgggggccgttaagtgccgggagagttcggatacgatgttccctc

SEQ ID NO: 237 - DNA, 437 bp, Homo sapiens
gaggagaggaaatggaaaagcagggcaccctccacccgaggaagcatcccggctccgctgcgggggtggttccagtgcagacacaaacagacagattcaaacacacacagacagattcagactcacacagaaacccacacgcccaacgggcgcttcccttcccctcaggcacccacctcccaccctcggctcccctgcctccctgcggaagccaaccttggcctgcctgggacagaagctgcactccctctttcccatcccttgcaagcgatgggttctggatgcctctagcttggtcccaagtcacaccaccatccctgcagaggggagccctgaatagccaggagccttcctcctaagggatgagcaaggcctagagccttctaccggttgagaagagtgggggtcctggggagcgggggtctaaaggagcgggc

SEQ ID NO: 238 - DNA, 654 bp, Homo sapiens
cccaaacgttaaaaacgccatatcccactttttatacaaataactaccccggcctgttctctttgtccccttcccgaagccttcaggcagaccgttccgcaaagtctttttttttgttttgttttgtcttctagacccagctcctcggcgctcatctccagctgctttaagacaaggggtgggggaaggggagggaggcaagaaaagatgagggtgggggaggggaaaagagggaatgcaaggggaaggagggaggagacggggagaaggaaagattggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggc

SEQ ID NO: 239 - DNA, 604 bp, Homo sapiens
cgctctgggccagcttttgtctaccctgttctggggtcgccttccccgccggacccgcgcctggcgctcacgtggccagtaaataacagtcaaatccagggggaggaaaatgcccatagaacaaagaggcttgctttctgcacctgacagtaatcgctggtggagaagccctaaatcccgcaggcgattcacacggaaaggggtgtgggagggcgcgtgtgagtgtgtgtgtccacgtgtgcgtgcccgcgcctacgcggctgttcactggaagggagtcacagcttgggacttaatgtgtttaagccgagagccacttgaattgcgaaagcgaaggcctgatttttaaaaaagactaatttttaaaatccacaccccattgccaactcaggaattggccttcagttgtgtgaatttcagcaaaggagatttaagttaccatattgtatttatcgggatactgtgtaaacaccctagatatatattttttaattgtatgtttgtgaaccattggtccacagttaaatgagtacaacgcgagggctactccatgccggctatagataaaacctgtctccaattcgtctgcagtctgtggacgaaa

SEQ ID NO: 240 - DNA, 514 bp, Homo sapiens
gcaccgcagccgtgcgcgtgtcgtcgttgcgggccgcgatgtgcagggccgggaggcgcaccttccccttggtgccgtagttgatgaggtgcgcgacgacgttctcatggccctgctgcagggctaccgccagaggcgtgaagccgtcctggccagaggaggaaaatgctttgctctgactcgtctgacgccagccgcccttcacacgggacacacccttgcccctcgggcttatgctcccagcagccccagcctgagggacctacctccaccaaagccctcttcatccgccaagtttagcctaaatgccatctcctccctgaagcccttcctgcctccacttcctttctctgcaatctcacagagcctgccgaatttttctttagcattaatttagttttcctttataatttgcttccagtttgacttgttttaagaaacttttgactgctcagattctttgccggctctctttctcaccccatctgctggtaccaggatagtgccttgacaa

SEQ ID NO: 241 - DNA, 376 bp, Homo sapiens
agttggacatcaggccacacgctgagagggtagtaaatgaaagggagtccggtttagtcgcgggcagggcatctcctcttctgcttgtctaaaagtcgctgggtgcaggggaagcaatactaagcgggaatttctttttagtggcatctacacaatttgcctttcctggtttttgttttgttttgttttgtttcctaaggagggggaggctctgtagaggaagcttcggccctgggtgcccctgggttgggagcagtcggggagcgctgagccagggcgcacgccctgtcggcgctgcgggtgtggggcgcaggagtccttctggccggcccctccgccccccggggaggctggagctctccaaggccggggagcc

SEQ ID NO: 242 - DNA, 429 bp, Homo sapiens
tgcctcggcctcccaaaatgctgggattacaggtgtgagccaccgcgcccggccacctggctaatttttaagttttagtagagatggggtcttgccatcttgctcaggctgatttcgcactcctgggctcaagtgatcctcttgcctcagcttcccaaagtgttaggattacaagcatgagccactgtgctgggccacagagaatattgagactagtctggtgtgtggcatgtgctgttcaagtgtttgtgatgatgctctttattactatggtcattctctatttcaatttctccttggcacttatcaccagctaacatactatagatacttccttacttaattgtgtttatcgcctgctccattagaaggtaaattccgggcagggcccggtggctcacgcctgtaatcccagcactttgggaggcc

SEQ ID NO: 243 - DNA, 568 bp, Homo sapiens
caaggagctggcaccccgggattccctctctgacttctgcttaaggggccggggtggaggggcggtaggtagcccacagagtcccagcaggcgggaagcaggcacaacagcggaccttagcacggggtgggacccaggctcggtccaggagcctcggcctgtccagcactacattcttacttacaatgcttgcaatttttggttaaggtgtccgtcagcttctgcagtttcttgcggttcatgtctggcgctcagagaatcgggccgcggcgggggtctctgggcgcgcggctaccgagaccctcgcgggacccccgcgagccctggtgctgggtcaacgcggtgaggtctcagttactgatctcgtgcgcggccctaacagacggtgtagccaaggcaacggctcagcccgccgcggattggctcccaggctgcccgcgacgtagccaaagctacagctgggccccgcccatcggctcagcgactgctcgcgctgggttctcggtgcgcggtgccgccggcagggtcccagctggccgccttgagggctgtattctgactgttgacg

SEQ ID NO: 244 - DNA, 566 bp, Homo sapiens
ggcacagcgttgtgctgccgcgcgtcggcgcaggcgggggcgcaaggcccgggtcgtccccctcgcgcggtcgggaaaacagctactgagctgcggcccacgaccctcggccgcgcaggggcggggcgggggcgctgcggggtttctctgttgctagagccgtgatgtcacccgcccctcctaacccgcggttggttgttttgtgtttcagctctgcctgcagttgaacacaaagacctggaggagactcccacctccttcagggagaagaaaggaggcagcgaagcagattaaaggaacttgtggcttgtggcctttttgtgcaagggccgcctggaaggggagaaaagtgtatgaaagccaccgtggcggttaatgagctgtgagatgaggcgctgctgcggccgctgctgctgacactcgctcccaggctgcacccgccgcccttggcgcttcatgtacatgtgtctattcaggccttgcggaggcgcccagaagagcatccaacacggctgccggggaaagaccgcccaccatgcccacggagaccctacagacaggtagca

SEQ ID NO: 245 - DNA, 355 bp, Homo sapiens
ctcggccccaaccgccaggcctgccgtttgcgcccctcaggggccagcccggagccagagaagcctcgccctgggccaggcgcagggcccagctcggaacagccccattttctcggttgggtatcgctctccacaaagacaggcgtctattcccgaccctcgaggtcaggcaaaggccacgagtctttagagtttggatcgatgctaaaagctgctgcgctcccctgcaggccccagcctctcctctcgcagtccccaacctcttctgacgcagactttagctgctccacttcttcgtctgctcccgggatcctctccctctcgcggcctgccccccgcctccactgcatcccca

SEQ ID NO: 246 - DNA, 477 bp, Homo sapiens
cgacgtcacgaacgtacaactgtaccgtcgcgagaggacgtgatgcgcccggtgattggcgccgccgctgcggctgcgcaggagacgacccccgcgggcgctcccacccccatctcgcgcggactcgctttaggtctcggcgagtttctctgatatgcgctcgcgggggtgctgccatttcatctcttccgcgcgggctcatcgtgctctcagggtctcgttgaacaaggcaacgctaagagccatcaccacctgtcccccatcacctgttttctaaactaaacatcttcagctcctccagttatctcttgtggcatctgtcatgaagaaagtgctcgggcgtgtgtgtctctctttgaacacctttgctgagggatttgctctggtaccttaactccagagaacgtaaaggtggatgctataatgccgggcgtcaaagcgatttggtacgcagtaagatgatagtagaaattataa

SEQ ID NO: 247 - DNA, 368 bp, Homo sapiens
ccccgccctgtggccatccgatgccacatgtcctctagagttcctgggccggctgctccacagcctgctcctccacaggctgcatggcctcctgcacgtagacgatgaactccgaggcctccccgccctgcgtgtagacagtcaccgtctcaatgccctccacgtcgtcggaggacaccaccaggtggtgctgctcggcaagctccacggacgcttgctggaccgtctcctggatcatgaccgtgtggttggagctgggctcctcctcggtgacctgcgggaggagggcaagagaaaggtcacccagcgcctacttcccggtccagcgtaacaacaaagtcacaactacagtttaggaggcaccagtg

SEQ ID NO: 248 - DNA, 342 bp, Homo sapiens
gtgacctgcgggaggagggcaagagaaaggtcacccagcgcctacttcccggtccagcgtaacaacaaagtcacaactacagtttaggaggcaccagtgcgctgcgggagcacgtccattctccacggatcctctctaccaggctgtgcttgctctcatgcccattttacagctgaacagaatgaggttcagcaatgtagggcaacgtggtcagggtccccagccgtggcaaatggtggaacctaagttcaaacccagagtccgaggttgtttctgatgccgagctgcttcccggggcttccaggaggtgtggggttacctagagaatgagggctggaaggc

SEQ ID NO: 249 - DNA, 303 bp, Homo sapiens
gacagccgtcgtcctcgggattcctgtttgaggagacagccgtcctccccggggtccctgtttgaggagacagccgtcgtcctcgggattcctgtttgaggagacagccgtcgtcctcgtggtccctgtttgaggagacagccgtcgtcctcggggttcctgtttgaggagacagccgtcgtcctcggggttcctgtttgaggagacagccgtcgtcctcggggtccctgtttgaggagacagccgtcgtccccggggtccctgtttgaggagacagtcgtcgtcctcgggattcctgtttga

SEQ ID NO: 250 - DNA, 442 bp, Homo sapiens
caatgaacgcggttccaggcacgagatgtggaggcccagaggcagatgccgggtggcggcggcgcgggactgtcggaggcgcgggcagggacctgggctcccaaggctgtggccctcccgccccagtccagggtccagttccaggggcgcgtctggccctctgttgctgtcttcttagggttctctcatgggaatcggttcttcagcctttcttttctccccagagagggtctgtctactcgttgtaagagggaacaagtccacattgcagctgtacactttgcacccatgatgaaaagcgtcacttttgcagcattccagctggagatgcgtgaccttcggtctgcggcaggcggaagggcgagagacttggacaggaggtgcatcttgcccggggaaggctgaacgaatgagagctcggtccctggtgagaaagagccca

SEQ ID NO: 251 - DNA, 328 bp, Homo sapiens
cgttttccctccttccgtttcccggagccgcctgcgtgaatgttcttcccggctctgctgccccctggaggctagtggaggtaaatgcgtgaagtcggcgttttccctccgcctgttagtcctcctcattcatctctcggccaaaccagaaagatgtgagggtatttcaaagatatctttgctatattttatttcactcaaattcaaggatccctctcccaggacagctgtgactacagaccatgggattcacatcctcccaccccacggcgcaggaccggcaggggatctccaagaagcccctagcctatgggtcgctgagaccctc

SEQ ID NO: 252 - DNA, 804 bp, Homo sapiens
ctggggtttttacgtctccgaatgcccctgccctttggttgccactgtccggagcgtgtgaactgggactggcctttccgccctctgcgattggttagcggtgcgatcacgtttaggttggtcacatggttccgcggactcttaagtgggcagcatgggcagtgttattttcaatggaaagtaggagactccacaggctcttacgggctgttctctcaaatgccttcccctgcctggactctttgaagaggacgaatgtgtagtgaacatggaatctgatgtgtaacaagcctgagagcctagaaaagctctgcttacaagagtttccaaacgctagcattaatcattaatagatgagtaatactaacttattaaacgatttctcagtatagtgccatgacttgcattatgccttttgtaacatttcaaaacgctaaggtataccagaaggagggacaattatccaggaaggtgagagaaaagtttcgtttttgctgcatgtcccttccactagaggttaactttaccttatagaatgctaggaatgcacctaaactactgacatgagttagtcaaagccagatgttcaccttgtattgggcaggtgaggatccttttcactttgtcacatgtgaaatatttgacatgccatgaacaatggaaaaacaaattcttcaaaggagaggaagcagattcttccagaactggaggaaatttgggctgaggcagttattgaccctaaaccaatcccaaccggcatctgtcttaagttcaaagtctcagggcatgtgggctgactttcta

SEQ ID NO: 253 - DNA, 401 bp, Homo sapiens
cagcccagcagggcgccatcgcggctgcaggagtcaaggtccagcgcgccggagcggaaggccaggcggaagacctgctctcccttctgattgcggatggagaagccgccagctttcaggtccagcagctccgcgcgaagtcgctccgccttgcgtagggagacgctgtagtagcaccaggccaccaccgcggccagcacaagcagcagccccagaaccgcggagcccagcagcggcttcaggtctttggaaggcttgggcttggcaggtgagaagttgtcgggcaggaaggtgtacatagctgcggctgcgatggcctcggggttctgacgctatgcgtagcagccaggccggcggcggcgggggtaggcctggctcttctcctgagggttctggagcat

SEQ ID NO: 254 - DNA, 339 bp, Homo sapiens
caatttccagaatgccattcggagcggagttacgaagcggaagcgaggccgggggcgggggggatccgatgcgcgccgctgccgctgcgtgggggtgaggggatcaggtacgctgtccgctggttaagggggctggctgggggctgcggggtctgtcctgggacgtccgcctccctcgcgggtggggcggacgttgagaaagcagcgtttagcagtcatgtggcctgtggtactccaccttcctgatactttcccttcctgcttccactcgcacgctcagcctgtgggccgggagagacccgcaggcggggtgctctcaggctgaattcgatgtgccat

SEQ ID NO: 255 - DNA, 429 bp, Homo sapiens
tttcaaacttcagcaagaggacaaatattcagccagagttatcagttcccggtttctgcttcgggtcctctctggtctcccacagcccctcctgcatcacccaggtctaaggggccacctggcctggcctgtgttccctcgtccctccccttccccacccagatcctgcagcgccttcctggggcaggggcggcgaaccgtccagagcgggaggctccatcgcccagggcagcaccgctccgcccctctccacacctgcccagcccctggcaagggagtcgcctgggcctggcccgctgcccgccccccaatacctgccctgtgcctgcaacggcgacgctgccaacaagaggtgccagagaccgcggcgcaacccgcccggagagcagggtcccactcacctgggagcgggggcacgtcccttccggt

SEQ ID NO: 256 - DNA, 375 bp, Homo sapiens
cccggagctgtgccccctcccggcgaacagcctgcgccacccgccgcaccgggaggtggctgggggctgccgagcgagtggtgaacgcgatgggtggagagcgcccgaaacgaggtggaaaagggggcgggggcgaggcacggcgccttcttgccccttcttcagctacagacgcggcggcggcggctgagtccctcagcctcggtcatgtgatagtctcgaagcgctagctgcgggggtctcggcgtctcggggaccattacaaaacgcagccaggagagcgcgtcgctgccactgccgtcgcctctcctcctcaagacaagcccgggagaaaggcggcagctgcggcgcggacggctgagcctccccggccag

SEQ ID NO: 257 - DNA, 591 bp, Homo sapiens
acgtagccttctgccctgtcccttgccagatggcgccctcggaaccgcccggagaaaccaggtgtggtttggcctccgagtcccccccaccgcctgctgtgggcacagcactcagccgcgagggggacagcgggtgggcagcaagactgcttcctttgcggcttcagaccctgttcgctcctaggttcctgtggtccacgcgccgtctccacacccacttcctatacttgagttgatggttagaaccttgtcgtcaccctgcagaagtacagtgccttgaatgccagcttttccgttccctgatgaaaagatatgttaaaaaaaattatcggaaaaggtttcatttgcaattggcttgtgcattgataatctttatttactgttttaagttgcagagatgtgaatggtttacaaatctgaagctgaagttcaatctttggttttctgttgtaaatgccttttacaaacattgaattagctaccttaagtattgaagagcttccattgctaggtgagccctgctttgtcctcagtagagtgccggttccctgggctcatccaggggctgagagacggcgggacgctggggca

SEQ ID NO: 258 - DNA, 273 bp, Homo sapiens
cgctgaagacgcccacgggtgctcgaacgcccgttggtgccaggctgcccggcctttagggccttggctgaggacacccctttcctccccaccagacggagcgaccgccctttgccaaccccaagcaaagctggggtcccaaccttacgtgcgctaccgccccctccaggcatttgacctccccgcgtccccattcttgatgggccccgccctagaagtctcccggggtgggagcactatcctcaggccagggcggggccgcgcgcccctgga

SEQ ID NO: 259 - DNA, 457 bp, Homo sapiens
ggagccgtcggggcttctccaccgtttgtgcctggacgcagaggcctcccggctgtgcccgctggaagctgcgggggaagaggaggtcgacagtgagtgggccatgacccgtccgtagacggctgcgggagggttgcaagggccaagctcccagagtgacttcaaggcaggaatggctctgctcccaggtgtcccccaccccctcctctttctccagctgcccaagacctccaggccccactcccctggtcccttcctgtctcctcactctggctgttccccagctgcctggcaccctccagcaaaccctcctccgcgggcgcacccagaggatggcccagctggcggagccacccagcacacaaaaagtctgcctttctttgaccttaggtttccccaccacctgccggatcagggcccgtggggcgggggccgctgcctttgtcaccccagctct

SEQ ID NO: 260 - DNA, 457 bp, Homo sapiens; identical to SEQ ID NO: 259

SEQ ID NO: 261 - DNA, 644 bp, Homo sapiens
cgggttggaggggttccgctttctccctgggtccctgccgctctgtctccggcaagtgatgagcaattttgcggagtggcagaacagagacaaaggtagctgtgggcttcacggtaatttcaaaggaagtgccctacttcgttgcccatttcgaaggcctctctacctctgcgttggacgatttctccacttcctggtgtgcacctgcgtgtgtgcgtgcctgtgtgtgtgtgtgtgtgtgtgtgcgcgcgcgcgcgtgctggggtgtgtgcacactatgccgagagtcgggcaaatttcgagtgaagggagaccgagtcccgcaggttctcttgctcctgcagtctgcggaggaaaaactcctttctggcacggcggcaggaatccatctaccgacgttaggaaaggacagggtgtgctttcgtcccagcttagttttctatggagaaacaggcagcagaggagagtgggccagagggccacattttgcccacgaccagccaggtttgagttggttcctcctaggcctcacggtctgattttctcctcctctttgtgtacccgacgggtgttgctgtgagctcaggaccatcccggcgggctgtggatgggatcatcgtgtgtgtatggccacagccctccgc

SEQ ID NO: 262 - DNA, 457 bp, Homo sapiens; identical to SEQ ID NO: 259

SEQ ID NO: 263 - DNA, 644 bp, Homo sapiens
cgggttggaggggttccgctttctccctgggtccctgccgctctgtctccggcaagtgatgagcaattttgcggagtggcagaacagagacaaaggtagctgtgggcttcacggtaatttcaaaggaagtgccctacttcgttgcccatttcgaagtcctctctacctctgcgttggacgatttctccacttcctggtgtgcacctgcgtgtgtgcgtgcctgtgtgtgtgtgtgtgtgtgtgtgcgcgcgcgcgcgtgctggggtgtgtgcacactatgccgagagtcgggcaaatttcgagtgaagggagaccgagtcccgcaggttctcttgctcctgcagtctgcggaggaaaaactcctttctggcacggcggcaggaatccatctaccgacgttaggaaaggacagggtgtgctttcgtcccagcttagttttctatggagaaacaggcagcagaggagagtgggccagagggccacattttgcccacgaccagccaggtttgagttggttcctcctaggcctcacggtctgattttctcctcctctttgtgtacccgacgggtgttgctgtgagctcaggaccatcccggcgggctgcggatgggatcatcgtgtgtgtatggccacagccctccgc

SEQ ID NO: 264 - DNA, 457 bp, Homo sapiens
ggagccgtcggggcttctccaccgtttgtgcctggacgcagaggcctcccggctgtgcccgctggaagctgcgggggaagaggaggtcgacagtgagtgggccatgacccgtccgtagacggctgcgggagggttgcaagggccaagctcccagagtgacttcaaggcaggaatggctctgctcccaggtgtcccccaccccctcctctttctccagctgcccaagacctccaggccccactcccctggtcccttcttgtctcctcactctggctgttccccagctgcctggcaccctccagcaaaccctcctccgcgggcgcacccagaggatggcccagctggcggagccacccagcacacaaaaagtctgcctttctttgaccttaggtttccccaccacctgccggatcagggcccgtggggcggggaccgctgcctttgtcaccccagctct

SEQ ID NO: 265 - DNA, 642 bp, Homo sapiens
cgggttggaggggttccgctttctccctgggtccctgccgctctgtctccggcaagtgatgagcaattttgcggagtggcagaacagagacaaaggtagctgtgggcttcacggtaatttcaaaggaagtgccctacttcgttgcccatttcgaagtcctctctacctctgcgttggacgatttctccacttcctggtgtgcacctgcgtgtgtgcgtgcctgtgtgtgtgtgtgtgtgtgtgcgcgcgcgcgcgtgctggggtgtgtgcacactatgccgagagtcgggcaaatttcgagtgaagggagaccgagtcccgcaggttctcttgctcctgcagtctgcggaggaaaaactcctttctggcacggcggcaggaatccatctaccgacgttaggaaaggacagggtgtgctttcgtcccagcttagttttctatggagaaacaggcagcagaggagagtgggccagagggccacattttgcccacgaccagccaggtttgagttggttcctcctaggcctcacggtctgattttctcctcccctttgtgtacccgacgggtgttgctgtgagctcaggaccatcccggcgggctgcggatgggatcatcgtgtgtgtatggccacagccctccgc

SEQ ID NO: 266 - DNA, 437 bp, Homo sapiens
actctctccaaaccacagccttaccttgacggcctccgcgtgggtcacccgggccagggatttgtcgttgacgcgcagaatctggtccccgacccgcagtccttccttctcagctagagagcctggttccaccagagacacgtagatgcccacgccgtgctccgagcccccacggatgctgaagcccaagccctcgtgggccttggcacgccgcaaactcaccaggcgcacctcccctggccccgcgctgtcggggccgccccaggcgggctgcctgtagggggtggtggcgggcaggtagaggccctcggccgtgtattggtcgaagagcagctggtcggagcgcgggatgaccagacgaagcatgggcagcaggcgccgcttgaccggactgtccagcagcacgcgcagggtgcgcaccaggtcgaagacgttgc

SEQ ID NO: 267 - DNA, 528 bp, Homo sapiens
agacctcacaggtgtgggcacacctgcccccgaccctcctgcctgcatccgggcccctgctgcaggtaacgggtggcagaggttgctgggtgcgggagcaggcagctgaatacccgtcctggggaggcagctgagactctgtgctgccagcatgtgctccattattccaccagactccacttaacacagattcaaagatgacatgaagcatttcaggacagtgaaagcacagcatgaagccctacgcttggggctcccttcttagggcagggccaggtcacctggacatgaagccagcctgatgggaggggccatagcacccacacacacatttcacacagagcaagggccttatgttggttctctccacagagctggggccagatctgatttgaagggaaagttctgtgttaagcactgtctgcagactgctggcccagcaggttagtcggccagccaggggctctccctcttcatccggagcctctggtgccaaagacacaccatggtcagccctccctgacttaa

SEQ ID NO: 268 - DNA, 137 bp, Homo sapiens
cttccgcccgccgcttctcctcagcctcggcggcggcggaagccggagccgggcgcccgtcctcatcgcctcagccgcgggccctgccggccgggccgccccctccccgcgcgggcggggagcgcgcggccgcctcc

SEQ ID NO: 269 - DNA, 505 bp, Homo sapiens
atccaaccccaggcggtggcaacaggacggcttgggggcagccaatggccggggggcggggaacctgtgggtagagtcagcgctggagggcacagggggttacgtgcagtcagaggctgggtctgagtccctgctccccacatgttagctgtgtggccttctccttcctcggtttctcatctaggaaggaggataagagctgcccctcagcgtccttctgggaattaaaaagcatgcgaagggcccaacacggcgcctaacgcacactgagtgctccagagatgctgggtgtgggcgtcactgtctctgacccaccagatcttgggaacgccccttctccttcctgggcctcggtttcaccgtctggctagtgagtgtgggtgcttcgggccgcgagtttcggctgggatgggggcccaaacctgcttaggccactcacctctcggagatgcggccggcggggtagaagacgctctgcatgacgatgaagtacgtctgggggccg

SEQ ID NO: 270 - DNA, 542 bp, Homo sapiens
gacatggggaccttaggggtggcagctgctgccctggaaaggtttctcccggcgggtgacctgccccaggccttacaactagaaacgtaggctggggttggaagcagatgcccgcggcaaggctaagcgcagtctccacctccaaccccgcccccgcagggcaaatccagactgggtgacttggcccaggggcacatcgctcccctgtgtctgctgggcttccctggggcatggatcccgaaagtccgctggagggaccatgaaagggcaggcctctggcttctctgggcttcagtttgttgaactctaaaacgggctagcgtgggagcctgcagagaaggcgtgtggggctctgcgggagaaggacgggcagcgcgtgggcggaactgcaggggctggcagggaggtctgagttacgcggatgggccacgctccgaccgcacccccgagtccgcctgtccgtgtgtctgtgccgcccgcaggacgtgcttccggcgctgctcgcaggactgcggcacgggcggcccctctacgctgcagta

SEQ ID NO: 271 - DNA, 393 bp, Homo sapiens
gccctgtctctgagatgatggactgtgcacgtggccacccccacagccccggagcctcccttgaactcttctctgcaccctgctgaagaagcatctcctggggacggtggcagccaggggagtggcagaggaagagaggacgggtaggccccatctccctgtcccaggtctggaaatgacgcccgcgccccaagcccttctctcctgcccagcttcgtctgtgtcgtctgtgtcgtagaaaggcctgctcagaattcacagcggctcaggggctcatgaattatgtacggctccagatcaatccgtcagcgctctgtcagggaggaatcaaaggccccgcggccggcagacagctctttctgcgtggggccgccgcggagagcccggcgccgg

SEQ ID NO: 272 - DNA, 475 bp, Homo sapiens
gccggcagacagctctttctgcgtggggccgccgcggagagcccggcgccggcgagttggtggagccccgtgggcgctcggctccccgtcccttccgaatgccccgtctctctgtttctctgccagacgcagatttgacaaatgtctttaaaggcggggtgggggtaacaacaaaaagccctggcccgtgtgttttgagcgcgttccaaggtgctcggcgtgaataattgatgccagcgccacttttgtcatgggaaggtcaccctgggtgcggtacagtctccgccaccacaacagcacaagcttcctgcagcactgatgagacggcccctcctcggccaggccaccattctggggctgggcaagctggccatgaagggatggcgaccttgctcctgagaaggggcgtcctgctctgctctgtccgggcctccaggcccagaagcgagggttgaggggggagccgcggggagcc

SEQ ID NO: 273 - DNA, 736 bp, Homo sapiens
gctgccccggggagcggggaccaggcaggcggcagagcgcaggcagccccgggccgtgggaaccagacagggctggactgggtggcggggcaggcctgaggacgcctggaggggctccccacggtccgcggctccctttggacccgagctgggagctgctatagcagtcttaacaataagacagcagtaatagaagccatcggttactgagcaacctcctgggccaggtaccatgaaacactctacatacagcgtcgggaggttttgcaggggaggaaagagaccagatgaggtgagggacttacccaaggtcacacgggaagtggcagagctgggagtccgactcagctctgagtgaattagtatgccagctacccaattctcctccatgacccctcgccccccagccaccagccgtcatcttagtccccatgataaaccagagcagagttgcaatgacacagaagagactttttcatagataaaatagaaacatataacttaactctattgtgtggctttttaaattataaaagtaaaacctcaatatcgaaaaaagggcccccatctataaccacacatcctcacacatcacagttatggggctgggtttccttccagtatctttttcaatgcattttttttttaacgtggttgtaacagtagtccatgtagaattttgaatccggctgtggttttttttttttttttcacttgacattactttatacgcctt

SEQ ID NO: 274 - DNA, 320 bp, Homo sapiens
caggcccgggacacagttcggatctgcccgcggtcctgggccccaacgccgggggctgaccctagaaaggcgctggactggagcccgtgacggggtgggaagggcgagggggcgcatgattcacaatgaaaaggccacgtttccagagttgccttgaaaatgcatccctggacgagctctggctctgcgtggaataagaagccgttagcaggcgtctacactcgcactcggaagctcttgcgtgggtcctgttcagaggtacaacagcaccggccccccaagaggtgcccatgggagaggccataagtggggggtgggcg

SEQ ID NO: 275 - DNA, 574 bp, Homo sapiens
atcaggagacagtgtgggcagccagtgcccgccgtgccagcgccccttccggcgcacttgtgtgcaggtcgctcgcggttacctgccgcttgctggtctgcgggaggctcttttgaaagtggcgccctttggctgctcagcctggcgttgcatgggggacatggtggccacagggccttcgtgtattgggggtgaatccaggacctgggaccacagagagaggagttctggctagaatctcctggaataaagagcccccttacaattcctgggactcattggattgagggactgcccccaggcaggctgaagctggggaccccgaggcctgccagtgggtagtggagaagcctgctggggggatgctagagccctgggctccctgcaggggctctgagcccgatgcagggcgagggttccgttggccctcagaattgcgggtgactcctgtcttttcccctggcttggggttcagtaatccaggaggcctggagagcttttggtgtgctgcccacctcggagcccggatgtggcgagtgctgcctcaccccggccctgccaggcatcttatctg

SEQ ID NO: 276 - DNA, 426 bp, Homo sapiens
ggagctcggccttgtgggggagccccacacgtccccccaggcaggcacccgggtcctgatccgtctgagctttaggtgaaaccccccttccctctgtgcatcctgctccatctcctaggctcaccttctgtcgaggagggctgagcgtcccccgcggagtcgagcacagcagggccaggatcttctcagggaccccccgtgccagctgcacgcacggctctccgcagggctggtgaacctgttccctggaggaatttacacaccacgggctcacacggccagtgccgatgtgcactgctccccttacgtgacatgggacggtgacaggtgttcggccgcctgctgtctttgcacgtccatgtgccctgagtgtctccggccagggccacctggcaccagctgctgtctgtgccacagcaccggttc

SEQ ID NO: 277 - DNA, 1432 bp, Homo sapiens
tgcctcagtctcccaagtagctgggattacaaatgcctgccgccacgcccggctaatttttgtatttttagtagagacagggtttcaccatgttggccaggctggtctcgaactcctgaccttaggtgatccacctgcccttgcctcccaaagtattgagattacaggcatgagccactgtgccctgccgaggtggaagtttggagatgggccacaaactcctggagatagggccagctcagtccccctgagcacccacacacaccctcctgagagccaacagaaggagtgagggccccgaggcggatgacccgtgtgcctcaacccgaacggggagaggcggggtctgcagcagggtacgggcaggtgatcccccaaggaaagattttcctgtattgagagagaaggggccaagaggaggagcttgtcaaacaccacagcccctccccctcctctcagctccagggggtccctggtgccagtgttcggctgatggagagaacggcaagcgggagagagagtgtgacccctgtgggcacatgacttcccttgctgcactgctgcacatagcagaggtgtggtgacgaccctgttttgtcccattgggggcgtttgctgttaggtctgcagaatcctcagttgctattggaaatggtgacatcactggcaggggcggagcttcagccatccttcaagttagggaggggcacgcacactccaggggtggagggggacaaagacagggtggtgtggaccagagggatgggtaaggctctggaaaagggggcgctgggagcgcattgcgagggggctggagagggagagaggagcggaagctgagggtgtgaaacggctggccccgaacacacctcgcggcgctccagtgattcctggtgtccgacctcagccccagtcagtgcgggtccagtttccaggctctcgcggaaggcctggctgagcacatgcggcagccacggtcaccctccctattcctcttagcccgaggaggggggtcccaagttacatggccacgcagatggggcctctccctcatttctgaaccttgtggggaggggaaccttgaagggagcgccccccagagccatggcttagggcctcccccacccctctggagctccagtctgcaagagtcaggagccgaaatatcgctgactgtgggtgacgactcttgcgcgcacacacacatacaagcgggcacgacgcgttcggtcctattaaaaggcacgcaagggtgcgggctgcacgcggtgacacggacccctctaacgtttccaaactgagctccctgcaggtccccgacagcacaggcccctgtcccaggacccctccaggcacgcgctcacacgcacacgcgcgctccccggctcacgcgcgctccgacacacacgctcacgcgaacgcaggcgcacgc

SEQ ID NO: 278 - DNA, 625 bp, Homo sapiens
agcacacagaccaccaaggggaccacagggcacgaccgccctctgcgcccggctaaggccagggccaccgcgggaggcacggtgcgctggagaaccgtcctggcaagcgaggaaccgcttctgtttcacttttgtttggcaaggcaagaaagttaaaaccaacagcgtccgtggcctacagtcagaccccaccttctgcagcacatgagggcacctgcagaggggaactcacagggacagggcgggggcagctgtcgccccatggctctgcgagaagagaatgagggagggggcgcaggccatcaggggatctacccagcactgccctgaagcctctcgccccagcattccagcccagccctctgctccccttctctcgggttcctcggcgtctatccctgacttaagcctctgctaagcaaggtccacagaaacatgttcactgttacatcactgtctcaaaaatcaaggcaagaaaaacacgaaatgaaaacaaaactaccaatagcagaatctccacaccgtttcctgcagcctgagcctcaggaaactttccttactgagtcaacaggacccggacgggcctcgggaagaacagaatcggggtttgtgcaggtgtggacat

SEQ ID NO: 279 - DNA, 854 bp, Homo sapiens
gaattcagacctttgtctgattggtcctctgcttgttgggggaccagcccggtccacacacagcagagtcagcactggtcagtgggagaggttgcagggggctctaaaaagagtttctggcctccattttttctcatcatgaaaaatactgtattcagagttgttaacattctacaatgtgttaaagcctaagacccaggtcccttcaggaaaatgacacggtagcttgtgctcacatgagggtggaaatgacaggtatgcaatgctggaaggcagaggcaagccgcagctctgtctggggctctgctccgtgcgggctgccttctgtcccttggggttccagtttctccatagcagagattctagtttaactcgtatcttttgatgcctgttcaaagtggaagattataggaactcaggggagttttctatgttgcaggagaggtgagaagatcaaaggggatgaagaaaagggaccctgggctcccaacgccagggtgcctacctgcagtttaaccagggcagaccccacaccaagccttggtctgagaagcccaatctgctcctcaaaggaaagaatgcatttacttgggaaaatctgccctgactaaatatgtattaattttcaatggaaagggaagtatgaatatgtttgcctccttcctggtcagaatctttcagccagggtctgtgctggcagacaacaagacacaaggccctttagtcaagacgccaatcaagaatttgcaggaaaagctacaagaagaagaatgttcaaggagaggcatgggctttgggggcagccggggtcctgggagacattcaggttcaagtcctagttttgctaaattcaa

SEQ ID NO: 280 - DNA, 625 bp, Homo sapiens
tacacataaaatgatgtgctcagggtccacggcgctgtgagcactaggccgggccgcaggtgaatcgcagcagccacacctaaagacagggcagctgaactggcctctgctggcctggcctctcaccttctcccagcaggtgtccacttagctccctgggttccctggggaactgtaggcaaggtctgaccagaaagtacagctcttccagcagagctctctgcactgacaagtcccagaacaggcctttccccttcagagacactgtccactctagcggcatctcccctcacaggctcacacaccacacagctccaggaagccaccaactcatcccacgctaggaaaaggcaacgattcctcactcagatccaccccttcctacctgtattgtatttaggcatctcttccccaaaagaaaagcctagctgtccagtttggacccaaggcaggaatgacaaggtcacctgtagatctgtctgtaacaagcctgtaggttccaggcccttccaaggcaaagcggtgctcaacagcaacctgaatgggtatatgaccacctcagggagagcagaggccggacttctgaaacaagggcaggcacacggatgcagccaggcagagagca

SEQ ID NO: 281 - DNA, 411 bp, Homo sapiens
gcccagcagcagcgggtgcacagggagcgccaggactccagcgtcttgccggaccagacccacacgcccgaggtgatgcccaccactaggcacatgaagtacttgagcatgaagacggcgtagtcgggcctgcgtgcctggtcgggctgcaggtcccgcaggcacgggcagttgtgcgtggcctcccagcgcgggcggttgtgctgctcgtagaagaggcaggcgaccaccaccgcggcgggcacggtgtagagcacggtgaacaggcccaggcggatcatcagcttctccagcttgtgcgtcttggtggggccgtcctgttgcttgatgaccgagcggatgcggaagagggacacgaagccggccagcaggaacatggtgccgatgaagaggtagatgaccagcggcgcc

SEQ ID NO: 282 - DNA, 705 bp, Homo sapiens
actggtaatccggagactcacagcggtgacctgtcagcggacgcggagccgggacggagccacggccgctcagattagcaggatctgattgacagcatcggggtagcagacgctgagccagggactcaggaatacagggttcttgcagggcgtcgcggaaggaaacgaagtttttgggagaaatccgtttgataaagccttggcggcttcgaggtcatgtgttgagcctctggctgcatgaacaaaagcgaagcccgctttcatggagcttctgtgaagagcaacaggaacacaggcagtccagtcgtcctgagatactgggaggagcatggttgcttttgaacacgtaggagataaagcctctctaataatgcctgtttttttttttccttcactctgtctcccaggcttgagtgcagtggcacggtgtcgcctcactgcaacctccgccttctgggctcaagtgattctcctgccccagcctcccaagtagctgggactacaggtctgtgccaccatgcctggctaatttttttgtatttttagtagagatgaaggttttaccatgttggccaggctgtctgatatgccttaagcctcttttaaacgcacttttccaggatcggggccacatcctggagctgttctgcctggccgggtgtatctctgggtccctgcctctggagcccctctccgtagatgtgt

SEQ ID NO: 283 - DNA, 458 bp, Homo sapiens
accagccgagcctcagtttcctcgcctgtacaaacaaagcagaaggcgccggcggctctcagtaaaagcgaatgtagcctttgtacttccgacctctcaatggtgaaatgagctaatcacaggcccaccccgcggagtgggacgggagattcaatgagacccgccccacgccgcgaaattctctgcatcgtgtctgacaagcagacagtactcaataaatggtagctgtaagtaagtcctttaaacctaagtacttactgccttacccaagaaaagctccgtgcgacccaaagagctgagaactcccccaccccgacctccgactcctaggacagtactactggcgtctgccttctgggtccagaaaactcttcccacggagccgtccctcggcggtgcttagccctccggatacccccgagacatcccagatggccccagtccggtggcgaagggag

SEQ ID NO: 284 - DNA, 791 bp, Homo sapiens
cagggccatctgagacactccagctggccttggatgctgtgatacaatccggtctatgtcaggacatctaatcacacccctgctccaatctcagcttccacccctggatctggcctgaacccaccaagaatgaagtctgccagcagggaatagtgaagccatgcagtgctccgtcatgctgggccttcctattcacttcaccttgaagcctcgcatcagtctcacttcccagatgagtcagggaggttgagtaatgaagccatactgcaccatcaaggcaggaagtgaacacatgcctacctcactcaaagcccatgctccttctgcagccctaagtgaaaatgccaaccttccccacccagatgccaagtctctgagcagtacccatgggagcaaacatgggcccagtctctggaccagacgcccccaggtgccttagtcctgtggggccaaactccacttgtcacatgggaattcaaaatgcagggttcctgctgataagtgtgagagagagggcactctgaggccagcagccaccatcccatggtgcttctgtggagctgagaggctggatttttggtggaggtgtctctgggttgagagcccagggcctgtcctgagtctgagctgcccttggatgggcttgagctggtgtcttcagacgagccccacagtctggtgcatttcacatcttacccttttgcctgcctgcttgggcctttggagctgtgcccagtgccccgggctaccttccctgtcccacaccacctgacacaaagcctctcacattg

SEQ ID NO: 285 - DNA, 453 bp, Homo sapiens
cccacccggactcccgaggtgtgggagcccaccctgccgctgcccactccggccaatgccagcgcctacacggccaacacctcggcgtccccgacagctgcgtggccagcgggctcagcccttcggccccgctaccctacggagagcgaagacgtgaagatcggggtgctgtttgcttccaaggctatcctgcagctgctagtgaaccccttgagcgggcccttcatcgaccgcatgagctacgacgtgccgctgctgatcggcctgggcgtcatgttcgcctctacagtcctgttcgccttcgccgaggactacgccacgctgttcgcggcgcgcagcctgcagggcctgggctcagccttcgccgacacgtctggcatagccatgatcgccgataagtacccggaggagccggagcgcagtcgtgcactgggcgtggcgctggccttcatt

SEQ ID NO: 286 - DNA, 1337 bp, Homo sapiens
aagcatacgatggcggcttccgagtgggagatgggcatggcctggctgccggccttcgtgcctcatgtgctgggcgtctacctcaccgtgcgcctggcggcgcgctacccacacctgcagtggctgtacggcgcgcttgggctggctgtgatcggcgccagctcgtgcatcgtgcccgcctgccgctccttcgcgccgctagtggtctcactatgcggcctctgttttggcatagccctagtcgacacagcactgctgcccacgctcgccttcctggtggacgtgcgccatgtctcagtctatggcagcgtctacgccatcgccgacatctcctattcggtggcctacgcgctcgggcccatagtggcaggccacattgtgcactcgctgggctttgagcagctcagccttggcatgggactggccaacctgctctatgctcccgtcttgctgctgctccgcaacgtgggcctcctgacgcgctcccgttccgagcgcgatgtgctgcttgatgagccaccgcaaggtctgtacgatgcggtgcgcctgcgtgagcgtcctgtgtctggccaggacggcgagcctcgcagcccgcctggcccttttgatgcgtgcgaggacgactacaactactactacacccgcagctagcatccccactcctcctccagcccacccaaccgccttgggtcaagggggctgctctgcaagcccactggccagctctggctcagggcccacctcctccagcgagtaccccagccactcctcaaccttgacttctgcccaaatcccctccctgtgacccgttccatatccctttctctcttgtccaatggggcttggagcaccgaggccagcgaagccatcgcgctccttgcggaggtgaagaggaccctgagtccccacctgcggctcccctgtgtagagcctgcatctgtctgtccttccttccattgctcccagtgccaaacttgggccgctgcaccgcggcgcctccgcccaaatcaataaactgtgtctgtcccaggaggccgagtctctttactggtggggggtgcgtggaggcgcgcagggccagagcagaggggagggtgaactgggtctccaagtcccaatccagacctaagccaaactaacacgtaggcacctgtagctgtttttctacctggaaaaggggataggaaggaagcaaacccaacaaaggctgtcacccacggtcaccaaggagcaccatgctcccctcagcccaggatagaccctcttttccaggcctagcgcagagcccggggatgccgcccgggggagcctgaggacccgctccagctaggcacgcc

SEQ ID NO: 287 - DNA, 504 bp, Homo sapiens
tgaagcgctgcttctccccctccctccaaaacagccggtatccagcggccggccatctctctgacttaagtcacccagagcacatttgggaaagtttctcgctcgcgctgtgcgcttttttccaattggggatgaaaaggacatggaaagaaatcgcagcaaggttagcaccctagtagataccccaccccagcaaaaatgtgcaaagtcctatttctcaagaaagaaagaaagaaaagaaaagcaagaaaagatagctgcgttcttaccacgtgcgccagcccactccgcctgcgctctggcgggtcccgcggcccccgcgctgaccatgggcaagacgacgcctcggaagggagtccgactcgcatctcgggccctgagtccgaaagaacgggggaaagccgaattatgcaaatgcggatccctgcgatggggctcgggttacggcccccgccggccccttaggtgaggcacccaccggcagcaagcgcgggcgaggcgact

SEQ ID NO: 288 - DNA, 829 bp, Homo sapiens
tgcctcagcctcctgagtagctgagactacaggggcccgccaccacgcccggcgtgtgtgtgtgtgtgtgtgtgtgtgtgtgtgtgtgtgtgtgtgtgtgtatttttagtagagacagggtttcaccgtgttagccaggatgaacttgcactgatctctttaccaggcttccccttaaccctctgcgcagaaattcggggtttttctttgtgtttgagacaagagtctccctcggtctcccagactggagtgcactggcgcaatcacggctcactgcagcctcaacttccagggcacaagccatccttttgcccagcctcctgtgtagctgggactactcgggagtcccatctactcccgtgcgctgccatcacgcccagctaatctttgtattttttgtagagacagcacctcactatgttgcccaggctggtctccaactcttacgctcaagcgatcctccccctcggcctctcaaaggcgtaagccaccgcggccagccttttttggaacttaaaaaaaaagccataatgtgtcattaggaaccttggtgacatgacagcaaaaccaaaatgtacttatttattaggtgtcacttggaagcgcaattttcaacaacgcaaaactataaaaaaaacccaacgtcggcctccacaggctaatcagtctccgaggcactgacggcgggagagatgaaaacgcgaaggccagaggagaaaaagaggaaggatttgcagagagaacggtaacgtggtggacatgaatacacccaagaaacccggatcccgcgcggcacattgggacgaagcgcgctggcgggcggccagca

SEQ ID NO: 289 - DNA, 725 bp, Homo sapiens
acatccaatagagcccacttcaacttgcatggtgcatggggtccaggaccggtggcccccacacagggtttcccaaagcctctccatttgtctgcactctccactgccgaccttgtaggaaatttatttccatgcccagaaatattgtttggacacagtggctcggttatcttcgcgcctgcttttcttaaatacatttatttcacctctgccacgctgcagatatttaaagatctgtagataccgtaaccctcagcgatgcgggactcactctggttatagataatattctctgggtggttatttgaacataagttctatctccagcccagatgacacccaagcaggggctgtaaaggacacttgctctagaatgttttcaactgaaatatatgtccacacacggagaatttaagagtatttttatatttctctctagatctaaatattcagatgtgttaattacatgccctagaagctggaagcgatcagtggtgttcacactggacgtggagctgtttgtataattttcatctccctgcacttaaacatgactctcagtctaataaattcaaccttgtcatttttagaatcgacgggatttctctggctgtcgtttgcgctgcatttatccgaatacatccagctcgcaggcatcctgcaagaaacggctcccggctcgcgtgtacgccgacacctcggcccaacgcaggactcgaggtggt

SEQ ID NO: 290 - DNA, 729 bp, Homo sapiens
tcaatgcccatgggatttgtagccagtccctgcccatcgcccacgaccccgggaagcgcgcacaactctcgcctttacctagatactccgttccctcgaagggacctcaagtcactggaattccccccagagcaactcccagacacaacgcagggggtaaacataaggggttttaggaaggggtctgaggagcacgtcctgcaagggtagaaaaggagcctggagcttggcccagctgtgagcccttgggccctcacttcttcactctgagtctgtttcctcatctcaccaataggaacaggagcatgtacattcccgtagagtagataccaggcccactggggatcttgttttgtaaacgcgccagccagggacaggaagttgtgatcaaaaggcagctggaaggtctgggtcagatcccagcccaggcccagaagttccagctctccaccctccgcctcgcccgcagggctcgcgcttccacagttctcgccgagagcgctatgggacagtgttcaagacgcacctgctgggcaggccagtgatccgcgtgagcggcgcggagaacgtgcgcaccatcctgctgggcgagcaccgcctggtgcgcagccagtggccgcagagtgcgcacatcctgctgggctcgcacacactgctaggtgcggtcggcgagccgcaccggcggcggcgcaaggtgagtggaaacgggaatggaccgtagatacgtcg

SEQ ID NO: 291 - DNA, 652 bp, Homo sapiens
gatgagcccagatactgcggcggcggcgctcgcttcgcgcgagccaacccggcacgggctgggcgcaccacctgccgctgctgtcgctccactccgccttcctttcttctcctcctaccactcgcccgcgcacctgaggctctcaggtgagtccagcaatgggattgcgagcgtcagtgttctctgctgcgtcgccgacctgggcgcgctcgaaggcaagtcttggcacttcggggaagaagggctcgccctctccaggagactcctccagctttcctgctgcctcgatttaaggacttccaaccttaaatgatggccgaccatcgcttttttggacaggcttccttggcccccgccctccctcaattcagttctcttattctctctccgtgcgccccgtcatacttttcagcatacctaccgtatttgtgcttttgcctttatttgtgcggcaattattgggcgattatatcattgatgtctggctccctcactggactgtgggttccagggggaagacgccgcagttttgctcaccaggggctccgatgtgcattcagtaattgtgtgttgaatgaatgagtgggcaacaggcttgtccccggtttgtttattggaatatctgcttcctgtgcaagcctcctggctgctt

SEQ ID NO: 292 - DNA, 377 bp, Homo sapiens
ctgcccctggaggagaggcggcagggaggtggttgggctcgcgggagcccggggccagggaggcctccgtggtagcggcgacggcggcgtcggactggcgggcaggaggaggccgtttcccgccgtgggcttgtcgctgagggctaacgggaggatctcggccctgctcgtgggggttcagctgctgcgctcgctgactcgcggggtgttgggcctgggacgcttcctgacgctctgctgcttgcctctgccgtctgtctgtctcccgctccagtggccctctcacccttcactgtaacacgccgtataggtcacccctatcagaaccggacgccccccaagaagaagaagccgcgcacgtccttcacacgcctgca

SEQ ID NO: 293 - DNA, 398 bp, Homo sapiens
cacaacggggaagggagagacaaagccgagccgcctctcctggccagcccggaaccgaaagtgggtccgtagagagcccagagtcctgggggaacagatgggcaaagggctctaggaggtgatggaactcaggcccgaagggcaggcagcccacaggataccaagccttaacgaactacgccagaagaatgggcgtagggcccacagcccctcacacaacccaggccaagctccctcgtacactcagaccaggagaagaaacagacacgcaatacacaagccccgctcccttggagcctacacttgggcgaacacaccactgctgtttgcagtatcccgagacaggcccggcaggcgtgagctaagaggcctcgggcccgaacttgggatttgaggct

SEQ ID NO: 294 - DNA, 435 bp, Homo sapiens
atgtgtcaatcttctctgcacaagaaagagttcagccccgggaatcatccgggaagcgttgtacctcttcctacatcaatgagtcccctgagatgtagaggatgtttacaggcaccttgcgagttcatgctttgggcagtaatacgcttacattttgcctggttatttcatctggtccaaacatcgagaggttttattatccctgattccgtttgtggccaatcatattttccgcttttcaagcatctccccgtaaatcaccacctgtcagacttgatgtgcattaaggaagtcaatgagttgtatttcacttgtcatgtttgaagaggtgcatgacactctatggttactgctaactaaagaaagagatgacttttgtgcgcaccggctaattggtgtcaactcacatcaccttgcattgaggtgacaggcaca

SEQ ID NO: 295 - DNA, 312 bp, Homo sapiens
cgcagcggaggcggcgccggccagggactctgcagggaccggggtgccccggagggagtggtgctggtggaggaggaggcggtggctgagggggagtaggtggggaaagaggaggaggaagaagaggaggaggaggaagaagaggaggaggaggagaagaagaaactggaggaagaggaggaggcgaaggaggaggagaagccgcagcgggcgccgcaggagcgagtgagctgggagcgaggggcgaaggcgcggagaagcccggccgcccggtgggcggcagaaggctcagccgaggcggcggcgccgact

SEQ ID NO: 296 - DNA, 352 bp, Homo sapiens
caccagccgcgcccctcccccatcgcctgagtcctcccttgggaataaccggccacagaacaacagcaaaagtagcaaaaagtgttgtcctctcacctctgtaaaataaacctaagcacggttagaacatacggtttatttaaacgcttccgcctctatttacggtcctcgcagtccacgcaggcagactcaggagcgcgatcctacctgggagtggggtttggagagcgggaaagcactcgcgggaagaaggggcagaatttctgctggccctcggtagtcctctgcccacgtcggtgatccggtctcgcttctccgcgggagctgaacggatccaaatgtttcccagtgg

SEQ ID NO: 297 - DNA, 318 bp, Homo sapiens
ggcagaatttctgctggccctcggtagtcctctgcccacgtcggtgatccggtctcgcttctccgcgggagctgaacggatccaaatgtttcccagtgggctagcagcttcttagagggactgggagcgcctcccctcagctccgccccccactccagctcccatgtatggaaaataggggtggagcgcgctctgccatgctcggtgcttagagaactagtgaagaggacagtgtggggaggatggaatccgaaacgggaggaggggccggcaaacctggccagagcagcgaagaggacgaatctctactgcaggagc

SEQ ID NO: 298 - DNA, 596 bp, Homo sapiens
gctcagagtctcagtaacaaacccgtttttcagaagtgtctgccaaccccggggtggggaatgaaaacaccagcccgtattgggaaagaaacctgggtggcagatcacgaaagaaaacccttcaaaagtaattttccatgatgtgaccgtagagataaacatttcccatcatctggcttttgagtttggggagatgagcagacagaaaaaacctcagatcttttttcatttcacagacgtttatacccttttgtttcttccagtcacctctgacaaaattctggggacgctgggaacactgaatcaacatgggcaatgagaacagcacctcggacaaccaggtgggtgtcaggaagcttctctctcgagctacgtggtgctcttggcagaccttgactaggttctttttacagcgtctcatcttcacccaggaaggagcaaacaagacagcttctgcgtagatgtgtgcgggcatttatggatctatgcagccatttttcatttcattggaattctcttacgattaaggctagctctagttaaagccggaaacgctaattctacttattatttattattatttttaaagacatggt

SEQ ID NO: 299 - DNA, 795 bp, Homo sapiens
caggtgtggcggactggcgctccccgcaacagaaggtcgcgcaggacaccggaagtcgtcctggagtcagtctgagcggatcgcaggggttccaggcgccccaggccccccacaccgcgagtcacaggaacaaggaggaaaaacagcctctttatgacttgggggaggcaattcaatatttcttcctggcataaaataggaattgctctatttacccagcccgcaggagcgtcgtgaagattaatgactgtgaaatgtttttaagatcaaaaacattcccagattttacttgtaggcacttatatgtgcatattggtatctgtacagcaaaatatactctttattgattgccctcctagtgtgacttctattccgtaatctgaaagccccgagataaaggaatcaggtctgtctctgcctgctcctgaaacactaggaaatatgggtaggaaatgtcaggtcagctgggcatctacgctgagcctctatccagtccgcagggcagatagagcaatccagagtgcggaccacagctgaacgtctggagaaagggaaaaaactttaagattattggtcaccccccactgggaagaaaacacagtatcctccccagctcattaggaggtgctcccaaagcctgagtctccatcagggtctcgggtgtttgcagagccttggctggcactgggagccaatggtcagccaagttctgcctgtgccaggagcccacgcagcccatctggcccggcagctccaacagcaaggcctccctgttccgagcagagtaaggccaca

SEQ ID NO: 300 - DNA, 796 bp, Homo sapiens
ctgcctcaggtcctcgctcatctgacggtgccgggattcctaatctgcccggaagtaacgcaaatgctctgtgaatgttgactccgctgcggttagccagagattgcttggcgtgatgtgcacgagactgagcaatggcacacggtaggggcaaggaggccctgttttctcttgacttattctcatcaatcatctgggagatgactatggatgaaatcatttttgtgttttgtgatgccagggtctcctgcaaatagaaaaagatcccgattattcaagcagtctattgaaatatagacagtaaactctctttcaagggccaccaggatgccagagcactgggttcataacactcagatgtcttgtccaaggctttttactgcacacctgatgctcagagatggcaggaagggcaggaaaactagatgtttatgacaagttacacgttctggaaacattggcctgagtcatgaattggttttctctgctgggcatctattcttaatttatctaatgatgtgtcaacggggatcatctaattcgataaccacctccgccaagcccaggaacccctgaatacaggctcatatcctcaatggagaggaaagacagaattctggaggtggctggaacttggggccacaggtttatgtgggagattagtcagtagtcctgaaaggaaacagcaacggtggttaccaggagccccctccctaccttggtcctttggagtcctccacatttgccggtttagactctaattcagttgctccctgctgcttggcatcatggaagt

SEQ ID NO: 301 - DNA, 543 bp, Homo sapiens
ggtggtctggcatgtggtcaagttacaccaatgtgggaaccaaaatggccggggttgagtgctgtgtcctgtaactataatttcaggccctctatcaagacaaagaaatggtatctaaattgggatatgcacaattgcaaaatgtcaacagcgagtactacagagggacaaggatgcaagaagcgagatgggaggagagctgagtggggactctgcaaagggcgctcccttctccctccacgtttaaatagaacacgggcagtttctccctgtgcttatcaaacccagatattttcagatgctgttctagctgcaaactcatgaagtgatccttgaaattatattaccaacagaaattttccttcattgcttattttgatataatttcaataaatgtacggtatataaaattatttcttcatttcctcctgattctgaaaatgaaattcaatttatgaaccagcctgtggaactccagcagagtgcaatacaccggcagtgtattgttggctgtgggaccaggtccccacgggagaacaaatc

SEQ ID NO: 302 - DNA, 531 bp, Homo sapiens
atattagtgctgtaacaatatgtctgctcagaaatggcatttaggcaaccggcatcatagccaggcggagtatgcccctcagagtatgcggcctgctactgtagcctggaagcctgcaacgctttccttgctattccttgcaactatatttcacatgccacacatacaaagtcaatgatgcatttttatttgccatgtaggggtgaaactcttgtggtagttagaaaaaacaggtgggaaattgctcttctgacagagatctcaagtaacgggcacgctatctaacaacgacacgaacgctgcatcgctgctctagggaagaggttaactgacagaatcacaatccaattctcacatacacgcgcgctatagtaagtacaaaagagtttcctttttatcaataacagacaattgtatatctcaggatgcgagcgcctgagaaggaagtagtaaaatgattccgagtgatccctgactcctccggcaccgcggacggatgcatcacccagacctgttttcatttttgactac

SEQ ID NO: 303 - DNA, 421 bp, Homo sapiens
gggagctttgctgccgctgcggccttgtgtgaatttggagggacaaggccggggcgcgaactgaatctactgtcatatgcggagtcaatgcaccatctccaggacctcgaaagcctcagtttccacgtatgcaaaacggtccatggtgccctcgtaccctggtcgctgctgcaccatcgaggtcagcgcgcggcaagtcccaggtaggccacgtcggctgcagacctcggcggcgggcggtggcggcgcgtgtccacgcgagggcgctgcgcaccgcgcttcagcccgacctcgccttctgcaggggcggtctcctggcgccagggggcgtgcgaggaccgaggagagcgcaagagtaagcccgcgggtgccggagcgcgcgccccgccgtgtgctgggtccagggcgcgcggccggcctg

SEQ ID NO: 304 - DNA, 578 bp, Homo sapiens
cctcatcaatgcatctcacacctctcaggacaccagaatggccaccctccgggcccctgccccgaggacatctcagaaagggagcacgtctactgcattaatgagctcaaatgcctttgtggcggcccaagcctctggtactccaagctgtcacagtgggtgggaccccagcccctgtgtccagcccagggccagaaggcatgccaagtggactgcctggggggagcagcacacggcttggctctgcgctcactcaccaggctccactccagcacgccagcctgcacgtcccgtgcaagaaaatcacaacttcctggggtgcttgcactcccagacgcatgcatgactccccctaaactccagttagtaaagagaagtctcatttcctcaaacggaagctttcaccttggctttacagcaggaagtgccagggtggacctgacaaggtggctcagtggagtcgggtcgatttgcagggtccgaagtcaagcaaagcccaaacggccccagccgaatgtttccctcaaccggagccgggcttcaggtcccacagcgcctgggcccaggccaggggcatc

SEQ ID NO: 305 - DNA, 360 bp, Homo sapiens
gcacaaatgggccaaacaggttcgtaaggcccccgcgggtcccctggcccggaagttgagccgcgggtgcctggtctgaagttccgcgccccacagtggcccgcgggcctcggcggctcctgccctacgccctccaaggcaccaggcaggtggccccagggcctttgcccacgacaccccctgggtggggcaggaccacccctctgtgaattcgtcctggatgcccccaggcacagtctgtactcctccctcccccagcacacgcggcctctgacgctgcacgccgagcttcctgggacccaggacgaaccgggctccaaggaccctcgctccgcccggaccacacgcaggcagcgccgg

SEQ ID NO: 306 - DNA, 434 bp, Homo sapiens
agctccagggctgggttgccaatgcgacaaagcagacgggcccccccaccggcacctacctggggcctgctgctccttcctccaaggtgctctggggtgtgagtcggagctgcaccccagaaacggctcccgaggaacagtgtctgtgaggctgggtgctggggccgcctgcaccccccttcccctcccagggccctgcgggagactgcaggtgggcacggactcacagcagcctctggaaagggctgcagaggggcttgctggctgactggccagggaccaggttgggcagagacccccaggcgggagggttggggctcaccaagatgctcaactcagatgagaggccacctgcccacactctggcaggccagctatcaagaccgggggcttctgtcctctttcacccctcctctccctctccctgcccacaa

SEQ ID NO: 307 - DNA, 425 bp, Homo sapiens
agctgacgaggcttggacccgccgctgccagctcggccctggcctgcgccggatggggtcttcggctgcccccgcgcggcctgctgctgctgggctggtgtcccttcgagggctccgcgcgcctggagccctcacacacctgctggccctaaggccctcgagtcgggaggggggacaggggtcagctccactctttccccgagggaccatgtgtgggacaccttgagagactcccaccagccccctggagctggagtctgaagccgctatctgcggaggcccaccctgcccagtagatggaacaggggctgagcccatcatgtcactgtcgggggtcccagcccttctcagccttgggccagaccactccagccccgggatctgcctgtccaggctggctggaagctgttgtactgggtaaaagt

SEQ ID NO: 308 - DNA, 666 bp, Homo sapiens
gagaaggagaatggagaccctctgtgcccagtgaggctgggccaccttccgggtactccactgatgttgggctgctcctgctagtctttggttggctgtgggcacagggggcctagaactgtggacaggggcctggctgtgtggccttgagagaataacccaacctctctgtgccttggtttccccctctcactggactgaccgtttatggacagctcgaagcctgggttcccaggcttgggactggctctcctaatgtgtcagccgaccactgcgtctgtgggtggtctgcaggggcggcccccaccccttcctaaggagaccccctcactgtccatctgtttgcctcagagcctgggctcccagccgttgacggattgttttccgcattttgctctgcagaacactggacagtaagatgttcttgataagagagtttttccttccacggactgcgttccgcagctcctgggtggggtgcagagtgtgtcagctcaagccagaaattcggtcgtaaagatgctgtttctcttcacgtttctcaaatggacttggcttcggaaatcacttctgcaacacaaaccagcagcctgtgcagctctgggcgggggcatgccggctacccagctcccggcattgttgcacatggctttgaaacgcacggca

SEQ ID NO: 309 - DNA, 697 bp, Homo sapiens
tcgggggcgcactttcccggcctccagcgacactgcgcacgcccctatccggagtgggtgtgtgtccctctgggcgtacgtgcctgaggctgtgccacctcctgtgcttgttcttgtgtttctgactgtcttgtttgcgtgtctctgtgggttcccctctgcgtgattgtgtctctgggtgtatccccgcgtctgggtccctctgtgtttgctgttggcatgggtgtctgtgaacgccctctccaggttcttgtgtctgaataccgctatttgtggatccttctgtaggcgtctctgtgttttcggtctgtgtgtcctcctctctgtgtgctagtctttaggtgtatctatgagcgcgtgtgttttctgtctgcgcctctgttttctagctacgtctgtctccgaatgtaggtccagtacctgtctgcgcgggtccctctgtggaggcgtgcttcctcgtctgtgatctgagcttctgtgatctgtgtatatggatgtgcgtctgtaagtctgtatgtgtgtctgtctgcatgtctgcgagtatctgctgtgcttgtgtcggtctgtgcctctttgctgtgcctgtctgtccatacgtgtgtatgtgttggtgggggtggtgtaggtggatcggatccgagccaagccgggggcaactaggccaggcctgcggttcccctggctctcgctctcccac

SEQ ID NO: 310 - DNA, 349 bp, Homo sapiens
ggccggggccggctgcggcggccgcgccagtctcgcttcaagacgcagccggtgaccttcgacgagatccaggaggtggaggaggagggggtgtcccccatggaggaggagaaggccaagaagtcgttcctgcagagcctggagtgcctgcgccgcagcacgcagagcctgtcgctgcagcgggagcagctcagcagctgcaaactgaggaacagcctggactccagcgactccgactcggccctgtaaggggcgccgcccgcgggggggacgcgcgcgtccgcggtccgcgcggggaccggcgtgtgaaccccgagagtgcccgcgccctgctcccgggggacccgca

SEQ ID NO: 311 - DNA, 420 bp, Homo sapiens
cctgtggctgcgttcaaggcagcttggaggtcatttaggccagctcccccgggttccccgtgggctcaaatcaggacccagacccttctctcttgccacagctctgggccacctgggactcgggacttcctgtctcccttcctactctgtcctaaaccattgttcagaggcctgccgcctccaagaatcgccgagggagaagctccaggggaactggcctgcccgcatccccactgccagtagcgctcagggtcccaggactcgggtcttgccagaccacagtgtcccctgggaccctagggtctctggctccgcactgtgttgcgccagtgcgcctggatttcccgtgagctttcggccaaccacgctccgggcttcccgcctggctccgcacctgcggcgctctcacctgcggcggcg

SEQ ID NO: 312 - DNA, 706 bp, Homo sapiens
tcacacctgttatctctcaccctattctctcttcacccctactcccgcccggcagctccccgctgttagcaccagaaatagttatctgctaagaaaacgaaaagtgtaatgctggctttaccccgcgacgctttaaaaaactccaaaaacccagcgtgatggggggtaggtttgcctagacacagctgaggcagccttaataaactgagaggaaggttaattgtcaaagagggttgagccactaggagcagcagcaaccttggactaacaggtgtatcttcagcgagcacatgtcccagcacatgtccccacgcccatccttccatttagcgccacaaacgcagcctcttaagagaatgggaagtttgggagtcacagaacggaagctaccattggccttaatggatgtcaaccattaaggtaagcttgggagtcggagaacggaagctaccattggttttagtgaatgtcaatcttcacccgccacttttcttcctggcctccttgccccttcctattctgctggctagttctctctcgtcatctgctggccgcactcgggaacataccaaacgcggaaacgcgccttagcttgccctgaatacctgcaaaccgagggctcggcgtggtttttaaaggcttggctagcgtgcccccggcggtactaaaagagggctgcttgcgccaatctcattcccacaggatg

SEQ ID NO: 313 - DNA, 328 bp, Homo sapiens
ccctgtcctgtgctccttgcaggtattcaggctccaggccaggtggggccggacgcccccagccatccaccatggtggtggcacaccccaccgccactgccaccaccacgcccactgccactgtcacggccaccgttgtgatgaccacggccaccatggacctgcgggactggctgttcctctgctacgggctcatcgccttcctgacggaggtcatcgacagcaccacctgcccctcggtgtgccgctgcgacaacggcttcatctactgcaacgaccggggactcacatccatccccgcagatatccctgatgatgccaccaccct

SEQ ID NO: 314 - DNA, 578 bp, Homo sapiens
tatgtctcacacactgactaggggtccttcggcctcagaaaagtggccccggacggaatacccactaagaagtgctctctgaaaaaggcgggcgccaacctcgaacgaagtcgttttgagggactggctcgccagctcctaagcaacttcggggctcgtgccgcgcacccaccaggccttctgcgcacgcgcaagtgcgcttcccaccgccccttcgggcgcagctccaaggcctggccgcggtctgcgcgtgcgcatttcattgtcgacaccgagaggacgccttggcgccttcaggtccctcctggatttaccagcccattgtttatttcgcgcctgctttagcgtggtgtcgtcgggaactagcgactgcagagtccttactgtgtgctaggcgttatgcagggtaccttcctttattcctcggcgatatgctctgcggcaggcattaatgccccaaatttaggatcgcttgagctcaggaggccgaggctgcagtgagccgagatcgcgccactgcactccagccggggcgaccaagcgagacctgttacacacacatacacacacacacacgc

SEQ ID NO: 315 - DNA, 509 bp, Homo sapiens
ggatctcctgtcgccttcggggcgctgcccatggtgctgacggctgcgccggccgtgtatggctctgtccatggttctgaacccacagtcggcttcggagctctgtccgcggttctgaaattcagagccgctttggagctctgtccgcggttctgaaattaagagccgagaggagccgaccccgctttagaagtcgagggcttgtgggctatggagatacagcaacaggttccctggccaagagctgcgggcagcgggtcaacaggtgtttgcagaggcaggtccatgagaaattcctctggattctctgaaactcagaccatgccttcctcacttcttctctgcctcccagtcttactcctgacgcactacgtcttctcgccctacaggcactcctctggcgggccccacggggcttctctcagctcaagagcttgaggacatcgtagcggcactacccggctttctgcttgtgttcacagccgaggggaaattgctctacctgtctg

SEQ ID NO: 316 - DNA, 458 bp, Homo sapiens
gttccctcgccccacaaacaccccggcagatggacacctcctggcccgccggcctctgtacccctccgaaatgggagtaccgcagcatcccacaggagcagccgcgcccacctgatgagcacaggtgtgtagagcagggcggcgatgggagtcacgttgatgcccatcagcattttgctgtcgatgtcgggcagccccatgttgccatacttcacctcgcggtaggtctcgtcgtagtgcaggatcagctgcatctgcaggaggcctggggacaggacagagagcggcgtgcagggagcagcctagctttgggcgccaccgagcagaagacggcatgcaggcctcgcagggagctccaatcaccgaaggcattcccgctgacagcgcccctcaggacagcggggttcccgggctgcgccacgccttctgccagcccgcggccacttggccagctaagc

SEQ ID NO: 317 - DNA, 584 bp, Homo sapiens
ttgcagcctggccgtcactcctcggtacgtgttttggacttaaacgctccggatgtttactgagtgcttgattaataacatggaaggcctggtctcattgctgtgggagtgaaggatgcacagccaggcctgacatgatgagaacaagaacctggagtctcgctgcctgggtggtaatcctggccctgccacttagcaactgtgtgactgtagccaggtcacttaattttgctagatcctgcctgcgcttcagtggatcttgctggttttccaaggtggccaaacactttaaggcattcatgtggtcgctaggctgcagggttgaaccctggctcaccccgcagggcgccgtgtgctctgtggcctggctgtgcctttgctgacaccgtgcccgtgtgtgttcatgcaggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcct

SEQ ID NO: 318 - DNA, 636 bp, Homo sapiens
ccaggcatggcccggcctcggagctctgcggccccgaagccgcctcacccggggctgcagctcggagcccaaccaagagctcacggctggcgccctggatgggaccttcccttcctggctcgcggccaggggtacctgttggggcagccagggtgagggtgatggtgggggtcttgggtgaaggccaagttggtcgccactccacgatgatggtaactgccaggccccactgcttcactatcctctggggccagagtcccgagtcctcacttgccagaaacgcatattgtgcccagggcactgggcctggtaccctggcattggcaccgaaactctcaaagcccagggtgtcaatcattggagattgctttctaccctcgcccctgtctcccctgggcgcccaggcctggttcaggtgggggcactggttggaagccagtcatgtaagttggggaccccatcctgaggtccccaatatccccaccaccctccccagggccagacccagaggtctaggtaaccctatgttttagagaggggggcagaattctcccaggaagggtttggcttgaaggacaaagtttgccggaagaggataggacagggctgggactggctatctcctcttggggtgag

SEQ ID NO: 319 - DNA, 765 bp, Homo sapiens
cacagactcctacagcaccagtgcagtgctgcggcgcccacagaaaggccggctggcagccctgcgagatgagccttccaaggcaaggtctttgtgtgaaatacctctgcttacgtgaaagttacctacttatcccgtggctttcagactactttgggataaaaatgttgataggtcattgctttcttgccctcctctcattttctattttctgactaagagtttagcttttagagcattttttaaaatagggttttgtttttgtttttgtttttgttttaaagaggtagggtctgtgttgcccaagctggtcttgaactcctggcctcacacagtcctcttgcctcggcctcacaaagtgctgggactacaggtgtgagccaccgtgcctggcctctgtagtttttttttttttttccttttaataggaattcttataaaaatacagtcacttttaaatacaatgataatcatggatttaaagtagtgattaaaaataaatgagtaggccaggtgcaggtagctcacgcctataatcccagaactttgggaggccaaggcgggtggatcacctgaggtcaggagtttgagatcagcctggtcaacatggtgaaaccccgtctctactaaaaatacaaaaaattagctcggcacagtggcaggtgcctataatcccagctactcagaaggctgaggcagaagaatcgcttgaacccgggaggcgttgcagcgagctgagaaagcaccattgcactccagcctggg

SEQ ID NO: 320 - DNA, 775 bp, Homo sapiens
aactaggaacttcagctctcacttcctagaacggggttgctaacaaggccggcatcggccctgatccctgcacagatggcccctcttgctcccccactgcggggccttaacatccttgtcacccgacatctcagggctggcctcagtggggaggagggaaggtggctctcagcactagtcctcagagagacagccaagttgatggggtaagtgtgaaggattttaaagtggcaattacaccaacctacagtgttgtaaaaacaaaacaaaaaactaagtttttaatgaacaaaaaaatctcaacaaggtaataggagatcttggcaaataaaaggcaacaataaaaacagataaacagaaaagctagcaaacatgatttatgaaattataaaactcaaaagacggacttaacagctacagggacatttcaaaggaaagggtaagtgaacttaaacaataaccaatagaaatgatctaatgtaaagaaaaatgactgaaaaaaatgaatagagctccagggacttgtttgatgctatcaaatgatgaaatatgtgtgcagcagaaacactggccatcaggcaagagggcggggctctgccatcattagaatgttgcagtaacccaggtcaagctgacctgatcagtgtgtcgcgcagagaaaggaactgagtcaattacctgctgggggccaggaggcagccagctggggaaggcgggatttgaaccgggcctactgacctctaagggccgcaccttccacccatgccactccaat

SEQ ID NO: 321 - DNA, 284 bp, Homo sapiens
cggaggggggcgcggtggctctaggagccgcaggtccacagaggaaatccggcctggccccgatttcccagctgctggcgccgccgcacttaacgcgggggcaaacaggcgaggtgacggcggggctgaatgcggaaggaaatgacccttcgctggccctggggcggccgcggagggggttccccgcaccaagccgtccactctggtgggtcctggcaggtcacttttcctctccggattgtgctctagtgtctggacccgtactcctccatcatctgcgggac

SEQ ID NO: 322 - DNA, 305 bp, Homo sapiens
cgacggtgacgcgctctggctctgacaagcctggctccccccaccctaccgggtggcgccgttgctgaccccacgcccagccgttcccactcccgcctgaaactcctccgacagggcttcgcttcgggggctcagtccccagcgcgtacacactcgcgtggtgtcggcttagcctcttgtaaaggaagcagggcaggcaggtgggaggaagcccacaggggcacttgggatgatgagggggcatgctggcaaagccgggtcagtgacaaggatatattccatttgtttccacctaagccttagcc

SEQ ID NO: 323 - DNA, 519 bp, Homo sapiens
gaggtaggtgttaattgtctcttgtgcttctctgggtatgtctggtagccggttttgctgttaacttgaacacggattagatgtgaggaagaacaaaaggatcaggaggatttctaggcaacacaatctcttataaaatggcatggctattgggggaagaagataatgctttgcagaattctaattttgaccctcttctgaaaaaggaatccaattttaataatcattgcccgatgaaatggtcacccttgcagtgctgcaggctgtacacgtgcctgaatttgattagacggcatcccgagaaagggttcatctgcttttgtctctgggtgattacgtctgtgcaagtgtctgtcaagtctgaattattctgcctcacattatatttctgcccagcaacatagtgctgcatgttgtgacaatgaccttaccttccaaacacctttcaaacaaaatgtctggttcaccccggctcattataactagatatgccaactgttatccttcaatctgaaaaga

SEQ ID NO: 324 - DNA, 328 bp, Homo sapiens
aaaattctagtttacgacggtcgtaatttaacaaacaaaccgggacttccggacccgaggaaggaggagcacggctccaccaacagggcatgcgcacatctgaggtcgcttagggctcggattaccgcagcccagattttaaagacccgaagggtgttggagggtttaattctttcaggtgttgggtgcgattgtgagcaggattaatacccgtatatatttattatgcactttttactatattaacagctttctcaccaatttcgtcttaaaatcaccggagtaagttacaagctcttggaagttaagttggtacctctcaaaagtt

SEQ ID NO: 325 - DNA, 274 bp, Homo sapiens
ccgccccctcagcccgcccggacgccccttccccaggcgtccgccccgccggccctggtcgggagaggctctggcccttccctctcgaaagtgcaaacactccattctcgcttcccctttgcccctcctcttccagggacgacttcctcctccgcaatttctcacttctttctttggcgcccatttcctgcgccccctttccatctttcctttcccgcctgaaccggggtcgtcctcgccgccccctcttccccaactctttcagcccctgtgg

SEQ ID NO: 326 - DNA, 715 bp, Homo sapiens
gccaagttctttggatgccatccgcgcggtgtcccataactttcctggccggtgggcacactgcctgcgtgagtgtttgttctctgcatcgaccattccctgctcaggcgtcgcccttcgtgctgggactgcggcctgctgagccccaggtaggagtccacgcatcgcctctctctccaggtgcacccgcacagctggcgaacttggctaaggtcagcgggagaggaggggtgagatgtggtgctagttttaggcacactgtgctcgttatcagattgcctaggggcccgactgaagtattcctccgcccagcgcctcgggcaaacgcgggatctcccagggaagctcggagggggacagggagctggttcccctctgcgactcgcccctctctggctggccgagtgtctggcgccaacctcgctggaggaggctgtcgggggcgggccagccagcggggctgccctcctcgcagtgacgtcccagggggcggaggggcgaccgactgactccgcgccctcccccccgcctcccgtgcggtccgtcggtggcctagagatgctgctgccgcggttgcagttgtcgcgcacgcctctgcccgccagcccgctccaccgccgtagcgcccgagtgtcggggggcgcacccgagtcgggccatgaggccgggaaccgcgctacaggccgtgctgctggccgtgctgctggtggggctg

SEQ ID NO: 327 - DNA, 605 bp, Homo sapiens
tgttacctggtggataatacttgttgcctcgagggtaaccactgttacccggtggataataagcattgcctcacaggtaaccactgttactaggtggataataagtactgcctcatgggtaaccactgttacccgatggataataagtactgcctcgtggataaccacggttaccctgtggaaaataagtgttgcctcgtgggtgggtaagcactgttacctggtggataataagtgttgcctcttgtgtaaccactgttacctgctgcataataagtattgcctcgtgggtaaccactattacccagtggataataagtattgcttcgtgggtaatcactgttacctgctgcacaataagtgttgcctattgggtaaccactgttacctggtggataataagtattgcctcatgggtaaccactgttacacactggataacaagtgttgcctttggtaaccacagttacccagtggatgataagtgttgcctcgtggggagccactgttacccagcaggtaataagtgttgtctcatggggaaccactgttacccggtggataataagtgttgcctggtgggtaaccacagttagccggtggat

SEQ ID NO: 328 - DNA, 648 bp, Homo sapiens
cgatcccattccatccgttcctcgtctcctcccggtctgacccgttgcccggccgtggttcgccacaccaggcatccaaagctgaggtcgctcctacggcctgggctcgccttcgctttagagatgtttggcctcttccctcccaaacagcccatcttcaaaacctggactcttggactggcacctggccacctttccctctaccaagactccacttccgtcttacccacttcttcctcagattcttggtaccccctgggttggagactgctcattttccttccaaattaatcccagaccccctaaaatattgacaaccttgacaaccccccaaccgaggagccagactttgttttggactaacttccatagccctatcatggaggcagtgtacctggtagtgaatgggttgggcctggtgctggacgtgctgaccttggtgttggacctcaacttcctgctggtgtcctccctcctggcttccctggcctggctcctggccttcgtctacaacctgccgcacacggtactgactagtcttctgcacttgggccgcggagtcttgctttcattgctggccttgatcgaagccgtggtccggttcacatgtgggggcttgcaggccttgtgtactctgctgtatagctg

SEQ ID NO: 329 - DNA, 375 bp, Homo sapiens
agcggccggccgaccagggctctccccaccgactgcctgctttggcacccggctaccccccttggcctggctttcctggggcctttcgcgttcgctcgctttctactgtcggtatttgcagcagcgctccctcaggtcccgagcagagctaagccagccagcacccaggggaaggggacccacagccccgtggaaagaggtctatcgagatgctttgcagcaaagtgctctgcacgtggccctggccaaaatgtgcaccaccccctttcagaaagaacataaggcagagggagcagaatccccctggtgccagggcctgcgcgcccgggctccgggaacccggcccaggaggctcgcggagggcaggaggctggc

SEQ ID NO: 330 - DNA, 715 bp, Homo sapiens
cacgtgtgtgtgcatgtgcacgtgtgtatgtattatgtgtgtgtgtttccggtgggcaggtgtggctggaggcatgaccccttttctctggttgatttcatctccagtagccaagtagaaactacagcatgacctgacttctatgcccccagcactatggccatgacagcctccagataggagaagggtctggcttcattgacatttccacggtagtgcatacagccctcgagacctactcaagagaggacaaaagagaggctcgggtagagcttatctagtggatgaacctgctcctagacacagagcactgcctggcatccctgctgcagaatcgggctaggatagggctgagtattctaattgcctgtgggataggatttcactatatccctgcacagctgttttcatgctgtggttggattttgacgctgtcgtaatttcccccgttgtttgatattctcgcatttacattaaagaagtacaggagaagaacagtgttcttgggagaagatttcagcatttaggattcatacaaataaatgcatggactgcccacgcttccctcacatccgtcaaacaactcagttaaaaacaccttttcaaaaggctcttctataattttacttcttttaaaaatagtgatggggttatgtctgatgccccggaacgtccgtcatgcattgattttatccagttcagtgccaggcagcaa

SEQ ID NO: 331 - DNA, 677 bp, Homo sapiens
aaatactggtggtgcaatgcagccaaccgctgcatctcccaaaagatgccgggaaagaggcagacacccaaacccaccctgggcctgggcagacccctctagcctctgacaagtccaggcactgctcagaccttcacatgcacacctagggccccttcattattcagcaagctcttcccaggctcccatctggcatcaggcatggtgctgggagctgctgggcacacggatgagcaggtggtgacccttttccatcagctacccagagccagctgcagaacagcgccgagagcacatgcctataatgtggggcagagggtcacaccgaggactctcccagaggggagttatttgagctgctctggagcaatgcagacaagagggtacagagcacagaggcctggggtggggactcccagggaccaggagaggcatgaggtgggaagtggggagccaggctagaaagggccttgtgagtcctctgggggagttgcagcttgtcctattagccatgtgcaggctctttggttttcagagaatgaggatcattcacaagggtgactggagctcctgcgggatgtgcacacctggcctgaggttgagatgacacagaaagagtcacagggccggggtctcccgcagagctagtcttctgtttgagtgtcatttatcacctc

SEQ ID NO: 332 - DNA, 378 bp, Homo sapiens
ggggtcagcctcccaaagtgctgggattacaggcgtgaggcaccgtgcccggccaccaaactgactttaaatcacactccttcactcggagtttttacctaggaatgagatgggagcctccactactgcggagagggtcagaagtcatttttgttctcaccctaaccccttcccagtcttgccccagctccgcgttaaaggggtttgccgcccgcagggggcgctgcggtgcagaggcgacgctgccctttcctctccagggccgagcggggggtttgccgcccgcagggggcgctgcggtgcagaggcgacgttgccctttcctctccggggccgagcggggggtttgccgcccgcagggggcgctgcggtgcagag

SEQ ID NO: 333 - DNA, 477 bp, Homo sapiens
gacgctggcggagaccgggcccctccagccgctccgggcttgcggttcccggggaggaggtccgtggggtccacgcgcgagctagggattcgggtgcgggatgccctcggccgtccacacccaggcgcgcgctccagtccgcgcagcccaaactttgcccaccgccttctgtttggctcactccgtttcctctcctgctgcatcttctaggggggttcttttttaccatacctcggagagtcccgtctcttccccgcttcccatacgtcgctcctctcccagcctccgtcctcggctctttgaatctcccacctcctcgctgcgccctctttctcctgttcgggccgccccttctgccacttttctgaaccctgagcaaatctaccaaactccccagtttaaggagatgatggacagcacagtggaccgggcagcaggtagattcctacttctgtttttaacttagccaaagccact

SEQ ID NO: 334 - DNA, 418 bp, Homo sapiens
cgcgctgggcactgatcagggcgattcgggacaaaaatatccaggaggccggcgcggagaggcgtgaggccgacgtttgatgacagttgtcctgaccgctgcctcagtccccaacttggtgcagcgtgtgttgacacggccgaggcagagtggagacaggcagccgcggagtcaaggacaggtagagacaaaaagccccatcagccccctccacttctcgcaggccaggaaggtcccagagccccagaaccgagcctgcgcaatcttggggcctgcagccttggcctcagacggggcccaggttccgagtcccgcagctgccctcggcctctcccacgcggtgttggagggcgtgctaggattgcctccggtggagccccggccccgtttcctccacacgaccgggttcacacctgaa

SEQ ID NO: 335 - DNA, 464 bp, Homo sapiens
tagaagcagcatgcatctgacaattgtcaatttcaaaacaaacacgctccgggacttgaacgcagcggggcattcagtagcgaatgctgtctccttgagttagggcaaagcctgcgtgcccgccgtcccctcaccacttcctcttccccagcccccacctgagagcagacattcggaatgatgtgtagtgcgaggcggctagcctcccagcagaaagccatccttaccattcccctcaccctccgccctctgatcgcccacccgccgaaagggtttctaaaaatagcccagggcttcaaggccgcgcttctgtgaagtgtggagcgagcgggcacgtagcggtctctgccaggtggctggagccctggaagcgagaaggcgcttcctccctgcatttccacctcaccccacccccggctcatttttctaagaaaaagtttttgcggttccctttgcctcctacc

SEQ ID NO: 336 - DNA, 512 bp, Homo sapiens
acatgccagctctgctgaaggcatcaatgaaaacagcagtaggggcggccgggctcctgcgaacaacaacaaaacaaacaaacaaaaaaccacgtcgcgtgcggggcaccaaggcggtgcggggagggacagacccagctggggctccttggaggggtgtgagccaggttggcggatcggttcttagcaacttccctggagaaggggtagaacccagacttggtcaaacgtgcctagcggaggcatccaggcagcgtgacaatcacttggtttccctccaggaactggctgtttttggaataagccaggactagagcagcacttccaaaagtggggtgttgtggacttgcctgcatttatggtagatgagagcggcaggagcacgcctccacgtgtgtcagtgggtggtggggagcgtggtagtggtcctagttctgaattccacacctctttgtcaagcaccggctctgtaccaggaaagctaggtacccaagttccaaacatgaggacca

SEQ ID NO: 337 - DNA, 414 bp, Homo sapiens
cactaagccgtggttttcatattggtccagctggacgagtgtgggattccggcagccgtgggttgcacggagacggcaggtagtctctgccttccaggttggagtcagcagtgcgaagaagcgttcgtattcggtaggagagagagggctgcctggagtggaggcctgagtcgaatcctgggctgcggcaggtgccagaggcaggagcagcactgcggagcgggcgaacggatgatggaaggaccgaaccccaaaacccgctccgagaccaggacagacagacccagactggcaaattaggaacggggtgagggttatccctgctgaccagggcggggggagtctagagacagaggcaaaggtccggccgcgggcggggggagatgggagtatcccaggacctagggagccgac

SEQ ID NO: 338 - DNA, 594 bp, Homo sapiens
tcgaagtcccttcccagggcagcacccggactgagatgtctaaactctccggcaggaaggatacctatccagacacccgcactggatgtcctgtctgtttggctttgagctggtcattacggtgtctttggatcctgcctaacttttctgtaatttttatcagaaataaggcttcaggagagcaaacgttgagttatgcgctccttctctggagtctcctgccctggtgtttgcgctcccactgcagcaagggaggcctgagtctcttagggcaggggtccagggtcttgggagaggccttagggcaagggggatgatctttttcagcctcccctcccctcctcccacccagcgtcctcatccccaccgcctgcgatagacttgggtcgcccattaggaagctgatccgcagggacggggcaggcttggctcctggggatcttaggctccatgggaacttcgggggtctcggggcccgtgagcggatctagctactacttcccctaggttctcctcgggcttcagtccctaagagtctggactccggactgccgcacagaatccttcttgcccggagcggtattattgaggtct

SEQ ID NO: 339 - DNA, 486 bp, Homo sapiens
cacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgagcgggactggacctcgcctctcctcgagccgcgctccagaccacaccagccgccctaactagccctgcctgcgcgcgggccaggcgctggggagcactgccgcccttctgtctttccgccagtgttggggacacgatctgggggtccttcctccttcgcatgaaccccgtggctattttatcccctcggtttccaagccctcccttccagcaagctgcccagctgcccctccctttcctcccggagtgcgggcctgagtcctgtagggctgccctcgctggctgcctgttg

SEQ ID NO: 340 - DNA, 874 bp, Homo sapiens
cctgcgagatggcggagggactgcagacccgggtgcgggagaccagcgccggcgaagacaggcgggcgagggaggcccctccagcgtcctgctggggttgagttggggcggctcgtcccgtggccgctgggtcgtctggtttccgctttccgaaagaaatgagaggagaggcaagtggagtcgctgaactttaattaaaccgtagagaagacagtggggggaggggaaaaaaaacgatcggagaaggaggagaaggcggccgagagatcgaggaaaggaagtcctggcggctcgggggaacttggagatctctccaaggggctgaaagctggaagttgtatgaaggtgttccttttccacaccccagctactctgccccagtcgaagaggctaattctggccctcttataaaaaaaagaaagaaaaggtaaagaaaaagaaaatctaattatgtggcatgtttcagccaggtgttcctggttccaatgactcagaccctattggagccccgaggatctggataattgggcctgcatagacagagatgaaggatgccatttctaaaaggaggaaggggaaaggaaagtccatctcttggggtcttgtagaaaatgccaaagaccaaacgagctaaattattgtgtccgtgtagatctatttgcctatttacatacagcaggctgtgtgggtggggggtgagggaaggcaaggaataaacgaccaggatggagcgggctggcagtagagaaaatgagccccccgccctttgcgggaacagccaaggggcctcctcagctcgcagctcaggcggccgtgcggcgggccggtccggggccgggggccggggccggggccggggccggggccggggccg

SEQ ID NO: 341 - DNA, 861 bp, Homo sapiens
cctcagtcttggagccctgccctgggtgttgctggggcagggcagagtccggaggaaaacgacatagacccttctccctggggaccctgaacttacagggatgacagaccctaatcttggacccaagtgaatagagaactcttcttgcagcttaatcatgaaaaactccccaagagacagaagtgtagagccaggattgggcagagtctgagaagggagagggttccaagtagcaacagctgagggaaggtgcagaggtgaggaaggatgagggtagtttagccccagagacttggcgtagggaaagaagagtcattcactcttgtatccatcattcacttagcaaacaatcgagagagcagctctacatcagcatggtaccaggaaacacacagatgaattacacaggcagggacagcaactggagaggcttccagaaactcagtcctcatccagaatactagcaccagcttcagagttctctgggtccatgctagctgagcttctacctccccgcctctccaatccaaaagaaatccattccctagcattttacttcttcctcagagtcccacccctgcctcagtccttgccccagacccactactcaaatatgccatgctcctcccctctgggcagaccctggagcaccttctggaccacagctacagcaggagggattggggctagacattttgagcaccttcccagtggcagagagcagtggtgctgaggagctgagcagagggccaaggaagggatgtgagcttgagtccaggcccgagatcctcagcctgactttgcatcttgccgggagaggcatcattcatccacccaggggtggagatggggcagggggag

SEQ ID NO: 342 - DNA, 511 bp, Homo sapiens
ggggttcagtatgaagcccccggaccgccccgcccctggccgcactgaccggatactgggggtcatggggggcatgctgcgcgcatgcgccctccctgggcaggagggggtaagaatgggggctcgcggtggggtcgggggtctttcggggccttcaggagggctgcacccacaactcaacttatccgaaaacgagttctgcggggcagtcgcaggaatacatgaattctattacgagggccatagccccgttctgggcgagccttttataacaatgtgggttttgacaggtaggagaaggggaagggggaggtcagggctgcctaggagggcaggttccttacagtggttctgtccccgcagcccccaaggagaagccctctagggttggtgggtaccgagccagagtctgaacgtacggagggagatcacagaagggatcgcgaacatgaggtcctcgccggggctctgcagcccggtaagaagctgggggtggggtggggcacagttt

SEQ ID NO: 343 - DNA, 398 bp, Homo sapiens
gctccttgcaaagttccttttgtggaaaatcgcccagcccaagggagcccggggtatttgcaacagcgtgttcatttccaggtgcctgtcacgggtctcctccctgctgcttctccaggacccatgatgagattatttttaaaaattgtttttggtcgtctcccccgccccctccccttctttatttttttcctcttcgctgcactcttctcggcttttcccctgacactactgatgggggtgcggggggacgtcggggatgggggtggccagcgcggtcctgggagtggcgggttcggatgggctggctgcggtgggccactttgggcatctcggcgtggcctgcgccggggtcacggggagggctgtcagcgccagggcggcggaacccgaggtct

SEQ ID NO: 344 - DNA, 469 bp, Homo sapiens
ccctgccccttacctcgtaggtgctggtaattcccagtttgtagaacaccgggaggaagacctccgcgctgatgaccaccacaaagaagtaggtgaaggcaaagatgctaaaaatggccccaaaacggtagacctcggagggggtgcccaggacagtgacggctgacatgaagctagcggtgagggacagcgccacgggcactgcggtcattctgcggccgcccatcaggaagtccttggaggtctgctggccgcccccagcgaaggcgtagtagatgccgatggcggccgagatgaccagcatgcccgcgaacaccacgtagtcccacaccacgaaggtgccgatgccccgtggcgtgtccatggccgcacggtcgcctgagccctgcgcgcaaactggtggccccgcggcgcgcagccggagcccggcgcgcacttcttatcccggatccctggcgcgcaggcgtgg

SEQ ID NO: 345 - DNA, 524 bp, Homo sapiens
gggaaaccaaggacagaagccgccccctaggacgacgcgcccgagccaccgggagggtctgaacggcgggggcctcctggagcgccttcagcgcctcgtgttttctttccttgggtccctgttagcgtcgccgcatgcacagcgctccctgtcatttggtccctgagcttgggtgtttttcacatttcatctaaagatgcagacccctgctaagtctagccagccttcaccctgaatcttgaacttaagatgctgcattttccacccacccccacccctctagtagctgcagattgacaagcggcttttcccaaccagatgacattttggctgcaagaatccgtggcccgttcttgcctctgtgcagccaagctccttcgctgtctttctcaaactgcagtcctgagttcagcttcgcttagcagtgaccatcccagaggtgggtaatccaacactggatccactcactgcaaccggagctggggatggcatgccctctgcttccactaaccgagtggcctgca

SEQ ID NO: 346 - DNA, 530 bp, Homo sapiens
ctataatcccagctactcaggaggctaaagcaggagggtctattgagcccgggaggcagaggtcacagtgagctgagatcacaccactgcactccagcctggatgacagagccagacccagtattaaggaaagaaaaaaaaatccatgcctctgcctcaactgaaacagtccagatggggtaaagagacccttcccttctcttgtaattggaagtcaggggaagagagccatcctttctccatgctaagctctgtgccaaattcatcacacacatgatcagtttggaactgtaattatcctcatcttccagatggggaaactgaggctcagagggttgatgggacctgcccaaggtcacgtggctggttgttttcaagggttaggggggtggcgctgctcttcttttcccatctgccctctccccgcctgcactgtgagcccctgagggcagaagcagtgcccgtttatcctggaatctccggcctggcacctggcacactggagttgccgaatgaatgaaggatgcagc

SEQ ID NO: 347 - DNA, 443 bp, Homo sapiens
ggagggatttgcctaagctcacagagttagggtgtaaagctcagacatccggatgtgaagtcggggacagacactgtcgctctgctaatgaggcagctggtgtgtccttacatcatgacccccccaggccaggtagggtgaccccaacatggccaccgcttgtctccctgccttgtaattacccctctgatgcctcccatccactcagggtctgtcactgccccagcatctagcacagggcctggcctggcacatagttggtgtttaatctgtcatgccgtccccagtggttggagtgctgacggaggcacatcgcagttgtcagttcccatggcgactgacatcactatgctctcaggctgtaccccgcccctttctccagggctcttgcgccgggagctggccgtggtgctgacaggcccaggaatggccacctaccccca

SEQ ID NO: 348 - DNA, 759 bp, Homo sapiens
gttctctctggccacgttagactcgggaagaggctctgtcgggattggccggaagcagaagggaggtcactggtgttgtggctgctctggaacaactggtgctgatggctcccttggcgaaggagtctgtttttcaacccaggaaggttagtgtgttcctgttactaagtggctgccgttcctcaaatgctgtcattgagctggtaggcattactgtgagatctaaaccttctcaagagaagaggggacaatgagtattttctgtatgcttgttattagttctttccccaggggtgttaaatcagtgaacacgacagctaagataaacatctagggagcttggctcttaggtctgcatttgccacattgctttcggaaagcaggagatatcctttctgttgacttagagtttgctgcaacagggtgtgctggagtatctgtctgccttcgatgaagaaaccacggaagtttgttctctggacactccttctagacctcttgctctccctctggtagaagaggaggaagcagtgtctgaaccagagcctgaggggttgccagaggcctgtgatgacttggagttagcagatgacaatcttaaagaggggaccatttgcactgagtccagccagcaggaacccatcaccaagtcaggcttcacacgcctcagcagctctccttgttactacttttaccaaggtgagggtgccggaagagagggctgggttgccgcagaggtgtttcagaggtgacccggtg

SEQ ID NO: 349 - DNA, 710 bp, Homo sapiens
agtcacctccaggccagtaaaacagaaacacaggcagttttgtagatgccggggctctaccagcttaaggtggtcagttcccagcaaggctctttcagtccatctgcattttagaattttccagtccacaaggtacctggtgactctctgggcaaaaccaattgacaaggggacagttacttggcatacagagctgctgttcccaccatgtaaccctgatgcatttcaacagtcgtctttactcgacgcagcacttggtcctgatatgcacgcagattgagaaaataaagaataacggctccccaggaaacagttccacaatatacacaaatgaaaaaggatttacacagtagaccctaaatagtttctaaggagccattatggaaacaggtaaattaaatttaatttaactgtattttcttcgtttaaacaaatatatctggtgcttggaaaacagcaggttataaaaatgcttaatgtttctgtgaatattaagcatatgcaaaaaagacttctttgtgacactttttctgaagcccaatttctcaccatggcaagagggatgatgcccacaaaggttgtctggctgatgaagatctcagagacgaccagctcactcgtggagtcctcagtcagggactccccagccaggtaatttgccgggccctggtaaggccacgtctggtgcaagatttaacagaaggagccat

SEQ ID NO: 350 - DNA, 681 bp, Homo sapiens
catgcaccggctgactgattcaagcaatacctgtgtggataaagccacccggagcctcatctcacttagggatttttgttttgccatttgtttgttctgggtaaggattatgagggcactgttagtcacatccccatttgttcttgtttaaattaaggtggtttctctgcccctgatagaaaacctgtatgatttctccccagatgcatgagctactgggtctggatttgaggtcctgctccctgtttcattgggcgtcctcctaggagggggtttctaagggaaaaggcatcccccaggtggtcttctaactgcctgtgtccctgcaggaggctgtattcagcacgtggctgcagttcagtgatggctctgtgacgcccctggacatctacgacaccaaggacttctccctggcagccacctcccaggacgaggctgtcgtgtcagtcccccagccccgctctcccaggtggcccgttgtggtggccgaaggggaaggccagggcccactgatccgagtggacatgacgatcgccgaggcctgccagaaatctaaacgcaagagcatcctggctgtgggcgtcggcaacgtcagggtcaagttcggacagaacgatgctgactccagccccggcggggactatgaggaagatgagatcaagaaccacgccagcgaccgcc

SEQ ID NO: 351 - DNA, 439 bp, Homo sapiens
cttcgatggtgtctgctggtggacagcagttttcaacgtcagtgcagtccggtttatcaagctcttctgtattcagtgcttttatgtcttggtggaggaatcctgcctaacccgaggccatgaaagtagtttcctgtgttacctggaacatttgttgttttcactttcgtgtttgggcctgtggtccttctctgcttgtttctgtgtctgcagtgcagtaggggttgagactgaccactgtccccagacggatatgtaatttacccagctccacgtgtgggaagaccaacctctcccctagtgctgtggtgccgcctttgccgtcagtcacgggaggggcgacgtggctgtttctggactctgctctgtccgtcatcccatttgcatgccggtacgattgcaactgccacgtcctatgccaaggcctgagaccggctcg

SEQ ID NO: 352 - DNA, 535 bp, Homo sapiens
agacggggtctccagccctgggggtaagagatggcccacagcaatgagccgggctttcctgggttggtttcatagctgagccgccttgagctgggggacgccgccctcgacatccccagcacagacgccgccgtcgacgttcccagtgcagacaccgccctcgacgttttcccagccaggacgccgccctcaacattcccagaggggacgccgccctcgacattcccagcaggcgcagctcaggagcaccttgccatctgcagctccctgggggacgagaagaaacagcagccacccctttctcgggcaccttcagctccgccgagaacgcccgacactcagagaccctgctctttgttaaatgggaatgagacatgacacagacgcggggcagcggccactgcagcacgggttgtccactgcccagctctggggccaaaccctcggcgtcgcgcaaaccatacctcccgcgtgacggccgtgtccggcctgctcacctgtgcgaggcagccctgcagagcaggtcaccaggcca

SEQ ID NO: 353 - DNA, 514 bp, Homo sapiens
ggaccagtcccgttgtaaaaccgacaaataacagcaggattaggaattccggctcaggattcacgcagccagacgccacaggactcctccccagccgctcctgtatatgacgtcaccgccgtaagaccacaggacaccgccccagccgcgcctgtagatgacgtcaccatcgtaagaccacaggacccttcccagccgctcctgtatatgacgtcaccgccgtaagaccacaagtcaccgccccagccgctcctgtagatgacgtcaccacagtaagaccacaggataccgccccagccgcgcctgtagatgacgtcaccatcgtaggcccacaggacccttcccagccgctcctgtggatgacgtcaccgccgtaggacctaagattgatgctggagaggttcttcagaccctgcgttctgacggctccgctggcaccacccagacgggtaaactagctcttccggtctgtggccctcacaggaaccgactcggtgcaggaggacagcttcag

SEQ ID NO: 354 - DNA, 618 bp, Homo sapiens
caagcggatgagggactaagatgtgcagagcaggctgggtggggactcccggggaggtctcccccaacccccgccccacctcgggcacccacttcgcgatttttgcagaggggagccaggtcagaggtgcagcctggtcccctcgcgctcacgtttttacccaggtcagttcgaagttaagtggaaatgatgattaatcctgacaagtcagatctggcctcagaatggatttcccgtgattgccaccattattagcattgacttttccttgaaaaattggcgccccgtggccatgggccgacctaggcagtttctgcagggacgagcgtgagttttgtaccgcggttaccacctactttccagctccaggtcttagtctaagagggagtgtctgctcatgaagaggcaaagccccaggagctgcgaaaagccttgcatggcccatctgagagatgtgctgagtcggcttgttaaaaatgacaggcaaagcctgtggggtggggcagctttcttggcctgagcgcatcttggttgagccagaggtgacttggggtggggagtggggcgccggttggtgggttctccctttaatttctcaaaggctgtggtgtttatgag

SEQ ID NO: 355 - DNA, 591 bp, Homo sapiens
tctggggaaggggagcgccagagcctgccagcccagcagccagccctgccggaaggcccccaagccaggatcctgtcagtctccatctagaaaccagacagaaagcacttttgtaaatgttaaccaaaaaggtccccctgtgatggctgtgctgtaaatcaaagtagacaatgtggggtgggggcagggcacacgtcttgtctctggagccatcgcaaagcagggaggggatgctccttcaactctcaggtggggactggagctccccgctagtccacgctggagaaaggtttttagggcctggagtcacccaggacccatctctgtccagggggtgctgtgtaatattcaccattaatcctacacatctgggaagcattctcctgctaaagataagataaagcccaaatgatttgtgttgggggctggctggggggctggggggaggccagacctggaaggctgggcacacaggagcaccctaaccagcaagcgggggacgcagcctccgcggcatactcccacagtctgtctgtgttgccgggcagattccctaagcatggtgcactgaggtacacagggaatctggaa

SEQ ID NO: 356 - DNA, 651 bp, Homo sapiens
gccggcccactctgagtgcacagccacactgcttctcgtgcccgcgggccgggcgccacctggcgcagaagtggtgctgatgccgccgccgcacgagagaggagagccacgcatcaggcagcgaatgggagggcagcttgaaccctccctgttctggtgggtgttttggccagcggctgcatccttctgctagtggccatcatcactgtgacttgttcttgccaaggcaaggctaggacgctggtgaagaagatccgtgagcactccccaagcagatttcaggcaacgagtgaccatgctggtccacgtgtcagagccataggggctgactctgggggcttctgctccaggttctttatagacacacggacagtttccatttccacaggggcccaggcgttcgagtctgtggaggattcctagtctgaggtcagtgaggtgtctcggagctcctcttgtcgggagaggagcaggactgcatgggcccaggtagggcacccctgccgttcttagtgtcctcattctcttggagtagtttttcatgctaggagccttatgaaaattcagtgcttgatttgggacttcaaatggctgatatttccgggcttgttttttttttttttttggaaaggaaaaattatccacaggcaa

SEQ ID NO: 357 - DNA, 286 bp, Homo sapiens
gccggactggttccccatctaactgatctaatcaatgactccgaagagccggggctcctggccccgcccccgccatcctcttctccgcctcccattggacgcgggccgagaaggcagggccttgctcctcgctcggattggctgacagggaatcagtatcaatgtttaagcggcggcgtgaggtgaatagagtcagtcagcaagagacgctggggagagagcagggatcgcgagcccggcggatgcggcagcggcggaaggcggctggggagacgctactccaact

SEQ ID NO: 358 - DNA, 574 bp, Homo sapiens
gcgcgacggcggcccggagttgggagtgggcgggcgggccggccagcgccgggaagcctgtcatattctgaagcggctggacctgagccgttgccaaatccgctaatctcagcgctggcgggttcctcttgctcaaagggggctccatcagaactacacaatcaggcccatagaccctcactggggggactttattccctctgcccgccttcaaaaacatgtatgtattgggcgctctttaacttcttttgtcctggcctcttaactctgcttattcctgttcccactctatcctccagcgattggcagagtgaacaccacatttgagctgcacagtgggaccgagattttggaaatggcacgggtgggattggagggagccgcgtgattggcagtagcctcggctgctcgattggctcccgttcaggccctcgacccagcggggattcgcccccgtgcttgcggccgcctgcgctttcgcttcgcgccccctcctccgctggagccacacgtccccgcctccccggggatgcccaagttgcacttgcagaaagtttgagcctggcctgcgcgc

SEQ ID NO: 359 - DNA, 571 bp, Homo sapiens
gcgcgcaggccacgccccccgggtgcgcgggccgggccgcagagcacgccgggagcgtgggtctcccagcccgccacatcccagggctgtacgcgcggactagcggagctaggggaaggtgccagggtggccgccgcctcagtagcacgcggacggctggaaagtcagcccccgtttccgcctcacccaggattgctccactcggggcctttccgcatcccagccaccactccattccgaggcgcgatcagctctgaccagtcttcctcacacccatagtcgattcgttactcccgttaaggtcaaacgctgacagaaaccagccgcttcgcacacttctacatggggtcaagccccctgttatctttgcggggatgattgtaacaatcacttaaatggtcgcttcgtaccatcttgcccccgcctgggccttgtcggcgacgccgcagccacgtggatgctgtggaagcctgtcccctggatcttccaaaggctccgcgctcattgccacagccagcggccggcagtggcctaggctgttgcacgcgatcggtcccccacatctcccctc

SEQ ID NO: 360 - DNA, 657 bp, Homo sapiens
aaaaaagactttttcttcccttctatattagcactattctagacgtttccggtagcatgatatgagaaaaatacacatacacacttttaggctggtgcgtttttgtttaaaatgtgagcctatctccaaatacatgtgtatgcaggaccatgacattgaggaccacatctgaccctccaatccagcccagtgctcagtggaatgccagcatgtggtagatgctaatgaaatatttttggatgattcagtacgcaatttatttgaccaagtgttagttatgagtaaatttcagagccaatattcaaacacagtttagtgtttatccaatgtcaaagtttaaacaactacccgatcttacttctctgtgatagaaaacagaatcactcatgatgcctggctaacctgatttatggggttgttaggaagactaaagagtattttattccatatgaatgtgttttgaaaactatgaaacactggacagatgtaaataattgaattattgagatacaataaatctatagcactcctttcaaacttaaaaaaaaaatcagtgtcatctagcaaaagagaattggaaaagagacttgtcttaaaataggttttccggttgggcgcagtggctcacgcctgtaatcctaacactttgggaggccg

SEQ ID NO: 361 - DNA, 759 bp, Homo sapiens
aatgactgcaaaaacaagcacctgcactccgggtgagcacgcggagctccgggcgccctgcttttcatgctctctcttcagtcactgcatggatgggcaaagaacctaagcctggaattctggtgctggctccagcgccaacccctgggacgctctgaaaatcagctttgccatcatctgtaagtcagggatgactccacctgcctgttttacctggcagcattattttaaaaatcaaacgtgctcaagtattcagttaaatgctgtacaaatggttctgaaagaaactggtctgacttcactgctttttaaacacgtaaatattttgtgtttattctcacgatttctatgtccaacaagccagctgcatttcctctgaagccacagaatgttttaaaagagaatgaaaaggccacactgctagtgttcccacaagtcatgacccaggaagtatctcattcctgtttttgtggctaagacagacataagaaaattggaatttcttgagtatttaaaaaagatgacagtttcaagaaactcccccctttatcttatactcaaggttttgtgttttttccttaataaatatctctgtaattcaggcagttcaagcatagaacattccatttattctgtcaggtcagcctgggtcttagtaaagccaccacactcctccatggttagcctggaagtagctaacgacatacaccggtctgaaatccccagattacttccttacccccaagcaagttacttgtc

SEQ ID NO: 362 - DNA, 321 bp, Homo sapiens
caaacctatctctatatcctcagcactgggctctgcatctccacatgaccggacctcagcaatgtttactggatgaatgtgagagggagggagtggatggcctttgaggctttgcagccacagttttctccattaccagctctgatacattttctgttgtttccaactccattcccctttcacctgctcacctgctctcctgctctccagcacccactcccctttctggagcagcactaatgattcctaagttgtgtcctgatgagctgcccggccaactggccgggcctctaacctcctgaagcgctcgctgatggcccc

SEQ ID NO: 363 - DNA, 388 bp, Homo sapiens
gaaacagcaatgttcatcacaaaggaatcatgatccaggggatgggcgccggttcctgaggacacagaaagggtgtgtctggggagcaagagccgtagtttcggcgccagacaccgaggcaggagctgccgcagcagcccttggggaccccgaacctgccagtggagagccaggtggggcgttctctgaggagggtgaatagagttcccgcccccagccctcccgttcctgcttcctttagggtcaccgtgcgttcctgcttcctttagggtcaccgcgcgttccctgcagagaacacgctgggaaggctttgttgggacaacagcttctgcagccgccggcgccctcctggccgggctgcccccacgtcttttccctctgtgtgctg

SEQ ID NO: 364 - DNA, 592 bp, Homo sapiens
tggatgcgcagagctgtgagcggtcggacctctgtcccatgaggaagcccggatgacctctgcgttctggaaactgcttctgctttagtcatttcatcggctgacctgttacagggcttctgtctggatgcaggggaggcatggcaggggcctgtggaggagcccaggagggaaatggggcccgactggagggtggtggagcgtcatggtgcctctttcatcaggcctttaggaaacacagggaagtctgctgctgtgtatggacaccattatgaccctgacttactctcaaggactcactcctaggctggagctgacctggcgttccacggccaccagcccaggttcatgaggcttcagctgccatcactagtgtttctctcccgcttcttcagtttcagaaacgatcatcatccccctttatcctgaaagccccagtccctgttttcacagaaataacaatgttcccctaaagaaaaggaacaccagagaattggattcaagatctaaatgacctcagtaaccacactgtttcggatttccgggaatgccacgtcagcaaggagggtggaggctgaacactcaccgcccc

SEQ ID NO: 365 - DNA, 405 bp, Homo sapiens
gtgacggggcgcggcgcgtgggacgccaagctgacgtcactgtctgtgccggtgtctcgggcgggggccagtgtggctgccaatggggcaaggtgcgtgggacgtcaagctgacgtcactgtctgtgccagtgtctcgggcaggggctggcagggctgcccaggaggctgtcggtggggcagttcctcatgaatctctgagctgagtttgtaaaagtgtgtgagagtggggaaaatatgggtgaaaatattttcagacatttggattcccctttggtgagctgtctcgtttcttcccgctggtcagggatgtggctgcttgaacagctgggcgcggtgaccaatgtgtggagagccgggaagcctgcggtgaggccgcgcgatgtccttcatcccgggcagtaaa

SEQ ID NO: 366 - DNA, 396 bp, Homo sapiens
actacaggatagaaaccccatgggtgttttcttgtttgcttttgtatcccggggactagaatacctcctgcatgaggtagctggtcagtaatcctgcagcctaagtgagtaaccaggctctgtacctgagaaactgtaaggtaaagaaggttgaaattgtcgtcatgggaggtgcagagatgattgaatagtgaactttggtaggttgactggagccatgaggccccagggggcataagctagtttagcagctctgtgacgtcggaaacctacaggtactgggtccaaggatggagtcattggaacaccccagtgtggcccccgagtagcagcaaggggcatgggccgggaacctatgattctggcacgaaagtgttgaaaagagggactgggtgt

SEQ ID NO: 367 - DNA, 493 bp, Homo sapiens
gctttgcggatggtggaactgaggtccagaggagggcaggggcaggtcccgggtgctccctaggcaaagggagccgatctcggggagggggtcacagggagcgtccctgcgacttctagggcccgaaagctggggaggatgagagaccaggggtctttcgtcgccccctggggctgggcagaggctcagcctgtgttgccacccaaagctgcttctggcaagtccgagccgcgtccctttaagagggggtggagcttcaacctggcacgagggatgctgccagcgtgcgtgtccctacggaagctgagactgcacttcctgcgaggcccctgcagcagcagcggcgtggtcagagcgagcttcggagaagcagtggtgggttccatgtgatggtggagtaggaggcaggtctccgcggtaagtggcgggggcgtggaccccaccgggaaccctcccggctccttccctgcctctccctgttttcgtgctttca

SEQ ID NO: 368 - DNA, 790 bp, Homo sapiens
caaggctgacgtgggacgacgtgggacgccgtgggccgcgagcacgccccggcactactgcggctaagtttggcctttcctggttgccgtcctcccctacaggctgtgcgccgtagcggctgttatccttcttttccgtctcaggaaaacagcgtgcttggtgggtttgagagaacttctgtttttctgtcctccatgtttgaggcggagaaagaacattttgttcttgaggtgatgatggattgaatgtaaagttacgcacaaagtgggatgtttagttggaactgttccctcaggaaatacgattttttgggcgtgggggacagggtttcgctctgtcgcctaggcctgagtgcagtggcgcaataacggctcactgcagtctggaactccaagggatcttcccacctcagcctccgagtagctggaactacaggcgcgcgcgaccatgcttggcttatttttattttgtagagaggaggtctcaccatgttgcccagcctagtctggaactcctgggttcaagcgatcctccatctcggcctcccaaagtgccaggattacaggcaaccgcaccgagccaggaagtgattaatttttttttaagtcacttcacattccagaaacattaatctcgacaactctaggttgctgtaagcagcttgtgacggtggcccccagactgcgtcccgctttagactgcacccagtgaatctccactctaaccaccgcggagtgaccgggtgtattcttcgccttgtatagggcacattggttcagtcttgaaagt

SEQ ID NO: 369 - DNA, 538 bp, Homo sapiens
gcccgcttcgaggagctcaatgccgacctctttcgcgggaccctggagccggtggagaaggcgctgcgcgacgccaagctggacaagggccagatccaggagatcgtgctggtgggcggctccactcgtatccccaagatccagaagctgctgcaggatttcttcaacggcaaggagctgaacaagagcatcaaccccgacgaggcggtggcctatggcgccgcggtgcaggcggccatcctcatcggcgacaaatcagagaatgtgcaggacctgctgctactcgacgtgaccccgttgtcgctgggcatcgagacagctggcggtgtcatgaccccactcatcaagaggaacaccacgatccccaccaagcagacgcagaccttcaccacctactcggacaaccagagcagcgtactggtgcaggtatacgagggcgaacgggccatgaccaaggacaataacctgctgggcaagttcgacctgaccgggattccccctgcgcctcgcggggtcccccaaatcgaggttaccttcg

SEQ ID NO: 370 - DNA, 376 bp, Homo sapiens
gaggcggcggcagcccggccccctcccgccgtcgccccgctaagagccccggccgctgtccccgcctgacaacccgcacgggaaggaagaagccccaggactcacgtcgctctccacctcgatgtcatcgttatcgctcatttcctacggcccagggagcggccactgcagcggcggcggggaggggaaggggtgaaggggagggggaagtcaccgacaacaacaagccgagtcccccccacacacacactcactcactcactcactcgctctctcactcacacacacacacaacacgggcaagaaccacctcctcactgcagcaccggatcaacggcggcacgcacgcccggtcggcccccgccacgtgaccagg

SEQ ID NO: 371 - DNA, 668 bp, Homo sapiens
ccagagttccagaggaggaccagggagaggctctggacaggaggcgatccggcctgagagcgggcagtgaggttagcatttcaaagatggagatattctggggacaggggagagctgtgccatggacaggtagctggagacggtctaggcagctaccaggatgacaccagggattggagggagaggatgttaggttggagccaaagccttcaggatacgaaaggagtgactgggggtggcagctgcaaaggtgggagcgggacactgggccgtgggggctggagaggtgctggggtgttagagacgtcacccgccgcccttcctgacacagagtccgtggaaagagcgagaaggagcccacgtcctgggagagggctaagtgggcgaggaggagctaggagggcagccaggacaggaggcaggaggggaggggtaggagcacaaggacgctggcactggggggcagggaggacaccctgagcaggctggagaaacaaccgtgggtgcgatcaggggacttctgtaggaagaaggctggcgaatgactcgggagagcgggattggcaggcacagggtcacctgactgcaactctgcaaagagcccagtcccatcagccccgggaatggtgggatgctgggggcactgagttctctcgggatcacttaca

SEQ ID NO: 372 - DNA, 660 bp, Homo sapiens
agctaacagctcctgtgggtgacgatgagtaagctgcagtggagaagcccggggacagggactgctcagaaggcagaggtttcaaggtctcatggagatgagatccacttcccaggttatgtactgatgggggtggtggtgacagacccaggttatggaggctactggatgaagagtgcattctagtctctgctgaccttagacgctggttctaagtagttcattcatagtagaaactctgctcttttggacctgtctgatcaacctaagggaaaaggatggaatatagtcctagcatagaaccagcgtgttgtgcagggaaactgaggcccagtctcctaacttcagataaggtggcatgaaggagggaagggatttggtgagcaggccagtcctaggggtccattcaggaaagccaacttccagggccagtatgtacagctgtgtgagtgcataattccagggataagaatgttcatgttgtgcttggcgtggtgcagtgcataacctgcacaactgtatatgatgatcctccttggaattcacgctaccacaattctggtccctacttattggtaggaatgatgacttgctaatcccagtgacaggccggtcccaattctacattctccaaggctgatctcaggaactggagaaggc

SEQ ID NO: 373 - DNA, 322 bp, Homo sapiens
acttcagcgataagtgtttcggtgttcctgcgtgcggatttgtgctctccggggaggtctgcggcccaggttcgattcctgcgacttgtcctaggcaggcctgtatgtgcgcggcggccgcgtgctgtacagtgtgagggaacgtgtaccaaacgctcgcgggatacctgtgcccgtctagccaagagtgcacccgtgtgcgcgagcgggcttctgggacgccgccgtggtcgggggcggccctgcgaggggagggggtcacagggactggccggcgccggccccgtgcgcacggaggcgggggcggggggcgggggccgcg

SEQ ID NO: 374 - DNA, 476 bp, Homo sapiens
ttgtatccccaggagtgccgtctgcctggcctccccattcctgcagtcccggttgtcatctatgtgggccaggccatgagccctccaggcaggctgtgcttcaacccatcccctgcaccaacagcagcctgctcatctccaagctcagctcagcccaagagcagaaagggagtgtgctggaagcaggttcaggaagatgagcgttcatgggccgtctgcatgccctgggctggggagctctcagtgggagccacgtggggtcgaggattgggggaggctatgcaggcccaggggcctcgggctttcgagctcaggctctggaatagccttcttcatgcacccccacacagggactgacactgatttgcaagaatgccatttcgaaatttcacatcacacttccaccatggccccagacagcaagcccggcccagccccagacaggcaggcaatgggcatctgctcatcatccagca

SEQ ID NO: 375 - DNA, 612 bp, Homo sapiens
gagcaggacctaggttcggggcggctgttcccctctctggcatggcctccggcagtggccaggagacggttttggacaaagcttttctcacagtggttgttccagttatacccactgtgactcggggctgttcagaatctgcccaggtgccctgagctctggggcctcctgggtgggggctgggcttgtgggcaggatctcctttgggggctctggaggctgtggctcactttggttgtggggtgagcactggaagccccagctagcagaacacccacagagactggggcctgcacacattccgccccagtgtgtggggtgggcccaggcccctctgcgcaggtcagcttcaatggggagggtgctcaggtcctgcttgttttcctctgggttaatgggattcatctcctggccccagatcctcacaggctgcccctgtccctccagcaatgcaggacatggcaggtcaccctggagggaggcatgttctggtctgggtgtcaggtgtggcacctcagattttccatgcatgctgtgggctgagcaggacagcagatgaccccgggcccccaccctgtctatggacattttttgctgcggcaactgtgggag

SEQ ID NO: 376 - DNA, 518 bp, Homo sapiens
cagggcggatgtctgaggggtctgcgctgggctttccggtggcttcgcccggaacgcgcgcttgttcctgaggcggtgcctgcctgtgtcgtcgtccctgtttgttcttcaaacgtcaggatttctccgtgtttcccagaccgtaacgtacatttaaaaagcttgctttctgtcacatatgcccttttatattatggacataacaattgttacttacctacgtttcctatttgtgaattattcatagataaagaatttaccaaaacttttatttttagttgacacattgcgtatatttatggataacagtgtgatattttgattcttgtattcattggggaagattcaatcaagctaattaacatgggcatcaactcaccagctaatactttatggtgagaacactaaaaatcttttatttagctattctaaaatgtacagcgtgacttcggaggaaatgatactgcccggcctcagtagcccgagactgatcatttctaaatcttgaggttcccatt

SEQ ID NO: 377 - DNA, 783 bp, Homo sapiens
aataaacggaatttgggccctaaagtcctttgttctggagaaccagatccggaatgttcagaggcttgctgttgtgccgttctgccccgatggtatcctgtccgctcgcattggggcgcgtcccccatccgcccccaactgtggtgtcgcgacaggtcctattgcgggtgtctgcggtgggaagggcggtggtgactgggagcatgcggggtaaccgcagtgggcaagggacatgggtggaggtgggtacatcagggtgattgcagttccgtgtggcgagggtacgtggggggaccagtgcatagggattttaggcggaggtaggtatattggagtgattgtggcggggcgaaggtacgtgaagtgatcggtatttagggggtgttgagcgcaggtaggtgtataatagtgaccactgcgtggtggagcagggtacgtggggcgactggggggggaattcgtcgcagtgaccgaggcgaggaggctatggcagtggaccagggggatgagtcggtgtgacagtggtgggggttgtgttggtggtcgcggcgcggggagagtccgtaggaagtggctgggaggtagggggaaggcggcgacagtgggtattggcggcggtgggcattggcagcgggtaggcatggcggtggtggactttggcggcggtaggcatggcggcggtgggcatggcatggaggcggtgggcatggcggccgcggggcctgtcgctgtccggagtgactaagggacgctgaatgattgctgtgtagaggagggggcatc

SEQ ID NO: 378 - DNA, 433 bp, Homo sapiens
cagggtgctttggagtttaagaacgtgcattttgccgatcccgcttgcccggaggcgcccatatttcaggatttcagcctttccattccgtcaggatctgtcacggcactggttggcccaggtggttctggcaaatcaacagtgctttcgctcctgctgaggttgttcgaccctgcttctggaaccatcagtcttgatggccatgacatccgtcagctaaacccagtgtggctgagatccaagattgggacagtgagacaggaacccattttgttttcttgctctatcactgagaacattgcttatggtgctgatggccttcctctgtgaccgctgagcaagtccagagagtggctgaagtggccaatgcagtggtcttgatccggaatttcccccaagggttcaacactgtggttggagaaaagggtgttct

SEQ ID NO: 379 - DNA, 152 bp, Homo sapiens
ggcgctcacatccgcagccccgggatcttagagacagagcgctccgctccggcagcggcgccgcgcccattggctgggctcctcctcgccccgcccagcgaccggcccgctcagagcctacgcatgcgcacgagcaccccgccgccgctgcc

SEQ ID NO: 380 - DNA, 524 bp, Homo sapiens
cttcctgcccaccccaaagggtgcttccctcatggacatctacctggaccggctggccaccccctgggggcttgctgacaactacacctctgtcactggcatggacctgggcctcaagcaggagctgctgaggcacttcctggtccaggacacggtgtaccccttgctggctctggttgccatcttcttcggcatggccctgtacctgcgctcactcttcctcacgctcatggtgctgctgggggtgctgggctcactgctggtggccttcttcctttaccaggtggccttccgcatggcctacttccccttcgtcaatctggcagccctcctcctgctgagcagcgtctgcgccaaccacacgctcatcttcttcgacctgtggcgccttagcaagagccagctgccgtcgggggggctggcgcagcgcgtgggccgcaccatgcaccacttcggctacctgctgctggtctccggcctcaccacgagcgcggccttctatgccagctacctgagccgcctgc

SEQ ID NO: 381 - DNA, 642 bp, Homo sapiens
cccacatgctctacctggctggaaaggtagtaggaggtgggggagggcccggggcagcctgtgggagagccctaccctcaatgcacccctgcacctgccccaccccctggagagtagccagcctaaccttccaagttcctgggcctcacctttcccatccacacctgccctcaagtctgacacccattcttactcaagtgtctgctaccaagaattccttcaaaggtgagctggagcgggcttttacattggaataaagatcagagcctgcagtgtagtcagtgatgaatgatggaatttgagatacccgccaggagatccttggtgaatacagccttgtttgtcagaaaggctgtctacacacacacactcacatcagcccatccacctccacctctccacccgcagtgagtcccctgtggtgcacccactaggagcctagaagccaggtttccttggtttttttaactgcccatgaggcacaggggagagctgtcttcagctggctcctgggaccaggccctgctctgccctccctacctgccaccttcctcaactccatctctcctgaccccagggtgatggaaacatccggtactacgagatcagcactgagaagccctacctgagttacctcatgga

SEQ ID NO: 382 - DNA, 397 bp, Homo sapiens
agaaggaccggaggcccgcgcagccacccagccgacccattccccggcccggcgcgggggctcgcagtcacgcgaggggggagggagtcggggcggggctcttaaagggccagcctcgcgcgcggcgtggggttggggtggagggagaggaactgtcgaagggtggcgggtgcgcgattgaataaggggatgcggtgcaaagggtctaggtctgcgtgtggcttctgcctgcgaggagaggcagcctgcatgtgtccgcattttcggtccacgcctgtggcacgacacgaagccccagtgccatttggcatggcctagctgcctgcctccgacgctgtcccgccctccggaaccttcctgttacagggtttccaggaaaaaaaaagttgtatttgcg

SEQ ID NO: 383 - DNA, 591 bp, Homo sapiens
ccgagggtagaggagcgtcaggcgtgcttcctgtctgtctgcagcagcccggctggcccaaagagactctagggacccaagctctgagtcatggcttcccaggccttagtcagaacggcccctggtggcactcccttccaaggggtggggagcagggcctgtcgacttgctcctgacccacacacggagccagtccctgtcccagctctgccccagctctgccgccgccgctctgctgcgtgactccaggaacccccagcctcgctgaatccatgtccgcagaagagaaaagcctgccaggccttggcctgccctgtctgggaatccttgctggagccaccccgaaacacccctatgggaacccctttggtctttatcattcttgggtgttttccactgatgactgcgggtgtgcccagtggcagctgggcctgagctgtcagcccaggcaggagacaaacaagcctgtgactcaggcagggcatggcacagctggcggaaggaggcatgcctgttctttgtggctgggcggggggttatccggacacagggagtctccagcttccggctttcccacctcggtgcccccag

SEQ ID NO: 384 - DNA, 323 bp, Homo sapiens
cgcccctccaggcagtctcacgggaatgcacgttcaggccgcctttccccggaacccgcagcggcctttctggaggctgcttcttggtgactgggggctccttggagccgtccccagcctcaagcggctgcattgcagctcagggaccccgaagggaggaccacgttctgccgctgaagactcggagtcgcctggcaagacggagcgagaaggggagggtggctggcatggggactctgtctgctgcctcgcaccgcaccttggaggacgctccggccaaggctgggaggggcgagtgcggctgcaggcgcgcggtggcgagg

SEQ ID NO: 385 - DNA, 386 bp, Homo sapiens
gaagaacttgtaaaaccaagtggtgcatttcattagaactcaagtggcccggagaaaagaaaaagaactcatttcccatgagctcttagagccactgaaagctcagaacaaaaccaactgagcagggtttcaagtattctttttggtttcattttctcctcaaattttctgaagaacagctgagagtttagcaaatacgggtgcaaacgattctgcctgccacgtcaaccctgcctcgggtgtcatctgacgaggatgccaagctacaagggctgcggtgagactcagagacacgctaccaatggcaccgcggccccacctcgccgaagcagctgccggggagacgcgataaaactgccccacgcggaaaagaaggaagggcggtg

SEQ ID NO: 386 - DNA, 493 bp, Homo sapiens
gagtggagatgacggggtgcgccgtgtgcacgccttggcgtggtggggccggtcagttttccagaaattaggactgcagagtgaagaacttgttgctcaggacaggccgcccccaccgagtgtcctctggttgggtttcaaatggttctcttggtttggggctctggttcccgtatcctgctcctcacctggaagaagggagcttggagcttgcgtatttgcacctcctgccttctgtctggatctagggcgcagccacggggcgctgtggacggcaggcccctgctcccccaagtcctgccgcacaggcggttggaggtgagcctcgggaagagtcccaaggagtgtgtcctgtggctcagtgaggacgggcgcctgggccctgccagggcttctgggtgtctgggaatggagggacagcagcccagctgcccgccgaaggccggcaccgttcccgggaagcctcgcttttgtccaccgccggctgggtcct

SEQ ID NO: 387 - DNA, 475 bp, Homo sapiens [sequence omitted]

SEQ ID NO: 388 - DNA, 444 bp, Homo sapiens
cttgccagccatggcgaagggagtgctggggactggggtcgctcagtgccggtcggcgaggggcagagtcctgcccgcgtgggggtcaaagacaaatggacaaactgtatgacgtgggcgagagccagcgctctgcagaaaggagacgggagactcgtgggagtcacctcggggggcggccaggacttaggacagggctggggaaagccgccaaggtggcgcccatctgagcgacaggagggagcggccctggcaggacggacgtgggaactgcaggggcacaggccctacgtgagctgcgtgggtggaaaccgaggctgggacaggccgtctgggacaggccgactgggcctgtggagggtgctgctcagagcagagctgggctgaccatgcccggggaagggtgttgcgggccaaggcagggaggatgtttctcctcatcag

SEQ ID NO: 389 - DNA, 614 bp, Homo sapiens
ctggccaacatgccaaaagcccgtctctactgaaaatttaaaaattatccgggtgtggtggcacaagcctgtagttctagctactcgggaggctgaggcagaagaactgcttgaacccaggagacaagaggttgcagtgagccaaaattgtgccactgcactccagtctgggagacagagtgagaccctgtctcaaaaataaaaacaaaacaaaacaaaacaaaaaaacaaaaaacaatcacttaggtaacccaaaatagtgcccatcatttgtgaaaagaaatactcactagttgtattctactcagaaaatggttgccttgcttaaaaagttttgcagaaaactgcaaaagttcctcatttctggtactattcggtaatatatatgtaggtttctttcctcagttcccactctggaaaaaaaaaaaaaaaggaaaacgaaactttttttttttttttgagacggagtctcgctttgtcgtgcaggctggagtgcagtggcgcgatctcgactcactgcaagcttcgcctccagggttcacgccattctcctgcctcagcctccggagtagctgggactccaggggcccgccaccacgcccggctaatttttt

SEQ ID NO: 390 - DNA, 314 bp, Homo sapiens
gtctctcggctgccctgagctggggctggatgggccggaccctccattccggctgcagcttcccgcgcagtgagaaggcagcggtccctgagagcccagcagtgccctgggagcctgcagaacgcaggggcggattccgcgtcgcactggccgtcaaggggcggccgcggagggaaggggtgggtcggtgggtctgacagcgggtctgcgtaggcggcagcgtctgtccctcccagcctctcgctccgcgccatgggcgggccccgggctctgctggccgcactctgggcgctggaagccgccgggaccgccgc

SEQ ID NO: 391 - DNA, 328 bp, Homo sapiens
cagccggagacggtcaaggtccaggacttcagtgactcgacgaagatcccggtcaagagcatccccagtgagcagaaggcgatccagatccagaacgccaccagtaacccgccgtcgttcaaggtctagaacgccaacaacacgccgccgctcccgttctagaactccaccagtgactcgcagaaggtccagatccaggactccaccagtaaccaggaggcgatctcgaagcagaacttcgcctatcactcgcagaagatcaagatccagaacatctccggtcacccgaaggagatctcgatctcgcacatctccagtaactcgaaga

SEQ ID NO: 392 - DNA, 777 bp, Homo sapiens
caaacacctccaccgccgcgtctgcctctgcggacgtggccctagatcccgggtggccatggaacttggtctgggcgtgtagttttcctttgagtgggtcggttctaagtttaaatttccgacgagcccagggtttcgaacttccttccttttttccttcgtttctctttcttttcttttctttctttctttctttttctctctttctttctttttctctctttctttctttctttctttctttctttctttctttctttctttctttctctctctctctctctctttctttctttctttctttttctcttttattttcctttttcttctttttttgttttttgttttttttgacagtgtctccttctgttgcccaggctggagtgctggagtgcagtggtgcgatctcggctcactgcaacctcaacctcccaaactcaagggatcctcccacctccacctcccaagtagctgggactacaggtgctcaccaccacgccaggctaagtgtgtgtgtgtgtgtgtgtgtgtgtgtgtgtgtgtgtgtgtgtttgtgtgtgtaaaaccgaggtctcgctgtgttctggaggctggtctccaacttgcactcaagcaatcttcctacctcggcctcccctcacaaagtgctgcaattacaggccagaaccaccatgctcagctggttccttttccttccaggtcctgaggtcgcagcggcgcgtccctccggtacatggcggggaagaggtgcggttccgggagcctgcgacgcctctg

SEQ ID NO: 393 - DNA, 288 bp, Homo sapiens
gctcaggagcctgcatctgcgctatggcgggtaagccccggtggctgaccggaagaagcgcccacggaggcgaaccccaggcgcccctcagcctacttccatttcccctcgcgacccttcgccccgcccactctagctggctgtgcctgactggctgggctgccgcatattcgcgctgccattggtccataatcagctgggggtgggcgtagggagacctacttcagcaggtcctggccggaccagcattgttaacggctgcggttcgaagtggctgtgggtattgct

SEQ ID NO: 394 - DNA, 867 bp, Homo sapiens
aattgtcccagacaaactaggatggcccgtcaccctagttacaggtgcccgggccaggtgggttccatgtggaggtggaggcagaagcccaacgggaggaggaacaaggggtagtgggaggaggagcccacggggacagtgacactcagagattcactgacggggtgacggacagatcatgcggggggccctgcaatggaatattccttagcagtagaaaggaaaggaactatcagttcccataacacaggtgaatctcagagcagttatggtgactgaaagagccgagcaaaaaagactacatgcaacatgattcctttatgtaaacattccagaaaggacgatcagtatatagggagggaccataagtgggtggttgcctggagccagggaaggaggaaggggagacaggggttaccaaggggcacgaggaaaccttagggtgataggtacgtttgttctcttgagtgtgatggcttcattggcgtagacacatgttaaaactgatcaagctgtacccgttaaatatgcacagtttattgtatggggtgtgtattctattccccgataaagctggaaaaagagttttgctgtaaagaggatgacacaagtgaaacagtagcaggagatgtgattttatcttaagcatggaaggaagagagttgaacctgcgtgacataggggcatttaggctggcagtgagagaggagagcgaggtggggaccatttgtgaaggttactgtatgtcatgtggagagctttggaatttatcccatcatcggcatggttctgcaccccgtcgtgtgaagaaatcatccggagaacttgttctgagtccagaagttacctacaggctgccttgaggca

SEQ ID NO: 395 - DNA, 233 bp, Homo sapiens
agggcgctttttcggcgtctacctgctctactgcctgaacccccggtaccggggccgcgtctacgtggggttcactgtcaacactgctcgtcgggtccagcagcacaatgggggccgcaaaaaaggcggggcctggcggaccagcgggcgagggccctggtgagagggggaggccttctgtgccgggaggaaggcgtcccagaggaggcggaccccgcggggcacaggcctgt

SEQ ID NO: 396 - DNA, 602 bp, Homo sapiens
tgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaaggtgcgggatccgcgcgcatcgtggtcggaggggctgttcggtggcacggggccgatggggaggtcgctgcagtgatttggattctgagtctctagagaggctcacgagctcctggagttgcggggggcgggggcggggcgaagcagccaggaaagagggtctgaggagcttaaggcggggctggcagaggaaagccgcgcagggatggagcctggcgataagggcgctgagcaaacgtaggtttcccggccttagtccctagaggaaactggagatgggcgggcggagccccaggg

SEQ ID NO: 397 - DNA, 662 bp, Homo sapiens
caaaaagccgtaaaaagccacagcatcggggtcagaaagccgcgacggccgggataaaaaccgcggtggcggggtaagaagccgcggcggcaaaaagatgcggcggcggtggaaaaaggcgcggcgggggcaaaacggtgcagcggcagcaaaaagccgcggcgtcgggggcaaaaagcctcaaaaagccacggcggagggggtaaaaagccgcaaaaagccgcgacagagggggcaaaaagcaggggcggcaaaaagccacgacggcggggacatgaaggcgcaaaaaccctccgcggcaggagcaaaaacccgtggcggcgggggcaaaaagcggctggggtgataaaaagccgcggcggtgggggcaggaagccgcgtagggggcaaggagccgcggcagcggaggcaaaaagctgcggtggcaggggcaaatagcagcaaaaagccgcggcggcggggggcaaaacgacacaaaaagccctgacagtgggggcaagaagccgcggctggaaaaacctgtggcggagggggaaaaaagccgcggcggcgggggcggaaaggcgtaaaaagccgcggtggcctcggccaaaagccatgacggcaaaatgccggggcggcagggcaagaagccacggcgggaaaaacctgcggcggcgggg

SEQ ID NO: 398 - DNA, 647 bp, Homo sapiens
cctctgccctgcgaccaacagccgggagcggaccagacaccagaactcccggaacggttgaagacggttccgctccctgtcccgcctttcgcagcccagcagtttcgccctgcggagaggagccttgctgtttccaaatctctcctgctgaagagacattggagctagggcggctagtttcacctggtaattgtgacaccctgtctcctcgagctgcaggcttttatgcttgtcatgttcgaagtttgataccttgcagatcaacaaagggtcggtggcctctcactgcctccgcggcagggttgtcaaggtaacgctcctcagacaagggtggggcgcggcccgccccttttctcaccccgcctcctcctcagccccaccctccttcgctcctcctcttgtcactccctttcagacatgcgcagtgcggcccgtccctagggctgggttaagggccgcggatgtggcagttctcaggcctcttgggatcgcctcaagaagccccctcacgagtgtctcgatttcctgtcagccaacaaagggccgttcgcctttcatggcctccacagcagcgttgccgtggtaacgatcctccgccggacgttggccgcaccgcgcccctattcttgcccatctcccgctccgcc

SEQ ID NO: 399 - DNA, 534 bp, Homo sapiens
cgcctttcatggcctccacagcagcgttgccgtggtaacgatcctccgccggacgttggccgcaccgcgcccctattcttgcccatctcccgctccgccccgtcccttctcgctcctccctcttgtcacacccgttcagacatgggtagtgtagcccgtccctagcggcgggataaaagtcctgccctttcacacatgcgcagtgcacccattcctaggggtggggctaaggggcctgccctttcggaaatgcgcagtgcagcccgtccctaggtgtagggtaatggcggccgacctggcaggtctcaggctccttgggatcccctcgagaagcccgttcatgagtgtctgaaactgtcacttgactgccagaagtgaaaacatcgtgtccctagtcacctgccatttgccttttcaaaaccatttcctctgttctctaggctgtcacaaattctctgcaccccaggagtgccgcttggacccccgggctcgctgcgtggtccatatctaggtcgggcctctcacggagacttt

SEQ ID NO: 400 - DNA, 526 bp, Homo sapiens
gaaagtctccgtgagaggcccgacctagatatggaccacgcagcgagcccgggggtccaagcggcgctcctggggtgcagaggatttgtgacagcctagagaacagaggaaatggttttgaaaaggcaaatggcaggtgactagggacacgatgttttcacttctggcagtcaagtgacagtttcagacactcatgaacgggcttctcgaggggatcccaaggagcctccaggtcggccgccattaccctacacctagggacgggctgcactgcgcatttccgaaagggcaggccccttagccccacccctaggaatgggtgcactgcgcatgtgtgaaagggcaggacttttatcccgccgctagggacgggctacactacccatgtctgaacgggtgtgacaagagggaggagcgagaagggacggggcggagcgggagatgggcaagaataggggcgcggtgcggccaacgtccggcggaggatcgttaccacggcaacgctgctgtggaggccatgaaaggc

SEQ ID NO: 401 - DNA, 424 bp, Homo sapiens
gggcggagcgggagatgggcaagaataggggcgcggtgcggccaacgtccggcggaggatcgttaccacggcaacgctgctgtggaggccatgaaaggcgaacggccctttgttggctgacaggaaatcgagacactcgtgagggggcttcttgaggcgatcccaagaggcctgagaactgccacatccgcggcccttaacccagccctagggacgggccgcactgcgcatgtctgaaagggagtgacaagaggaggagcgaaggagggtggggctgaggaggaggcggggtgagaaaaggggcgggtcgcgccccacccttgtctgaggagcgttaccttgacaaccctgccgcggaggcagtgagaggccaccggccctttgttgatctgcaaggtatcaaacttcgaacatgacaagcata

SEQ ID NO: 402 - DNA, 322 bp, Homo sapiens
tgaggagcgttaccttgacaaccctgccgcggaggcagtgagaggccaccggccctttgttgatctgcaaggtatcaaacttcgaacatgacaagcataaaagcctgcagctcgaggagacagggtgtcacaattaccaggtgaaactagccgccctagctccaatgtctcttcagcaggagagatttggaaacagcaaggctcctctccgcagggcgaaactgctgggctgcgaaaggcgggacagggagcggaaccgtcttcaaccgttccgggagttctggtgtctggtccgctcccggctgttggtcgcagggcagag

SEQ ID NO: 403 - DNA, 322 bp, Homo sapiens
cctctgccctgcgaccaacagccgggagcggaccagacaccagaactcccggaacggttgaagacggttccgctccctgtcccgcctttcgcagcccagcagtttcgccctgcggagaggagccttgctgtttccaaatctctcctgctgaagagacattggagctagggcggctagtttcacctggtaattgtgacaccctgtctcctcgagctgcaggcttttatgcttgtcatgttcgaagtttgataccttgcagatcaacaaagggccggtggcctctcactgcctccgcggcagggttgtcaaggtaacgctcctc

SEQ ID NO: 404 - DNA, 424 bp, Homo sapiens
ttatgcttgtcatgttcgaagtttgataccttgcagatcaacaaagggccggtggcctctcactgcctccgcggcagggttgtcaaggtaacgctcctcagacaagggtggggcgcgacccgccccttttctcaccccgcctcctcctcagccccaccctccttcgctcctcctcttgtcactccctttcagacatgcgcagtgcggcccgtccctagggctgggttaagggccgcggatgtggcagttctcaggcctcttgggatcgcctcaagaagccccctcacgagtgtctcgatttcctgtcagccaacaaagggccgttcgcctttcatggcctccacagcagcgttgccgtggtaacgatcctccgccggacgttggccgcaccgcgcccctattcttgcccatctcccgctccgcc

SEQ ID NO: 405 - DNA, 526 bp, Homo sapiens
cgcctttcatggcctccacagcagcgttgccgtggtaacgatcctccgccggacgttggccgcaccgcgcccctattcttgcccatctcccgctccgccccgtcccttctcgctcctccctcttgtcacacccgttcagacatgggtagtgtagcccgtccctagcggcgggataaaagtcctgccctttcacacatgcgcagtgcacccattcctaggggtggggctaaggggcctgccctttcggaaatgcgcagtgcagcccgtccctaggtgtagggtaatggcggccgacctggaggctccttgggatcccctcgagaagcccgttcatgagtgtctgaaactgtcacttgactgccagaagtgaaaacatcgtgtccctagtcacctgccatttgccttttcaaaaccatttcctctgttctctaggctgtcacaaatcctctgcaccccaggagcgccgcttggacccccgggctcgctgcgtggtccatatctaggtcgggcctctcacggagacttt

SEQ ID NO: 406 - DNA, 322 bp, Homo sapiens; identical to SEQ ID NO: 403

SEQ ID NO: 407 - DNA, 424 bp, Homo sapiens; identical to SEQ ID NO: 404

SEQ ID NO: 408 - DNA, 526 bp, Homo sapiens; identical to SEQ ID NO: 405

SEQ ID NO: 409 - DNA, 578 bp, Homo sapiens
tgctgggcaccctggagcgcgtcgggatgcccaaagtggggcgtgaggccggtgggctctttgctcctccgcttcgcccctgcgcctcccgcgacccgtctcacttccctgcgtctctgagcggcccgagaggcgcagcgctcagagtggccttgatggaagaacctggccctcggcgggaacgattttcttttgctttacctcttctggatcagccgttttccacctgacttgagcagccccctcccccatgccttttttttctcgttccctccgtctgtcctcacaatttcctctatcttcgatttctagatcattcgcttttcttctctttcctccgcttcctcattgtgctgtttttatgcctttgccctggaccacttttcagttcacttttctcccttctgctccctccctcacccccttcttaatgtctcttttttcctcgccctgagtctgtgactgtgtccctttcccccatctgcagttgccgtttttgattttctttctttcctccccacttccatccggatcctctcctatttcctccttggatttctgtctctcaggttctgtct

SEQ ID NO: 410 - DNA, 551 bp, Homo sapiens
ggtcctggaagcctggaaccccggcctgagccaggcatcccctcggggccggcttccacctgccagggctgagccctatgccctgacgtttgggcagcggggaacgggggcacagggagggtgacggggtgaaggaaacaggcctgctggtggctccgaaggaaggtttgggccaggcctagctggaatcccaaaactcacctgccagcaaccgcgagcgggaagccctgccaggctcgcctgcccctgccagcggcccagccacgctgggtccccctctccatcccgcactttgctctttgcccgcgacaggggtcccctgctcaaatgcccagcctagcctcggtccaacctgtctggctgtccttccctgacacctcgtttgtctacccttctctgcacgtgtcagtggctccccgctcgcgtccgtgtgtcattccctacacacgcctgtgtgtcctcttcacacgtctgactggagcgtcggcctcccgcatttcccggcctcctcgccgccctgccccgcctgctgctgtgtgggtgtcgggctg

SEQ ID NO: 411 - DNA, 488 bp, Homo sapiens
gcagcgcggggtcgcggaggtttagcagctccctcagggcgagcagcaccggcgcgcagtgcgcgttctcctccaggccctgagcacgtcggggtagggtctcgcgccagccctctctcccacccagccactgagactctcgcccgtctgcccgcccaagaagctctcaccaaactgaggaaaagctgctgaagctgggcagcatcgtgccgcaggcgctcggccgcctgggtcctctcgtcggctccgcggcacaccaggcggccttgcatcagcgcgctcaggtactggagcaccacggcacgctcggcctcagccagcagcagctgcggggaggcaatcaggcgggtgcaggtcgcctctcacccgcgccgccccttccccaatcttttcccccaacccacctgaaccgtggggttccgcacgcgccagaagtcccggcagaagcgccccgtccgttcacacacactttgcaggagctcagggct

SEQ ID NO: 412 - DNA, 193 bp, Homo sapiens
cgcaatgcctcctggggcttgaagtccacaccgcccaggccggtgaacccggcgggttttgcagtcaacttcgctccgaatttacagaaaacagctactatgctagggacgcggaacagtgtattagagactgaaaaacaggccgggatcggtggctctacgcctgtaatcccagcactttgggaggctgagg

SEQ ID NO: 413 - DNA, 884 bp, Homo sapiens
gaatggaatggctagttttcaacgtgccttttagaaatacttcactagccggggcgcggtggctcatgcctgtaatcccagcactttgggtggctgaggtgggaggatcacttgagcccacgagttcgagaccagcctgggcaacatagcaagatgccatttctaaaaaaaaaaaaaaaaaaaaaaatttaaaaaattagcgggcgtggcagcctgcacctgcagtcccagctactccagaggctgagacgggaggatcgcttgagcccaggagtctgaggttgcagtgagccaagatcgcatcactgcactccagcctagacaacagcaagaccctgtctttaaataaaacagaacagaacaaagtaaaacaaagagaatagggacgactcctgggttgtgttttgttagtctgtttgtttcttgtttgtctagtctttcaactgaagatgtgaatgggtgaagatactttcaacgagacggtttgtctggggaagaccaagaagggggaacagaaagatttcttcagtaagattcattctcaccccgttcgggttgagatccctaaggaaagaaagttctgagaggagaggatgggtggacgtcacaatgaggcaaaaagcatcaaagggggaactttctatcagaactttctaacaaccaggcggagagcggacagctctatcttggacggggtggtggccagcagctctctcgggccagggctgaaaaaaacctccaaggctcggactgccgccccgacttctgaagcagcggtgtcgttcggggctgaccgacgagcgtccctagcgacaaggggccgtgtccatgtgccggttcccgcgctgcaagagggggcggcggccggagctgcccaacgggag

SEQ ID NO: 414 - DNA, 659 bp, Homo sapiens
ctctggcagtcggcagtagcgaccccttcctgggaacagaactttcctccggaggccagggtctcctcatctgcaactgccatttgccacacacacacacacaaaagttgggagagaatgtgagaatgcttgtgttcgtggacgcgtgcgcacgtatggagcggcggtacctatgattatttgcgcgtggcgcctggctatgcgtgggcgaacgcgcagtggagaacatgtgggcgtgcatgaagtgtgcacttaaaccaaagtgtgccctccccgttgggcaggcctgtgtgttggtccaagggttgggagcgcctgagctgtgtgagggtcgctgggaagggtgtttgaggccgtgggcggtgcggagcgtgtctgtgtgcgcggaggctgagctggttgtgtgaagtggtagcgggtctgccagagagcgtttacctgcgtgaatgcacaaccacacactacgggtcacgtcccacactcattctttccctaggcgggccaggctgtctgcacgcggagacccagagaggccagcggaagggcagcgcgggaaggcggctctcttctcccgcgagcgcagatctggcggaggaggcggcgactgcccggcaggctgcgccggcgagccccccagctcctctcaccgacgtgccagg

SEQ ID NO: 415 - DNA, 483 bp, Homo sapiens
cgcgtccccccgtgctccgggggggtgagtcccgcgtcccccggtgctccgggggggtgagtcccgcgttccccccgtgctctgggggtgtgagtcctgcgtcctcccgttccctgggggtgtgagtcaggcatcctcccgttccctggtggtgtgagtcccgcatcctcccgttccctgggggtgtgagtcccgcatccccccatgctccaggggtgtgagtcccatgtccccctgtactctgggggtgtgagtcctgcatcctcccattctctgggggtgtgagtcccgcgtctcccatggaccagcgctgtaagtcccatgccctcctgtgctatggtggtgtgagtcccgtgtcctcccattctctgggggtgtgagtcccctgtcttcccgttctctgggagtgtgagtcccatgccctcccgtgctccggtgctgtgagtcccgcgtcccctcaagctccggagctgtgagtcccgt

SEQ ID NO: 416 - DNA, 309 bp, Homo sapiens
acctcggacccacccacgtccccaccccggcctggacctccacgtctgccggccacagcctttcgtccacctccacaaaagcagctgtgtgtatgtacggggtgccctggcggcggtcctcaaattcccttcaggtggcagacagaacgagggtgacagtgaaaacgtgtccgcggcattggccctcggggaggaggcgaggggtccacccagcccgctaaaccctctccagggcataagccaggtggggcttctccaccggcctccaacgggtgggcaggccctggacaggcgacagggggctggaat

SEQ ID NO: 417 - DNA, 320 bp, Homo sapiens
gagtttgtcatcctcctgcctctgctgcagctgccgctccagctggcgccggtagcagatcaggtagagcgggaggcagaagcccagcaggctgagaaggagcagccccacgttcacctggggaggcagggaggccgcgcatcacagggcgtggtggtgcgccgaggctgcagacccgccctccactcgggtttgggaaaggggagaaggtcacggggtggaagggggaacgaaacaggaaggcccacgaggtcccatatgccagcgtcccggttcccaactcttcacaggccatgtggccaccctcagggcctgggccc

SEQ ID NO: 418 - DNA, 837 bp, Homo sapiens
ccctaaagcacactaagccaggaaagtctagcccaaatattaatgtgtccggaaagtcttccatgacaactctagcttatactgatgctctctttctctgaaattgtacgaacgttagaacaattgataattcgtttcacaaatattgaagtattatgatggttactgctattgaaaagactgaatcaaacatgttttgtcctcgaattacttacagagatgtaagaaagatatgttctatagagcaatgactgaactggaaggtaaagcatagtaagttgcatgaaataatatgtgtgggagtttaggagagaagcagcaggagggaggcaaagacagggaaagagagaaacagagagacaggcaggagtgagagggagagtgatccttttgagatgggaaagtttcacggaaagatgagttcagcagccagcaactggcgtcaagtcttgaaatatttgggaagtggacttggaggaaaactatcgcaaaaatatcttcttcaaaagaacatatgtacagagctgttacctacccactgatttgccaatttgcaaagcatttacaattaccaatttcatcaagataatcataccagaagatgatttcttcccactcccaagtcatgtgatcttagaattttaaggacctggatggatgcggtggctcacgcctgtaatcccagcactttgggaggccgaggtgggcggatcatgaggtcagttgttcgagaccaacctggtcaacatggtgaaaccctgtctctactaaaaatacaaaaattagccgggcgtggtggcaggcacctgtagtcccagctgctcgggaggctgaggc

SEQ ID NO: 419 - DNA, 448 bp, Homo sapiens
ggatgacacgggtccgtacgcccagtcggtgaacttcctggacaggctccggagccgcttctccttgatctcacgactgaggatgtacttcaggatctgggacaggaggaggaacaaccatcagctgcagaacaggggcttaaggccaacagggctggaccagccagaggctggctgggcccaggggatacacccgcccagaatgggtggagacctgcctctgcgcctggcgccatggcccctgggccccgtctttatctgtggaatgcgcacaaagcttgctgttccgcccatctcactgggggttgtgaataccagggacaaagggtgtgaaggttcttcgcaggctgcagcaagctgcacaatcagcttgaaatgcctcacgccagctggggacccggttcggtgaaaaatccaggcactgatgattttttagaaacagtttcca

SEQ ID NO: 420 - DNA, 355 bp, Homo sapiens
tgtgcccctcccacactcctgtgcccgccctgcggacaccaccaaggcccggtcctgtggcctgtgacacagccttctctgcacagccctgcaacgccaagacttgagcacaccccctcccctgacaggcacacgcccgtgtccgcgtgacccgccctcacacaccactccatcctgtcctgcaccccccacgcagacacaggcacactcccgcgtccacgtgacacacgcacacccgcctccaaacagcacacgcaggtccctgcgcacgtggacacccccccagccaccagccctgcgcctcccgggccccgaagccgcagcgcccgcctctgccgccccctctcgccccagc

SEQ ID NO: 421 - DNA, 162 bp, Homo sapiens
ctatctccccgatctccctctcctttccatctccttacttccgcccctccggtgtctctccgagaggtgtccccccacgccccgcgccctccgcaccgcgggcctcgcttcccggtcccccctggcttcctcgccacgtccgccccactctaggtgcaggac

SEQ ID NO: 422 - DNA, 573 bp, Homo sapiens
tccgccctctccgtaccccgcccggccagagcccccctttttgtaggcccgggtagagtagcctgtcattcacaccacaccctccccgtctgagaccctacgggagcccactgtcgtctgcccaaacctgctgtctcgttgggctcccccttttctcccttaacacagtacaagagagggagagggggttgccgtatgcactttgtcctgcagcaaaggttggctcctggcaaaccctcccaaaagccaacacaccaattttcactggcccaggccttccgacgactcagactcccgtattgccaacctcgatatccctaacctcctccccaattccaatatccccgtcctcgtgtccacttgcagtgacccaagaccccctacgttacgaccaccccttgtttctgaggtcccgccctcgagacggagaccccgccctggactcgccatctggagagcgagggaggaaggaaggatacagacccaggcgttcaccccacctccacccccgcccccgacatcccggccagataaaggagccgaggccaagaggggagagagaccccgcccccc

SEQ ID NO: 423 - DNA, 385 bp, Homo sapiens
acgccagtcccacggggattgaattatgtacacacacaacccccaggcccgggggaggggggagcggcgagaaatgaatgcagaggggccctgccctactcacacgcatgcacctgccgcctgctgcgcgcaggggagacacgtggattgcaaaccccagcgagcccagggtccaccgccaacgcgtggaaatagggatggtccccaaaacctgaggggatgagtagttttgttctcaggaacgttgccgaggaccaggtaaatctcggatatcaatcagatcacagacctccaccaagtgccagctctgtgcttagaataacagacccccaggccgggcgcggtgcaagcctgtaatcccagcactttgggaggccgatgtggg

SEQ ID NO: 424 - DNA, 849 bp, Homo sapiens
gctgggtggctgcgcgcgcctcaggcggcaggcgaacgacgtgcggcaccgggcggagctagggggcgggggcggagctgataccgaagctcctattggccgtcggggacgccccgcctctctctggcgttgcagcccaggagcctccaccgacatggcagcgaggttcccgagccaacgccatcctccgttccttccgtcactcactcactaatcactgttcctgagttccttcacttctcacccgtcatttattgcgcacctattctgtgccaggcactcggccaagcactgcgaggccttcaaagatcatttctttgtctctcccaaacccttccgcccagtcccacccgcagagctgcaacgctctgccattatccctgattcttaaccgccgcctttctcctttccgttcttgcctcactgggcctccctgagacacttgatttgccccaaccttcttcctctgtcttggttctcccactctcatcagctctgcatcccctgggtcctctcccacttctccccattccgttggggctagtccaatgtggtgccatcctctttccacctgcgtctctctctcttctctctctctctctcaacaaatgtcatccctgactcttcccttctccttccctttcccacacgccaacaatggctgaatcctctgatttgacctcattgtctctcagacacctcaccctctctcccttctcactatccctccctcggctcagttccacaccatttgtaatggtcaagaccttcttaccctttccccagcgtccagtgttgccggtttccaggccatcctgtgctgggccacgacatcaacttgtgatacta

SEQ ID NO: 425 - DNA, 468 bp, Homo sapiens
ctgctcctgctcctggatcaggcgctgcagcgcctccagctgctggatccggaactgcagcggacgggtcctgcccgagctgaaggcggcgcgggcgcgcttcacggcctcgctgatcttgctcatggcgcctggggacagagagcacctgcagctggctgaggggcacgagcgcgccctgcctcccaccctgcctgaattctataggaagggacttccctgggctcggccttggggaaaatggcctttccgctggaaggtccactttctgcctcgcctcctctcccgcccctcactcagacgcctgggccctggctgccttgctagcccctgagaaggtgacacccgccgcaacccgagtcctaagccgaactgctgctgggggctcttggcagcgtgctcgcggcagggcctttcccggccttttccgctcccttacttgcttgcaaagctgatggcacaatccca

SEQ ID NO: 426 - DNA, 400 bp, Homo sapiens
ggcagtgcccaccagggtagagaggggctgataattggctggattatcccggcggggaggcctccttggggaagctatttttaactctgagagatgctgcagccacttccctccctctctttattgccctttattcctggggcctgcaggctttgcccgccttcctggaagctctgtccctttagaggcagggagggatgaaacgctcccctcacataccatagagtgtggtccccacatggttctggcagggttctcatgcgcccctttttagagaaggagagagagcttcagaaagcttgaggggcattttggaggacttgctggtctttttacgtctattgccttcccggcagccccttgaggctgttagaaatttcctaaattaacaaaggggtgg

SEQ ID NO: 427 - DNA, 480 bp, Homo sapiens
gcttgccagcatcccagccaccccgatgctggcttccagcagggccatccggaaggtgcggctgcgactggagctgacatctgccacggacgcaaagctagcagccagaaggccaccgaagtcgccgaggagggcacaaaggatgcgacccagcacgaagtagccgacgtggagctgcagctgcaccacaaaaacggacactagggcctggagcagcaggcccagcgaggccagcactagcagcgggcggcggcccacactgtcgctccaagctcccagcagggtggacgagaagagccccaccaggaagccgcccacgttcatgtagagggtccagtgggaggtaagggtctccacttcctgtaggggcacaatgaccagggtgcggttcctcactctgggttccacaatccccaaatcctcgccatcccgggagctacagggatgaggacttcctttggagccctaaacctcagagaa

SEQ ID NO: 428 - DNA, 324 bp, Homo sapiens
gcacagcgatactagctgagtccatcgatgacctttaaacccgcttccccggttctgtctagattacagggtttggaaggctttggaagatgtgttcagagaactttgcccaaactggcccacgcctgttccaatgctatgcatagctgtttctgattcttagccagctcaggccctctattctttccatgctggtctgcggcctccgttacccacctgctgagggcgatcttctggtggagcagaaagccccgctcgtagggccagggcaggccggcctcgaaccactctcggcagcgcagcacgggcgagatgcaggcggcg

SEQ ID NO: 429 - DNA, 787 bp, Homo sapiens
cagcagcaggcccagagagcccacgaaggccactgcccacagcacacgccggatggtcagcggcccatacacgaagatgtggcggatgccatggagggtggaggtgttggcaaagatctggatgctagaaggttgcaggctgccctcactggggctttccttgaggtccatcgggaccccgtgcagttccgcaactggcttcaggttgatcgaatttcagagaagagctcccagtcctcgggctctgcttaaaccttgacgttcaggggagagaacgcaaggcaagcatcgcgccagatgctgtgagtttatcctgagagcccagccgcacgctgacaggggtgagtgtttatataaaacccattcaaactctagctcttgattttttccaggcgataaggagggctggttttatttagggagacaccaaggtgatgtggaagagatgtgggtgggaagggagagcttagccaatggaaataaagtcctcccccctacacacgccactagagacaagattaaaagcgagagcgagagagaaaatgcttaaaacaagtcgctgagcagctcggagcctgttaaccagcgcataatgccccatgttaaccatcctgggggatgtcagggcggccacgctgatgcactgcggtcgggcgcagcggcacagccttcagagccgccgccgctcggttccttttcccatgatgttttggactccgaatgccgtttctcgccgccggcctccgtccctcctctcctcctcctccaggcccccttttgtgggcag

SEQ ID NO: 430 - DNA, 790 bp, Homo sapiens
ctggacaacatggggaaaccccgtctctactaaaaatacaaaaaatagccggatgtggtgggggcgcctgtaatcccagctactcaagaggctgaggcaggataattgtttgaacctgggaggtggaggttgcagtgagctgagatcatgtgccattgcacttcagcctgggcgacagagcaagactctgtctctcccacccccgccccccccaaaaaaattccagatggaggcttagaaatattattataacttagtatgtctgcaacatgatctctctctctttctatgtattataaaatacatgggaattgaagacatcgatgagggatagaggagagccaggccccattaggagcatgattgctttttttgagacagattcttgctttgttgcccaggctggagtacagtggcatgatctcagctcctcactccaacctctttctcctgggtttaagcaattcttgtgcctcagcctcccaagtacctaggattacagacgtgcaccaccacacctggctaatttttgtatttttccgtacgccacatttcccgcgccccaccgcggggcggggattcggcgctgggctcttccctgttcactcgccattactgagggaatcctggttagtttcttttcctccgctgacaaatatgcttaaattcagcgggtcgccacgtctgatctgaggtcgcgggcctcgatcagaaggacttgggccccacacgagcggcgccggggagtgggtcttccgtacgccacatttcccgcgccccaccgcggggc

SEQ ID NO: 431 - DNA, 624 bp, Homo sapiens
agtctgcacagccacccagcctatgtggcgcactgcgttctggatcacccggatctgggtaaggcgggggccgctgggaactcctgagcgcccccgctcccagcctttgtgccctccatcatttcctggccccagaccccctaccgaccttctctcttggagcgcggagccctcttcgacgcattccgcaggaccgccccttctcagctgctgcccagagcgcccccatccgtcaagaaggcccactgttggtgcttggttcccatctgtcacctagcgcccccagtgcagagcccagcataacctagagcccgctgtcaacagcgcccaccgagggctccccgcccccacgcagcaccagcgccaccgcacacctgactctgtccgctcacacagcctcaccttcacctcttgggtatttcagatccgctctggcccagtgggcagaggcatcgcctagagaaaataacctcagggttccctcatccctctgacaaacctgcctctttccctgaaaccattcaataaaacctctgatcaaagtgtcagtgctttgaggtggcgcaggtggctccgggtggggatgatcctcctggggttcaagccagaggtaaaagtgtgggc

SEQ ID NO: 432 - DNA, 585 bp, Homo sapiens
gggattgacctgggattggggattggcggggtggggagctggtggggtccggcggaaaggggaggacgtgaacttgggcgggttgccctggagaggcctgtagatgctgggcaggtgggaaggcaggtttccaggtggcagcgggtggagaggaggtgggggacttgtggtcagagagcgcccctggcggggatttggggatcagcatgcaggaagctctggtgatgacaccccaggggcgtgtgtgaaacggattcaggctgccaagcgttattcactgtggagagattgtcatcaccagagccgtgtctaaaggatttagccagggctggatacggaaaacagaatggaagggggctttgggagaccagcccacctcacaagaaagagctgagagcctagatttgggccagcgggggtagtctctggacggagggcggcacggggctggaggaggagcgttttatgatgcggccgtgggtgctggccttggctggggcttgtggcgactgggtgccgtgacgtgggggtggaccgggtagagcggggtcggcagggggccgagtccgggcgccccccgcatcc

SEQ ID NO: 433 - DNA, 329 bp, Homo sapiens
cgcccgccccccatcccctcccctcccccgccggccccagcccggcacccggcggcccctccgcccagtcctcgccgcccaactttgatttaggaggtcgcctggtcccgccccctgccctacgccgctcccccgcgcagccctgcaaatcaggctctcagctattggctgcctcggtggccaggagggcgaactgggcggagtcttggggggctcgacacgatggggcggggcttggcggggaaagaagcggaggagggagtttcacactgaaaaacccgggatggaggggcgtgaccagattgagggaagcttggggagatcatagt

SEQ ID NO: 434 - DNA, 565 bp, Homo sapiens
tctgagattactaaactaacagtttaaaagattctgggaaccaggtgaccggggttctggtcccaccagtatcctgtattgtggctttgggaaagtctgtttcccatctctagacctcagtttccacactttgaagaggaagtttgtgtggtctctaagatccatttctgcgctctgtaatctccataagccaaggggttcgaacctcccaggcttcacactgtgcagctggccagcaccattcatcgcccaccaacagccctgggcattggcctctgattccccgtcacccagagggaagacccttttggggccgaggcgctcaccttcagccagtccccgtagtggacgtagcccccgatgtaggcggcgtaggtgctaatggccaattggagtgcggcccccagcgcgaaccagcgccgcttcacgccaaaggacatgaaactagcgcacagcacggctgcccccatgtcgaaatacaggtaaggcactgggatgtcgggcttcctgcgagccgggagtggggaagcaacgaggacagaatgaagcggtgggggaagaagct

SEQ ID NO: 435 - DNA, 559 bp, Homo sapiens
tgttcctcctcagccaggctgtgggtgggaacatctggaagggatcccccggaactgggggaatttccaggcacatgaggctctgtcaacccagccaggaacatccgcccctgccatctgctccagacgtcattgcagagtctgtgtgagaggaacccgacagtttccagctcctccaaggagcgtctgtctgtgtctctgggtcctgccctgggcatgtgaggggcctgcctctgctactgctgggggagtctgttgggggcctaagttcccaagtgcaagtcctgcgttttatctgctgggagatgaggtgtctccaaattccaaagatgccctgggggtcttccctaggacgatgaaggagggagaagctgccctggttgttgagaagcctcagcccagaattttctcaccctcatgaggcaaatttgtccaacacacgctgtgctctgccctgtgcaacatctcagtaataaatgcccagttttgtgcactgatggcctgtgcaccggaggctttacagcttgacctcacttgatctgcatggaaactgtgaaac

SEQ ID NO: 436 - DNA, 306 bp, Homo sapiens
ggtgtccacacccgccggcctgcaggcacctcggggcccacgtgtcctccggccacatcccaccctggggccacctgtgtccatcccagagcgtgcgcgcaaggtgaggcctgggggcaggtgcctatttttgcccccctcaacctgcatctttgctatcctgggacttactgctcttcagaactttcgggcccctggtagaaggagatgtctctcctgtgtctgagatgggtggggcaggaactccccacagccccgggcttcttaagtggagaaggggtgggaaagtcaggagttggactttct

SEQ ID NO: 437 - DNA, 536 bp, Homo sapiens
tctccagctcccaccatttctagccagccctgaggaattgtctgagacccgggagcccagagatcatcagcgggggccaacagccatgacctttgagttttcttggccgctgggccacagaaagggtgcaagaggcagctggacaggtagacagaagtaggcacaaagaaaggggccctggacggttgccagagaaaagacggagaatgacaaagagagaagtgaaatagctggactggaggcgggaactggagcgcagcaggcaggtggagtggccaaaacccgcgtgctaagaagagcccacaggggcggcaggggcggcaggggcggcagaggcggccaggcgcgtctccgttccccatctttacggggaggttaatgacctcctcagcgccccctcgtcccaaaccacccaattacaactcctcaggctccttaatgatctgattaattggttggttagggcggatggaggtgcttctctgccggggacctggcggggtttcgctagatcatttgttaattgtgggaaggaa

SEQ ID NO: 438 - DNA, 471 bp, Homo sapiens
aggggtagcgctgttcttcaccttggcgagaaccttcttctctttgacccggcggttctgaaaccagatggtaatctggcgctccgagaggctggtggctgccgagatcttgcgcctcttgtccttggtgatgaacttgttagccgcatactcccgctccagctcccgcaactgccccttgctgtacggaatgcgtttcttgcggccgcgacgaaaggcgcaggcgtcaggagggtgctgcccgctggagtctgcgcggcgtgaaagggagggaggaaaaggcatggtcagatacccacccatgcagacccaggccttgcaagccccaagctaagtcatctcacaggtgcacacaggtcaccctacaggcgcacgtgcaatcctgttcctccaaagcataccaggacagcaccctggcttccggcttgttctctcaccaccgctcagccttggctcctgaaggccaccctc

SEQ ID NO: 439 - DNA, 647 bp, Homo sapiens
ttatgcacaatgaccccggcaggatcacactccttacatgagcatcctccggtgcatgcagacccacacagcatgtgtgcaaacacactcgcctatacatgcacacagacagtctgcacacatgcctagcctggagggaaaacacgatgtggactcctactaagtgactagcactgctgggggacttgggagctgggaattctggagtaggggaacatacacagttgcacgggttcacaactatgctcacttgtacacgtgctaacgcagaaccttcccaccgaagttgaacctcgcaggcccccatcccaaaggcaagagttccaggagcccgcagggcccttcagtggccagccaggctcggcagcgcgcggggaactacggggtcccgcgcgcccccgcggccctggacagccaaacaccaacctgtgtctgcgtgaggcccagctgcgcggccagctcggcgcgctcgggcagcgccaggtactgggccttctggaagcggcgctgcagggcggccagctggtagctggagtagatcgtacgcggctttcggaccttcttgggcttcccattcaccatgcgcacctctgcttccggctcctccttcaccgacactgcggggaacgcaccgggcggaagagggg

SEQ ID NO: 440 - DNA, 498 bp, Homo sapiens
ttcttgttcaaataactggcgtggtttctgtctcctgactggaccgcaccggatacactagcaatctctttaggggaacttcgggcttgattctggctgtagctgggatcctcttgggctagggcaggctggccttcaagtcttccactccagggtcactttgctgcttgccatagaaatgcctggtcaagcatgcccaccaagactgtcgccttccgtgtatctccctctccactgagcacggataccttctgggtcttctctgtgtcaggtgcaagctgctaggggggcttctgccctctcttgtctggaaatgcttgctgccatctctttattgtcctgcccatgtcttgtactatgcatggcattggtaggactctcaatttctaatgttaatcaagaaagagcacagtagggagcttggtgcagtggctcatgcatgtagtcccggttacttgggaagctgagtggggaggattgcttgagtccaggagttca

SEQ ID NO: 441 - DNA, 509 bp, Homo sapiens
cagtagaactgggtcagctagagggaaaggtctttatcccttgggtagccggagtctgaggcattcccaagggcgagtgggtacagtgtggttcccatccacagagtggtggcatgcatgccatgccagctactgtaccagggaggggcgatttcaagaagagactcagtcttggcctcaagcatttctcagtccagagagagagagagaaacccagctcttgcaggcagagctttataaattacaatgcatcttcctgcccacttcctttctgaacccaaacaatagcccttctgtgaattaggccaggatgctttgaaactgtctttcagacaggaggagctgaggacccaaagtcactcggttgtttatcaacagatctagacttaaattcaggtctctgattctcaaacacagtctaatttcgatctgtagacttggtcaagctgctgttttgtccggtgtactgtcctgatattgagaggtgacaatgtgctagcaacccttgc

SEQ ID NO: 442 - DNA, 516 bp, Homo sapiens
tcccagagccgcgccgaagcctcgtgcctgcccaggcttcccggctctccggaaggtctgcgcgctcccatgtccagggcggggccacgggctgggcagcctccctccccgtggcagtggtcagtgccgtgtaagcgttcctagaatcagagttctcctcctcagatcacccgcccttctcaggggcacccagcaagcccttatgccctaggcgctcacccaggctggcggcctttccgccttgctctttagccctgcacctgctcaaaggggtcactccaggacccaccgctgcccaccctcggacagcagggcttccagtccagctggggagtcagagcccctgtgagcccctactgtggtgtgactccagaactggcttttgtctgctgccaaattaaagaccctgcctccccagagtcctccagctccagggtggccaccctccccatccctgctgggtgaccgggagctgctgggagtgagcccatccctaacagcagcagcctgatgagg

SEQ ID NO: 443 - DNA, 635 bp, Homo sapiens
agcccaatagaaataaggtgttatacagacaattgcactattgaatctccggagacagagttgagttaaggaaaaatgcactttcggaaagctttggcgactgaacgacctgagttctccaacgccaaggctggaagtttggagaccttgaaacaagtaaattaccattaatatcataattttcagtttccttttaagaaatgagagtaacaccactttcttcacagaatcaagtgagagagaacctatgcaaaggaacttatcacttggtaggtatttgaagctccctctgcctgaaaactacttattcaaagactgcaacaaaaggtgttcacagtgtggtatctttcaaaacatacgctaaggcaaggaacagataaggaaatggaagtacatccaattgggatccaagcaacccaagtgggaaccaacagctagacttggagatgtcttagctcactcttaaccacgtgagggtagggtatccaaccaataaagacattcagagttggagagcgggcgagtggagtgtcctttagactccgcggactcgacgggagggaccctgcaatgcttaattagaaccggttgccatggcgacagtctctaggcagcgtgggctgagctctccgtag

SEQ ID NO: 444 - DNA, 530 bp, Homo sapiens
aaaatgttcaggcttggagaacctacccctggaggagtgttagggtttccgggctcggatgtccgcgagcttgacgcctgacctagttgtgtctgtctgggcggtgacgtttggtctgagctgacctttcctcacaatcgtgaatttgggctgtgaggatcaggccttgtcagaacccgagatgagaggagagaggtgcggagagtctgcgtggggtggaagtagaagttagagttgaaaaacctgtcttgcgtggggtatagaggaccctactgccctcaaaataattatgttaatctaaaactgttctaaaattaaaaggttattaagaaaacgaagtttaggtcgggcgcagtggctcacacctgtaatcccagtattttgggaggccgaggcgggcgggtcacttgagctcaggaggtagagaccagcctggccaacatggtgaaacccgtctctattaaaactacaaaaattagccgggggcgttggctcacacttgtaatccccgctacagatactcgggaagc

SEQ ID NO: 445 - DNA, 390 bp, Homo sapiens
gctggaggaaggatgggcagaggggccacctggcaggcgccctggcagccggggtgatctgtcttctcaggagatgtcaggacctgggcagtctccttcctctttggtcctggggcctggggtggtggcccttgggcagacctacagaggcctggctcataggtaaggggctgccgcacctccgccctattcaacatacactctctgtgcactctgtctgagccagaccctgggccagatgcgaggttcaaagagcaaggccattcccccacctccaggggctccgctcagacgggcaggcaccgtcccaccgtcactaggacagagaatgtggagaggccgggaagtgcgccaccgggagcctgcgcaggaaacggaggggcgctgcgg

SEQ ID NO: 446 - DNA, 484 bp, Homo sapiens
tcctgcaggcaacaaggaagagggccgcggaagtcaagaccactgagcccggaatagcccagcactcgaggagagacttgggttcgagtcccgatgctgcacttgccgttcgccttcgggcctcagttttcccatttctaaaatgaggacttgaataaaatcacatgcaaggcttccattcttacactcagtgctggaattaggacaacctcttcattccgtggggttgattctaagatttcagcctcgcttcagtcctgcggtggggagattgtgggtagtcttggtccaaatcgactgcgagggaaactttggctttaaaacgaggggtgattacctgctctaaggtcgacaccacgcttgcttgatagttgaccgtgccttagttttcccgtcagtcaagtgggaagagcagcccatttcctgccgctccccgggccctgtcccgcctggacgcctccctccaggagcctgcgccccggcc

SEQ ID NO: 447 - DNA, 551 bp, Homo sapiens
gactctgcggattctctgcgggcgggaggcgtccaggcgggacagggcccggggagcggctggcaatgggctgctcttcccacttgactgatggaaaactaaggctcagtcacctgagtaagcgtggtgtcgacctcagagcgggcaatcacccctcgttttaaaaccaaagtttccctcgcagtcgatttggaccaagactacccacaatctccccaccgcaggactgaagcgaggctgaaatcttagaatcaaccccacggaatgaagaggttgtcgtaattccagcactgagtgtaagaatggaagccttacatgtgattttattcaagtccccattttagaaatggaaaaactcaggcccgaaggcgaacggcgaatgcagcatcgggactcgaacccaagtctgtccccgagtgcggggctattccaggctcggtggtcttgacctccacggccctcctccttgttgcctgcagaaaaacgccgcggccacgatgccggcgtatgtggagggggatgtctacctggtggtggagcaccccttcgag

SEQ ID NO: 448 - DNA, 830 bp, Homo sapiens
tgcccagcacatggacagcaggggtgggacacctaggggagggggcacccggggtgtctcggcaaccagcttgttttcctttccattacaggcagtgggctgcagccgccagttgctgctggcccatttgttgctattggcatcccaaggccccaaattatgatgactaattcccaccgctttaggcagctccagggctgtctgtggcaacatgctccagggggagcggggtagaaaccccgcagggcgcacactaacacccgacatcaattccaactcagcctcctgcctcccagaggggctgggagtccgtagggacagggagagggttccagccaatcagaggaccccttcaaccacagccagatcctaggaaaaccagctataaggggacaatttcttcatcagcataaaacatagctaacagccagctggtgttgagcagtagtgagtcctccatcatcaagggtatgcaagcagagaccagatggccttgtgggcagacgacggtcctagatgacttttcgtgtgtctttcaagtcaaatgcaatgattcccattagtccttaggacagaagataagcccattctcccctccttgttagcttacagcccgactgcgtcctgtggtacaaagtcaaatgtggaccaatatgcaggttaacacttcattttaaagtcagccattcaggcgctgtttaaacagaggatcgctgtaaagaatttggcctccttcccagcctcctttcccttgtagaacaaggttcctggcccctatgccggccttccagtagaggacacgaggaagaggcagtcgataaatatttgcc

SEQ ID NO: 449 - DNA, 756 bp, Homo sapiens
aggccccccattgtcgcagaacttcaggaataaagctggattgggcacccggtctgtctctttctggttctgtaattgaacaggctgtttaatccagggatcggcaaacatactctgccaagagctggacagtaagtcctttaggctttatggaccacatagtctcagtcatagctactcaactctgcccttgtagctcaaacacagccatggactatctataaaggagtgggtgttgctgtatgccaataaaactttattgacacgaacaggaggtgggccacatttagcccatggaccataggttgctgaccctgatttacccttctgacccagtttcctcacctataatatagggacaataatgccttaacatgaccactgagagttgatacaaacaaaagtacttacaactgcacaatcatgttgggtttttgtgttttatggcaatccctcaccttgcatccttgcccagaggagcggggcaggccccagcagtgcctcgctgagtgctgtggccaagcacatcactttgacttcgtggctgggagactgcgaacctcagactacctgcgttatgatttatttcagcgtgactagtatacatcttggttctgcagcgagtggagggaatgaggagggcctttcaagggaggcgggattgcattcgcacctgattacgctgcgcggacacaacagaggcgcccggacccgcagccctgccttcgccgcctttgctgggctgtttgcgcttcg

SEQ ID NO: 450 - DNA, 783 bp, Homo sapiens
ctgcacccttgaccttctgggctccagcgatcctcctgcctccgcctcccgggtagctaggaccacaggcatgcaccaccacacctggctaattcttgtattttttgtagagacagggtttagccatgttcccaggctggtcttgaactcctgggctcaaatgatcctcccacctcagcctccctagtagctgagactacaggtgcgtgccaccaggcccagcttttttttttttttttttttttaattttttgtagagatagggttttcccatgttgcccaggctggactcaaactcctgttctcaagttatccgcctgtctcagcttcccaaagggctgagattagaggtgtgagccactgtgcccagccttcacatcatccagcagctccagtgcctggggtaaggtccagtccccatgctaaggctctgctgccaggtgtttggctgaggtccccttgtcacctttggaaatgcaacagttgtgggtggccccagtctctgtcccaagaacatcagcccagacccttggtcagaaaagactcagaacagattcagagaagccaccagttctctctgcctcacagtgtcccactgggtctgccaagtctaattccgaattgtatgaactgaccactggtttgggcatcgactcgtccaagcttcagagacaaacagcacaggtttctggggccctcaccgcgctgtagctcccttacctcatccacgtgccgggactaatcgacagcttagcctccccactctcaccggttgcattaaga

SEQ ID NO: 451 - DNA, 544 bp, Homo sapiens
tgccctgacttcccaaagtgctgggattacagtactgagccaccacacccggccagctcctggccaagtttctgagcgcccctgcactcaagcctgactccttccttgcatgacatgatcttgtgtccacgaagctgcatggcctgggaccagcaacagccttgttctaacttgttgtctcatgatttaaagtggatggcacatggtcttactcttctcccagcccccttggtacaagttgggactcatgcccacagtacagcaagttaatcagagctgcagggccaacgcagtacccctgtactggcttcaactccacctagagaacgaggggtcctctgcagaaagggatccatggctgcttcgttgtgagaactttcctttttttttcttttcttttctttttttcttttttttttttgagacagagtcccactctgtcgccaggctggagagcagtggtgcaatcttggccattgcaacctccgcctccccggttcaagcgatcctcctgcctcagcctcccaagtagctgggattacag

SEQ ID NO: 452 - DNA, 549 bp, Homo sapiens
ggaggaacagtgacctggctaaggcgtggcctcagggtggggcctctaccgggggcgtggccttgggctgcttggggacggactcttagcgggcgtggtctagatctgcatggggtgggaccctagtggggcgtggtctcagggtgcgttgggggcggggcccttggaggtcgtggtctcgggcggggacgtggaaggagtggcatctggctgcgttgtggacgggactcctgagggcgtggtctagggctgcatcggggcgggaccctggtggggcgtggccctgggagggtgtggcctcgagcggggcgtggcctcgggcggggcgtggcctctggctactttgggtgcgggctggcccatgggccgcagagttgctgccgctgctgtcctcacacccgctcgtgggaagcagcgcctgtcgcaactcgccacttgttctcctcacagcaggttcaggagaagtggcacctggtggaggacctgtcgcgactgctgccggagccaggcccgagcgactctccgggccccaagtaccctgcctcccaa

SEQ ID NO: 453 - DNA, 482 bp, Homo sapiens
ccatgctctacgggacaggtgagggtggcctcacaccgggccccctgtccggggctctgctggcggatcctgctgggcgcgtgtagctgggctcagcaacagggccttccgcctgctccccagggaaccctggagggaagcccttggggccaggatcctgctgggcagcaacatgctctccacggggccaacccaggcagggcgcgaagtccatcctggcccctgtccaaacgggtcggctgtatttatagaccgtctcagaggacaggctcctgagagccgttcctgaaaaggtggctgttcctgagctgcagggacccaaggggcccagcaggcggcgctcacgtgtcatcatggcctgcgctcggggagcctgcccacggggtgcccctggtgccccgcggggggaggggtggtgtgggaaggggaggccggctccaggctgtgagctcagtcggctggcgggggtagagctggctgca

SEQ ID NO: 454 - DNA, 462 bp, Homo sapiens
gagcccaagggggcagcacggcgaggagcagtcgccacccctccctgcccggcagcaggcccagggggcagctctcctgctctgccagctcccagacacccacaggctgagaaagaacaaacagccatctcaaagaagtattttcacacagttcacctcaacacagtgaaaactctcaagctgatgaaataaagaccacatggaaaggggagggaagaaaggtagaagtcattatgaatttattatttacacgattgttaaagtacacaaatacagtggcgatacaaacgcacagctcggagactggccgtcagtgcacagctgacacgacgtcctacctacgtctcctgcacggggcgacggggactagatactgggcaaggactgtcacaagcactccgaagacgcgacccggcgaggctcgggctggaacccgggcgcagagctgcctcgcacaaacgt

SEQ ID NO: 455 - DNA, 359 bp, Homo sapiens
ccttgtcccagctttcctgggctgggggctgggctccacctgcctttgccggtgacttccgtccctgcagactcacaattttcaaagccctctccccgcctcctctggtagtcacaacccagttattcagacttgtacaggtcgttcgcaagtcctcctggaaggtatcttggctggaaaaggggacaaggccaagcttccccaccgctggatgctgcgctcggctctggaactgacaccaggcgtctcgggcagggccactgacccgcagcacacagaagccagctttgccatcgcagcggggaccgccgggcgggccagctccgccctctcctgcctggggggccctcggcaccctc

SEQ ID NO: 456 - DNA, 314 bp, Homo sapiens
ctgccgtgcaatctaagtccccgcagcagcgtccccaaccccaccctgccggtccctcccaccctgttcatgctagctcctgagagacctgcctgagatgtccacaccccagccctccccctccgaggaggatggaccaggattccaaccctttgccgaccctccccaccgccctggagacgaggtgcaatgtgtccgtgagcagcgcgttccgcagcctttatgagcctggctcctgttaataattgtctcccgattgtctcccggcgtccaccggcgtccaccctgcattctccgtgaaggtgacttctgct

SEQ ID NO: 457 - DNA, 808 bp, Homo sapiens
tcagacacttctcgcatctacgtaagcaggagcctcagaggtcctgagccggtgcgtactaacgactctggtgaattggtgacatctgcatgctcaattagctgagacactggtttgttttggcacaaggtatttggtctaacaccttgaaaaaaagtgtaccatagccaccctgtcactgggatgaagatgacctaagtcaacagcaattctgatcactgcacatttttagcaccgagaaaggatcctctcttttggcagaaacagcaagtcctgactctccccttcctgtgcacaaagtaacctcttcaagaaaatagattcctgggatgacggggtggtaaggggactcagggacatcggttcagccaccatcggaagcctgagtcctttccctagaatcctgtgatctttggtgacttttggtgagggttcccatgctgtgtgcatcagccgcagacctgtcctcgggccccagagactgggaactcccaggggaaagtcattcaatccaatcacccacttaggttctctgtccagaaatgacacaggaaaaagagttttattaatattataatgtactggctgggcgcggtggctgaagcctgtaatcccagcactatgggaggcgaaggcaggtggatcacctgaggtcaggatttcgagaccagcttgcccaacatggtgaaagcccctctctactaaaaacacaaaaatgagtcgggcatggtggcacatgtctgtaatcccagctgctccggtggctgaggcaggagaattgcttgaacccaggagacagaggttgcag

SEQ ID NO: 458 - DNA, 477 bp, Homo sapiens
gatagaaaaataaaagccctatatttaaaagtgagtagcagcaagtgaccggtcagtaacctccttcaaaggaaagaccacaaaattcaacgcaatggttaatccaggcacagcccattcatcaatctggatgtttcaatcaatttccctcaaaagtctcagtcttcatgaccactatagaaaacagaccaagagcccagagtttagagtgggaaaatctgggaagattggtaactgtctcttaactggatatttaactttgaacaagctgtcaaattctctgaacctcaatttaaagaagccataccattatctgaggggatcactgtgaacattaaagtgagatagtatgtaaaggttccctgctacaacattcctgattaagttcttgtctgtgacggagcaacgacaagagtatggttagtcccgggttgttaccattcttaaaacgttgctttgtagttttacttaggtaga

SEQ ID NO: 459 - DNA, 670 bp, Homo sapiens
aaacagacctagcgcaaaacgagcactgacacagcgggggcccacatcccggaggttctttagggagaggtcggggagtgcggttacctatggctccgaagtccccgctggcatcctgaaaggcatcgtatttctcgcttaaggcgttacccttgtggtagtgggcgcatggacgtttacagttgggcggggcaggatttaaattccgccagtggtggaccagtattggtgataggagagatctgggaagaattattaagctgtcgcttttaaataaccctaatagtgtttgtgacaattttacccgttgacttttctcagcttgcttaggacagttctttgtggggaactgaactgtaggacgtttagcgtccctggccccacacgttcccaaagtcggggtgagaactagtgggacgggggatcatggagtgcatggccgctgtccccacctttggtagcaccaactgctaactgcctcctgaaaggttgttggcgccatcatggtttctgggagccactttcttttttattctttttttttttttttgagacggactctagctgtgtcacccaggctggagtgcagtggcgcgatctcggctgtcagctccgcctcccggattcacgccattctcctgcctcagcctcccgagtagctgggactaca

SEQ ID NO: 460 - DNA, 613 bp, Homo sapiens
ccttggaacttcctttccgctgttacttccgtcttgatgcttgaaaacccggcctggactccaagctgtagctggcttctctcggtgcaatctaaaaggcggagctcggagtagcctgtgccgtcgggacgggaagatgtcagcgggcgttgtcctgccactcgggcgctctgggcgtgggccttgggtggggcacgtgcctggaaatccccattgatagaggaatagaaaatctcccaatctttttaagtaactacgttttaaaaaattaatgagcgtgttgtcttttctcatatagaagagtgattacttttgatgtctgtaagcctaaagctggggtcgttctcagtagaccgccaagctactggcttctccctctatccctccctgccttcccacgcggaaaaggtttgtatataaatagcgcccaaatggacatgaagggtagagtggttcttaatctctccctttttttttttcttttatttgagacggagtttcgctcttgttgtccaggctggagtgcaatggcgcgatctcggctcactgcaaactccgcctcccgggttcaagcgattctcctgcctcagcctcccgagtagctgggatcaca

SEQ ID NO: 461 - DNA, 826 bp, Homo sapiens
cactgattaattctaactgggccacaccccacctcctcgccagggaatccggatttgacagaagaatatactcaaggggtgacttttcagtgctgtcctttaggtgtggtgacaaggccaagagagtggggatgggggagtctttctgggagttcccataagctgtccagcatgcccagtgtcaggccttctttctctgcctgcagatgtggctgaaggtggcttgggaggcctgcaaggctcaaaaccatggggaaagcccaaaagctcactatggcggaagcaggcttgaatgttctggtgaacagtcacctcagaccttttcaagccacccatggccccagagaaggcaccctgtggaatggctgcctctcttgttcctctctctctcacgtgatcttatccaaggccacatccccaacaacctgtgttcccttctgcccccgcattcctctgctctcaccacctcctcacctggtatacccctcttccccatgttcacttctccgaaacccattgatctttcaagaatctgatcaagttctacctactccaggcagccctccttggctggtctagtccatccccagccagtctccacagccagccatttggtcattgccatggccatttatttcctgagtgctcgctctgtgccaggctctgtgccagacctcgtggcccaagccctcctctcatgaggcccacaggctgctggtgccagtatggtacctatgtcagcccttgtcaccaggcttgctttagactctggttgccggccacagaatgctcccggtgtcctctgcagctcctgactcttccacca

SEQ ID NO: 462 - DNA, 576 bp, Homo sapiens
acgggttccccgggctgtggcttaatccgttcatgtgcccggcactgcccgggctgctgccgagcccgttggtactgaggcttgcgttgtcagcctgtccgtttgctaacgtggccatctttatatacatagagaaaatcttctttccttttattctttctcgctcacactctctcgctcctctctctcgctcgctcgcgctcacacacgcacacgcatacacacactcgggttctctccccctcggtttctctacacctcgctctccgctcgctctctgctctctctctttctcctcttctctcgctcgctcgcgctcgcgctctctctctgctctcccccctctctctctccctccctccctctcctctctctctccctctctctctccctctctctctccctctctcctctctctctcggtctgatctgattttttttttctttttttcttttttttttttttttttttttttttacattgaacagacaggatctctgtcctttccgtttaaacaggctggaccggttcaagttcccagccagaccaggggtgggggtggcgagtgtgcgcgc

SEQ ID NO: 463 - DNA, 441 bp, Homo sapiens
tggaaacaccaggtgcacgttgtgaccttgttggcaaaaagcaaggtgccggaaatccatgcacccacagcagataactgtccccctcctgtgcccatcctgaagctggcccaatctgagctccccacaggagtttatttttgatagatcgagaagactagaactatccaaggaacgagaactgccccgcttgggcctcctcctcacctggtgcgttcagtcaggctgcagcatgccctgaggatgtgctgcaagtccctggggccaggctgagagagctgaggcccttgtcttgatgaactgcaggagttgctgtcagggcctgctgggccctgaaggcctccagtctgccaagagggaggtggccgtgagctgagtggtgccaagtctccggtgccaggcattggaaaggtcatcttgacatccgtcttcccttccctg

SEQ ID NO: 464 - DNA, 479 bp, Homo sapiens
atattccccttccacctcttgatccgttttaagctttacaaacacactccggggatccgcggcgggatgcctgatgggctcgggaacctggtcgcggcgcacccctagtcctgcctcagtggggccgacgcccttgggctcatctctccccttgcgtttgtctccctctacttcgggcttaccctctcacttcagactaccccctgggggtcacctccctccttggacgcacccctccccagcttcagactcgcccctctagccccctctggctcacctccgcggggccgccaccctggcctgtgccccctggaagcgccgagacccagccgaaggcttcccagccccgcactcgtcgcagtttgaatttcccctcgctggctcccttttcgggacccactcctttcttggctgggttgtacgaagtcccggacctcgcgtttagtttgtccgtctatatctgttgtaactcctcccag

SEQ ID NO: 465 - DNA, 437 bp, Homo sapiens
ctctttatcagcggaatgagtaagacacacacacagcccccagagctcccggtctgctgcgggaagcttattaacgctgataatagatgaggctgacttacagacccacatttttcgggtaaaaggctggttccctctggtccctaattgcgggggtccactggctttcctcaccacacacataaactgctgcttttaaataccccactcggctgccatttaacaaacttattcatacaaagtttcgttaaaacttgaggcggtttaatctaaatactgatcgtaactattcagtctgttagttctccctggaaatggcagcggaggcgcctccccccacctcccgcccaaccacacacaccccaccgcattcttccttcatacacccgggttcatctcgccaccgaggcctcccccacccgcacccggcggggcgc

SEQ ID NO: 466 - DNA, 586 bp, Homo sapiens
ggtgtgctgggcaacagcctagtgatcaccgtgctggcgcgcagcaagccgggcaagccgcggagcaccaccaacctgttcatcctcaacctgagcatcgccgacctggcctacctgctcttctgcatccccttccaggccaccgtgtacgcgctgcccacctgggtgctgggcgccttcatctgcaagttcatccactacttcttcaccgtgtccatgctggtgagcatcttcaccctggccgcgatgtccgtggaccgctacgtggccatcgtgcactcgcggcgctcctcctccctcagggtgtcccgcaacgcgctgctgggcgtgggctgcatctgggcgctgtccattgccatggcctcgcccgtggcctaccaccagggcctcttccacccgcgcgccagcaaccagaccttctgctgggagcagtggcccgaccctcgccacaagaaggcctacgtggtgtgcaccttcgtcttcggctacctgctgccgctcctgctcatctgcttctgctatgccaaggtgcacgccggtcgcggggccgagacgcgcgagggagggcggagggccggtgggggcc

SEQ ID NO: 467 - DNA, 431 bp, Homo sapiens
cggcctcctcgcgcctcagccctctagcgccagcgttttgtcacatctccggtttcctttctttgcttcagcttccatgcgaccccctagctgcggacactccacacgctgagagtcagctgagcttgaacaaagagacctttctcctctgcagaaatcgctcgagccgccagcgccagaattcacagcagctttctcttgaaatgcttggcatttccccacaatttcttaggtatacttggtgggcgaagtgcgtcatctctgtttgaatttatcgcacagaataagccattttctccatttaatgttatcccctcggtaaccaggaggtcggaggccacaacgcaaaaacagcaaatttgtcaacggaatgtgcaaagccgggcataaatttatcttgtcctaatccccggccccctttcatgcggtga

SEQ ID NO: 468 - DNA, 462 bp, Homo sapiens
tccctgggtaccacagatgtgccctggttcctccgtgcccaccagcagccgggagaggcgaaggcactggcctggcctggcccctgctttgagtcttatcatttctgcatcattctgagctggaaacctccccaaaatcattcaaggacccacataaccacacaatggaagccttgcagccacctaacagcaggaaaggccatagccaggtccccccagctgcctcctgggctccgacccacccaccgtagccacagcctatttacacacgtggcgtcaaagccacggccgtcaatcactgcgtgtcgctcccagctgcagacgctggacaaaagggcgacggggtggcagcccccagcgaggacggatgggccatagggctgctctctgggcatctctgagcaagaccctccggcgccacagcagaaatggccagacgggcctgtacccaaaaagccagcg

SEQ ID NO: 469 - DNA, 587 bp, Homo sapiens
tgggcgaggaggcgggcaggacgggcctgacactagggacctcgggccccgggaatgcctctgggggggcgtgtacacccgttgctcccaggaggcacacactgcggttcgcttcgccaagaatgtttaattgcatttgatgactacggtttccattcattcatttgtagagatataacactcagaccacaaaatgcataaaatgcggtggcttttagtattaacagagtgctgcacccgataccacagcctcactccagaacattctcatgggcccaaaaggagacctggggtgttagtcaccagctcactccccgtccccagcccctggcaacccacgctacttagtcattatttaggtgtttaggagttgcaaagtcaaatctttaaacccacatatggccaggcgtggtggctcacgcctgtaatcccagcactttcagaggccgagacgggcagatcacctgaggtcaggagttcgagaccagcctggccaacatggtgaagccccgtctccactaaaaatacaaaattagccgggcgtggtggtgggcgcctgtaatcccagctactctggaggctgagac

SEQ ID NO: 470 - DNA, 788 bp, Homo sapiens
ccgtggggctgctggacacgtccgtttccacctgcggggtgggcaatgccggcacagcgagtggccagacacaggcatgcccagcagacacccaaccgctctccaccacctcaaccttctgtattttcctgaaagcatttaccactccttgaagttatagattaatttgtctctgcattttgttgtccaaaccaggagctcggtgagggtgtgatggggtctgccttggtcaccgtggcaccccagcaccccgcagggtccctctggccaaaagagatgttcactgatgccctagaagcaggaacagaggggctcccctgcgctcgccaagggcgcctgacaaatctggaggcccccatgacagaatcagctccagagtaggaaatgtgtctggctgcacgaaagccccctcgggcgcagcgcctccaggcccacgcagatgtcgctgtcatacattgttggaaacgcctctgctggaaacagtgtttgctgaatcttggagggagaggaaaaaacctagtaaacattcttgttgaggtgatctgggggaatgtctgcaaaggcttggagggctgatggggcagagctgcccaggggcccaggacaaaggccaggcgtgcgcgcgcacacacacacacacacacacacacacacgcagtggcctccctgggggccgtggctgggcgaggcctgctgttgtccgagatgctggaaggagctgtgtctctgggtctgtcccggccgcaccccgttctgtcccacacccaaccactccccacccacaggcc

SEQ ID NO: 471 - DNA, 450 bp, Homo sapiens
tgcgggctcagggcctcgagtctctcctggagcacgggctgcggtgcgccggcagcttacggggcggccagtccttgcccacaacgatgtggagccctgtgaaagtcggattcgaataaagggccacgtgtgcacccagaaagccgagtctgtggttcaggggggtctgtcggcggagcggggccactggaagaaaagcctgcggacctcggttcagcgcacgagtaggacccgacagggaagactgcaagggtcattgtccgagcagtgaccgcggggggctcgccactgagggggttcgcagcgcggagactccagtctcgcgggatctgaggcgcactcggcttcgagggagcggcggccgcgcagccgctgtcaggccccgtcttgggccgagtcccgggttccctgtagcaggcttgggagcggggcgccaccttcctgggccct

SEQ ID NO: 472 - DNA, 580 bp, Homo sapiens
tccctgcagcacccgcggcccttggctggggagcccctgggacccccgccggcagcgcgtgctgcagacatcaatcacagaaggcaccacgtcactgaagcgccaggcctgggccgtcccaggctccgctgggtccctggtatgccgtcggtgctcaataagcacttgctgcagaaatgtgtgaaggaaaaagggggctagggaagggctccaggaaagtgtaactcccatgtcagagctgggaggggcttcgagggcatcactcactgagcgggaaactgaggctggagaggggctgcacctggcctggggtcatctgcaagatcaggggccgtgtccaccaatgtcctgcaccccttgccgactccctctgcctcggccgcctccttgcccttcaccctctctggttgctccctgcctggtgggggtagcttgtccgtgcccagtggacagcggggacccatggaccccaggcaccccaggccccagggagaaatgcaaccactggtcccacacagagtgggttgtcccgggtggcagcatgagccatgcatcaggcgtcagaggcagagggttggca

SEQ ID NO: 473 - DNA, 490 bp, Homo sapiens
gtgcgcccttatggtcggtgcgggagaccccgtatcctccagctgcttccggtggcccttcctcaggtcatgtcccgcagtcccctgcctctgaaccgcgctctcgcgagtttccgtagccttcgcgtccttcctccccagctctgattcgcctttatgtggagattcccctaggccttggtacatccccagaattgaccatcgttcctcatcgttacctagagtatgagtttttcaggtttttgtatcacaggcccccagttatctcccacacactaccccgaggaccgaggttccccgctgtcttgcagagaaggcctccttgtagggtggccagaaacagaggatcgactccgtggaaacaaggacggctgggtgcagttcctcaccctataaccccagcactttgggaggccaaggcgagtggatcacctgaggtccggagttcgagaccagcctgtctaacatggtgaaactccatctctgccaa

SEQ ID NO: 474 - DNA, 517 bp, Homo sapiens
tagatacacaaatccttattgtggtggagccccttccttccgagctttccggaaaaccaacgacacaactccccaaacaggaagcacaacccgcttgcacctgtgcaccatgctcggttggagctcccagctgcctccagggcacagcggctttggaggaggggccgcgttgcatgctgggagcaatctgaggggcgggaccctagcgtttaagggctgtccttttttgaaccacatttccaaagcgctcgggttccccgagtggtgccccaagcagtggacgagcaaggtgcccgagaaggaggggaccacagacaccgcctggtgtccacggactaccaggaagccagggggcggtggcctccgtggggaatgcgcatgtgcggtcccgagcctcaggctagcgttcgggactagagtggggcgtcgccgccgcatgttgcacctgtcggcagcgcggcctctcccggatgtgacgtagcgtgacgcctgcaggattgatgctgaaatgacgagg

SEQ ID NO: 475 - DNA, 504 bp, Homo sapiens
tgggcggccctgcggggcaaagccccctgtgcgcgcagagggggaggcccgggaatgaatggagggatgcctggcgcctgaggtcacctacacgccgcccgaggtcagagtgcagggaggattttctgccctgggggtctcggtgtccccagtgggaaggggggctggacactgagcccagtgtttcattttttaaatggaaatgattcccgccaccttccaattatactcagcataaaaaaaaaatagaccagacacggtggctcaggcctggaatcctggcactttgggaggccgaggcgggtggatcacctgaggtcaggaatttgagaccagcctggccaacacggtgaaaccccgtctctactaaaaatacaaaaattagctgggcacggtggtgcatacctgtaatcccagctactggggaggctgaggtgggagaatcgcttgaatccgggagatggaggttgcagtgagtgcgatcatgccacttcactccagcct

SEQ ID NO: 476 - DNA, 621 bp, Homo sapiens
ttttaagggtccgttttgttttattttgctccctggtctagctcagctccggaatcggtgcagacacgcaatatcctctcagtagatatttatggaataaataaatctgtctcatctttggcattgctatgatggggtctattttgtctttttctctatttatccccatgtttaaaatatttttttattagaaaaaaaaacggaagtcaccttaggatgaaagcgcctcccctcccaaccccccagtggggtttggcatgtctctcgctgtccccatcacctccctggcccgtgcaggtggcctggccgtggttctgcacgggccgccgcgtttaggcggcactgcagctaatgacaccccgcagcgtgtgcctggctggccgcccgagatgtgcctgcgcatggcatggggacgtttgcaagcctggatcctggcgcgggggcaggtagcgggagaacaggcacgcagatggcagaggggctggggcggggacgctcgcctggcgtgggccacagtcacgacccaatcgtagcccccactgtgtctcttcatagttgcatccttggaaaccggcggggctgcaggaaaaaaattaaaaatcagatgaaagaaaataaagc

SEQ ID NO: 477 - DNA, 608 bp, Homo sapiens
ctgaatctggccccccaggagccccgtgtgaggcactttcaggggccgccggaggcctggaagacccgagtgtggcagtaggaatgtcccgagcggctttcagtgcctccttttcaaccctccccgcctggagccctccttctggatttgcgtctcggcagattgtgggatgtcacttctgtcctcttccgccctgcgggattttcccagagccttcgggtttgagcccagctggaagtggccctcgttcacgctgcctgtcagcacctgcctgggctgtgtcacctagagtgtggctccgtgtgtgcggcgcagacaccgtgggggtgtttggagcttcctgcgtggctcccagatgtctttactacacagtgcccgccgtggaagggacagatgagcaatgtagcccgaggcgttctttcaagagcagaggaatcgggagcgaagtgtttttgttcatttagcagactaggatcatgggcgagtttcctctaatagcactgaagaggcgaattcacggcttcccctcggccagcggctttggcgtggatttggagccgggccaggttcatggaaccctcctgcagaagcagactggggcttcctgg

SEQ ID NO: 478 - DNA, 430 bp, Homo sapiens
tgccctgcactgtcctccaagggccgctaggtggcgctcccgcccttcccggggccgcgctcatgcctggaatcttcccaggccgtcctcgcgctggaggaggacggagcctttgcctggagggcgtgggagctccgccttgcctggcccttccctgggttggagttgtaagcggggagaggggggggcgggggagaatgctgtacctcttcgttaaaaaaaagcacaatgagcaggaaggggcgagctgtgcgcatcctcacctgcgtccgaagcgcagatggagcccaagggaaaggccctgtagaggaccccgtgtgacttggggcaaagcgtggcctcccaggggtgcgtgggcagcgggacgcccatggtgtgaccgggtttgtgcctccatagggaccgtctgccctgtgcagagagcccctgt

SEQ ID NO: 479 - DNA, 471 bp, Homo sapiens
caccacccccagtcaccacctcctgtcccagccccccatgtcccaggcccgggaggctgtcatcgcagctgcatttgccaagctggaccgcagtggggacggcgtcgtgacggtggacgacctccgcggggtgtacagtggccgtgcccaccccaaggtgcgcagtggggagtggaccgaggacgaggtgctgcgccgcttcctggacaacttcgactcctctgagaaggacgggcaggtgggtggctgcgcggggggccccctccccaggcagttcctcggccgtgccccctcacggccctctgttcccaggtcacactggcggaattccaggactactacagcggcgtgagtgcctccatgaacacggatgaggagttcgtggccatgatgaccagtgcctggcagctgtgagcagctccggctcagccctgctgccctggcctgtcactccccacccctgccggagac

SEQ ID NO: 480 - DNA, 802 bp, Homo sapiens
tgtagtcccaagctactcgggaggctgaggcatgagaattgcttaaacccgggaggcggagggtgcagtgagccaacattgcactgcactccagccagggcgacagagggagactcaaaaaaaaaaaaacaaaaacttttgtacttggttaacgtgaacgctgcctccttaaaaaaaaacaaacaaaaaaaaacccaggaacatttaacgtgaatgtcagggactttgggatgagtccagggacctagaagtgcggaatgcacaaaggagctgggaaagtgtgtgagcctgagcccagggctgcaagttgggagtgcctgagtgcacagggaggccactccaaccctccagctgcctctaggggtatcagagagggagcccgtccccactggccaccaatacctcttaggccagggcgaacacgtccacgggggtatattcaacttgtgaccccagtctctgcgagagcctgcagtagcatgggggggagagacctcgcgttctctaaggatggaggacaaaggaggagagactgggagggcagggacggttgcggaacggtcacaaggcctgggcgcgagggtttggaaggcaaaggtgcgagaggcaccaggtgacccgtcctgcgtgattagcggtcggacaatctgagcctgctaagagctttcctgaccacgctggtatgaagggcggcttggaggtgacaatgggggacgacagggcacagcaggtgtggagacggtgtgcagggccggagcaaaaccgttaaaggcaggtggagacaccctcctcccctccccca

SEQ ID NO: 481 - DNA, 311 bp, Homo sapiens
gtgctgcgctcccggggcactcacgccgtcgctggctactccgcggggccggctcagctgccgctgcagcgctacgcacgcctccctcagacgcgggctcagctcgcacctgcagaggaggccaggagagactcaggatggatcggggacagcgccgaccaggaggagggcagccgcctcctctccaaacaactagcgagcgccgctcacctcccctgtgccacctgctgaggctctgcctccgcctcgggctcagggtcccggatgtccccgccgctgggagggccaggggtgcccgagtcggatccccc

SEQ ID NO: 482 - DNA, 470 bp, Homo sapiens
ggcttggcccggtgccactgtcgggtacacaaagtcctgtctggggggccggttgttcaggcagagccccaggcccgagctgcctcgagagaaaagcaactgtcatgctaggtggctgcaaatcaagagctcaggagcctcagctggatgggggtcccagggagcatccctgatttctatcgtctcccgtgtaccctgccccaccatgagtgtgacccgctctgagggacacccaggtgcatcctcactctagaaagctggtgatgtagtcacggctgcaggatgaccacacgaatgggttggtcttcatggtaatgtgggcagccatgagcttggctgggtcctgaccacgggccccacagctgtttcccacgccgtcatggttcatgccgaatctggggaaaggggtgtcggctctgccgggcgctgagggacccgcccagctcctcccctcctccccctcttgtgtg

SEQ ID NO: 483 - DNA, 452 bp, Homo sapiens
ccctccgtttttttttaaatacctggacttaattgtttaaactggagtccggaccaaaacatctctgacctcgggggtcttcctttggtcaggaagaggggagggagcaattggacccctttccctctgcggctgcagagtccaattttcctctggggacatttgctttttcccttggggaagacagtacggattctagttctgtggactcttttacttagctttgagacccaagcaagttaattggcttgtgcctcttattttactctaatgcaatgaataaagacagtcccagccttcgccctaagggagcaggagcacctgcgatgccccgttcccaagtcctcagggcgaatccgccaaatgtgcgagctgggcagcccacccctttcagctgctggccggaagcggaagtgggcgtccgtcgcctcgccatctcccatagctgtcgc

SEQ ID NO: 484 - DNA, 510 bp, Homo sapiens
tcttacgtctaagcctctgtaaccccacgccctgcccgcagacgctccccggctagacgattcggactgccccaggagttggacgtggcccgagggcccagagcagacgctgcgctgcgaggcccgcgggaacccagaaccctcagtgcactgtgcgcgctccgacggcggggccgtgctggctctgggcctgctgggtccagtcactcgggcgctctcaggcacttaccgctgcaaggcggccaatgatcaaggcgaggcggtcaaggacgtaacgctaacggtggagtgtgagtgggggtgcgcagggtgcatttctatctggttcaaggtctggagggtggccagcctccagggaagagtaggagtagggtatgaggtgtccctttgggtgaggttttgggaaagggaagaggctggttagtggggttggagaaagatcttggaggatggaagggaccgggtgggcgtgcccctagcctagggcgtggtatttgggcggagtcgtgg

SEQ ID NO: 485 - DNA, 501 bp, Homo sapiens
tgcctcagcctcccgagtagctgggatcacaggcatgcgccaccacgcccggctaattttgtatttttagtagagatagagtttctccatgttggccaggctggtctcaaactcccgacctcaggtgatccgcccgcctcagcctcccaaagtgctgggattacaggcgtgagccaccgcgcccagtctaatttttttttaagagacggggatctcgctgtgttgcccaggctggtcttgaactcctgggctcaagcggttcttccgcctcgaccttccaaagagttgggattacaggcatgagccactgctggcctccttttggatactacctcgagaaggcgtcccagccccgacgtagtaacgccttaccctcgatttggaggcgttgttatcccagcctgatgtttggggtcgcgttccagcggtgatcgcgggagtgttcgcgccccggcccatagagggcgtgccagccctggacccgcaggtttccctccagtg

SEQ ID NO: 486 - DNA, 395 bp, Homo sapiens
gctccgccacagcagctctgcgagggactggcttctgctcacctgagaccggagggggcgtggcctcacctcacctctgcccaacaaaccctgcgcgtccaggccagggtggtatcgcgagtcacctgggccccacaggcaccacgtgaccaggccaattcacctgtgtcctcgccccacgtgaaccaaccacagaggggcggggcctcacctgtcctttccacgcagacttgccacgtgacgggggcgggtgctcgggccagagtccaacggtccagcaagtcgacgaccccgcacctcccagccccagcaccaagagactgaccgaaccagggccagggatcccgggcaagaggagcgggcgctcaccttcccctagccccgccccacgacct

SEQ ID NO: 487 - DNA, 334 bp, Homo sapiens
gtgagtgagtgagggagagagagagagaataggtgtgtgtagaggctcccggtgcctctgtctggctgctgaggctgagatgggagcaagtggctggcgaagctggtggtggcttcaaaccacactttcgtagaacaatcgcaagagaaaattgttggggggagggaggaggaggagaaggcggttttccttgtgcccccccttctaacgctgcttttctccttctctctttccccctcatcccgtcttcccctcctcccgtcctccctcgccccgcatgctcccggcttgccgcctgcaggatgagttccacccgttcatcgaggcactgctg

SEQ ID NO: 488 - DNA, 326 bp, Homo sapiens
cggggctgaggctcaggtagttggggagtaggtgcgcgtttaggaaccccggggagatgctgcgtctcaggaagttggggagggcgctcaggcttgggactcctctggggacaaggctcaggaccttggggagggagtgttcgctgggaaaccttgagagattccgtgtcttagaatgctggagagagggtgcatgcttaggacccgtcggggagcgtggctgacaacagtggggagtggaccttgcgctcctccgaccccctgggggtgaggatccggattgtggggggagttggggatgtagggcaaggatccctcaggggcgc

SEQ ID NO: 489 - DNA, 398 bp, Homo sapiens
gaggctccaggcggcgaacaccctgccctgtggtcacccagcacaggtccggagactccgattgtcacttctgtttattgagttacaagttgagatgtgcaggttcggtggcgccagcccccccatcccccccccttgggcaaaaatagctcccagcgccgaggaatggggggtaggaagggtctcggataacgggatggggcctcgagggtccctgtggggctcgtgggtcccaggatcaagcacggctgacacggaagacagcggggtggggggcctgccttggccgtggcgttggggggaaggtgagggagagcttctgtacaaggtcatcttccgtgagggtcccggctgcggccccaaaacgccgatgggccccgcgggacggaagcggagag

SEQ ID NO: 490 - DNA, 481 bp, Homo sapiens
cgcttctcccggccctccagcacgccacgccgctcccgctccatcgccccggcccgctccgacctcaccgttcttggagccctcgctccttctcaaggacccgaggcctggcacgccttggcacagtcttgggaccatcccgtggccctcccgcattcaggaggcgtaggctcaatgaaacattctcattcttcaaaaggacacggcccacgccagggcgttgggaggcgcacctagtggagacgcagctctctgccctacaccaggagcttggcaatggggttccagcttggacgcgggtaccctgggcacttcccaggcatcttcttctgcaggtctgcacagcgggctactgggacaaaggtgtgatgggggctgctgagattagggggttctctgagggtcacttgctgctgtgaccctggggaacccggggggggggctccccaacatcaggttaatacatgagttgcagagaggc

SEQ ID NO: 491 - DNA, 346 bp, Homo sapiens
ctaaagagcatccgctgcagcgcaaccacgaagacactttggcaaacgccggggttctgtttgataaggagaagcgggagtacgtgttagtgggagcagatggctccaagcagaaaatgcctgctgatttggttcacagcactaaagtgggcagccagagagacctgccaagtaagctcgaccctttagaaagcagtcgggattttttgtcacacgggctgaaccagactctcgagtataacctgcagggtcctgggaacatgaaggagaagcccaccgagtgccccgactgcggccgggtgttccgcacttaccaccaggtggtcgtgcactcccgtgtccacaa

SEQ ID NO: 492 - DNA, 432 bp, Homo sapiens
cagactcgccatcccagaccctactcccagttcattccttcctgaggtccggggtttgaggcatggggagggatgggttggaaacagcgcccaggaactcgtcctagcaaggattggcgggtgggagtggcggcagatcctccaccacccacagcttacgcgaattcacctccctcctctgcgctttaatagcgggtaatggtcgctgaaatgagaatatccattaccagcactaaaaaagccgctttttactgtgccacacagtcaactcgtttgatcctcacgcaagagttagaatgaacaatagccccattttcccagtgaggaaactgaggcccagaggggatgagtgatttccccagggccacccagcacgggcctccggaatcctcaccaccgaccctcccctgcaggccgtggttggggacgccc

SEQ ID NO: 493 - DNA, 207 bp, Homo sapiens
accgacctctcccagaccctggccttctccttcccctccccttcccatccggcgtctggctcaccttgcgtccgaagacctgcacagactgcagcgggcccttggacggcatggctccgagcgtggactagacaacctcaccgcgcggcgccgcaaccggaaaaggaaagctaggggccaccctggccgcttttcagggtctgcgca

SEQ ID NO: 494 - DNA, 378 bp, Homo sapiens
caccatgttggtcaggctggtctcaaactcctgatctcaagtgatctgccggtttcagccttccaaggtgctgagattacaggcgtgagccaccacccccagccttaagacagactattctagactgagtccaagttccaactgcttgatttggggcaaatgtcttcctctctctgagcctcggtctcctcttctgtttaagggacacattgacagcccctccctcatacatgttgggaatcccagactcagcacggtgcttggcacacagtagtcactgaatcagttactccccaaaggactccatccaccctcccaaaccccaccccggaagtacctcgggaagcacgcactcttccattttgcacgccacaaaca

SEQ ID NO: 495 - DNA, 402 bp, Homo sapiens
ctggtgccttcggaagatgcacgtcgggaaccttcggcacttgcatttccggcagccgaatctctgacactttcggcagctgcacctcggggaggtgcacatcgggcacagccatctcaggcaccttggggagttttatctctgggagcttcatgtcagggactttcatttcacagactttgggcagctgcacctctgggaggtgcacatcgggcacggccatttcaggcaccttggggagtttcatctctgagacttttggcagctgcacctcggggagtcgaacctctggcacagccatctcaggcattttagggagtttcatctctgggactttcgggagctgcacttccgggaggtgcacatcgggcacagccatctcgggcaccttcgggagtttca

SEQ ID NO: 496 - DNA, 824 bp, Homo sapiens
caggtcccaccccaggtggagtcagggcagggccagtcaccaggtgagccgggagcagagcagggagcagactctgagctgctcctccctcgtccaaggggtttcctcctctcatctggggaaaaagtgtgagatgtttcaaagcttccttcttccttgtagctcatgaattaggcaaaagaacacaaagaggtgacaaaaagggacgtattgatgaccgagagaacccttaaggacccttcctgcttttccctctgtgaagctctttccactacctggggctttggggctgagggagccccctccccacattccaggctggatccataatcagccatgagaaattactgaagtacagggaggagggtctgcagagacagagtgagagggttcagcggggagaatttgggatctgcttgtgcccatgggacacaggctggaaaagaaaagttttttcagctcttccttaacaccagagagaccccatctcaaaatatgatgccaacggtccagggatccatgtaccacggactgtgatcatcttgactgtggtactattgcggtcagtggccaatttgtgggcatagcaggtataggatacactattctttgcagtgctttggggataaagagctcttgcatgtgttgcagaaggtttccattactcagccaaaaatacggagagggtgggttagaggccacatggatgaagagtctaaggtttgctcctaactcgtaagatatatctggggtagaaggggtgtccaagccacccggagcaaagagaataaagccgtaagtgatgtcctcagagggaagcggaa

SEQ ID NO: 497 - DNA, 781 bp, Homo sapiens
gccccaacccctatgccccgcccaccacgagtcaagtcaggctcccgcccggttcggcttacctcgctgaacgccccgcgtccgatcaccttcagaatctcgaagtcgtccctctgcagtcggacctccttaagcctcaccacgatgggctccgctgggggggtggtgggggaaaagaaccgagggtcaccagaaagggcactggagacaagggggaaagccccaccctctgtctgtctccccttctctctgcctctcagcttcaccctaggactgtctgcttcccaggggcttccccacataaacaccgtgtaaggttctgggggccaaaaatgccctcccatagaggtgagatgcctgagaagccctttgcgggacagggcattggcccagagggctccaagggtgtgcaggatggttagggtggggtaacggagtctgcagaaggacagaccctaaagaacaaggggagaaggaaataagacccagttcttccaccttccaccctgactccaggtgacagttcaggtgcagccagggccccacccccacgttctcatgtagaatgtcctgggtaacggcccagacgtggggttgtatccagtacctctagattcagatgcaggtggttcttgaaccacactttggaaaaccctggatttggggctgggggaaggaacaggacagggagacaaaggtagcatggtcacatatcccagactcaagtgctccggtccagcccatctctcagtcctccaggaggagtgctttagtcctaccc

SEQ ID NO: 498 - DNA, 618 bp, Homo sapiens
aaggtcttcatgggggggggggggtgtggactgtaccatgttgcgaagccgggggtgtgtgagattcctgggctaaaccacagggaggcagaggtggagggaaggggttgtaccttgtagattctcagtaagggggaagggagcatcagattgtggaacagcgtaaggtagtacgacttggaaagggagagaggatccaaattgtaccattttggaaattcacagtaataggaagcacagaacagtaggtaattggaccataggaaggcaggggtcaagggttatagaccaagttacaaagcagtgaagaggaaagacaaatcgtgggctagcaatggcctagggctggcaggtggactgtaccacatagacatgtagggaaagagagaaaaagagacagctcacgaggctagaccatatagacgcaggaaggaggatgggactgaatcacactgagacaccaaggggagacacatataagagggttagcccaagacttagagggggtgcagactggaccataccatgcaggcggtagttggggcctcggcagcgcctctcggggtgccgggggtccctgttgcccctcagtgccctgtgggccaagggctgcagggg

SEQ ID NO: 499 - DNA, 319 bp, Homo sapiens
caaggccgccctgctggtcaccacgcggcccagggccctgagggacctccggatcctggcggaggagccgatctacataagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggatcgtgtgcacgactctgaagctgcagatggagaagggggaggacccggtccccacctgcctcacccgcacggggctgttcctgcgtttcctctgc

SEQ ID NO: 500 - DNA, 296 bp, Homo sapiens
cacgcccacttccggaaaggacccaaagccgggcgagtgcacgtcccgccggttgctgaggagaagggaggtctgggcggggcagcggccgaggctggactggggcgcggcgggggcggtgtcctggttcctgtgtgggcggcgggcgtgagcgtgcgcgtgtggcctggtggctgtgagtgcccatacgtggtgagcgtgcggtgctgtgctgaggaggggcgagcgcgcgcggggatggcggcccggtgtgtgactgtccggtgcgtggccgcgaatctgcgcctgcgtgcatg

SEQ ID NO: 501 - DNA, 800 bp, Homo sapiens
gaaggaatagattttacccctgcatctgctggttccattgcccctcatccggctcccaggaaagctttgaattgtgggcaaaaccaggtggcactggcagagacattcttggggttggtgtctcccctaaaacacactgtgccttctagagaggttccctagaagtcgcgatctttgctgagaaatatccacacatgctttgccacattgtcaatcataggaacatgctgcccaacaaatatttgtggcatagtaaaatgtttttaaaagcagttgctatggattgaacgtatccccccccaactccccgccatcaattcatgtgtcctgatgtgatggtatttagaaacaggccttctggaggtgattaggtcatgaggatagagccctcatgcatgggattagtgcccttacaagacgagacagaaaagagcttcctctctctctcaggaataatggtgctcaggagaacccagccatactggcaccctgatcctagacttacaactcccagaactgtcggaaatacatgtttgttgtttaggccacacaatctgtggcagacaagctaactaaaactgcagtcttgcaggctagtgtctccctagttaggatttgatctcagagcctcaagggctgggcatggctcttgcctcaaggattgaagttgcagggagggacatggaggcctgtccaggacctgggagttgtgccttggtacagtgccctggccgctctgtcctgtgcccccggggctgcccatcccatgtgtgattcctactggctgcactgtgcaggca

SEQ ID NO: 502 - DNA, 812 bp, Homo sapiens
ggcctcactctccatcctgaagatggagcacagactggctggctgaggccgggagctcatcacgggatggagacagattggcagacaaagaggaggcgggcggttggggctgggtgcccctggggaggaaggcggggagagagcgtaggagcaggaggaaagaaggctgggcttatgtgcagcctcacagactgggcctgcagagtctccagctggaggttgggactggaccttcctcctgaatgggcagggagggaggcttgttggcctagccctcggggttcttgaaataaatgagggagggccttggggggtggtaaattgctgaggctgtgataaacattctccccttcccacaaaacacacacaggcacgtgtgcgtgcatacacatacacacacacaccctggccacactgctgtgcatatcttactgattgttgtcctctgtgacagcagttctccaagttgagtgcattggaaggatcagttaaaatgcagatttccaggcatttccatctctaaatgtagggtgtggcctgagaatgtgtatgtcttacaggatcctggttgatgctgatgctgggggttctgggacacactttgagaaccactgctccattgagcagcctttcagtttgggtgcccctcggtgctggtgctgggagagagttaggagggcaagggggttagtgagggctgctacctgtgaaacacaaagcaggagggaatggaattggccaggagagcctcagaccacagcccgggcctgcagagtcaccctctaggggtctctggagtaaagattatccac

SEQ ID NO: 503 - DNA, 386 bp, Homo sapiens
gaccagggtccctggctacctgctacgggggccggcagatggtggagcccggaaaccgagcgctgtggagcgcctggaggccgacaaggccaagtacgtcaagagcctgcacgtggccaacacccgccaggagcctgtgcagcccctgctgtccaaacagccgctctttagccctgagactcgccgcacagtgctcacgcccagccgccgagccctgcctggcccctgccgacggccccagctggacctggacatcctcagcagcctcatcgacttgtgtgacagccccgtgtcccctgccgaggccagccgcactcctggacgggccgagggagccggccgtcctcccccagccacccctccgcgaccgccgcccagtacctctg

SEQ ID NO: 504 - DNA, 519 bp, Homo sapiens
gaaagctcagaaggcgagcttaccccggacccaaacgcgcaatactaaccgggagtgtaaggaggccacgccctcctcgcgtatttgtgcccaaatgagctgcctccccagcgtgcaccaaattcctgagccacctgctccgagctcgatttccctttcaagctgtgtgttctgcgggggcagggagagctgcaagggctcatctttcaggacgtgctgcaaaggccctttgaagtcatccgccctgctcttggcaccttgcgcctggagctgagctcttccaaaaatgacccctgcgcattccgtggtgcggccgcccagcccaggcagtcgcggtccagagcaggggaagctgcagcgcggccgacagctgggagagccgaggccctgagggaggaccctgcctcaagccaagcaaccaggccctgcggccctctccccacccctgtgcggccctggcgcagggaaccgggacggggctttcaggccttgaagggcagacttactccaccaaacatt

SEQ ID NO: 505 - DNA, 551 bp, Homo sapiens
ggaggcccgccagtggtggccgtagacacgccagagagggcggcggcgccggcgacgacggcactggcagcgcaggagacggaggaaggcgcgcttgaactcgcggctggaacaggggtagatgagcgggttcacgcagctgttgaagtagccgagccagaagatgaccttgaagacgccctccgatggcttcagctgcgggaacaaggagcctgtagggagcagagaccgatactatttagctgcttggggagggggaggccaggcggctctgggcgcaaaggggagacccttcagtagccttggctgagtcattgactaggagttctcaagggatccgtgctgtaaatcaggaggtccatgaacttggatagagaaaaataataattgttttcactcacctctacctgatattgaggattaccttcaatgaatgtaggcaacaaaccagagtagagttagacaaaaccacgtgcaattgctgtttttgtagctcaaaaccggtgcaatattggcagtttcgtattatgagacaaagtagcaaacatagc

SEQ ID NO: 506 - DNA, 421 bp, Homo sapiens
tcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaa

SEQ ID NO: 507 - DNA, 580 bp, Homo sapiens
aaggtctgcccagaaggctgccgaccaataacgctcaccctcctgcgcccggtagtgaggacctgggacaaatcgcagtctgtgtcttcgttacaaaattgccatcattgcccagtggtccagtgacgtcacggaagaggccgaggctcattctcctccctctaatcaccccctcgccagttagccatggaatagaagaaaacagcttggggggtgcggggagggtcttctccagctgtaatgccagctaaggtgctttggtagcaaagctgggggtgaggaggcacaggctcctggacggagccaggctcctggacggaggccgctcagagtctctgcggcgtccgctcggtccgcacgtgctgcagcactacctccttggaaggggccgcgcacttgtactccatcttggccctggcggcgccctgcttcttgcgcaggtggcagaggagcgccaaaagcgccaccaccaggcacaggctcgcgatggagatgcctatgagcaagcccgaatgcacgagccccacggccggaggagtcaaggtggagccgggcgtcgggctgggcgggggctcgccag

SEQ ID NO: 508 - DNA, 491 bp, Homo sapiens
tcgggcccgcagatgcactcgaaggtaccggggaggttgtggcacaccccggagcagaagccgccgttttcgcactcgtcgatgtccgtgcagatgaaaccgtcgtccaggatgtagccttcagggcactcacagctagcctgggtgttggggtcgcagtcggctggacaggcagtctggttgcaaaacatctggcacctgtgcggctcgtggggaatgggcgcgaagccctcggcgcagacgcagaggtagctagtttggttcaggggctggcactggtactcgcagttggctctgaagcacgggtccacgggctccacacactcgccgtccaccaggtcgtagttagggtagcagtggcactcgaagccaccctgtgtgttgacacagcgctgcggacacggactgggctccagtatgcagtcatccacgtcctcgcaccggtgttggtcggccgccagccggtagccggtctcgcacatgcacgagta

SEQ ID NO: 509 - DNA, 810 bp, Homo sapiens
actccaaagtgtccattcattttggttgctaagaattgctcttcgtccccggtgggtgtttaaaaagaaagatctgcccttcctgtcctagctcttacaatcttaaacctcctcatttgttcccaggaacttttgcttagagaatgatattgatgacagtagtttctattcatgagcacttactacaatccaagagttagaagctgttactcactagctatgtgatcttggataaagttcaccttcctgaaccttaattcctcatttttaaagtagcattaatggtttcacacgcagacatgtttaggagaacttactatgaaatacattttggaaaaagatgatgatggatggatggattgatggatggatggatggatggatggagagttgggtagatacataaatatagcaaaatgataatggtacaccaaagtggtgagtatatgagtgttgactgtagtttcttttaacttttccctatgttaacataaaacattgctgaggaaaacagtattgataatggtacctactacatggtgttgtgcaatggaagaaatgagatgaggcaagtaaagtgttgtccgtatggtgtttgacataaagctgtaagtcagtgtgttattgttgttagtatttgtcgcaaccagcaaaataggtattataactctattttagggagaaaaataaaactagatactcagataaaatagtaacaggacgtaaactagttaacatacgtgctagacttgagctgggccggcctataccaaagctgtgccctgcccacctcgccaccaggcagccaag

SEQ ID NO: 510 - DNA, 516 bp, Homo sapiens
ccagccttccagaggagcctgtcccctgctctcctggggaccccagcaccggctgaagcctccagagtgactcatggctctatgtccatcactgcagctctggaggagtaaccacggcacacatacccttctatgggacacctgcaaatacttttcaataagctcgagtccatttcatcatcaacacaaacagaaagaccctccactaggtctctcacccgcttgacataaatttgtttttcatattcctcatggaaaatagagctacgcttgatcaggaaggtgaaaatggaatgggtgagggtatttaaattctctcttctctctcttccctccttgctatgtgtacttggaataagtgcacacgctcagctttaagagctgtttgggacttgggaaccagcttgcctggacatcagaggccacaaagtccgtccctgtctctttgcagtctgtgcagttgccccggttggtggagccatggtggctgtgccctgtgggttgtgaatgattgca

SEQ ID NO: 511 - DNA, 497 bp, Homo sapiens
tggaaagcctaaggggcgagggcggacgcgggcgccgtgcctgcagccccgggtgccgaccaagtggcctcacctgggacaggtcttggggtctccctcccctaggtctccctggaaggtaggcgatcacgggagtgacagaccccgagtagatagccagtgatccaaagacaccaaaaggccaggcggtaggagcccagcctcagacactctctatagtgaccgcggcgcctcgggaaggatcgtcttaaactcattttgcaggtgagagcaactgggacttattaacccagtcacatcggacacagagacgtggcgagggcgacacagagcaggagactcaatcaaggagcccgtattggagctctaaggggactcagagatgcagaaggaacgaacttctaaggggtcttgcaattcctggactggggcgttcctaatgcatcccgggaccccaatgccagggaggggcctgcaggaccccagcggtgggcgag

SEQ ID NO: 512 - DNA, 738 bp, Homo sapiens
ttctgggggacacaaaccactttctctggtttgttctctgctttgtccccggttctggcactgtaggccctcaataaatggtggctgttatgcactaggtttaaaggagactgagctcccctctaggcaccagccagggcaatgaccccaagacctctcttgcaggtaagatgccaggatagttgcagcttcttttacattcattgtcacgtcccaggagccccctgagccgaacgggacttagcgctggagtcctacaggtgagaagtgagaagcaggatgcaagcccatgggcaagcttcatggggacagaatagacaggaggcatccgcctgccagctagaggccagagtccagttccccattgtgcagatggggccgttgaggcccaaaggcacacagagaggtgaggtgaactgaacccaaatgaggggatacagaagctggcatgggaggagtcaaagcaagagggccaggctgcaaaggttggaagacccagaaaatgtgcagtgggagccccagttcagagctgggaaggcgcgcaagccaaaatccagctcccattgcacagatggtgacctccgagacttagagacacacgaggactgctccagggcaaacagcaccgcggcatcggacttcgaactcgcgcctctgaccccaggcaggacatccctcagccccagcccggcccgacggccccactcacacagccccgttccggaagcccttaagcac

SEQ ID NO: 513 - DNA, 892 bp, Homo sapiens
gtgggaaaagagatgtggcctttcctgcactgccggtttctctggcccccgggcccagcatttcggcgaaatctcccaaggtccacattctcccatttctctcgccgatttgggcaggatgcaggcagctggccaagaaatgatcctgggcttcaggtcaggagtcaggccctcgagtccgcttcctgcctgcttctctctacacgtgactcaccgcggacatcgaccaagtcatcgcgctctgccactgcgcctgtgtcctcgtcaggtcactccatctatagattctgggagcctgatcaatcgtagttcctggggctttctctgcgcccgacaccaaaccatgtctaggggaaagggagggtgtctgggatacagtggtgagcagaacggcctgggatcctttatcacgaaatgcaggttacaggggaagacagcagatccatacacaaagaaacgcagtctcaaactggataagttgtgtaactgggtaggttgttgtgaaattaaataatggaagggaaagggattagtaaattgtgacatcatattctctgtcagatgaaaaccctgtcctaatttctatccaccagatagcatgttgtctctaatactcattttttcagctaacttctgtggcaggatggcactgactccccccacccaacacacacacactttttcccttcctagcagggggcctgtaataaaaatgaaagaggagagctaagtgtgatgtaacagggaatggtgaagattgtgactcataactaagatgactgtaacttactgctcaaaccagaacactttaaagaatgaaaagacacaataaataattacaccgggacggtaggcataaaagtgactctcccaggtgaactggcactatggt

SEQ ID NO: 514 - DNA, 685 bp, Homo sapiens
gcggggaggcttagtgcccccttgagaaacggcttggcaacgctgtgcccggggccttgcacgcgggctgagggggctagagacctcgcgacttttactcccccgaccccgatctccgactcctgactttggaagcccccaaagctgcttttcggttgcctacaaagtgtcggtcattgtgccacgtccttggaaccgtgaactcattttctcctcaataacctttaaaaagtagctgtaaaacctccattttacagaccagttggctgaggtttaaatggaggacgctcccagggccccagaggggcgaattccagactcctttgccaggaccttgtactaagccccttccctacgcttttccctgcctaggcctcgtgggctgtgcggctatcctggagacagatgacagctctcccttggatggctttgctggttccgcaccagccagcgcccccatttttcctgcagcaccctgatctgcactccctgaggggctcccactgtccgcggtgtgaggatgtccctgggtaagtggtgctggtcatgagcactttgggggacccttcaaattcccttctgggggaccccagcggagggcatggtcatgaattccgagaactgtatggcaaagccgggtgcagaggagagcctggctaagttctttcattctcctgagcttgac

SEQ ID NO: 515 - DNA, 339 bp, Homo sapiens
aacaaatcttacttgtatctgcttgagtggatagaggtcctttggtatccggcatcttcccaaatggcccaggtgccaggatggcagatctttaaatgatattctttatgggcccagaaaacatatgtattcctctgattagatttggaggccagcccctgtcccttctgagctttgctttctctgctagaagtggctttcattttagatgcttaccagaggctttcgcattgtcagataactgggaatcactatttggatgtcatggtgtacatgtgcagttaggaaccggtacaaccatttgcccctgtgaaagcagcagccccaggataaagccaa

SEQ ID NO: 516 - DNA, 484 bp, Homo sapiens
atgcccggcagggccaggaccagagtccccgcacgagggttcctctctccggtcgcacgtgcccttcacccttcctgaggccgttttctgacgctcctgagctcccctccagggtgagggcaggcgcggtgggaaggcgttggacagagcagacactcggctggggtgcatgcctgggtgcagcctcgtcttttgtcactaaccactcagctcggtagccctccaggctctctcttcgagctccgcggtccctgcgcccccgctgcgctctgctagctgtgatgcaggggttcgggggcaggaaaaatctcgacccgccacgcccgtgcggcaaacctgtcctaggttgagggggcatgaggcgcgcacaaccagggcctcgaaggcaggcaagagggcgcgggcgccacagaagcctgggcctgcgcctgggccgggcagatgtgcccggcgctcgggccccgcgctctgcgtcctgcaggga

SEQ ID NO: 517 - DNA, 695 bp, Homo sapiens
aggctggagtgcaatggcgtgatctcggctcactgcaacctctgcctcccgggttcaagcaattctcctggctcagcctcccgagtagctaggactacaggcgtgcgccaccacgccccgctaattttgtatttttagcagagacggggtttctccatgttggtcaggctggtctcgaaacaccgacctcaggtaatccgcccacctcagcctcccaaagtgctgggattacaggcgtgagccaccgcgcccgaccaacacttgaattttaatacttactttgctactaactgtgtcgttgaaacagtatgttgctttccttctctggcccttagcttaactatctgtacagcgggaacattggcaaactcctacgtggaggagcttgcaggtccacttcaagttatcagactctaactaggagaaaacattgagaatttaggctgaaaataacgacacagtttatcacctcgttaattcccaaacatcattaccgcgtcctaagccaccccacttgcctctcccctagccgccttcctttaaaaaaagaattcccctctagagggcgcgcaagctctgcggccttcgttcctctcttccgtttgccttcactcttccttctgcgcatgtgctcagttcgctatccggctgcttgtacctggttcaggaggttcaggcagaggttgcacccacta

SEQ ID NO: 518 - DNA, 837 bp, Homo sapiens
ggcgacggccccggggaggcggcggccagtccagaggccgagcagtttccggagagctcagagctggaggacgacgacgccgagggcctgtcctcccgactcagcggcaccctcagcttcaccagcgccgaggacgacgaggacgacgaggacgaggacgacgaggaggctggccctgaccagctgcccctcggggatgggacgtcaggagaagacgcaggcgagtgcaggaggacggaggcgggaaccctggggctcgattaggcctccatcccagccgcgttcccagagcatccagggcgctaagttgatccagctgaaactcccacacttcttaaagcgcctgggccacctcttggctgtgtgaccttgagagagtcttaacccccctctgagccaagttttcccttgtgtcaagtgggagaactcactctcatctcagaattgctgcaggcattaaatgtacagggcttgggatccgaatggaaatgatgtattaacgatattttgagggtggagctattaatttagcccctactgtctgccaggcacggtgtttggctcctcggtgcctctctgtccctcagtttccatttctgtaaaaagggggtaagtcctgcctcacactgggaggatgagtgaggccacgtcagtggttgatgtcgaccctggccctaggcccattgatatgactggtcatgtgtctgcagaacggagccccccacctgatgggcagtggggcagtcagctcctggcgcggcagctgcaggatttctggaagaagtcccggaacaccttggcaccccagcggctgctcttcgaagtgaccagcgctaa

SEQ ID NO: 519 - DNA, 283 bp, Homo sapiens
aagcagaggtgcaatttgaagctagattctcccactcttaaactagtgccggacagacgaaggcctagcacatgggctacggagccctagtagattccagtccccacactgagccaggaggggtcgctgcccagggatacctacagccatactcgagctggacaaggcgcacgctctttgtggaagcaaacacgtccaccaccgcgtagaggggctgcgcagctggcagtccccgggcgctcgggcccatgtcctcgccgttgatgatgatgtgcatgtcggc

SEQ ID NO: 520 - DNA, 380 bp, Homo sapiens
gggacatcgtgcttgagagctttgcagtttggccttttcttttctctaccggagatgggaggcctctgtccgtgtttctgctgtgttcaccaggaggccagtctggggcaattactgctccccatctcccaaaggagccacatcagcctcatagcagtgtctctagcttcgagctgttgcctgtggcaggcagaacagtgtgtactcgcaacaagcaaatccccagatgtgagcccattcggggctgccacgttttttcctgatgaaattcctgacttggtgtgttgtgtttttttaaaaaacacgcccactgcctccaaaaacctcagccggggaagagaaacatttctcgttgaattcccagtttttgaaacggtgtc

SEQ ID NO: 521 - DNA, 413 bp, Homo sapiens
agtgtttcatggcaggttgatcagggtttgcatcggatgccccatctgccgggctagttctggctgggagggtgttaggaggagatgctgtgctcggacctgcgaagtcacgggggagatggggacccacttgggccttaattataacacatcccataatgatatggtgttataatacgcacacaaagtgtcctaatctgatattatatctgggttttgtacaatagatacaatcggctggctgcagtttgctaattcattctcgacctatctccacgctccacctagatgactatcggcttccttaattaagtggaaggcaagctctgcaggcgatgcttgcagtcccagcaccgacgagtccggcctggccctcccataagctgggataatagctactcccacatggggcg

SEQ ID NO: 522 - DNA, 534 bp, Homo sapiens
tgggctgcggtgggctcggctgtccactagcgcagtacttggcagcggccggcgtgggccgccttggccttgtggactatgacgtggtagagatgagcaacctggcccgccaagtgctgcatggcgaggcactggctggccaggccaaggccttttcggccgccgcctcgctgcgccgcctcaattcggcagtggaatgcgtgccgtacactcaggcccttacgccagccactgccctagacctggtccgccgatatgatgtggtggctgactgctcggacaacgtgcccactcgctacctggttaatgacgcatgtgtgctggcgggtcggcccctcgtgtctgccagtgccttgcgcttcgagggccaaatcacagtctaccattatgacggtggcccttgctatcgctgcatattcccccaaccacccccagcggagacagtgaccaactgcgcggacggcggggtgctcggtgtcgttaccggggtcctgggctgcctgcaggccttggaagtgctgaaaatcgctgcgg

SEQ ID NO: 523 - DNA, 444 bp, Homo sapiens
gggacagacagtgatgaggccaagtctcagccccctgggaaacacattccggcgggaaagaggctgaaacagggaaactgtggcaaatgataccagcgagggctaggtccctgacacaaggaaacccaaggaagcatttggcttaagcggtgacaatatccctctcctcagatgccaaggtcaaggcgagccgcggcaacaccatctccccagacacacacgcaaaatcggcccttctcggggaaagggcaggggctgaagggggaggcggggtgggcggcggctggaaaaaagtgaaatttcctctcctcctaaaataagtcattggagcgaggagagcgagcgctatctcagtaggctgtgggcgaggtgcgggggtggcgggggcacgctccgggtccgccaggaggggagggatcggaaggggaagcggggagggaaagg

SEQ ID NO: 524 - DNA, 479 bp, Homo sapiens
taaacttgccagcacagacaagacagcttgttcaagctgcacctcaggccgggtcagagaataaaaccgagggctagaaggcccagaatgtcggacagcccagcggcacccgtcagggagtcccaggcgcccgaaagaggcgccgcacctctggcgagtctaggacccatcttcctggacctgtgctctggagtgcctgcgggcctgggtctaatttctgcttctggcaggtgtccccctcccccgccgctaataccaggagcgctgcttctgcggtaacttattttacccccagaagcctgttttgggaccgaagtgtcagggtcctgtgtgtctttttatgcactgttctccttagtgcaaagccctgcagatatgcttggccgaaaagtctcagtggtttcaacttccagattttgttcccgcgtccggtcggaaaatcacctgggatttggctgctgtctaaggccgggggaaag

SEQ ID NO: 525 - DNA, 436 bp, Homo sapiens
agaccgcccgggacccgcttctatcgcggcaccgtggtgtagctccctccggggtcgtatgctgtatgtttactagccgaattctcagtcctggacgctccgtggggccagggaatatgatctgtgctccgctgcagcctcgcgcccagagtaaatatcggtcggatgaactaatcttacatataaaacagaaatcatttcccttttttaatatggcacgtggctcttccctaagtcgccctgggttaaataccgactcaattaacaagatgctaaaaaccgtttacgtttttttacgttttttacgtttaattcaccagacggtgcgctccacgagggaaggggcctggcctccctagttcagggttgtgcttagtgcctgcaaccggccgggcccgcgggggcagccaggagagaaaggcgcgcccagcgcccc

SEQ ID NO: 526 - DNA, 507 bp, Homo sapiens
tgggctctgcctcgtgcaccgcgtgctgcagcccacagccggccgccgccgggctggcgcgagagtggagggtggcagtgcgcaggcgcggagcggaaaagagggggcgtgaagggggcggagccaggcagggttgggggagggggtgatgtactgtgggggcaggggggaaggggagtagggagggggagggggagcacggatgaggaggaccccagagggggcactggtggacctgggggggtggatgaggaggggaggagggtcccatacagggcactgggagacctggggtgggggtggataaggagaggaggagagcctgcagggcactggaagactgggtagtgggggagcgcctaaggaggggaggagggtcctgcaggggcacggaaataccttggggttgaagtggatgaggaagggaggagggccccgcagggggcgctgggagacccgggggggaagtaaggagggcacgagggcctgcagggggcctgggagatc

SEQ ID NO: 527 - DNA, 449 bp, Homo sapiens
cctcaaagagcgtgcgcacctgcccgcgcgcgccggagctgacctctcccggcggcgggcggttaggggaaagtacctgggggaaaggtagaggaggtaaggggacccttggggatgcccctacgggctaccagggttgaacgcacaggcatggtcacgtcggggtattgccaagcttttggcgcagctggtcgggtggccaggctgcatgcggcttagcaggagacgtcctgggctgtttgcgcaggacctctggaggccctcgagatcgtcgcaagtggaaaggtaaagcggaacaagggacaggctggagacgggggtcgcgtctaacatcaggataacttacaattcgtttccaaagagcgcggtacgcgcagagctggggaaaggtgttggatccggcgccagtccttggacgatcagtcgtcgaaaaggaggcaggggccggc

SEQ ID NO: 528 - DNA, 527 bp, Homo sapiens
tgaaccccggggaggtttgacgatctgcccaaagtccctgggaagtttccggccgttctagggctaaagctcagctctctcgactgcatctccttcccacccccatgtggctacccccatagaatggcgcatccacgtagatggcccagatgttgggtttaagggagagagagaaaggaagtttggggcatctgtttatgagagaaaatcgtcagccgtgcgtggaaatcttatgcatagcaaataaatgatgggcaaatacccacttgcatattcagaggcacatttcagatgtgaacatcctgtctcaccaaaacaaccccagctaagaagttccgtcgggggtggaggactttttccaagcctttgtttctgtggagtctctgtgcatggcctgctgtgatgaggtaggggttgacccagaagtctaaggaggccaccaccccgtaggaaagaggagccagcagacagcagcgccgggggttattggggacccccaaatggggaaaggtaggggtgtagacaaa

SEQ ID NO: 529 - DNA, 793 bp, Homo sapiens
aggaagtcaagactgcagtgagccaagatggcgtcaccgcattccagcccgggaaacagggagactctgtttcaaataaaaaatgttgctagtattctaaacacttaggttttaaagggcttgctagctttgctatcggggtattatctgaaggcacgaaaactcttaggggagtgtcctgcttaatgatagccagcattgatccatatttcatagtcttattaattggagatgtttgggatgatttcatcaatttaattagatcttcacttgtctttacccctaatgtggctgacagatagcttggattaggactggatgtggggccgccaaatttgtttgagtaaataatacataagttttattggcgctcaggcccatccgtcgtgccgtctttgactcttatgctatgacagcagcagcgtcgagtagttgctgcagagaccgtatagccctgaaagcctaaaatattatagttaccaggtggccttttgcagaaaaagtggttattcccaaactcaggtcggggtggagggtgagggccacatcctggtaccatcctggctacatcggaatttccagggcccttgaagaccgaggtcgaggccccttccttggagacctagcaggcagggagcctgggactgtgtatggaactgagcctctggggccatcatggagctcggcaagaggctcaggaggatctggaaagcacgtggacttcccaggatgctggcagcgtccggtgtttctcaaattgcgttctgcgcatagaccacctggggtgctgccc

SEQ ID NO: 530 - DNA, 539 bp, Homo sapiens
gcttggaaagggccctgcgcccagcacacagcccccagtgaaagagaaccggttccaggaactccgttcctgtaaacggatttcttttgataagtgccctgtcaattaaaacccaaactgattttctgttctttctcaactggtgtctcccttccagaaagttcagcagtacacagtcatcgttcaggccacagatatggaaggaaatctcaactatggcctctcaaacacagccacagccatcatcacggtgacagatgtgaatgacaacccgccagaatttaccgccagcacggtgagtccctcgaagctgcccagtgacgcatggcccgtgcagggcaggaatgcactggcgtgcggcacagcctcctctctctctgcggctctgcctgacgggcacttggcagatagcgtgtaagaaaggccctgggcagcggaggctgctgtgtggagtggctcgattcctgatgtgcgcagtaggagagaggccggggctcagtcagggaggggaccggcggcggaggtcggttgccacctgg

SEQ ID NO: 531 - DNA, 433 bp, Homo sapiens
ctccctcattcacccctccccagactttgatgaaaccatctgtggctcccggcctctgcaggacccagcccctcagctcctcagcccctgcaggtgtccctcggtactgcctcacgccatcctgccccaggtgataactatgccctgagcccccaccatttccactgggcacatcccagaaatacgcattcacagaccaggcacgcccatgactgcggccagattttaaaaggaatcaccagaaatgtaatctccagtggtcctagagccccctgggcacccagcttctggcaccctgtggacacccaggagtgggggtcggcccaaccagctgcgcagccgcccagccctgccagcggggagcttcctccctccctgccctccggatacaccctcaccccaactgagcgagcccagggttgggggaggtgca

SEQ ID NO: 532 - DNA, 403 bp, Homo sapiens
agacagtctctgagctttgccgcccctggaagcagaggcgggaaacgaccggataaaacagaacgatggggagtttcatcctgtcatttattttgagtctgactacagatcattatttctcattttggatttcagatggtttgaaatgcaagtctagctgctcaaccatggggctctagaaagaggtgcctggctatcactcaaggggttgtgggagcccaggacggtcggaggcgctaggagctctgggattgcctgtgaggccgcgcagagacaggagtttaagatgaatgaagcaccccaaccttgcgctctgggaatgcgccgtgtttaaggctcccctggtcctctcccgggtgccccggacccttcccaagggctgctctccctctcaccgagagcc

SEQ ID NO: 533 - DNA, 403 bp, Homo sapiens
cctgcagcccctcctcacgtctctgctcttccggctgccaccaggctcccggcacgcggcccttcttccttctgagttgtttttcaggggctgcgctggggtcccacggacacctcgccagaatcagctgggttcccatgggaaggggaagtgccgctcctgcgtccctgcacccttgccctctgtgtggtgtggcgtgtctatcccgactggcgaggccgcgagcccatagtttgctgctgctccttcaccgtccgtgctcattcctcagcctggtacagccccaaccccctcccacctttgtgtggttccaggcccatgcatgacacgcgtgttccatttccacgtgctaccggcaccagagcccattctgtacatgctcgtttttacaaccgctttttaa

SEQ ID NO: 534 - DNA, 383 bp, Homo sapiens
cgcaaggaggggcacccctagcacatctgggttactccatcgcgccagccggtgtctgcagcctgaaaatcgcgtctctggtcttgttccccagcgagccgttcaggactctctgtccgcctgcagcccccgccttccctggtacctccgcagagacccaagaggcgtccagaggggagcccaggcgcacggtccccagggaggggccccgccctcaggtgggtgccgaggtggccgcccacagagcagcaggtgcgttggaggaggggcgccttgtgcggctgaatctcgtgtgcaggaggagagggcgaggctctgcagaaacatcagacccggccatccctgtagccgcgcccctggcccagcctcaggctctgcagctg

SEQ ID NO: 535 - DNA, 437 bp, Homo sapiens
agccacatgtggctggcagctcctgtatggggcaacgtggtctgagagccggggagagctggcagggaggcgctgcaggcacagccccccaagacccccaaagctttgtgggggacccgtgcagctgcacctccccctggggtccgcagctggcactgggcttccttcctgccccatcagccacagggagctccacactgcgtgagaaggccctgtgacccgaggtcggacaggcggacaggcccacagtgagctcgagacccacagacactgaccgagacccgacaggacagacatgcaccaaaaacacgcggacacgtgtgcttgagaagaggagggggagcccgaacaggcacgggggagcccgcacaggcatgagggggcagccggtgttgggggctcctcctccagcacgggggcaggaggcagtgagatgg

SEQ ID NO: 536 - DNA, 425 bp, Homo sapiens
caccctggaggaggagatggaaccctcctgtttgctggggctcggcccccggtgaggctggctgcaggtgaccacagagacctagcctggtgtcgtgcggccgctgggtccgtgaaggggagtgaggcactcctcaccctgtggcagctcctagtcaggaggcctctcccctccacctgcacgtcatgtggctgtggctcccaggaccagcgcctggagcgggtgagtgaacgtccctctcctccgtgggcactgcctccgctgtgagccacccttcaggctacgtgcatttccgcctgttctcagggtttaaccgaaaccgaacattaaccgtagccctaacgcggctgagggatccaggccttgcctctgggccgggtgagagacagacactcgccaaccagtccgcagtggctccccgtgtg

SEQ ID NO: 537 - DNA, 465 bp, Homo sapiens
tgccaagacagctgccgctcctgccggcctagggccgcagccggaatcccgggccacatcccctgccccgttgtgccttccacacctcgggcagtcactaggaaaagggtcgccaactgaaaggcctgcaggaaccaggatgatacctgcgtcagtcccgcggctgctgcgagtgcgcgctctcctgccagggggacctcagaccctcctttacagcacaccgagggccctgcagacacgcgagcgggccttcagtttgcaaaccctgaaagcgggcgcggtccaccaggacgatctggcagggctctgggtgaggaggccgcgtctttatttggggtcctcgggcagccacgttgcagctctgggggaagactgcttaaggaacccgctctgaactgcgcgctggtgtcctctccggccctcgcttccccgaccccgcacaggctaacgggagacgcgcaggcc

SEQ ID NO: 538 - DNA, 507 bp, Homo sapiens
agtgtccttttccgagggcccgaggctccccctccagcagggagagctccgggacggcccccaccaagggttgggtttcttccttcacaattccacagaggcatccctgtccttcctacctgggaaacctcgaggtgcggtgcccgtgtacttctggtactttgcgtggtgccatcagggaccccagagccacagctgcgtgtgtgtgtggatgtgtgtgtgtgtgtgcgcgcgcgcgcgtgtacggcgaaaggatgtgcttgggggagccgagtacacaacgtctgcttgggcagctgctgggcaggcgttgggcctggaggtatctcacacccacgtatcttccagtcttcaaacacggcattgctctgcctcccgtagcgcgcttcgaacctgcctcgcggacacgtgaacagaggctgtccctgggaagataagtgcgctttcccgtaaaatccgggaaatttgccttgaggaaagtttccgttcttgttacttgtcgggttt

SEQ ID NO: 539 - DNA, 323 bp, Homo sapiens
gtgcgcggaggactcctgggccgcacacccatttccaggctgcgggagccggacaggggagggcagaggggggacaaaaggactctttaggtccaaaatgaccctgaaggagagtccagaatgcccagtggccgcgtctgcaacggagtcttctttctccaattgccttctgccccatcaccatgggccccacctgcgccacctgcgcccaccctgtgaccctggctcagcgaccttggcccttaatcgcccaacgccgattcctcaaaattccggctgcgctgaatcgggctgcttttgccgccgccccggcagttgggccc

SEQ ID NO: 540 - DNA, 582 bp, Homo sapiens
gcaggcagaccctgcctgggacagtcagggtgcaccggttctgtggctccggctcttaagcactgctctggaccactcctagcttgagggggttgggggaggtcaccacaggctggcggattgcttgggcagagtcagaggtaggattgggcttgggaagaaaggtgcagggtgcctgaagcaggggcagccaggcagtgtggaaccagggggctctgaggacagcgtccactctcttggcagaagtgtgcaggtcctgctgtgtctgtcccgtccctcctcccatccacctcctacttcattgggcaaactgtttccttctttggctcactcgggatcccccagcaatgtcctttgttctcgaactcccagtgactcgaggggtgtcctctgattgctcacctcaggtggagcctcgaaaggatggcgctgctgttcctacctcgggtctccccagtgcacggcatccttacctgtgggctccacagggttccatgcaggcattttgctcccataccccgatgtccaagcccggctggacaaggagaagcaatgtgatgaccgaggggctgagatggctgg

SEQ ID NO: 541 - DNA, 842 bp, Homo sapiens
cccgcccttctgccccttccctggcctgcagacctgtgacctgccacaccggcctgctgctgatggattatcctgataatcaactctctgctattgtttagcaaaagcctttccctttgtcctattatcagaatccccctgcacttctcagtccctggtgacagatccacctcccactgtggctggaatttcccctgaacgttcttgtgcaggaaggcttagttctctatcaaaaataagtctctctgctccttaatagtgggtctttttaaaaatttccctgaagctgtttctcgttgtcctttgccttcctgaccaagacaatgtcagcatggggttctgtccttggtcactgctcagaggtggagcttgaccctgggatgtcgggaagacgctctgggaaaagctgtggctgaagctgctgagtccaaagggggtgtctccttcccagtagtttcagacgaggatctctcagccaggactcttctggagcctggaaaccaagccacaggacgaagggctgcgggaagaaggaggctgagcctggggaacctggtggctgccttcgggtgtcagaagggctttcccccagcacgcgtttccccagaggctggattggggccactgaggcggtgtgacagggaggtagatttcagctcagcaggaggaagggtcccctgaaagccagggctcccagtgacacactgggcgcctggtgattactcagcttctgggtgctgagggctctgaaaggcaggcacacttgtcccactgtatcataacagaaaaaaccggaacggcccatgtgcccattagctggtggatggataaacacagcgtga

SEQ ID NO: 542 - DNA, 414 bp, Homo sapiens
gttgggactgtctaggcgaaagctcagtgtgaattgtctgctgtgttcccggtgtccaaggcccccaggggctgggtgagtcccaagagggatgtaggccgagcagagttgggcactgaacccccaggctccccttgttgaccacccagccagaaattcccagggcagaaggtggggtctgctcacccatggggcaggcggtctggggatgtctgacgcctgggcctctggccagcaggatgtggacagaagtggccgctgggtggggctgagtgcagagggcagtgcccatctgcagcgtggctatgcgccccacagagtgtgggcctgaagcggggaaaggacccccctcaacacctcaggccggcgggtcccattccagctgccacccggactcagcctgaggcctcctca

SEQ ID NO: 543 - DNA, 601 bp, Homo sapiens
gcttcagcctcccggagtagctgggattacaggcacttgccaccacgcccggctacttttttgtatttttagtagagagggttttgccatgttggccaggctggtctccaatgctgtcttcttttaagatttgattttccgttgctggctggtgtggggatgtggagcgccctcaccctgtgaaggcatcccttcccgtcactgtgtcggtccctgtggttttcgctgcccgtgtcccctgtgctgctgctccttgctgctggcctctggctgtgtcaccagctctggagtggccctagactcacgagagagcatgcaggtcccaaaccgagagtggctggaggcaggagtgctgcaggagtgcctgggccagccccctgtccaagtagggagctctgcccagctcccaaaagagcctcctcacaaagccagtcactgagcccgagcagcgcggtcgcccctgacgcggcaccttccttccaggctcctacctggctcagggcgtccagcggcagccagacaggctgggggcccctccatgttgcgcgctccggctgactcaccggggctgggggcccttccacgcggcgcgctccagctg

SEQ ID NO: 544 - DNA, 588 bp, Homo sapiens
agctgccatgtgtgagggcttgctctgagctaagcatgattctgagcaccggcagggagtacagcaagtggatgctgaggcctggagggcaggccccagagagctgagtgcccctccaggtttcatggctggcactgtggaccagtgtggcccactgcctgcttgccaccccacaacaaagcttcagaggtgcagaaccagtggcctgagctccagggacaggaagcatcttggcgtggggttctcatggtggcacttgcatgccctagtgcacaggggcggaaggggaagctctgttgcctgggccctttctctgtggcaacagatggggaagtctgccgtgaggatagagagtgcatggccatcccatcacagcacaggttgagtgctgagccaccccgagccctccccagcccagcggctctccccaaggaggcttcaaaaccccgtgctccctttgtacccccaagcccatgcttggctctcagatccacctcgggccctttgcaccccagactcctccccagctgcacattcccgggattccaggcatctggtgctgtgtgaggtcactaggacagggcagtc

SEQ ID NO: 545 - DNA, 446 bp, Homo sapiens
caagtcgagtctgcgctatacaaacaagagactcttaaggcgattagcccgggctgctgtgagtttctgtctgctcccctccccgtgggcacctgggagcccaggctcctaattaggctgcatgggcccttgcaaaacgctctcactgttcccaggtcaccgcctggcagctgccccagcctctcccgacgttaccgtgcaggctgagcaggaggctggttcactggcccctgacagagcccaccgattctctctccttcctggtgtaggctttacatacgaaccagtgtgagtgatctcaggcatagccatggttctaagactcgcccaggagcacctagggatttgccacccacttgagaaatgaatgtgatgtgcgtcctctgagggcagctccgggaaacctggctcccccacctggacgccccggtgtcccctgtgcatcg

SEQ ID NO: 546 - DNA, 399 bp, Homo sapiens
gccgcccacggcgtcgctaacgaggatgccgcccacgcgatcgctaagccggacgccgcccgcggcatcgctaacgaggttgccgcccagggcatcgctaccgaggacgtcgccgacggcatcgctaacgaggacgccgcccacggcgtcgctaacgaggtcgccgcccagggcgtcgctaacgaggacgccgtcgagggcgtcgctaacgaggacgccgcccagggcgtcgctaaagaggtcaccgcccacggcgtcgctaacgaggacgccgtccagggtgtcgttaacgaggacgccctccacggcgtcgtcgctaacgaggatgccgcccaagcgatcgctaagccggacgccgcccatggcatcgctaacgaggtcaccgcgcagggcatcgct

SEQ ID NO: 547 - DNA, 318 bp, Homo sapiens
gccgcccacggcgtcgctaacgaggatgccgcccacgcgatcgctaagccggatgccgcccacggcatcgctaacgaggtcgccacccagggcatcgctaccgaggatgtcgccgacggcatcgctaatgaggacgccgcccacggcgtcgctaaagaggtcaccgcccacggcgtcgctaacgaggacgccgtccagggcgtcgttaacgaggacgccctccacggcgtcgtcgctaacgaggatgccgcccaagcgatcgctaagccggacgccgcccatggcatcgctaacgaggtcgccgcccacggcatcgcg

SEQ ID NO: 548 - DNA, 396 bp, Homo sapiens
ctccacggcgtcgtcgctaacgaggatgccgcccaagcgatcgctaagccggacgccgcccatggcatcgctaacgaggtcgccgcccacggcatcgcgaacgaggacgccgcccagggcatcgctaacgaggtcgccgcccacggcatctctaaggaggacgctgtccagggcatcgcgaacgaggacgccgcccagggcatcgcgaacgaggacgccgcccagggcatcgcacacgaggacgccgtccagggcatcgctaacgaggacgccgtccacggcgtcgctaaagaggtcgccgcccacggcgtcgctaacaaggatgccgcccacgcgatcgctaagccggacgccgcccgcggcatcgctaacgaggttgccgcccagggcatcgct

SEQ ID NO: 549 - DNA, 426 bp, Homo sapiens
gccgcccacggcgtcgctaacgaggatgccgcccacgcgatcgctaagccggatgccgcccacggcatcgctaacgaggtcgccacccagggcatcgctaccgaggatgtcgccgacggcatcgctaacgaggacgccgcccacggcgtcgctaacgaggtcgccgcccacggcgtcgctatcgaggacgccgtccagggcgtcgctaacgaggacgccgtccacggcgtcgctaacgaggacgccgcccagggcgtcgctaaagaggtcaccgcccacggcgtcgctaacgaggacgccgtccagggcgtcgttaatgaggacgccctccacagcgtcttcgctaacgaggatgccgcccaagcgatcgctaagccggacgccgcccatggcatcgctaacgaggtcgccgcccacggcatcggg

SEQ ID NO: 550 - DNA, 777 bp, Homo sapiens
ctccacagcgtcttcgctaacgaggatgccgcccaagcgatcgctaagccggacgccgcccatggcatcgctaacgaggtcgccgcccacggcatcgggaacgaggacgctgcccagggcatcgcgaacgaggacgccgcccagggcatcgctaacgaggtcgccgcccacggcatctctaaggaggacgccgtccagggcatcgcgaacgaggacgccgcccagggcatcgcgaacgaggacgccgcccagggcatcgcacacgaggacgccgtccagggcattgctaacgaggacgccgtccacggcgtcgctaaagaggtcgccgcccacggcgtcgctaacgaggaccctgtccagggcgtcgctaacgaggacgtcgtccagggcgtcgctaacgaggttgaagtccagggcatcgcacacgaggacgccgtccagggcatcgctaacgaggacgccgcccacggcatcgctaacgaggtcgccgcccacggcgtcgcgaacgaggacgccgcccagggcgtcgctaacgaggacgccgcccagggcgtcgctaacgaggacgccgtccacggcgtcgctaacgaggacgccgcccagggcgtcgctaaagaggtcaccgcccacggcgtcactaacgaggacgccgtccagggcgtcgttaaccaggaggccctccatggcgtcgtcgctaacgaggatgccgcccaagcgatcgctaagccggacagcgcccatggcatcgctaacgaggtcgccgcccagggcatcgct

SEQ ID NO: 551 - DNA, 399 bp, Homo sapiens
gccgcccacggcgtcgctaacgaggatgccgcccacgcgatcgctaagccggacgccgcccgcggcatcgctaacgaggtcgccacccagggcatcgctacccaggatgtcgccgacggcatcgctaacgaggacgccgcccacggcgtcgctaacgaggtcgccgcccacggcatcgctaacgaggtcgccgtccactgcgtcgctatcgaggacgccgcccagggcgtcgctaaagaggtcaccgcccacggcgtcgctaacgaggacgccgtccagggcgtcgttaacgagtacgccctccacagcgtcgtcgctaacgaggatgccgcccaagcaatcgctaagccggacgccgcccatggcatcgctaacgaggtcgccgcccacagcatcgcg

SEQ ID NO: 552 - DNA, 720 bp, Homo sapiens
ctccacagcgtcgtcgctaacgaggatgccgcccaagcaatcgctaagccggacgccgcccatggcatcgctaacgaggtcgccgcccacagcatcgcgaacgaggacgctgcccagggcatcgcgaacgaggacgccgcccagggcatcgctaacgaggtcgccgcccacggcatctctaaggaggacgccgtccagggcatcgcgaacgaggacgccgcccagggcatcgcgaacgaggacgccgcccagggcatcgcacacgaggacgccgtccagggcattgctaacgaggacgccgtccacggcgtcgctaaagaggtcgccgcccacggcgtcgctaacgaggaccccgtccagggcgtcgctaacgaggacgccgtccagggcgtcgctaacgaggttgaagtccagggcgtcgcacacgaggacgccgtccagggcatcgctaacgaggacgccgcccacggcatcactaacgaggtcgccgcccacggcatcgcgaacgaggacgccgcccagggcatcgcgaacgaggacgccgcccagggcatcgcacacgaggacgccgtccagggcatcgctaacgaggacgccgtccacagcgtcgctaaagaggtcgccgcccatggcgtcgctaacgaggatgccgcccacgcgatcgctaagccggacgccgcccgcggcatcgctaacgaggttgccgcccagggcatcgct

SEQ ID NO: 553 - DNA, 828 bp, Homo sapiens
gccgcccatggcgtcgctaacgaggatgccgcccacgcgatcgctaagccggacgccgcccgcggcatcgctaacgaggttgccgcccagggcatcgctaccgaggatgtcgccgacggcatcgctaacgaggacgccgcccacggcgtcgctaacgaggtcgccgcccacggcatctctaaggaggacgccgtccagggcatcgcgaacgaggacgccgcccagggcatcgctaaggaggaccccatccagggcgtcgctaacgaggttgaagtccagggcgtcgcacacgaggacgccgtccagggcatcgctaacgaggacgccgcccacggcatcgccaacgaggacgctgcccagggcatcgcgaacgaggacgccgcccaggacatcgcaaacgaggacgccgcccagggcatcgctaaggaggaccccgtccagggcgtcgctaacgaggaccccgtccagggcgtcgctaacgaggacgccgtccagggcgtcgctaacgaggttgaagtccagggcgtcgcacacgaggacgccgtccagggcatcgctaacgaggacgccgcccacggcatcgctaacgaggtcgccgcccacggcatcgcgaacgaggacgccgcccagggcatcgcgaacgaggacgccgcccagggcattgcacacgaggacgccgtccagggcatcgctaacgaggacgccgtccacggcgtcgctaaagaggtcgccgcccacggcgtcgctaacgaggatgccgcccacgcgatcgctaagccggacgccgcccgcggcatcgctaacgaggtcgccgcccagggcatcgct

SEQ ID NO: 554 - DNA, 814 bp, Homo sapiens
gccgcccacggcgtcgctaacgaggatgccgcccaagcgatcgctaagccggacgccgcccatggcatcgctaatgaggtcgccgcccagggcatcgctaacgaggtcgccgtccacagcatcgctatcgaggacaccacaccgtccagggcgtggctaacgaagtcgctgcccagggcattgctaccgaggatgtcgccgacggcatcgctgaggacgccgtccagggcatcactaacgaggaggccgcccaggccatcgctaagcaggacgccgcccacggcatcactaacgaggacgccgcccagggcgtcactaacgaggtcgccgcccagggcgtcgctaacgaggacgccgcccacggcgtcgctaacgaggtcgccgcccacggcgtcgctaacgaggacgccgcccagggcgtcgctaacgaggtcgccgcccagggcgtcgctaacgaggacgccgtccacggcgtcgctaacgaggacgccgtccagggcgtcgctaacgaggtcgaagtccagggcatcgcacacaaggacgccatccagggcatcgctaacgaggacgccgcccagggcgtcgctaacgaggacgccgcccagggcgtcgctaacgaggtcgccgcccacggcgtcgctaacgaggatgccgcccacggcgtcgctaacgaggtcgccgcccagggcgtcgctaacgaggacgccgcccagggcgtcgctaacgaggtcgccgtccacggcgtcgctaacgaggacgccgcccggcacatggctaaggaggacgccgcccaggacatcgctaacgaggacgc

SEQ ID NO: 555 - DNA, 372 bp, Homo sapiens
ctggacccccctctgtggcaggtacaagcccccaaccccctcccggtcccggggcactcactggggcccgtggacgccagggctgcacaggcagcgccccgactggaagtcgcagtgtccgctgccgcagtcggcgcacacgaaggcgcagtcctcgccgtaagtgccattgctacacttggtctcgcaccttggaaagaggggaggaggcgtcagcagaaatggaggcaaacggaccacagcgccctcgacattggggcctttgttagggaggcagggcgggggactgcgtgtctgggccgacgcaggcagggctgctcaccggtcgccgatccagcccgcgttgcagcgcgtacacttgccggtgacatg

SEQ ID NO: 556 - DNA, 504 bp, Homo sapiens
catttcacattcacacaagcatttcacaaaagccgtcatgtgtgtctaccggatttgctcattataatcgtctattttttttgaaataggcttgctcatatttaattagcttgaattgtgttacaataaatattatagtgctttaagaaaaatgtctgaaacctttgaggcaggtacagagggaaggttttcccatataggctgcacgtgtgcatgttcgggcccacgctaccttcaggctgatgtctgtccagtagatgtggttgctggacacatcaaagtccagggccgaggcctccttgacgccgagagcaggatggccacatcgttgttgttggtctcgagggagatcctgtggatggcggctctactggtgaagaccaagaaggccttgggcatgatgcaggtcttcatgtcactcagcagttccaggccctagatggggcagccaaaccgggttgtgtggggcgtgaagaagcacaggtggctgcacccccattcctg

SEQ ID NO: 557 - DNA, 422 bp, Homo sapiens
tggggccgccccagcgggcagcagccaaggtggtgggctgcggggcgcccggggcacagccgtgacctgcccacacctgcaggtgctgaggagccacgtgatggtgcgagtgggtggtggctgggacacgctggagcattacctggacaagcacgacccgtgccgctgctcctccactggtcagtgccagggtggggctggggctggacgggcaggggacttgcttctgtggctctgtccctcacatgctgcctgtcctctctccccgcagctcatcgcccaccccagccgagggtctgcaccttttctccacagagggtgtcgcccaccaccagtccccgccctgctagcccagtccctgggagtgagcgccggggctcccggcctgagatgactcccgttagcttacgaagcacaaagga

SEQ ID NO: 558 - DNA, 714 bp, Homo sapiens
ggccagcggctgcagtccgcggctttcagcacccctcccttctctgtgccgggcttttcggccgaacgttctcgctccttctgcgttgactgttttatgtgtggttaacgcgcggctcaccaggggggattttgaagcacctgattcggtcgggcgcaaattgccttggttagtgggcccgaccgtgccgcactgtccagggagcgtcaagggtaggtgtctcgctgggagtcacatttcaagtggaaaggagaatcctgttgcagttccttggtgtcgcatttcggttcactacacacaagccactgccttcttgcctgggggctccccgctcttctcacggtggctttcctctctgcagagacctctgcgcgaggtatacctaagcgcattgtgtgcgtcttcgggaaaaggcgaaaactactcgcagtcccacgtggatttcattttttcccctttacctactcttagggtgcgtggtaggcatcattatccaccttagctgcccttcttttgggttttctgtaaaactggaaaaacgagaagaaactgttgcacttcaaatttagttgttttgttttgttttttggagacggagtttcgctcttgttgcccagactggagtgcaatggcatgatctccgctcaccgcaacgtccgcctcccgggttcaagcaattctcctgcctcagcctcccgagtagctgggattgca

SEQ ID NO: 559 - DNA, 687 bp, Homo sapiens
gaaatggtaaggggaggctggggaaacatccagctgtcagcggcctccccggggcacgctgggatcagccaagagaatgcaacagccaggggtgggagccccctccgcggttgcacacgccaggtccctccagtcttgcaggttcaaatgaaggcttatgtgaagggggaatcgctctggacagctcagtgcccctcagagtcacatcaccaccattttccaccacgcggaggtggttgaaaaaaaggagtcacagatgggtaaggtttgaggggcgtcgggctgacgaggcaaaaaaaaaagtaggtgctgtggtgctgatgggaggttctccctccctcacaggacgccaggaaggaaatggggccgctacagaggtggtgaggtgccagggcccacggagggcccacctgttgcgcgggggcgagcaccaggcgctgggtgcacgggggcccgtgcgtgcctgtgtacgtgcgtgggtgtgcgcgcacgtgtgctcaggccgcgcgtgcaggggccgtgcgtgcgtttgtgtgcggtgcgcgcgcgtgtgtcccgcgtgtgcacgggcgtggcagaggcgggcgcgcgacctggccgcggtagtgcgtgcccgtcgctctgcgcgcccgcccgccggagcgcagcatcgcctccggccaggagtgtgcatgcgtggggtgagtg

SEQ ID NO: 560 - DNA, 546 bp, Homo sapiens
tcagcgagaggagccgacccttgggggacagcggggtcgggttccaagccggggctgctggggctggtcaggccgcccgccctgcttgacccctctcacctgatggctcccttctccaatcttcaagaaggtgaaacgtcactgtggtccgcagagggcaggccgcgtgtgcggagtcgctgtcatcccttagtggccacgcccgcctccctgtccacttcccgtcttcgtcgttgcggttcaggtgttcgctccactctgcggcaccaaaggcccgtcacgtgcttcccctgcccagacaccccaaacaccgaaactgtcatccgaaggccccgcacctgccgcctgtcggatgctgaagaacgaatcccaggtggagaggacagcaccccaaaagggatgggggatggattggggcgtctgagccccgtggagtatgtcagcctttcagagcgacccgctccacgctgcgatgtggtccttaagaagttagggccgggcacggcgcctcacgctgtaatcccagcactttgggagccaaggtgg

SEQ ID NO: 561 - DNA, 817 bp, Homo sapiens
acaaaaaattagccaggcgtggtggcgggcgcctgtagtcccagctacccgggaggctgaggaaggagaatggcgtgagctcaggaggcggagcttgcagtgagccgagatcacgccactactctagcctgggcaacagtgagactcttgtctgaaaaaaaaaaaaacaaaaacaaaactctcatttcatcatgagttatgcggggaaatgggatttgaaatatggacattttgccttctgccagttagaagcatgtctgttccctaaatgtgggcgccccctgcgtctgtacgtagatctccacctcgctgctttattttccagaggttactgtagctccttactttcagtcctcaatagcgtcatgaaatttcttccattattttttcttttctggattccagttagcatttccccccaactcatgacttctgctgaataagagaaaagagcaatatttaggttcatttcctgtttattattaaagtgattttcacaaatgtttcattacatgcatatgggggacatgatggataaaacaaaactgaatatttgtggcaactaaaaggaagatgtggttatgttggtatttgccacttgcaggcagagtcgttgccaagcgtctggacaatacagtgccctcacaggagccagaagtgaagtctgcgggccgctagcacagcgccctggtgtcctgttccccgagcaatgtggcctgcgtctgctcggcatgttggaggttctgatctcagcggagtaagacacccggcatcggtcagctccctcttcctcctcctgctttctgccacccatctg

SEQ ID NO: 562 - DNA, 280 bp, Homo sapiens
gccaggctggagccaaggtacggcgcgcacacacccggccccgtcctgccggcagcaaacgcccagtgctgtttcatctcagctttcgacctgcccacgtggggacgtgtatcacgcccctttcatagaggatgccactgagtccgcctggaggaacttcctggggtcgccccatacctgcctccgactaaccctactgccccacagaggggcccccacaggacaggccccggcagagagccccaacagccgcgaggctgcccttcctggcctctcgtca

SEQ ID NO: 563 - DNA, 469 bp, Homo sapiens
tgccacgatgactcatactagggcaagttgagggcccgcactgtccagccggagtcggttagagaccctcaggaggctcacaggctccagggcctggatgttaggatgtgaggcctggggttaccgagatcccacacccaggagatgctcgcgtggagccttcgctcacagcggcctgcgcgaatggggcttgctccgagaggttcagctggcaccgagagctggcaccgagagcgtttccagctgcaatctgttcccagcaattaataactgtaaataacaagtccgctcctccgcctggctccgcgctactctccgcttccctcccagcccctcagctgctccccttcccgtgcccaccctccctctcctcccctctttccttccatgtcgtcgagaggtccctgccgtcccttccccgggtccctctgagaacacccagctcccttcccgtaggccatgggggaag

SEQ ID NO: 564 - DNA, 363 bp, Homo sapiens
gagcagcgaccctgccatatgggccccgcacacggagcccccgccagcccggggcctcggcctcgggaagcacctgggaggcggcggggaaggttggccctggctggtctgacacacgcctcggggctcgtccttcatgggccaggcctggtgctccacctaccactgctgggaagccatggaggcttctcaagggtgacctgcgaggtgggggtgcctgccttccctcttgaggtaagaggggtctggtcccaccgcttgccgtaaaaaaggaggagcagctcaggtctgcacgtggccctgagcagctcgccggaaggttccagcctggtctttttattccgtttcccttcccagggccgg

SEQ ID NO: 565 - DNA, 563 bp, Homo sapiens
taccaggagcccgggaagcaccgagctcagggctccttggctcagcacccggctttgccagaccactcgggcctctgcgcctcccgacagcgccgtctggggcattcccctgccagctgagcccagagattgacacgggctgggccaaaggggagccattccagcctcgctcttccctgccagaaccagaaagccacctctctgccctcgtcctggaaaaatgggccaaattccccaaggcaaagattaaacgacaaaacaaatttcaaagaacaagtgggcacctggttctgagccaaatgatgccttccagcatgacaatgcggccctgagcacctgccaagggacaaccatatgttccctggcccgacgtgtccacgagccactgccagcccaagagagctgtggacaataaagatgagaatttttttttttttttgagatggagtctcacactgtcgcccagtctaaagtgcagtggtgcgatctcggctcactgcaacctctgcctcccgggttcaagcgattctcctgcctcagcctcccaagtagctgggattaca

SEQ ID NO: 566 - DNA, 426 bp, Homo sapiens
gctcaccagcggagccgacgtggcctgcaccaccagcacatactcccgccggacctcaaagtccagctccaccatggcacgcaggtccccgttgagcaggtccagctggaagaaatgccgcatgtccccttccacaatctgatacatgatctgggcattagggccttcatcagggtcgttagcacgaatctttgccaccaccgaccccactgggttgttctcctcaacaaacagctccagttcgtccttctcaaacatgggggcattgtcattaatgtccaagatggtcacctggatttctaccgaggcgctaaggggagtgggactgccccgatccacagccagagcccaaaggttgtacacggccacattctcccggtccagccggcgctgggtgcgaatcacaccggacgtgggctcgatgta

SEQ ID NO: 567 - DNA, 666 bp, Homo sapiens
gtccccaccctggaaggtgtacagcagacgcccattgggacctgagtcccggtccgtggcagagacctggaggatgctggtcgagggtggagcatcctcaaagatggaaccctggtagaaatcccacaggaactggggtgcattgtcattggcatcgaggatgaggatctctagggtggtggtgtctgatttctgcgggatgccgttgtcctgggccatgatggtcagcgtgtaggcgacctggttctcatagtccagctccatcatggtgtacatggtgccactgtcggggtcaatgcggaactgcggcacggggtcctgaatcacgtaggtgatgcgggcattctctcctgtgtcctcatcgttggcactgagggtagcaatggaggtgcccacaggcctgtcctcactgacactcactgtgtaatgggagctctgaaagacaggcctgtgggtgttggcatcagtgacgttgattaggacatgcgcagtgtgcgaccgtgtgccgtcggatgctgtcaccgccagcacgtactgctgctcctgcttgtagtccagaggtagcgccagggtgatgaggccgccccctctctggctgctgagtgcaaagcggttccgggtgttgccgcctgtgagctggtaggtaatcacactgttggcgtcacg

SEQ ID NO: 568 - DNA, 473 bp, Homo sapiens
atctagctgcagatcccggctgtctacacagccatctagctgcggatcccggctgtctatactgtgatgcggggacacttgtcatttcggaaggttgctaatgaaaagtcagaaatttcccagagaccccacacaagctagaaaaatgggccgtttgcatgagagatcctccgtcagcaataataacttagcaggagagattccatacgatgggaaatgattgtttggatggtaatttgcttagggaaatcaaaaggagggagagaccctcatgctggagtcagtttccaatgaaaaccaggaggattgcatctggttctttaaggtaggcatgaaagttctcgggagaaagaggagggaggaaggagggctggagagagggaaggatggaggacaagagagagtggcaggtggcctcatccccggggctcttgggctggagcagagatcctgaaagccatcgggtgccgacg

SEQ ID NO: 569 - DNA, 428 bp, Homo sapiens
ccgaccccagccttgttaggggactggccacactcacagacatgccagccggcaagagctggtcatgggtgtgtggaccgtctcagggaatcagtcagaggtggctggggtagccaggagccaccacactgcctgccgacgtggctgggcctgtttcagatccaggaagtgtgggtctgcatggggaaccttctgcttttgtgatgagctgagcggcccctggccaaccctttcggccttgcatggagctcagatgtgtggccgcaggagctgagccccagtcccaggcttgtcctgccctggctctgcctcttgggagcctgtcctgggcagggtggggctgtgatgaagactgggcaggtgaaagctctggggcaccggcctgcctgtccccttcctggagcggacagtgagaggggctttcttgc

SEQ ID NO: 570 - DNA, 505 bp, Homo sapiens
tgcgaagcggtcctgacctcccggccgacgggtgttatctcaggacagccggtgtgcttggctacatcacccctgtctgctggtgggcccctgtctggggcccccaggcgccctctcctgaagggcctgcgcagagctgggggcccgaaccattcctcaccccaactcttcattgtgaaaattgtcatagatacaaaaccgtgaagagaatcacactgcagacacccatctgccccccacaactgtttatgccgtgtgtgggttccacagctgcttaaaaaattgctaaatgttcttcattcctggatatgttgatacacagacgtttacccattcacagatgcactgatgcattcttagacacccttatacttgggagacccacatgtcctggtttgcacgacacttgctggtttcaaagctgaaaatcccgccttgtaccaacccctaagtcccgggcaaggcaggaggaccccgggcaggagacaagtgccctcccccaggg

SEQ ID NO: 571 - DNA, 454 bp, Homo sapiens
cccgcagccctggcagcaaggcggagggcctggctgacacacaccttcccggatttgattatcctcacggctgatccaatcacttgggcagtcaggaaccacgatcatagccaaaaacctcaattccaccaaaaatcgagtttcccagatggcattttcactctttagtcctgaaccactcaccttcacaggtgctaactgtgtttgcctctggagtgatgggaccaaccagaaggcatctgggatagaaagggcactcagaggagcatgaggccctcgatgaaggggtctgggatagaaagggcactcagaggagcgtgaggccctcgatgaaggcgtctgggatagaaagggcactcagaggagcgtgaggccctcgatgaaggcgtctggatctccaaggccgggagataagcccgctaaccggcaccttagtgctccaggacgctgggct

SEQ ID NO: 572 - DNA, 608 bp, Homo sapiens
aggaaaattgctacaaatctggattaatggaagtcaaatggtagtgagccggatggtaattaagctgaggttaaaagctttgaagccagcctttcgggagcggactttttcaaacacctcagggacctaaacaaactcctgatatttattcaggctgaaattctgactcgcatttagactgctaaattaataagcttggagacctctccccttccctgagtagacactgcctttcatttatgccatagaggtgcattaacgctccgtaatgccatctattatttgggattattaattcatcagttactcaattgaaaaatctgaggagtcaatacagcaagtcgttgggaaagtgtgggaccctagtgaaaaatcagcagattgttgaggaaataatgttaaagaaacactgagtaacaaaagacattctttattaaaatatcaaagaggataaatatctccttaagaagctctggggtcctaagtaaactcccaaggggtgggggtcctgacagcaggacatggtggtctcacagtttaacctttgcacgaagccaccggtcttcggctttctgggcacaggactcggtgcagcaccctgggaggaa

SEQ ID NO: 573 - DNA, 508 bp, Homo sapiens
aacactgacacacaggatggtcccgctgcttgcctgtggtcacagtatccggagcatggctgcagctgggaaccacctccaggcgacaaagcaacccagcacgggtgggtacggaggggccgttcgtctcgctcacagtctgggggtggcctcatggtgctgctcatcacagcgggcatgttggctggggctgactgtgagaacccagctctgccccagaggcctggccttggctggttctcacagggaggaagagagcccagagcagtaagcacagcccgtcagagagcgggcagagacgcactgcgcttcctgagaccgcggctcaccgctcccctgcattctgctcttcaaagacacatgcagaaccttcagggtgttgaaaggggccgcaaagatcacagctctcagcgggggcgcggtcagtcggccacagcggtggagatggcgttgaaagccggggatgatgactgctgcgctgggatgactgacggctgagccaggccct

SEQ ID NO: 574 - DNA, 474 bp, Homo sapiens
tacagtttgcagaacacaccctcctgccctgtggaagcttccaggctcccgggcaggtcgctgaaggatcctcttcctggcccctggagtccacaggtgcgacatcccgtcccgacatgggtgcctgccccagccactcagtcgtcacctgtgagcacctgaaaaccaggccacagcccagactcacaaggagccacgggcctgggagatttattaaacattcgcacgtaattttccattaacatttcctattttggcatcttaaagagtgaaagttgatttcggttcaacatcctgtggggaaattagttcattttgctaacgcatcttaataattacagtgacatcaaggttaattgcttaatctggattgccacagtgcaggccgccgaattccccacgcaatctgtgctataattagtaccggggcatccagatttagaaggctctttcaccggaacagcggaatcaggc

SEQ ID NO: 575 - DNA, 632 bp, Homo sapiens
gtgtgtccggggacacgcaccgggtggaagtagatggcgtcgaactgtccggacagcgcctgcagctcctccttgcctaggacctgggtctccctgaccagggatacatactctgggctgcatgcagatgggcaggcaggagaggcagggtcaccagcaggagcaggggggcgggaggcggggacctgggattcatggtgggagtggccaccctgtcacttcctcaagcctgtgagcccacccgaggtgcatagggagaggctctgtattcgaggctggcaggctcggggtaggcccaggttcttaggtttctagctgggctgcccgccccgccccccatcgtggggcctgcccccgtcctgacctgtgcaccaggcccagctcctcttccgaggcctcgcgggctgacaaccgcagacacctctgttccaggccgcgctgccgcaggcgatccagggctgcggtcaggcgctcaggacgctcgatctcgcactcggggctggggcagatgaggagctcagttcagaggtccctccccgcaccccacctcggccaggtcccacttactcgtcccagagcagccgggtggccgtcatgtcctcatggtacacaagcgcggtccccatggctgc

SEQ ID NO: 576 - DNA, 536 bp, Homo sapiens
tcagcccccgggcgcagaccccgcccttcctgggcacgatcccatgggccggtgccagagctgagcccacgccaggactgaccgtcatgggctgcagggattcagcctcccgcggcggctgccaggactgcaggggctccgcgatgaccaccatggcaaacctctggatcaggctgaagccctgacccgtgacgttgatgctgcggccaccactgcggagggcagtgccttcgtgggctcggctggcaggggtggtccctgcagcctccgccctcccgtccccgcccaccctggccctgaccacacggggcttccaggtgcatgtgtgggtctcggtgggcaaaggggcaacgcggtgggacgtgctgagcacgagactgtgccgcaagtgggcggggggcacacggggcctggtgagcgtggagcctctggtgtgcccatctgggtttctaggaggaagtggatctggagtgcagtggggttttccggaggccgcctcccgctgcacagaccagccccaccacattcctttcagc

SEQ ID NO: 577 - DNA, 333 bp, Homo sapiens
ccgtgacaggaagagggggtctcgggccgtgacaggaagagggggtctccggccgtgacaggaagagggggtctcgggccgtgacaggaagaggggatctcgggccgtgacaggaagagggagtctcgggccgtgacaggaagagggggtctcgggccgtgacaggaagagggggtctcgggccgtgacaggaagagggggtctcgggccgtgacaggaagagggggtctcgggccgtgacaggaagagggggtctcgggccgtgacaggaagagggggtctccggccgtgacaggaagagggagtctcgggccgtgacaggaagaggggatc

SEQ ID NO: 578 - DNA, 359 bp, Homo sapiens
ccgtgacaggaagagggggtctcgggccgtgacaggaagagggggtctccggccgtgacaggaagagggagtctcgggccgtgacaggaagaggggatctcgggccgtgacaggaagagggagtctcgggccgtgacaggaagagcgggtctcgggccgtgacaggaagagcgggtctcaggttgtgacaggaagagggagtctcgggccgtgacaggaagagggagtctcgggccgtgacaggaagagggggtctcgggccgtgacaggaagagggggtctcgggccgtgacaggaagagggggtctccggccgtgacaggaagagggggtctcgggccgtgacaggaagaggggatc

SEQ ID NO: 579 - DNA, 333 bp, Homo sapiens; identical to SEQ ID NO: 577

SEQ ID NO: 580 - DNA, 615 bp, Homo sapiens
ccgtgacaggaagagggggtctcgggccgtgacaggaagagggggtctccggccgtgacaggaagagggagtctcgggccgtgacaggaagaggggatctcgggccgtgacaggaagagggagtctcgggccgtgacaggaagagcgggtctcgggccgtgacaggaagagcgggtctcaggttgtgacaggaagagggagtctcgggccgtgacaggaagagggagtctcgggccgtgacaggaagagggggtctcgggccgtgacaggaagagggggtctcgggccgtgacaggaagagggtgtctcgggccgtgacaggaagagcgggtctcaggttgtgacaggaaggagtctcctgtgtgtagggactcatccattctgtacaactcatgtgcatcctcccatgtgcatcctcctctgtgcatccttccatgtgcatccttgaaggggtggcctgcccctccacacctgtgggcatttctcatcaggaggaacgagagacttgaaaaaagaaagagacacagagataaagtagagagaaagaaaaatgggcccaggggaccggcgctcagcatacagaggacccgcgccggcacctgcctctgagttccc

SEQ ID NO: 581 - DNA, 618 bp, Homo sapiens
acgccatccatcattcaaataatcgcaatttagagcccgcgtttcatgccgggctggtttaatcaccagcagcagtttgtgaagggcctcattcgcctgggattcggggatattgcgtgatttagcagacgtgacccctgacctctgcggggggcggagagcctgccttggaggcaaaagcggaacagcgagggtgactcactcagggacaattattatgcttaacagcctcactgtcatcttagaaggaaaaagagccaaaaaaaaaaaaaaaatccataggatgctgagcattccttccagatccttcagcattccttccagatccttcagcattccttccagatccttcagcattccttccagatccttcagcattccttccagatccttcagcattccttccagatcccagtttagaagaaggcagtggggtcggtttaaatgcacccagcgtgctcccgaagataatgtttttgtttctgtagacgcggtggcagctttctggcgatttctaggcaatgtacaggaggaaagaaagaaggaaggggagggcacggagaagctccggaaggctggatgcttggggaaggaggtcaggaggcaggaccggccggc

SEQ ID NO: 582 - DNA, 478 bp, Homo sapiens
gtgcctcattttccaatttttaaaaaattgcctaatcatcaaagaggcccggaagctaggctaccaaggcgcgacgctgtttacaccccagacccaggttctcagctcggctttgcttgggaaaggctcggaacctgtctgggcctgggggtttagcacagaaggctttggttagctgcactgtgctgagcgcagctctgcagcgaggtttagggagctgcggtgcgttaggcacaaggcacgtggcgcgcctcgctccagcactttgcaaacgcacagcagcgcagggccagagcggctggcccccaggggtgggggcgaccagggacaggaggtcccatatggtgtcgtcgcgttaaaaacgtaggtcctaaggggcgagccgcgtccgacctccgcgcccctgcccgacgcggcgcctcgctcgccggccccgctcgctggattcatttccatctggagcgcggcgcgtcccaac

SEQ ID NO: 583 - DNA, 437 bp, Homo sapiens
tttttttgaaaaagtgaaagcaattttattaagaaagtaaaggaatagccggctgtgtggcagcccgcgaggcggtccgcgggagcacactctgtgcagagactgggcggccagcctgcccttgctgtcgctgacggtgactgcgggaggccaggttgtttttcatcattcagaacattgcctgaagcaggtccaccgtgccgttagtaacgaggaacatcgagccaaggcacctgtgccgtcagacgttgcctagcgttagaagcgagctggaatgcgtgaccaacatcaccctggcaaatgtcatccgacagctgggcagcctgagtaaatatgcagaggacatttttggagagctctttactcaggcaaatacctttgcctctccggtaagctcccttgctgagagggtcgatggactacaagttaaagtcact

SEQ ID NO: 584 - DNA, 516 bp, Homo sapiens
ccatcagccactgtgcctgaccgtgcgcgagcgcgaagacccagggacccggctctacgagtgcatcccgctgtactctgtaagagccgcactctctaggtgagcttcccagagcggtccttgatgccttcctgccttgatcgtcccacaaccgatcgactgcccctttgcaggttcctaatttattcagatctgtagaacggagacgggcgtggccaaaaatgggactagaggggtctccgagaagtcggagaatgggtgtcagcttgggcctgggtggaaaccaggaggacggattgccgcctggggcgctcgccgccgcgcccgccctactgtcgaaaggtggggctgatttcgttggtgggcgggggtgggctttattgtccctccacacacagcatttgtattcttacaaaatagaagctcccttcccctacggtatctcgcataatggtgtgttcatttccggtacccgcaatggtatgttacatcctccacgcctcatccccagctgca

SEQ ID NO: 585 - DNA, 480 bp, Homo sapiens
ctccgcctgagccccgcccatgacggtgggcgcctgcgcctacgcgcgccgggaagcagaaggtgatctctgccttcgagacacggagtgtgctgcctcagtctgtgatccctcgcgtttcaatccaattcaaaccatgctgcagccaattcgttcgcgcgtgcgtgtgccatcattgtgagtggaggagactgcccgatatgatcccagtcattgcggcgacccctggagatctccaacgcctgcgtcccccaaccataccctgccccgcatccacagatatcttcccttccttgatcgccagtacctggcactgaccgtgctttgaattcaaacaccctgggaggcatagtccctgtgatttaaactaacattgaagaagcgaccagacgcggtggttcacgcctgtaatcccagcactttgggagaccggggcgggcggatcacgtgaggtcaggagttcgagaccaacctggctaa

SEQ ID NO: 586 - DNA, 595 bp, Homo sapiens
aagatcacccgccgctcattgggctacgcgtatgtcaactaccagcaaccggtggacgccaagcgggccctggagaccctgaactttgatgtcataaagggcaggccagtgcgcatcatgtggtcccagagggacccgtcgctccgcaagagcggggtgggcaacgtcttcatcaagaacctgggcaagaccatcgacaacaaggcgctgtacaacatcttctcggcgttcggcaacatcctctcctgcaaagtggcctgtgacgaaaaggggcccaagggctacgggttcgtgcacttccaaaagcaggaatctgcggagcgggccatcgatgtgatgaatggcatgttcctgaactaccgcaaaattttcgtcgggagattcaagtcgcataaagaacgagaggccgaaaggggagcctgggccaggcagtccactagtgctgacgtcaaggatttcgaggaagacaccgacgaggaggccaccttgcgatgaagacatcccaggagctagccagccagcagagccaaaccttggctcacacccggtttacaaccccccacccccagccctcccccgccaacccaccagcagt

SEQ ID NO: 587 - DNA, 595 bp, Homo sapiens
cactgctggtgggttggcgggggagggctgggggtggggggttgtaaaccgggtgtgagccaaggtttggctctgctggctggctagctcctgggatgtcttcatcgcaaggtggcctcctcgtcggtgtcttcctcgaaatccttgacgtcagcactagtggactgcctggcccaggctcccctttcggcctctcgttctttatgcgacttgaatctcccgacgaaaattttgcggtagttcaggaacatgccattcatcacatcgatggcccgctccgcagattcctgcttttggaagtgcacgaacccgtagcccttgggccccttttcgtcacaggccactttgcaggagaggatgttgccgaacgccgagaagatgttgtacagcgccttgttgtcgatggtcttgcccaggttcttgatgaagacgttgcccaccccgctcttgcggagcgacgggtccctctgggaccacatgatgcgcactggcctgccctttatgacatcaaagttcagggtctccagggcccgcttggcgtccaccggttgctggtagttgacatacgcgtagcccaatgagcggcgggtgatct

SEQ ID NO: 588 - DNA, 699 bp, Homo sapiens
ccagctgccgcctctgcacagagcctgcacccggtgctccgagagcccccggatcacggcgaactgggctcgcaccattgccaggatcactccgacgaggagacgccaacctctgatgagttggaacagttcgccaaacaattcaaacaaagaagaatcaagttgggcttcacgcaggccgacgtggggttggcgctgggcacactgtatggtaacgtgttctcgcagaccaccatctgcaggttcgaaggcttgcagctgagcttcaaaaatatgtgcaagctgaagcccctgctgaacaagtggctggaggaggcggattcgtccacagggagcccgaccagcattgacaagatcgctgcacagggccgcaagcgcaagaagcggacctccatcgaggtgagtgtcaagggcgtactggagacgcatttcctcaagtgtcccaagcctgccgcgcaggagatctcctcgctggcagacagcctccagttggagaaggaagtggtgcgtgtctggttctgtaatcgaagacaaaaagagaaaagaatgactccgccaggggatcagcagccgcatgaggtttattcgcacaccgtgaaaacagacacatcttgccatgatctctgactggaggaagcgaggaggcggccggccgcactgggagcagcgcggatttctctttctctctcactctcttcc

SEQ ID NO: 589 - DNA, 319 bp, Homo sapiens
ttttttaaatcttggtacagaggaagcgggaagttacttagcacggttccgggtttctcgcgccccgcctgtcccccctccctatcactgctactggctcttggtccctccgttggactgtcctgcggagagaaaccccagcccatcggtctgcgctgggaccgcccgccgcgcatctgcccttcttcgctgactccgccccgcatctggccagacccgcctcgcgtcagagctgacccactcactgcgcgtttgccagtcagtctctccggacctgcctcgagcctcaggctgctgaaatcaccgcgcctcactcggt

SEQ ID NO: 590 - DNA, 342 bp, Homo sapiens
gactgtgttcccgcagcagacgttaggccgctgcccacgccttgagtgccggacgaggtcaacataggctttccgtcacagaatatgtttgggcaggaagatcggaacacttggggctggggcatctaccgctccccacggcacacacgagtcgtcagggaaatgccgcctctgtgtgtgtgttagcagcctggcgtagtgtcttcccactgttttgtctgtcttgagaatagcattcaacgcgactgtgttcccgcagcagacgttaggccgctgcccacgccttgagtgccggacgaggtcaacataggctttccgtcacagaatatgtttgggcaggaa

SEQ ID NO: 591 - DNA, 579 bp, Homo sapiens
cctatcgtcgtcagtcatggctagcatcattgcacgtgtcggtaacagccggcggctgaatgcacccttgccgccttgggcccattccatgctgaggtccctggggagaagtctcggtcctataatggccagcatggcagacagaaacatgaagttgttctcggggagggtggtgccagcccaaggggaagaaacctttgaaaactggctgacccaagtcaatggcgtcctgccagattggaatatgtctgaggaggaaaagctcaagcgcttgatgaaaacccttaggggccctgcccgcgaggtcatgcgtgtgcttcaggcgaccaaccctaacctaagtgtggcagatttcttgcgagccatgaaattggtgtttggggagtctgaaagcagtgtgactgcccatggtaaattttttaacaccctacaagctcaaggggagaaagcctccctttatgtgatccgtttagaggtgcagctccagaacgctattcaggcaggcattatagctgagaaagatgcaaaccggactcgcttgcagcagctccttttaggcggtgagctgagtagggacct

SEQ ID NO: 592 - DNA, 800 bp, Homo sapiens
cacttccaagctgtgtctcttgacattgtacagcagagcatgctgcctccgggctgcacagaacaccacggagtagcaggcaatcatgacaatcagtggaatgacgatgaaggacaccacgctgagaatagtgtagctggggctggccccccagatcatggagcagagagcattgcgctcatcaaaggcagcctggccccagccgtagagtggaggagtgctctgcaggatggccacaatccaggtgccatagaggagcaggtaaccgcggcgctgggtcatcttggacgggtaggagagagggtggatgatggacaagtagcgatccactgacaccacgacaatggtgttgacgctggcgaaggcgaacaggtgggtgaggctaaccagggccgtgcagaagtggctgttgaggggccagaagagaggcacagaggtggccaccacccagggggccacgagcgaaatctgcagcaggtcggtgacgaggaggttaaagataaaacggttggtcacctgcagcagctgcggcttgcgctgcaacactagcgccagcactatgttgccgacgaaagaggcggcgaggaagataaccagcacggttgagcggatgatgccgtgggccaggctgatgggcattttggagaggggcatgcacgtgtggctgctgttactctcgcgcgtgctgttggtgcaggtggacgtcatggcaacagccagagggcgtgagacaggttgcaggctcagtgccggcaccggtgggtggcaaaggggtgcacagaccgcactcagtgcctatg

SEQ ID NO: 593 - DNA, 853 bp, Homo sapiens
ggggcgggaagccttttcctcaccttggcctcggccccaagtggtgtcccggagatgcagcagcgtctcccagtcctcaccgcggacgccgccccaggtgtccccaggtgtccccaggtgtctcgaagccgcggtctctcccatgcgggtcctggggagaggaagctcagtcctcatagtggccgcaaagtgactgccgctgtgtggatgagacgcggtgcgtgcacggcgccccgagttgccaggggccatgcgcccccttgcggcagccatggggagtctctgaaaatcagcgtctagatttttccaggctgtggattttggaaattacgactgaaatggagcccaccatcccatgccttgatcggaactcatggagcaaggaagcaaagaagatggccagttcatgcctgtaatcccagcattttgggaggctgaggcgggcggattacttcaggtccagagtttgagaccagcctggctaacatggtgaaaccgcatctatactaaaaatacaaaaattagctgggcatggcggcacgtgcctgtaatcccagctactcaggaggctgaggcaggaggattgctcgaacccaggaggcgcaggttgcagtgagccaagatcatgtcactgcactccaacctgggtgacagagccaaactccatctcaaaaaaaagatggccagtaggcaaaacttaattgattttctttcttttctctttaagagacagggtctcgctctgtcacccatgctgaagtgcagtggtgtaatcacagctcccgagttcagcctccagctcccgggttcaagaaattctccagcctcagcctcccaacacactgggattaca

SEQ ID NO: 594 - DNA, 333 bp, Homo sapiens; identical to SEQ ID NO: 577

SEQ ID NO: 595 - DNA, 359 bp, Homo sapiens; identical to SEQ ID NO: 578

SEQ ID NO: 596 - DNA, 333 bp, Homo sapiens; identical to SEQ ID NO: 577

SEQ ID NO: 597 - DNA, 615 bp, Homo sapiens; identical to SEQ ID NO: 580

SEQ ID NO: 598 - DNA, 618 bp, Homo sapiens; identical to SEQ ID NO: 581

SEQ ID NO: 599 - DNA, 195 bp, Homo sapiens
cgaaataagaagcaaatcttgctgggaccgagttcaggtcgaggaactccgggaccctctgcagcctcagccctgccgcgctccccgcagcttcgggcctgttgcggggagttggggaggaattcagggaaggggggaaccaggccgggataagaggctccggcttgctgtgggctgcatgacggggtgcaaagg

SEQ ID NO: 600 - DNA, 448 bp, Homo sapiens
tcgtccacttgaggacgtcgaagcaggtgatgatgcccagggcgtgcaccgggtaggtgagatcggtgcgcgccagcggggacagggcggtcaggagcagcagccaggtccctgcacacgcggccaccgcgtaacgacggcggcgccagcgcttggagctgagcgggtacaggacccccaggaagcgctccacgctgatacaggtcatggtgaggatgctggaatacatgtttgcgtaaaaggccacggtcaccacgttgcaaagcagcaccccgaatacccagtggtggcggttgcaatggtagtagatttggaaaggcaacacgctggccagcatcaggtccgtgacgctcaggttgatcatgaagatgaccgacggggatctgggccccatgcgccggcacagcacccacagagagaagaggttgcccgggatgctgaccgccgc